SEQUENCE LISTING (285 sequences)

SEQ ID NO: 1 - protein, 1228 aa, unidentified
  REGION 1..1228: note = Description of Unknown: Lachnospiraceae bacterium ND2006 sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1300 aa, Francisella sp.
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 5 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 6 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 7 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 8 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 9 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 10 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 11 - protein, 1224 aa, synthetic construct
  REGION 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 12 - protein, 500 aa, synthetic construct
  REGION 1..500: note = Description of Artificial Sequence: Synthetic polypeptide
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 13 - protein, 507 aa, synthetic construct
  REGION 1..507: note = Description of Artificial Sequence: Synthetic polypeptide
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 14 - protein, 529 aa, synthetic construct
  REGION 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 15 - skipped

SEQ ID NO: 16 - protein, 726 aa, synthetic construct
  REGION 1..726: note = Description of Artificial Sequence: Synthetic polypeptide
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 17 - protein, 517 aa, synthetic construct
  REGION 1..517: note = Description of Artificial Sequence: Synthetic polypeptide
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 18 - protein, 481 aa, synthetic construct
  REGION 1..481: note = Description of Artificial Sequence: Synthetic polypeptide
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 19 - protein, 358 aa, synthetic construct
  REGION 1..358: note = Description of Artificial Sequence: Synthetic polypeptide
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 20 - protein, 507 aa, synthetic construct
  REGION 1..507: note = Description of Artificial Sequence: Synthetic polypeptide
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 21 - protein, 534 aa, synthetic construct
  REGION 1..534: note = Description of Artificial Sequence: Synthetic polypeptide
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 22 - protein, 537 aa, synthetic construct
  REGION 1..537: note = Description of Artificial Sequence: Synthetic polypeptide
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 23 - protein, 540 aa, synthetic construct
  REGION 1..540: note = Description of Artificial Sequence: Synthetic polypeptide
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 24 - protein, 542 aa, synthetic construct
  REGION 1..542: note = Description of Artificial Sequence: Synthetic polypeptide
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 25 - protein, 564 aa, synthetic construct
  REGION 1..564: note = Description of Artificial Sequence: Synthetic polypeptide
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 26 - protein, 610 aa, synthetic construct
  REGION 1..610: note = Description of Artificial Sequence: Synthetic polypeptide
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 27 - protein, 369 aa, synthetic construct
  REGION 1..369: note = Description of Artificial Sequence: Synthetic polypeptide
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 28 - protein, 552 aa, synthetic construct
  REGION 1..552: note = Description of Artificial Sequence: Synthetic polypeptide
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 29 - protein, 534 aa, synthetic construct
  REGION 1..534: note = Description of Artificial Sequence: Synthetic polypeptide
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 30 - protein, 577 aa, synthetic construct
  REGION 1..577: note = Description of Artificial Sequence: Synthetic polypeptide
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 31 - protein, 613 aa, synthetic construct
  REGION 1..613: note = Description of Artificial Sequence: Synthetic polypeptide
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 32 - protein, 615 aa, synthetic construct
  REGION 1..615: note = Description of Artificial Sequence: Synthetic polypeptide
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 33 - protein, 775 aa, synthetic construct
  REGION 1..775: note = Description of Artificial Sequence: Synthetic polypeptide
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 34 - protein, 777 aa, synthetic construct
  REGION 1..777: note = Description of Artificial Sequence: Synthetic polypeptide
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 35 - protein, 610 aa, synthetic construct
  REGION 1..610: note = Description of Artificial Sequence: Synthetic polypeptide
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 36 - protein, 632 aa, synthetic construct
  REGION 1..632: note = Description of Artificial Sequence: Synthetic polypeptide
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 37 - protein, 625 aa, synthetic construct
  REGION 1..625: note = Description of Artificial Sequence: Synthetic polypeptide
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 38 - protein, 517 aa, synthetic construct
  REGION 1..517: note = Description of Artificial Sequence: Synthetic polypeptide
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 39 - protein, 410 aa, synthetic construct
  REGION 1..410: note = Description of Artificial Sequence: Synthetic polypeptide
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 40 - protein, 486 aa, synthetic construct
  REGION 1..486: note = Description of Artificial Sequence: Synthetic polypeptide
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 41 - protein, 602 aa, synthetic construct
  REGION 1..602: note = Description of Artificial Sequence: Synthetic polypeptide
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 42 - protein, 494 aa, synthetic construct
  REGION 1..494: note = Description of Artificial Sequence: Synthetic polypeptide
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 43 - protein, 526 aa, synthetic construct
  REGION 1..526: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 44 - protein, 649 aa, synthetic construct
  REGION 1..649: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 45 - protein, 414 aa, synthetic construct
  REGION 1..414: note = Description of Artificial Sequence: Synthetic polypeptide
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 46 - protein, 413 aa, synthetic construct
  REGION 1..413: note = Description of Artificial Sequence: Synthetic polypeptide
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 47 - protein, 449 aa, synthetic construct
  REGION 1..449: note = Description of Artificial Sequence: Synthetic polypeptide
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 48 - protein, 711 aa, synthetic construct
  REGION 1..711: note = Description of Artificial Sequence: Synthetic polypeptide
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 49 - protein, 574 aa, synthetic construct
  REGION 1..574: note = Description of Artificial Sequence: Synthetic polypeptide
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 50 - protein, 400 aa, synthetic construct
  REGION 1..400: note = Description of Artificial Sequence: Synthetic polypeptide
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 51 - protein, 743 aa, synthetic construct
  REGION 1..743: note = Description of Artificial Sequence: Synthetic polypeptide
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 52 - protein, 769 aa, synthetic construct
  REGION 1..769: note = Description of Artificial Sequence: Synthetic polypeptide
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 53 - protein, 666 aa, synthetic construct
  REGION 1..666: note = Description of Artificial Sequence: Synthetic polypeptide
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 54 - protein, 564 aa, synthetic construct
  REGION 1..564: note = Description of Artificial Sequence: Synthetic polypeptide
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 55 - protein, 565 aa, synthetic construct
  REGION 1..565: note = Description of Artificial Sequence: Synthetic polypeptide
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 56 - protein, 499 aa, synthetic construct
  REGION 1..499: note = Description of Artificial Sequence: Synthetic polypeptide
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 57 - protein, 560 aa, synthetic construct
  REGION 1..560: note = Description of Artificial Sequence: Synthetic polypeptide
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 58 - protein, 404 aa, synthetic construct
  REGION 1..404: note = Description of Artificial Sequence: Synthetic polypeptide
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 59 - protein, 392 aa, synthetic construct
  REGION 1..392: note = Description of Artificial Sequence: Synthetic polypeptide
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 60 - protein, 500 aa, synthetic construct
  REGION 1..500: note = Description of Artificial Sequence: Synthetic polypeptide
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 61 - skipped

SEQ ID NO: 62 - protein, 507 aa, synthetic construct
  REGION 1..507: note = Description of Artificial Sequence: Synthetic polypeptide
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 63 - protein, 529 aa, synthetic construct
  REGION 1..529: note = Description of Artificial Sequence: Synthetic polypeptide
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 64 - protein, 726 aa, synthetic construct
  REGION 1..726: note = Description of Artificial Sequence: Synthetic polypeptide
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 65 - protein, 517 aa, synthetic construct
  REGION 1..517: note = Description of Artificial Sequence: Synthetic polypeptide
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 66 - protein, 481 aa, synthetic construct
  REGION 1..481: note = Description of Artificial Sequence: Synthetic polypeptide
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 67 - protein, 534 aa, synthetic construct
  REGION 1..534: note = Description of Artificial Sequence: Synthetic polypeptide
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 68 - protein, 537 aa, synthetic construct
  REGION 1..537: note = Description of Artificial Sequence: Synthetic polypeptide
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 69 - protein, 541 aa, synthetic construct
  REGION 1..541: note = Description of Artificial Sequence: Synthetic polypeptide
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 70 - protein, 542 aa, synthetic construct
  REGION 1..542: note = Description of Artificial Sequence: Synthetic polypeptide
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 71 - protein, 564 aa, synthetic construct
  REGION 1..564: note = Description of Artificial Sequence: Synthetic polypeptide
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 72 - protein, 610 aa, synthetic construct
  REGION 1..610: note = Description of Artificial Sequence: Synthetic polypeptide
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 73 - protein, 552 aa, synthetic construct
  REGION 1..552: note = Description of Artificial Sequence: Synthetic polypeptide
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 74 - protein, 534 aa, synthetic construct
  REGION 1..534: note = Description of Artificial Sequence: Synthetic polypeptide
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 75 - protein, 577 aa, synthetic construct
  REGION 1..577: note = Description of Artificial Sequence: Synthetic polypeptide
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 76 - protein, 613 aa, synthetic construct
  REGION 1..613: note = Description of Artificial Sequence: Synthetic polypeptide
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 77 - protein, 615 aa, synthetic construct
  REGION 1..615: note = Description of Artificial Sequence: Synthetic polypeptide
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 78 - protein, 775 aa, synthetic construct
  REGION 1..775: note = Description of Artificial Sequence: Synthetic polypeptide
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 79 - protein, 777 aa, synthetic construct
  REGION 1..777: note = Description of Artificial Sequence: Synthetic polypeptide
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 80 - protein, 610 aa, synthetic construct
  REGION 1..610: note = Description of Artificial Sequence: Synthetic polypeptide
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 81 - protein, 632 aa, synthetic construct
  REGION 1..632: note = Description of Artificial Sequence: Synthetic polypeptide
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 82 - protein, 625 aa, synthetic construct
  REGION 1..625: note = Description of Artificial Sequence: Synthetic polypeptide
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 83 - protein, 517 aa, synthetic construct
  REGION 1..517: note = Description of Artificial Sequence: Synthetic polypeptide
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 84 - protein, 410 aa, synthetic construct
  REGION 1..410: note = Description of Artificial Sequence: Synthetic polypeptide
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 85 - protein, 602 aa, synthetic construct
  REGION 1..602: note = Description of Artificial Sequence: Synthetic polypeptide
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 86 - protein, 494 aa, synthetic construct
  REGION 1..494: note = Description of Artificial Sequence: Synthetic polypeptide
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 87 - protein, 649 aa, synthetic construct
  REGION 1..649: note = Description of Artificial Sequence: Synthetic polypeptide
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 88 - protein, 414 aa, synthetic construct
  REGION 1..414: note = Description of Artificial Sequence: Synthetic polypeptide
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 89 - protein, 413 aa, synthetic construct
  REGION 1..413: note = Description of Artificial Sequence: Synthetic polypeptide
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 90 - protein, 449 aa, synthetic construct
  REGION 1..449: note = Description of Artificial Sequence: Synthetic polypeptide
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 91 - protein, 711 aa, synthetic construct
  REGION 1..711: note = Description of Artificial Sequence: Synthetic polypeptide
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 92 - protein, 574 aa, synthetic construct
  REGION 1..574: note = Description of Artificial Sequence: Synthetic polypeptide
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 93 - protein, 743 aa, synthetic construct
  REGION 1..743: note = Description of Artificial Sequence: Synthetic polypeptide
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 94 - protein, 769 aa, synthetic construct
  REGION 1..769: note = Description of Artificial Sequence: Synthetic polypeptide
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 95 - protein, 564 aa, synthetic construct
  REGION 1..564: note = Description of Artificial Sequence: Synthetic polypeptide
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 96 - protein, 565 aa, synthetic construct
  REGION 1..565: note = Description of Artificial Sequence: Synthetic polypeptide
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 97 - protein, 499 aa, synthetic construct
  REGION 1..499: note = Description of Artificial Sequence: Synthetic polypeptide
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 98 - protein, 358 aa, synthetic construct
  REGION 1..358: note = Description of Artificial Sequence: Synthetic polypeptide
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 99 - protein, 369 aa, synthetic construct
  REGION 1..369: note = Description of Artificial Sequence: Synthetic polypeptide
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 100 - protein, 486 aa, synthetic construct
  REGION 1..486: note = Description of Artificial Sequence: Synthetic polypeptide
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 101 - protein, 400 aa, synthetic construct
  REGION 1..400: note = Description of Artificial Sequence: Synthetic polypeptide
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 102 - protein, 666 aa, synthetic construct
  REGION 1..666: note = Description of Artificial Sequence: Synthetic polypeptide
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 103 - protein, 560 aa, synthetic construct
  REGION 1..560: note = Description of Artificial Sequence: Synthetic polypeptide
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 104 - protein, 404 aa, synthetic construct
  REGION 1..404: note = Description of Artificial Sequence: Synthetic polypeptide
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 105 - protein, 392 aa, synthetic construct
  REGION 1..392: note = Description of Artificial Sequence: Synthetic polypeptide
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 106 - protein, 492 aa, synthetic construct
  REGION 1..492: note = Description of Artificial Sequence: Synthetic polypeptide
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 107 - protein, 477 aa, synthetic construct
  REGION 1..477: note = Description of Artificial Sequence: Synthetic polypeptide
MRISKTLSLRIVRPFYTPEVEAGIKAEKDKREAQGQTRSLDAKFFNELKKKHSEIILSSEFYSLLSEVQRQLTSIYNHAMSNLYHKIIVEGEKTSTSKALSNIGYDECKAIFPSYMALGLRQKIQSNFRRRDLKNFRMAVPTAKSDKFPIPIYRQVDGSKGGFKISENDGKDFIVELPLVDYVAEEVKTAKGRFTKINISKPPKIKNIPVILSTLRRRQSGQWFSDDGTNAEIRRVISGEYKVSWIEIVRRTRFGKHDDWFVNMVIKYDKPEEGLDSKVVGGIDVGVSSPLVCALNNSLDRYFVKSSDIIAFNKRAMARRRTLLRQNKYKRSGHGSKNKLEPITVLTEKNERFKKSIMQRWAKEVAEFFRGKGASVVRMEELSGLKEKDNFFSSYLRMYWNYGQLQQIIENKLKEYGIKVNYVSPKDTSKKCHSCTHINEFFTFEYRQKNNFPLFKCEKCGVECSADYNAAKNMAIA

SEQ ID NO: 108 - protein, 477 aa, synthetic construct
  REGION 1..477: note = Description of Artificial Sequence: Synthetic polypeptide
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 109 - protein, 472 aa, synthetic construct
  REGION 1..472: note = Description of Artificial Sequence: Synthetic polypeptide
VPITKTISLRILRPYYPPEIEAKIKAEKEKRKENGDTGSLNSSYYRELKKEYPSIIINDEFFPLLSEMQRNITSIYNRTISHLYHRLIIKKESISTAKALSEGPYRDFKSTFNSYIALGLRQKVQSNFRKKDLMAFKIALPTAKSDKFPIPIYMQTNFKIKESPDSDFIIELPLVEYIAKETKGKNKMFTKVEILSPPKVKNIPVILSTRRRKESGQWFSDEGTNAEIRRIISGEYKVSWIEIVKRTRFGKHDWFVNMVISFEESQEGLDPDVIGGIDIGVSKPLICAINNSLDRYIVKGDDIIAFNRRALSRRRSLLRRNRLKRSGHGSRNKLEPITVLTEKNERFKKSIMQRWAKEVAEFFKSKRASIVQMEELTGIKEREDFFSKTLRMYWNYGQLQKTVENKLREYGIEVRYASPKDTSRRCHSCGHINDYFTFEFRQQNNFPLFKCMNCGIECSADYNAARNIAIAR

SEQ ID NO: 110 - protein, 455 aa, synthetic construct
  REGION 1..455: note = Description of Artificial Sequence: Synthetic polypeptide
MAKKGTNRKKMIVKVMKYELKYESGCADFNEMQNELWKLQRQTREVMNRTIQLCYHWSYVQADYCKQHGCARRDVKPCDVYETNATSLDGYIYQLFKDEYPNFLMANLIATLRKAHQKYDALLFDIQEGNSSIPSFKKDQPLIFSKEAIRLPECLSDKRQITLFCFSKPYKSAHPTLDKITFAVRARSASEKSIFDHIISGKYALGESQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKALESEKPYLKHWTYFDLQSKIIYKAEEKGIRVVKVNPKCTSRRCSACGYISKENRKNQVEFLCVNCGYHHNADYNAAQNLSIPQIDRLIEKQLKEQESEENEAGANPK

SEQ ID NO: 111 - protein, 410 aa, synthetic construct
  REGION 1..410: note = Description of Artificial Sequence: Synthetic polypeptide
MAKGTLSKVMKYELRYLDGCGDFQNMQKELWTLQRQSREILNRTIQIAYHWDYTDREQFKKTGQHLDIKAETGYKRLDGYIYDSLKEDVQNFASVNVNATIQKAWAKYKSSKIDVLRGDMSLPSYKSDQPLVLHAQSMKIFSSDDDDVLQVTLFSNAYKKACNYSNIRFIIGLHDATQRTIIKKVLSGDWGIGQSQIVYKRPKWFLYLTYNFSPEQHEVNPDKILGVDLGESIAIYASSIGEYGSLRIEGGEISAFAKQLEARKRSLQKQAAYCGKGRIGHGTKSRVSDVYKMEDKIANFRNTVNHRYSKMLIDYALKHMYGTIQMEDLSGIKKETGFPKFLQHWTYYDLQQKIEAKAKEHGINFIKVDPAFTSQRCSKCGNIDSENRPSQAVFCCKKCGYKTNADFNAS

SEQ ID NO: 112 - protein, 426 aa, synthetic construct
  REGION 1..426: note = Description of Artificial Sequence: Synthetic polypeptide
MNVTKVMRYQLIYQGGGGDFESLQNQLWEFQRQTRAILNKTIQTMYLATANQEKFSEKALYHDLCAEYPDMISSTVNATLREATKKYRSSVREILAGRMSLPSYKRDHPILLHNQSVALKQGNQGSYFATISVFSRKYQQGTPGVKQPSFQLIAKDNTQRTILQRLLSGEYKLGQCQLIYIRPKWFLNVAYSFTPSEKALDQEKVLGVDLGCVYAIYASSYGNHGIFKISGDEITSFERKQAAIQNRAFKNDLTRIREIEERRKQKLEQARYCGEGRIGHGVKTRVAPAYQDEGKISRFRETINHRYSKALVDYAEKNGYGTIQMEDLSGIKSSTGFPKRLQHWTYFDLQQKIKYKAEEQGIKVVKIKPAYTSQRCSRCGHIDPANRKSQSEFKCIACGFSSNADYNASQNISMRNIEKIIQGKAN

SEQ ID NO: 113 - protein, 432 aa, synthetic construct
  REGION 1..432: note = Description of Artificial Sequence: Synthetic polypeptide
MAKGTITKVMKYELRYLGGFSDFHEMQKEVWQLQRQYREILNKTIQIALHWDYVSAQQFGESGTYLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 114 - protein, 435 aa, synthetic construct
  REGION 1..435: note = Description of Artificial Sequence: Synthetic polypeptide
MSKGMLTKVMKYTLRYVGGCGDFHEMQSILWELQKQTRAVLNKTIQIAFEWDYRSREAFQETGEYLDVHAETGYKRLDGYIYNCLKNEYADFAGKNLNAAIQTAWKKYNQSKRDIQTGKMSLPSYRSNQPLIIHNDNVMISQDMQAAPSVRFTLLSLEYKKAHDLNTNPTFEVLINDGTQRAIFEKVRSGEYKLGQCMIQYDKKKWFLLLTYSFQPEKLTLDKNKILGVDLGETIVICASSVSERGRFVIDGGEITRFATQIEARKRSQQHQAAYCGEGRIGHGTKTRVDAVYKTEDRIANFRDTINHRYSRALVNYAVKHGFGTIQMEDLSGIKSSDDFPKFLRHWTYYDLQSKIESKAKERGIAVVKVNPRFTSRRCSKCGYIDEGNRKDQAHFCCLSCGFRANADFNASQNLSIKGIDKIIEKEYNANSKQT

SEQ ID NO: 115 - protein, 442 aa, synthetic construct
  REGION 1..442: note = Description of Artificial Sequence: Synthetic polypeptide
MGKPITKTMKYQIHYIDGCGDFHNMQKELWDLQRIVRQILNKTINESYLWFVRSEQYYRDTGENLSVEEQTGYKTLDGHIYNLLKQEYTQKLVSNSLNASIQAAYKKMKDSRRDVMIGTMSLPSYRSDQPIIIYNKNIKFSSHPEHGFVVDCSLFSDAYKKSQGYEKSVKFQVSVDDNTQRSIFENILTGNYKHGQCSIVYEKKKWFLLLTYSFVPEETKLDPDKILGVDVGVVYALYASSKGNHGTFKIKGDEAITFIQRVEARKHSRQLQGTYCGDGRIGHGTKTRVQPVYNERALISNFQDTINHRYSKALIDYAKKNGYGTIQMEDLSGIKEVQQYPKYLQHWTYYDLQLKIQYKAKEAGIGFVKVTPKYTSQRCSHCGNIDEANRPKQDVFRCTVCGYERNADYNASQNLSIKGIDRIIDDQLKQMNKANPKKTENA

SEQ ID NO: 116 - protein, 435 aa, synthetic construct
  REGION 1..435: note = Description of Artificial Sequence: Synthetic polypeptide
MSGGAITKVMKYDLTYKDGYGNFKDMQEAVWKLIRDTRTILNETIKIAYHWDYLNEKSKRETGEHLDLLEETGYKRLDGYIYDDLKDRFPDFASSNLNAAIQTAWKKYKQSQKDVYIGKMTLPSYKSDQPLPINKQSIKIYDEEREHIVELNLFSTKHKKEHGLASNVRFRINLHDNTQHAIYERVLSGEYTLGQCQLLYDRPKWFFILTYSFKPAQNKLDPDKILGVDMGETCALYASTFGEQGSFVINGGEVSEYAKREEARKRSLQKQAAVCGEGRIGHGTKTRVSSVYKEQERISNFRDTINHRYSKALIEYAVKNGCGTIQMEDLSGIRQSTDFPKFLRHWTYYDLQQKIKTKAKETGIAVSMIDPRYTSQRCSRCGHIDKANRKDQAHFHCLKCGYSCNADFNASQNISIRGIDKIIQKELGAKAKQTD

SEQ ID NO: 117 - protein, 431 aa, synthetic construct
  REGION 1..431: note = Description of Artificial Sequence: Synthetic polypeptide
MKEIAKVMKYQLIYLDGGGDFYELQQTLWDLQRQTREILNKTIQSMYLATATNTAFEENALYHRFGAEYPMMAALNVNATLRTAKKRYTSTIKETLRGTMSLPSYKRDQPILLHNQTIHLALEDGQYSALFSVYSEKFQKAHEGVARPRFALMARDGTQRAILDRLLDGSYRLGQSQMTYEQKKWFLSLTYKFVPEVRELDKSKILGVDLGCVYAIYASSMQQKGIFKISGDEITEFEKRQAAMQNREPVSTLERVEQLEQRRWQKQQQARYCGEGRVGHGTGTRVAPAYRDADKIARFRDTINHRYSKALVEYAEKNGFGTIQMEDLSGIKEDTGFPKRLRHWTYFDLQTKIQYKAAERGITVVKIDPQYTSQRCSRCGYIDKANRASQEKFLCQSCGFEANADYNASQNISVEKIDKLIAKDKKKLART

SEQ ID NO: 118 - protein, 436 aa, synthetic construct
  REGION 1..436: note = Description of Artificial Sequence: Synthetic polypeptide
MGQVTKVMRYQLIYQDGGGDFYTVQQELWELQRQTREILNKTIQTMYLADANKEKFDNAAERTLNRRFCVDHPDMYTKTVTATLRKAKAKYNASQKEILAGRMSLPSYKRDQPILLNPQGFKIEEESDSFFAAIAVFSDKYKNKHPDVDVKRLRFRLVVKDGTQRAIIRRVISGEYKLGRSQLLYSKKKWFLNVTYSFEPAEKKVDPDKILGVDLGCVYAIYASSFGSPGVFKISGDEVSSFERKQAAIQNRSPKSTLERVEKIEERHKQKQQQARYCGEGRIGHGTKTRIAPVYQDEDKIARFRDTVNHRYSKALIDYAEKNGYGTIQMEDLSGIKSATGFPKRLKHWTYYDLQTKIEYKAEERGIKVVKIDPRYTSQRCSRCGYIDSGNRKSQAEFCCMACGFSCNADYNASQNISIGGIAKIIADKRKEADAK

SEQ ID NO: 119 - protein, 368 aa, synthetic construct
  REGION 1..368: note = Description of Artificial Sequence: Synthetic polypeptide
YLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 120 - protein, 437 aa, synthetic construct
  REGION 1..437: note = Description of Artificial Sequence: Synthetic polypeptide
MAEKTIVKVMKFELRYIDGAGEFSEMQKHLWELQKQTREVLNKTIQMGYALECKRFAHHDKTGQWLDDKELTGSKYKAVADYINAELKEDYNIFYSDCRNSTVRKAYKKFKDAKNKIFSGEMSLPSYRSNQPIIIHNRNVIIRGNAESALVGLKVFSDGFKALHGFPAAVNFKLCVKDGTQRAIIENVISEIYKISESQLIYDNKKWFLILAYRFTQKKNDLNPDKILGVDLGVKFAVYASSIGEYGSFRIKGGEVTEFIKRLEKRKKSLQNQATVCGDGRIGHGTKTRVADVYKARDKISNFQDTINHRYSRAIVDYARKNGYGTIQLEKLDNSIEKKGDYSPVLVHWTYYDLRTKMEYKAAEYGIKVIAVEPKYTSQRCSKCGYISSENRKTQESFECIKCGYKCNADFNASQNLSVRDIDRIIDEYLGANPELT

SEQ ID NO: 121 - protein, 434 aa, synthetic construct
  REGION 1..434: note = Description of Artificial Sequence: Synthetic polypeptide
VVNVAKGALSKVMKFELSYLDGCGDFQNMQKELWTLQRQTREILNRTIQIAYHWDYTDREHFKKTGQHLDVKSETGYKRLDGYIYDELKETVQNFASVNVNATIQKAWAKYKSSKTDVLRGDMSLPSYKSDQPLVLHAQSIKLSEDKDGPVLQVTLFSNAHKKACDYSNVRFAFRLHDATQRAIFKNVLSGEYGLGQSQIVYKRPKWFLYLTYNFSPEQHGLDPDKILGVDLGESIALYASSLGDYGSLRIEGGEVTAFAKQLEARKRSLQKQAAHCGEGRVGHGTRARVSDVYKAEDKIANFRNTVNHRYSKKLIEYAIQNRYGTIQMEDLSGIKQDTGFPKFLQHWTYYDLQQKIEAKAKENGINFIKVDPSYTSQRCSKCGNIDSDNRPSQAVFCCTKCGFRANADFNASQNLSIPEIDKIIKKERGANTK

SEQ ID NO: 122 - protein, 455 aa, synthetic construct
  REGION 1..455: note = Description of Artificial Sequence: Synthetic polypeptide
MAKKGTNRKKMIVKVMKYELKYEKGCADFNEMQNELWKLQRQTREVMNRTVQLCYHWNYVQADYCKQHGCAHRDVKPCDVYETNATSLDGYIYQLFKDEYPNFLMANLIATLRKAHQKYDALLPDIQEGNSSIPSFKKDQPLIFSKEAIHLPECLSDKRQITLFCFSKPYKSAHPTLDKITFAVRAHSASEKSIFDNIINGKYALGTSQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKAMESEKPYLKHWTYFDLQSKIIYKAEEKGIRVVKVNPKCTSRRCSACGYISKENRKNQAEFLCVNCGYHHNADYNAAQNLSIPQIDRLIEKQLKEQESEESEAGANPK

SEQ ID NO: 123 - protein, 500 aa, synthetic construct
  REGION 1..500: note = Description of Artificial Sequence: Synthetic polypeptide
MTERHDNESSKIKAEVSLLNSSVPDFEKKRHVKVLKLHILKPAGDMKWDELGALLRDARYRVFRLANLAISEAYLDFHKWRSGGNEQPKLKISQLNRNLRSMLEDEVTGKQTKMIKSDRYSKSGALPDSIVSPLSMYKLGGLTSKSKWSEVLRGKSSLPTFKLNMAIPVRCDKPGDRRIERTKNGDAEVELRICLQPYPRVIIATGRNSLGDGQRAILDRLLDNTKYSEQGYRQRCFEIKEDQRSGKWHLFVTYDFPAIEPAKNLSRERIVGVDLGAACPLYAAINTGHARLGWKHFSPLAARVRALQNQTIRRRRQILRGGKVSLSEDSARSGHGRKRKLKPISKLEGKIDRAYTTLNHQLSATVIKFAKDNGAGVVQMEDLKGLRETLTGTFLGERWRYEELQRFIRYKADEAGIEIRLVNPQYTSRRCSECGHIHKDFTREFRDKSREGNKSVRFLCPDCGFTADPDYNAARNLASLDIAAIIERQLEIQGLRKHDP

SEQ ID NO: 124 - protein, 471 aa, synthetic construct
  REGION 1..471: note = Description of Artificial Sequence: Synthetic polypeptide
MKEKSKTLVKVARLRILKPAGDMKWSELGEMLRTVRYRVFRLANLAVSEAYLGFHMYRTNRATEFKAETIGKLSRRLREMLIEEGVDEKDLSRYSQTGAVPDTVAGALGQYKIRGITSPTKWRQVVRGQAALPTFRNDMAIPIRCDKQYQRRLEKTEAGEIEVELMICRKPYPRIVLGTADLGPGQRAILERLLQNTDNSADGYRQRLFEAKQDTQTKKWWLYVTYDFPRLKEGKLNQEIVVGVDLGFSIPLYVALNIGHARLGRRHFQALGNRIRSLQRQVLARRRSIQRGGRVNISHSTARSGHGRKRKLLPTEKLRGRIEKSYSTLNHQLSASVIDFAKNHHAGTIQIEDLANLKEELAGTFIGARWRYHQLQQFLKYKAEEAGITLNQVNPRYTSRRCSECGFINIDFDRAFRDAGRTEGRVTKFLCPECGYEADPDYNAARNISILDIDKLIRVQCKKQGLTYDAH

SEQ ID NO: 125 - protein, 475 aa, synthetic construct
  REGION 1..475: note = Description of Artificial Sequence: Synthetic polypeptide
MPERPKTVNKVIWFQIHKPAGDMTWKELGNLLREARYRVFRLANLAVSEKYLSFHMWRTGQEYKSETIGKLNRRLREMLIEEGVEEESQKRFSATGALPDTVVSTLAKGKLAAITSKSKWKDVVNGKTSLPTFKLNMAIPVRCDKAEQRRLRRTESGDVELELMICKQPYPRVVLKTGKLKSGQRAILDRLVENNDNSKEGYSQRVFEIKQVENNDGSKEWRLYISYTFPKKAVEANADVAVGVDIGFSVPLVAAVNNGLERLGYNDFRALNERIRSLQRQVLVRRRSMQSGGRDYVSTPTARSGHGRKRKLLPIQTLRKRWDNAYTTLNHQLSHAVVSFAENHGAATIQIENVKSLKDELRGTFLGQRWRYFELQQFLKYKADEVGIELREVNARYTSRRCSECGYINMAFTRQARDKGRVDGKPMEFVCPECGYKAHPDYNAARNIAMLDIEQKMQVQCKQQGITYADDSEVL

SEQ ID NO: 126 - protein, 448 aa, synthetic construct
  REGION 1..448: note = Description of Artificial Sequence: Synthetic polypeptide
MTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 127 - protein, 477 aa, synthetic construct
  REGION 1..477: note = Description of Artificial Sequence: Synthetic polypeptide
VGKEGKRNVKVMKIRILKPCDGMTWNELGQLLRDARYRVFRLANLTVSEAYLNFHLWRTGRSQEFKKQTIGQLNRQLRNILQQEKYDDEKLNRYSKTGALPDTVCSALWQYKLMAVMKKSKWSEVIRGKSSLPTFRNDMAIPVRCDKPEQKRIEKTEQGQVEAALQVCVQPYPRVILGTHTLGDGQDAILKRLLDNQNQAIGGYRQRSFEIKYDEQKRWWLFITYDFPATEVATDKTIAVGVDLGVSVPLYAAVNNGPARLGRREFGGLGRRIRDLRNQTDARRRSIQRSGREGQSDDTARAGHGRKRKLLPIHILEGRLDKAYTTLNHQMSAAVIKFAAEQGAGIIQIENLAGLQDELRGTFIGGRWRYRQLQDFLKYKTQEMGIELRQVNPKYTSRRCSKCGFIHKDFDRDYRNRHSENGKPAQFVCPNPDCKYESDPDYNAARNLATLDIEEQIRVQCQKQGLEYDSKKDKNAL

SEQ ID NO: 128 - protein, 472 aa, synthetic construct
  REGION 1..472: note = Description of Artificial Sequence: Synthetic polypeptide
MKEKSKTLVKVARLRILKPAGDMTWSELGEMLRTVRYRVFRLANLAVSEAYLGFHMFRTQRAAEFKAETMGKLSRRLREMLIEEGVDEKELNCYSLTGAVPDTVAGALHQYKIRGITSPTKWRQVVRGQAALPTFRNDMSIPIRCDKPYQRRLEKTEAGEVEVELMICRKPYPRIVLGTADVGPGQEVILERLLQNKDNSSDGYRQRLFEAKQDRQTGKWWLYVTYDFPRPEEGELNPEIVVGVDLGFSVPLYVAINNGYARLGRRHFQALGNRIRSLQRQVLARRRSIQRGGRVNISHDTARSGHGIKRKLLPTEKLRGRIEKSYSTLNHQLSASVIDFTKNHHAGTIQIEDLANLKEVLAGTFIGARWRYHQLQQFLKYKADEAGITLKEVNPRYTSRRCSECGFIHKDFDRAFRDSGRTDGKVARFVCPECGYGPVDPDYNAAKNISTLDIEKHIRVQCKKQGLEYEVH

SEQ ID NO: 129 - protein, 470 aa, synthetic construct
  REGION 1..470: note = Description of Artificial Sequence: Synthetic polypeptide
MKEKAKTLVKVARLRILKPAGDMTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 130 - protein, 468 aa, synthetic construct
  REGION 1..468: note = Description of Artificial Sequence: Synthetic polypeptide
MAKKAKTMFKVTNFRILKPAGDMTWKELGQLLRDARYRTFRMANLALSEAYLNFYLLKKGDLKEYKNVKIGQIAKRLRDMLIEEGVDEEVQNRFSPKVALPAYVYSALDQFKLRGLTSKSNWKKVLRGQASLPTFRLNMSVPIRCDKPEHRRLEKTENGNVEVDLMICRKPYPRVVLETLKLDGSSKAILDRLLENEDNSPGNYRQRCFEVKQNPRSNDWWLYVTYEMPVDKDKKLDPKVIVGVDLGFSVPLYVAINNGHARLGRRHFQALGKRIHNLQNQVLARRRSIQRGGQVNLSHSTSRSGHGRKRKLQPTEKLQQKINSAYSTLNHQLSSSVIDFANNHKAGTIQIEDLETLKEQLTGTYIGRQWRYYQLQQFIEYKAKENSITVKKINPKYTSRRCSMCGHIHADFDRTFRDRSSNKGFVTKFICPECNFEADPDYNAAKNISTLDIENKIKLQCKKQKIDY

SEQ ID NO: 131 - protein, 457 aa, synthetic construct
  REGION 1..457: note = Description of Artificial Sequence: Synthetic polypeptide
MPKITRKIELLFDRSGLSEEECKEKWRFIYQINDNLYRVANRLVNQLYLADEIDDILRLSDQEYIALRKKLANKKLDEATRISLEEQMSQVMKRVNERRSAILQRPQQSFAYSVVTDSDTEGLTAKILDVLKQDVLSHYKADTKEVLKGEKSISNYKKGMPIPFAFNDSLRLYKEDGFFYLKWYNGIRFLLNFGRDASNNQLIVERCLGISKDEISYKACSSSIQIKKKGNHSKIFLLLVVDVPVEQYAQKPNMVVGVDLGLNVPIYAASNSTLERKAIGSREAFLNQRGAFQRRFRALQRLQTTKGGRGRLHKLEPLERVREAERNWVRTQNHLFSREVINFAIDVGASTIQMEKLANFGRDAQGEVREDKKYVLRNWSYFELQNLIEYKAKRAGIKVKYINPAFTSQTCSECGQLGERDSIHFKCTNPDCPNCGKDIHADYNGARNIAKSKDYIK

SEQ ID NO: 132 - protein, 468 aa, synthetic construct
  REGION 1..468: note = Description of Artificial Sequence: Synthetic polypeptide
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 133 - protein, 468 aa, synthetic construct
  REGION 1..468: note = Description of Artificial Sequence: Synthetic polypeptide
MPTITRKIELTLCTDGLSEEQRKEQWGLLYHINDNLYKAANNISSKLYLDDHVSSMVRMKHAEYLSLLKELARAEKQKTPDADAIAELKKKVAATEKEMTDQEHAICKYATEMSTQSLSYRFSTEFETKIFAKILDCLKQGVFATFNSDAKDVKRGERAIRNYKKGMPIPFAWTDSLRIKKDNKDFYLLWYNGLRFLFNFGKDRSNNRLIVERCLKMDADYDGEYKLCNSSIQIAKREGKVKLFLLLVVSIPKEHVELNKKVVVSVDLGINVPAYVATNITEERKAIGDREHFLNSRMAFQRRYKSLQRLKGTTGGKGRTKKLEPLERLRKAEHNWVHTQNHLFSREVVDFAVKTHAATIHMEDLSGFGKDNDGNADERKEFVLRNWSYYELQNMISYKAAKYGIKVEKIRPAYTSKTCSWCGQHGFREGVTFICENPACKQCGEKVHADYNAARNIANSKEIIKKNE

SEQ ID NO: 134 - protein, 469 aa, synthetic construct
  REGION 1..469: note = Description of Artificial Sequence: Synthetic polypeptide
MPTITRKIELTLCTEGLSDQERKDQWNLLYHINDNLYRAANNISSKLYLDDHVGSMVRLKHAEYLSLLRALEKAKKQKAPDEEVIAELSQQVATAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNCLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDNDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 135 - protein, 468 aa, synthetic construct
  REGION 1..468: note = Description of Artificial Sequence: Synthetic polypeptide
MPTITRKIELTLLTEGLSEEQRKEQWGLLYHINDNLYKAANNISSKLYLDDHVSTMVRMKHAEYLSLLRELARAEKQKKPDVDAIAELREKVTAAEKEMSDQERAICTYATEMSTQSLSYRFATEIETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIVKREGKVKLFLLLVVSIPQEHVELNKKIVVGVDLGINVPAYVATNITEERKAIGDREHFLNSRMAFQRRYKSLQRLKGTAGGKGRTKKLEPLERLRKAEHNWVHTQNHLFSREVVDFAVKSHAATIHMEDLSGFGKDNDGNADERKEFVLRNWSYYELQNMIAYKAAKYGIKVERIRPAYTSKTCSWCGQLGFREGVTFICENPECKQCGEKVHADYNAARNIANSKDIIKKNE

SEQ ID NO: 136 - protein, 463 aa, synthetic construct
  REGION 1..463: note = Description of Artificial Sequence: Synthetic polypeptide
MPTMTRKIELKLCTEGLSDEERKAQLGLLYHINDNLYKAANNISSKLYLDDHVSSMVRLKHAEYLSLLNEFEKAKKKGDEEQIVELSLRVAAAEKELTDQELAICKYATEMSTDTLAYRFANEIEINVFGQILACLKQGIHSTFKKDAADVKRGERAIRNFKKGMPIPFPWSKSIRIENEGSDFYLRWYNGLRFRFDFGKDRSNNRLIVSRCLNLDPDFEDEYKLSNSSLQMVKRDGRPKLFLLLVVNIPQENVELNKKIVVGVDLGINSPAYVATNITMERQRIGSRDTFLNARMAIQRRFQSLQKLQNTAGGRGRKKKLEPLERLKETERNWVRTQNHLFSRDVVQFAVKTRAATIHMEDLSGFGKDDDGNADEKKEFVLRNWSYYELQTMIKYKAAKYGIKVEKIRPAYTSRTCSWCGHEGDRKGETFICENPECEKYGKKENADYNAARNIANSTDIIK

SEQ ID NO: 137 - protein, 468 aa, synthetic construct
  REGION 1..468: note = Description of Artificial Sequence: Synthetic polypeptide
MPTITRKIELTLCTEGLSDEQRKEQWGLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNNSLKIESDSGEFYLRWYNGLRFLLTFGKDRSNNRMIVNRCMKMDEDFEGEYKLCNSSIQLAKRDGKPKLFLLLVVNIPQEHVKLNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDLSGFGKDKDGNADEKKEFVLRNWSFYELQNMIAYKSAKYGIKVVKIRPAYTSKTCSWCGQQGDRKSTTFICENPKCKHYGESIHADYNAARNIANSNDIVKENE

SEQ ID NO: 138 - protein, 467 aa, synthetic construct
  REGION 1..467: note = Description of Artificial Sequence: Synthetic polypeptide
MPKITRKIEMTLCTEGLSDEQRKEQWGLLYHINDNLYKAANNISTKLYLDEHVSSMVRMKHADYLSLLKELAKAEKKSPDEDLIAELREKLAAAEQEMTDQELAICKYATEMSTQTLAYKFATEIEINVFGQILACLKQAAQSNFKSDAKDVKRGERAIRNYKKGMPIPFPWNDNIRIDADGDEFYLRWYNGLRFHLTFGKDKSNNRMIVKRCLKMDKDFEGEYKLCNSSIQMVKRDGKPKLFLLLVVNIPQEHVELNKNVVVGVDLGVNVPAYVATNITEERKAIGEREHFLNTRMQIQRRYKSLQRLKATAGGKGRTKKLEPLERLRKAEHNWVHTQNHLFSREVVNFAVQTHAATIHMEDLSGFGKDDDGNADEQKEFVLRNWSFYELQNMIAYKAAKYGIKVEKVKPAYTSKTCSWCGQLGFRQGVTFICENPACKQCGEKVHADYNAARNIANSKDIIKKNE

SEQ ID NO: 139 - protein, 469 aa, synthetic construct
  REGION 1..469: note = Description of Artificial Sequence: Synthetic polypeptide
MPTITRKIELHLCTDGLTDEQQKAQRLLLYHINDNLYKAANNVSSKLYLDEHVSSMVRLKHDEYLSLSRELARAEKKHDDELTTELRGKLAAAEREMTDQELAICKYATEMSTQSLSYRLVTELETKIFAKILDCLKQGVYATFNSDARDVKRGERAIRNYKKGMPIPFAWNDSVRIEYDEKEKDFYLRWYNDIRFKFHFGRDRSNNRLIVSRCLKLDKDYEGDYQLCNSSIQIVKRDGSTKFFLLLVVKIPQEHVELNKRIVVGVDLGINYPAYVATNCTEERMYIGDREHFLNTRMQFQRRYKSLQKLKGTAGGKGRSKKLEPLERLRNAERNWVHTQNHLFSLKVVNFAVQTHAATIHLEDLSGFGKDDDGNADERKEFVLRNWSYYELQSMIEYKAKKYGIKVEKIRPAYTSQTCSWCGQRGFRQGVTFICENPECKKCGEKENADYNAARNIANSKDVIKDKNE

SEQ ID NO: 140 - protein, 491 aa, synthetic construct
  REGION 1..491: note = Description of Artificial Sequence: Synthetic polypeptide
TPFVLYFQNYSLSLRQHITLYSMPTITRKIELTLCTEGLSDQERKDQWNLLYHINDNLYRAANNISSKLYLDDHVGSMVRLKHAEYLSLLRAMEKAKKQKAPDEEVIAELSQQVAAAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNRLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDRDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 141 - protein, 469 aa, synthetic construct
  REGION 1..469: note = Description of Artificial Sequence: Synthetic polypeptide
MPTITRKIELHLCTEELSDEQQKAQRLLLYHINDNLYKAANNVSSKLYLDEHVSSMVRLKHDEYLSLLRELARAEKKADDELATQLREKLVAAEREMTDQELAICKYATEMSTQSLSYRFVTELETKIFAKILDCLKQGVYATFNSDSRDVKRGERAIRNYKKGMPIPFAWDKSVRIEYEEKEKDFFLRWYNDIRFKFHFGRDRSNNRLIVSRCMKLDKDYEGDYQLCNSSIQIVKRDGSTKYFLLLVVKIPQEHVELNKKIVVGVDLGINYPAFAATNCTEERMSIGDREHFLNTRMQFQRRFKSLQRLKGTTGGKGRNKKLEPLERLRKAEHNWVHTQNHLFSLKVVNFAVQAHAATIHLEDLSGFGKDDDGNADERKEFVLRNWSYYELQNMIKYKAKKFGIQVEKIRPAYTSQTCSWCGQRGFRQGITFICENPECKKCGEKENADYNAARNIANSKDIIKDKDE

SEQ ID NO: 142 - protein, 460 aa, synthetic construct
  REGION 1..460: note = Description of Artificial Sequence: Synthetic polypeptide
MPIITRKIELHISKEGLSAEDYKAQWQYLRQINDNLYMAANRVSSHCFLNDEYKYRLCLQIPDYIDIEKQLKDSKRARLSKEELGQLKKRKKELENTVKGRFQDEFEKNSLYTIISNEFGEIIPGQILTCLRQCVQSKYNRAKEELEKGERAISTYKKGMPIPFPINKSIRLQKQGEDFVLKWYNKIVFKLHFGRDRSNNRVIVERLIQSALNDKQKGEDYVMNNSSIQLVEKDKMTKIFLLLSMDIPTQKRKLDSELVLGVDLGLNFPLYYATNQSANIHDHIGDKDIFLKERMVFQRRFKELQRLQCTQGGRGRKKKLEPLEKLRDKERNWVRTKNHIFSREVIKVALHLGAGTIHLENLHNFGKDGNGELKNSKKFVFRNWSYFELQSMIEYKAKMEGITVKYVNPAYTSQTCSVCGMIGERKEQAVFRCMNSSCLEYGKEVNADFNAARNIAKAKM

SEQ ID NO: 143 - protein, 466 aa, synthetic construct
  REGION 1..466: note = Description of Artificial Sequence: Synthetic polypeptide
MPTITRKIELTLCTDGLSDDLRKDQWQLLYHINDNLYKAANNISSKLYLDEHVASMVRLKHAEYLGLIKELAKARKRADDEAVRDLCSKLAVAEQEMNEQAKAICDYATEMSTQTLSYNFAKEIETNIFGQILTCLRQGVLLNFNSDARDVKRGERAIRNYKKGMPIPFPWNDTIKIVSEGDEFYLRWFSGLRFHLNFGKDRSNNRMIVRRCLKMEQDFDEEYKISNSSIQVAKRDGKQKLFLLLVVQIPQEQVVLNKKIVVGVDLGVNVPAYVATNITEERKAIGDREHFLNTRMQFQRRYKSLQRLKTTEGGRGRAKKLEPLERLRKAEHNWVHTQNHLFSREVVNFALQTQAATINMEDLSGFGKDNDGNADECKEFVLRNWSYYELQNMIVYKASKYGIRVQKIRPAYTSKTCSWCGHMGFREGVTFICENPDCKQFGEKVHADYNAARNIANSKEIIKNDE

SEQ ID NO: 144 - protein, 444 aa, synthetic construct
  REGION 1..444: note = Description of Artificial Sequence: Synthetic polypeptide
MSKTVTKTVKIALICEHTNKYGEKVDYKDINKLLWKLQKQTRELKNKTIQLCWEYNNFSCDYYKEHHEYPNMEDILKYKRINGFVENKLKTVNDLYSSNCSTTILSTCNEFQNYRSEFLKGTRSINSYKSDQPLDLHKGAIKLEHDGKDFYVSLKLLKRSAFNAMEFKGSDIRFKLNVKDKDKSTLKILESCYDKIYSISASKMTYDRKAGKWFLLLAYSFTPAKTENLDPEKILGVDLGIKIPICASVYGDLDRLTIEGGKIEEFRRRVEARKRSLQKQGKQCGDGRIGHGTKKRIKPITDIGDKIARFRDTENHIYSRYLIEYAVKKGCGTIQMEKLEGITREKDIFLKNWTYFDLQKKIEYKAKEKGIKVVYIEPAYTSKRCSSCGFIDTDNRLDQAHFKCLKCGFNENADYNASQNIGIKNIDKIIKEEHKSASDKLTSE

SEQ ID NO: 145 - protein, 439 aa, synthetic construct
  REGION 1..439: note = Description of Artificial Sequence: Synthetic polypeptide
VIILTKVVKLYLISEQINKEGQKIDYQRINSILWDLQKQTRDIKNRTVQLCWEWMNFSSDYCKTQEEYPKERDILGYTLEGYVYDYFKTGYDLYTGNISTSSREVCSSFKNVKKEILKGERSILSYKANQPLDLHKKAISLEYDNFNFFVKLKLLNRTGKKKYDITEDINFKIQVNDKSTRTILERCYDKEYKISGSKLIYEKKKKLWRLNLCYSFENSQVETLEKDKILGIDLGIVYPLMASIYGEYDRFSIKGGEIEEFRRRTEARKRSILQQTKYCGDGRIGHGRNKRTQPAYKINDKIARFRDTANHKYSRALIEYAVKKNCGIIQMENLTGISDNTDCFLKDWSYYDLQTKIENKAKEMGIKVVYIKAQYTSQRCSRCGYIDVNNRIRQALFKCQNCGYETNADYNASQNIGMYDIENIIEETLKIQSANVKQS

SEQ ID NO: 146 - protein, 437 aa, synthetic construct
  REGION 1..437: note = Description of Artificial Sequence: Synthetic polypeptide
MTKVTKVYLISEQIDKDGNKIDFKKISELLWNLQMQTRDIKNKCVQLCWEWLNFSSDYYKKSEEYPKEKDTLGYTLSGFVYDRIKNGSDLYSSNLSTSSRDTCTAFSNYKKEMLKGERSVLSFKANQPLDIHNKAIKLSYENGNFFVALKMLNRAGKEKYGIKDDLRFRMQVRDKSVRTILERLMNDEYKVSASKLMYDKKKKLWKLNLCYSFDNHVISTLDTEKIMGVDLGVVYPIMASVNGDYARFSIKGGEIEAFRSRVEARRRSLLNQSRYCGDGRIGHGRKKRTEPATQIADKIARFRDTTNHKYSRALIDYAIKNGCGTIQMEKLTGITSSAEHFLKEWSYFDLQTKIESKAKEAGIKVVYINPKFTSQRCNKCGYIHTDNRPVQARFCCQKCGYEENADYNASQNIGTKHIDVIIEETLKMQCEPETPTE

SEQ ID NO: 147 - protein, 428 aa, synthetic construct
  REGION 1..428: note = Description of Artificial Sequence: Synthetic polypeptide
MNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 148 - protein, 443 aa, synthetic construct
  REGION 1..443: note = Description of Artificial Sequence: Synthetic polypeptide
VTLLVKVVKIYLISEQFDKAGNQIDYKEVNKILWELQKQTREAKNKTVQLLWEWNNFSSDYVKASGIYPKAKDIFGYSSVHGQANKELRTKLALNSSNLSTTTMDVCKIFNTYKKEVWEGKRSVPSYKSDQPLDLHKESIKLIYENNEFYVRLALLKKAEFAKYGFKDGFRFKMQVKDNSTKTILERCFDEVYKINASKLLYDQKKKKWKLNLSYSFDNKNISELDKEKILGVDVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 149 - protein, 428 aa, synthetic construct
  REGION 1..428: note = Description of Artificial Sequence: Synthetic polypeptide
MNKVVKLALICEQSDKNNSPVDYKKVNEILWELQKQTREIKNKTIQYCWEYYNFSSDYYKKFNKYPKEKDILSYTLWGFINDKFKTGNDLYSGNCSATTKKVIKEFKNSKKELIRGSRSIINYKSNQPLNIHNKCIHLQFKNNNFYVSINLLNRRSFKKYNFANTAIKFKILVRDNSTKAILERCISNEYKISESQLIYNKKKKCWFLNLSYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKNNCGTIQMEDLTGITDNANRFLKNWSYYDLQTKIEYKAKEASINVVYINPENTSRRCSKCGYIDKENRKTQSSFICLKCGFKENADYNASQNISIKDIDKLIKEDVH

SEQ ID NO: 150 - protein, 443 aa, synthetic construct
  REGION 1..443: note = Description of Artificial Sequence: Synthetic polypeptide
VTLLVKVVKIHLISEQFDKAGNRIDYEEVNKILWELQKQTREAKNKTVQLLWEWNNFSSDYVKASGIYPKAKDIFGYSSVHGQANKELRTKLALNSSNLSTTTMDVCKNFNTYKKEVWKGKRSVPSYKSDQPLDLHKDSIKLIYENNQFYVRLALLKKAEFAKYGFKDGFHFKMQVKDNSTKTILERCFDEVYKINASKLLYDQKKKKWKLNLSYSFDNKNISELDKEKILGVDVGVSYPLVASVFGDRDRFKIKGGEIEKFRKSVEARRRSMLEQTKYCGDGRIGHGRKKRTEPALNIGDKIARFRDTTNHKYSRALIEYAVKKGCGTIQMEKLTGITSKADRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 151 - protein, 449 aa, synthetic construct
  REGION 1..449: note = Description of Artificial Sequence: Synthetic polypeptide
LIWKDALGGIILTKIVKLYLISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDKKEHFLKEWSYYDLQTKIENKAKEKGIKIVYINPEYTSQRCSKCGYIDANNRELRAVFKCQKCGFEADADYNASQNIGIKNIEDIIENTLKISSANEKQTKNT

SEQ ID NO: 152 - protein, 462 aa, synthetic construct
  REGION 1..462: note = Description of Artificial Sequence: Synthetic polypeptide
VFYSTFLCYILTKYIDFSANECYNINTSSEVKQLMNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 153 - protein, 430 aa, synthetic construct
  REGION 1..430: note = Description of Artificial Sequence: Synthetic polypeptide
LISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDNKEHFLKEWSYYDLQTKIENKAKEKGIKIVYINPEYTSQRCSKCGYIDANNRELRAVFKCQNCGFEADADYNASQNIGIKNIEDIIENTLKISSANEKQTKNT

SEQ ID NO: 154 - protein, 429 aa, synthetic construct
  REGION 1..429: note = Description of Artificial Sequence: Synthetic polypeptide
LVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 155 - protein, 707 aa, synthetic construct
  REGION 1..707: note = Description of Artificial Sequence: Synthetic polypeptide
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 156 - protein, 757 aa, synthetic construct
  REGION 1..757: note = Description of Artificial Sequence: Synthetic polypeptide
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 157 - protein, 765 aa, synthetic construct
  REGION 1..765: note = Description of Artificial Sequence: Synthetic polypeptide
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 158 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 159 - protein, 812 aa, synthetic construct
  REGION 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 160 - protein, 812 aa, synthetic construct
  REGION 1..812: note = Description of Artificial Sequence: Synthetic polypeptide
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 161 - protein, 793 aa, synthetic construct
  REGION 1..793: note = Description of Artificial Sequence: Synthetic polypeptide
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 162 - protein, 441 aa, synthetic construct
  REGION 1..441: note = Description of Artificial Sequence: Synthetic polypeptide
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 163 - protein, 812 aa, synthetic construct
  REGION 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 164 - protein, 812 aa, synthetic construct
  REGION 1..812: note = Description of Artificial Sequence: Synthetic polypeptide
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 165 - protein, 761 aa, synthetic construct
  REGION 1..761: note = Description of Artificial Sequence: Synthetic polypeptide
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 166 - protein, 717 aa, synthetic construct
  REGION 1..717: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 167 - protein, 781 aa, synthetic construct
  REGION 1..781: note = Description of Artificial Sequence: Synthetic polypeptide
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 168 - protein, 793 aa, synthetic construct; identical to SEQ ID NO: 161
  REGION 1..793: note = Description of Artificial Sequence: Synthetic polypeptide

SEQ ID NO: 169 - protein, 717 aa, synthetic construct; identical to SEQ ID NO: 166
  REGION 1..717: note = Description of Artificial Sequence: Synthetic polypeptide

SEQ ID NO: 170 - protein, 761 aa, synthetic construct; identical to SEQ ID NO: 165
  REGION 1..761: note = Description of Artificial Sequence: Synthetic polypeptide

SEQ ID NO: 171 - protein, 765 aa, synthetic construct
  REGION 1..765: note = Description of Artificial Sequence: Synthetic polypeptide
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 172 - protein, 766 aa, synthetic construct
  REGION 1..766: note = Description of Artificial Sequence: Synthetic polypeptide
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 173 - protein, 870 aa, synthetic construct
  REGION 1..870: note = Description of Artificial Sequence: Synthetic polypeptide
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 174 - protein, 830 aa, synthetic construct
  REGION 1..830: note = Description of Artificial Sequence: Synthetic polypeptide
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 175 - protein, 790 aa, synthetic construct
  REGION 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 176 - protein, 790 aa, synthetic construct
  REGION 1..790: note = Description of Artificial Sequence: Synthetic polypeptide
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 177 - protein, 782 aa, synthetic construct
  REGION 1..782: note = Description of Artificial Sequence: Synthetic polypeptide
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 178 - protein, 774 aa, synthetic construct
  REGION 1..774: note = Description of Artificial Sequence: Synthetic polypeptide
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 179 - protein, 769 aa, synthetic construct
  REGION 1..769: note = Description of Artificial Sequence: Synthetic polypeptide
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 180 - protein, 767 aa, synthetic construct
  REGION 1..767: note = Description of Artificial Sequence: Synthetic polypeptide
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 181 - protein, 763 aa, synthetic construct
  REGION 1..763: note = Description of Artificial Sequence: Synthetic polypeptide
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 182 - protein, 761 aa, synthetic construct
  REGION 1..761: note = Description of Artificial Sequence: Synthetic polypeptide
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 183 - protein, 746 aa, synthetic construct
  REGION 1..746: note = Description of Artificial Sequence: Synthetic polypeptide
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 184 - protein, 735 aa, synthetic construct
  REGION 1..735: note = Description of Artificial Sequence: Synthetic polypeptide
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 185 - protein, 725 aa, synthetic construct
  REGION 1..725: note = Description of Artificial Sequence: Synthetic polypeptide
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 186 - protein, 718 aa, synthetic construct
  REGION 1..718: note = Description of Artificial Sequence: Synthetic polypeptide
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 187 - protein, 708 aa, synthetic construct
  REGION 1..708: note = Description of Artificial Sequence: Synthetic polypeptide
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 188 - protein, 491 aa, synthetic construct
  REGION 1..491: note = Description of Artificial Sequence: Synthetic polypeptide
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 189 - protein, 848 aa, synthetic construct
  REGION 1..848: note = Description of Artificial Sequence: Synthetic polypeptide
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 190 - protein, 781 aa, synthetic construct
  REGION 1..781: note = Description of Artificial Sequence: Synthetic polypeptide
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 191 - protein, 767 aa, synthetic construct
  REGION 1..767: note = Description of Artificial Sequence: Synthetic polypeptide
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 192 - protein, 760 aa, synthetic construct
  REGION 1..760: note = Description of Artificial Sequence: Synthetic polypeptide
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 193 - protein, 752 aa, synthetic construct
  REGION 1..752: note = Description of Artificial Sequence: Synthetic polypeptide
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 194 - protein, 747 aa, synthetic construct
  REGION 1..747: note = Description of Artificial Sequence: Synthetic polypeptide
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 195 - protein, 733 aa, synthetic construct
  REGION 1..733: note = Description of Artificial Sequence: Synthetic polypeptide
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 196 - protein, 702 aa, synthetic construct
  REGION 1..702: note = Description of Artificial Sequence: Synthetic polypeptide
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 197 - protein, 661 aa, synthetic construct
  REGION 1..661: note = Description of Artificial Sequence: Synthetic polypeptide
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 198 - protein, 531 aa, synthetic construct
  REGION 1..531: note = Description of Artificial Sequence: Synthetic polypeptide
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 199 - protein, 479 aa, synthetic construct
  REGION 1..479: note = Description of Artificial Sequence: Synthetic polypeptide
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 200 - protein, 452 aa, synthetic construct
  REGION 1..452: note = Description of Artificial Sequence: Synthetic polypeptide
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 201 - protein, 425 aa, synthetic construct
  REGION 1..425: note = Description of Artificial Sequence: Synthetic polypeptide
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 202 - protein, 735 aa, synthetic construct
  REGION 1..735: note = Description of Artificial Sequence: Synthetic polypeptide
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 203 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 204 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 205 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 206 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 207 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 208 - protein, 1285 aa, unidentified
  REGION 1..1285: note = Description of Unknown: Herbinix hemicellulosilytica sequence
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 209 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 210 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 211 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 212 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 213 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 214 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFAVFFKPDDFVLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLDEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELRLLDPSSGHPFLSATMETAHRYTEGFYKCYLEKKREWLAKIFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 215 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 216 - protein, 668 aa, unidentified
  REGION 1..668: note = Description of Unknown: Cas13c sequence
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 217 - protein, 796 aa, unidentified
  REGION 1..796: note = Description of Unknown: Cas13c sequence
MEKDKKGEKIDISQEMIEEDLRKILILFSRLRHSMVHYDYEFYQALYSGKDFVISDKNNLENRMISQLLDLNIFKELSKVKLIKDKAISNYLDKNTTIHVLGQDIKAIRLLDIYRDICGSKNGFNKFINTMITISGEEDREYKEKVIEHFNKKMENLSTYLEKLEKQDNAKRNNKRVYNLLKQKLIEQQKLKEWFGGPYVYDIHSSKRYKELYIERKKLVDRHSKLFEEGLDEKNKKELTKINDELSKLNSEMKEMTKLNSKYRLQYKLQLAFGFILEEFDLNIDTFINNFDKDKDLIISNFMKKRDIYLNRVLDRGDNRLKNIIKEYKFRDTEDIFCNDRDNNLVKLYILMYILLPVEIRGDFLGFVKKNYYDMKHVDFIDKKDKEDKDTFFHDLRLFEKNIRKLEITDYSLSSGFLSKEHKVDIEKKINDFINRNGAMKLPEDITIEEFNKSLILPIMKNYQINFKLLNDIEISALFKIAKDRSITFKQAIDEIKNEDIKKNSKKNDKNNHKDKNINFTQLMKRALHEKIPYKAGMYQIRNNISHIDMEQLYIDPLNSYMNSNKNNITISEQIEKIIDVCVTGGVTGKELNNNIINDYYMKKEKLVFNLKLRKQNDIVSIESQEKNKREEFVFKKYGLDYKDGEINIIEVIQKVNSLQEELRNIKETSKEKLKNKETLFRDISLINGTIRKNINFKIKEMVLDIVRMDEIRHINIHIYYKGENYTRSNIIKFKYAIDGENKKYYLKQHEINDINLELKDKFVTLICNMDKHPNKNKQTINLESNYIQNVKFIIP

SEQ ID NO: 218 - protein, 897 aa, unidentified
  REGION 1..897: note = Description of Unknown: Cas13c sequence
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 219 - protein, 919 aa, unidentified
  REGION 1..919: note = Description of Unknown: Cas13c sequence
MEEIKHKKNKSSIIRVIVSNYDMTGIKEIKVLYQKQGGVDTFNLKTIINLESGNLEIISCKPKEREKYRYEFNCKTEINTISITKKDKVLKKEIRKYSLELYFKNEKKDTVVAKVTDLLKAPDKIEGERNHLRKLSSSTERKLLSKTLCKNYSEISKTPIEEIDSIKIYKIKRFLNYRSNFLIYFALINDFLCAGVKEDDINEVWLIQDKEHTAFLENRIEKITDYIFDKLSKDIENKKNQFEKRIKKYKTSLEELKTETLEKNKTFYIDSIKTKITNLENKITELSLYNSKESLKEDLIKIISIFTNLRHSLMHYDYKSFENLFENIENEELKNLLDLNLFKSIRMSDEFKTKNRTNYLDGTESFTIVKKHQNLKKLYTYYNNLCDKKNGFNTFINSFFVTDGIENTDFKNLIILHFEKEMEEYKKSIEYYKIKISNEKNKSKKEKLKEKIDLLQSELINMREHKNLLKQIYFFDIHNSIKYKELYSERKNLIEQYNLQINGVKDVTAINHINTKLLSLKNKMDKITKQNSLYRLKYKLKIAYSFLMIEFDGDVSKFKNNFDPTNLEKRVEYLDKKEEYLNYTAPKNKFNFAKLEEELQKIQSTSEMGADYLNVSPENNLFKFYILTYIMLPVEFKGDFLGFVKNHYYNIKNVDFMDESLLDENEVDSNKLNEKIENLKDSSFFNKIRLFEKNIKKYEIVKYSVSTQENMKEYFKQLNLDIPYLDYKSTDEIGIFNKNMILPIFKYYQNVFKLCNDIEIHALLALANKKQQNLEYAIYCCSKKNSLNYNELLKTFNRKTYQNLSFIRNKIAHLNYKELFSDLFNNELDLNTKVRCLIEFSQNNKFDQIDLGMNFINDYYMKKTRFIFNQRRLRDLNVPSKEKIIDGKRKQQNDSNNELLKKYGLSRTNIKDIFNKAWY

SEQ ID NO: 220 - protein, 1110 aa, unidentified
  REGION 1..1110: note = Description of Unknown: Cas13c sequence
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY

SEQ ID NO: 221 - DNA, 5417 bp, Homo sapiens
  source 1..5417: mol_type = unassigned DNA
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 222 - DNA, 17 bp, synthetic construct
  misc_feature 1..17: note = Description of Artificial Sequence: Synthetic oligonucleotide
  source 1..17: mol_type = other DNA
cctacgccatcagctcc

SEQ ID NO: 223 - DNA, 17 bp, synthetic construct
  misc_feature 1..17: note = Description of Artificial Sequence: Synthetic oligonucleotide
  source 1..17: mol_type = other DNA
gagctgttggcgtaggc

SEQ ID NO: 224 - RNA, 36 nt, synthetic construct
  misc_feature 1..36: note = Description of Artificial Sequence: Synthetic oligonucleotide
  source 1..36: mol_type = other RNA
ctttcaagactaatagattgctccttacgaggagac

SEQ ID NO: 225 - skipped

SEQ ID NO: 226 - RNA, 17 nt, synthetic construct
  misc_feature 1..17: note = Description of Artificial Sequence: Synthetic oligonucleotide
  source 1..17: mol_type = other RNA
tggtagttggagctgat

SEQ ID NO: 227 - RNA, 17 nt, synthetic construct
  misc_feature 1..17: note = Description of Artificial Sequence: Synthetic oligonucleotide
  source 1..17: mol_type = other RNA
gagctgatggcgtaggc

SEQ ID NO: 228 - RNA, 17 nt, synthetic construct
  misc_feature 1..17: note = Description of Artificial Sequence: Synthetic oligonucleotide
  source 1..17: mol_type = other RNA
cctacgccatcagctcc

SEQ ID NO: 229 - DNA, 17 bp, synthetic construct
  misc_feature 1..17: note = Description of Artificial Sequence: Synthetic oligonucleotide
  source 1..17: mol_type = other DNA
tggtagttggagctgtt

SEQ ID NO: 230 - DNA, 17 bp, synthetic construct
  misc_feature 1..17: note = Description of Artificial Sequence: Synthetic oligonucleotide
  source 1..17: mol_type = other DNA
gagctgttggcgtaggc

SEQ ID NO: 231 - DNA, 17 bp, synthetic construct
  misc_feature 1..17: note = Description of Artificial Sequence: Synthetic oligonucleotide
  source 1..17: mol_type = other DNA
cctacgccaacagctcc

SEQ ID NO: 232 - DNA, 17 bp, synthetic construct
  misc_feature 1..17: note = Description of Artificial Sequence: Synthetic oligonucleotide
  source 1..17: mol_type = other DNA
tggtagttggagcttgt

SEQ ID NO: 233 - DNA, 17 bp, synthetic construct
  misc_feature 1..17: note = Description of Artificial Sequence: Synthetic oligonucleotide
  source 1..17: mol_type = other DNA
gagcttgtggcgtaggc

SEQ ID NO: 234 - DNA, 17 bp, synthetic construct
  misc_feature 1..17: note = Description of Artificial Sequence: Synthetic oligonucleotide
  source 1..17: mol_type = other DNA
cctacgccacaagctcc

SEQ ID NO: 235 - RNA, 37 nt, synthetic construct
  misc_feature 1..37: note = Description of Artificial Sequence: Synthetic oligonucleotide
  source 1..37: mol_type = other RNA
attgctccttacgaggagaccctacgccaccagctcc

SEQ ID NO: 236 - RNA, 37 nt, synthetic construct
  misc_feature 1..37: note = Description of Artificial Sequence: Synthetic oligonucleotide
  source 1..37: mol_type = other RNA
attgctccttacgaggagaccctacgccatcagctcc

SEQ ID NO: 237 - RNA, 20 nt, synthetic construct
  misc_feature 1..20: note = Description of Artificial Sequence: Synthetic oligonucleotide
  source 1..20: mol_type = other RNA
gtagttggagctggtggcgt

SEQ ID NO: 238 - RNA, 20 nt, synthetic construct
  misc_feature 1..20: note = Description of Artificial Sequence: Synthetic oligonucleotide
  source 1..20: mol_type = other RNA
gtagttggagctgatggcgt

SEQ ID NO: 239 - RNA, 20 nt, synthetic construct
  misc_feature 1..20: note = Description of Artificial Sequence: Synthetic oligonucleotide
  source 1..20: mol_type = other RNA
ttggagctggtggcgtaggc

SEQ ID NO: 240 - RNA, 20 nt, synthetic construct
  misc_feature 1..20: note = Description of Artificial Sequence: Synthetic oligonucleotide
  source 1..20: mol_type = other RNA
ttggagctgatggcgtaggc

SEQ ID NO: 241 - RNA, 20 nt, synthetic construct
  misc_feature 1..20: note = Description of Artificial Sequence: Synthetic oligonucleotide
  source 1..20: mol_type = other RNA
gtagttggagctggtggcgt

SEQ ID NO: 242 - RNA, 23 nt, synthetic construct
  misc_feature 1..23: note = Description of Artificial Sequence: Synthetic oligonucleotide
  source 1..23: mol_type = other RNA
gtagttggagctgatggcgtagg

SEQ ID NO: 243 - DNA, 17 bp, synthetic construct
  misc_feature 1..17: note = Description of Artificial Sequence: Synthetic oligonucleotide
  source 1..17: mol_type = other DNA
tggtagttggagctgat

SEQ ID NO: 244 - protein, 478 aa, synthetic construct
  REGION 1..478: note = Description of Artificial Sequence: Synthetic polypeptide
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 245 - DNA, 21 bp, synthetic construct
  misc_feature 1..21: note = Description of Artificial Sequence: Synthetic oligonucleotide
  source 1..21: mol_type = other DNA
gttggagctgatggcgtaggc

SEQ ID NO: 246 - DNA, 17 bp, synthetic construct
  misc_feature 1..17: note = Description of Artificial Sequence: Synthetic oligonucleotide
  source 1..17: mol_type = other DNA
gagctgatggcgtaggc

SEQ ID NO: 247 - DNA, 21 bp, synthetic construct
  misc_feature 1..21: note = Description of Artificial Sequence: Synthetic oligonucleotide
  source 1..21: mol_type = other DNA
gttggagctgttggcgtaggc

SEQ ID NO: 248 - protein, 822 aa, unidentified
  REGION 1..822: note = Description of Unknown: Cas13c sequence
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 249 - protein, 845 aa, unidentified
  REGION 1..845: note = Description of Unknown: Cas13c sequence
MIKKPSNRHALPKVIISKVDNQNILEFKIKYKKLSRLDRVEIKTMHYDDRAIVFDEVIINGGLIDVEYRDNHKTIFVKVGDKSYSISGQKVGGKERLLENRISQTKVQLELKDEATNRVSKTERELIVDDNIKLYSQIVGRDVKTTKDIYLIKRFLGYRSDLLFYYGFVNNFFHVANNRPEFWKIDFNDNRNSKLIEYFIFTINDHLKNDENYLKDYISDRGQIVDDLENIKHIFSALRHGLMHFDYDFFEALFNGEDIDIKMDNQGNTQPLSSLNIKFLDIMIDKLDKLNIDTKKEFIDAEKITIFGEELSLAKLYRFYAHTAINRVAFNKLINSFIIENGVENQSLKEYFNQQAGGIAYEIDIHQNREYKNLYNEHKKLVSRVLSISDGQEIATLNQKIVELKEQMKQITKINSIKRLEYKLRLAFGFIYTEYKNYEEFKNSFDTDIKNGRFTPKDEDGNKRAFDSRELEHLKGYYKATLQTQKPQTDEKMEEVSKRVDRLSLKSLIGDDTLLKFILLMFTFMPQELKGEFLGFIKKYYHDTKHIDQDTISDSDDTIEEGLSIGLKLKILDKNIRSLSILKHSLSFQTKYNKKDRSYYEDGNIHGKFFKKLGISHNQEEFNKSVYAPLFRYYSALYKLINDFEIYTLSLHIVGNETLSDQVNKPQFLSGRYFNFRKLLTQSYNISNNSTHSVIFNAVINMRNDISHLSYEPLLDCPLNGKKSYKRKIRNQFRTINIKPLVESRKMIIDFITLQTDMQKVLGCDAVNDFTMKIVQLRTRLKAYANKEQTIEKMITEAKTPNDFYNIYKVKGVEAINKYLLEVIGETQVEKEIREEIERGNIANS

SEQ ID NO: 250 - protein, 809 aa, unidentified
  REGION 1..809: note = Description of Unknown: Cas13c sequence
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 251 - protein, 1238 aa, unidentified
  REGION 1..1238: note = Description of Unknown: Cas13c sequence
MNQYIHANKKENKKRPNKSSIIRIMVSDFDDEYIQEIKVLYIKQGGVDTFKINKMSYDSASKKIIFEEVATQNMLSVEDSNLNFKRPMVECKNGDIYVVKPSEKVNEKGQAIEPLRSYKIHGKYLDLTEEIGKDNAEQSGKKQIYLHVEDLLGMHTTADSIDRRRLESETQRTLLSKEVMENYALIMGHEIKLDESDETYKLASSKEIYKANRFLDYRSRLLYYYSFINHFLVGLSKGATYEYAGKSLVAKIPDGEVWQLCELEVTAYHGIYINKKRNELVNENSLKIDAIYAQMAKNMKETINEFVDNYNAMIEKQNELKDNKSYKINKKAQNTTKFENFTEEKINQDLHNIVYILSDLRHKLMHFEYHYFELLMTGKKMGEKSEVIVCVPDRNSELPASKNEPSKDKFRKEKKLSELLDLNILKELDTFVKVKESYQTTYLETNDKIEILGKLKTAKSIYQIYHQICQRKNGFNKFINSFFTVDGEENTEVKDCINEVFRKEIHYFEMVIAKSNEDTLNDKNKNKSKKTRDRMKNQIQECKRYQDDESNIDTWVAYHKDIHYSKRYKKLYCEHITLVDNLNSAVSNGLNGQVIKEINDDIAIKKREMNEITKANSKSRLRYKMQMAYGFLFVEYGLKIPKFLNDFDLSHVRTASKIKGYKSPVKVTQYLTNDEGNKDNFNLETLMEDIDKKSKINFEFLKSNEDNNLIKLYILIYQLLPRELKGDFLGFVKNNYYDLKHVDFQTRETEAKDQFFHNMRLFEKNVKAFDLIQYSIGDEMSQLGNETFNFSQALSKIVSDDTILNSATEIPNINRLVYGSLLKYYENAFRLSSEIEIRALIKIARGKQIDNHSIDEAYKDALIFQKSGQTVKFSSILKYFNIDDLNKKNDSKIYNKASNLRNKIAHCDYTVLFITNIICEAENINVKAKYLIDVSNKIGLNTVDLGNDMVNDYLMSYDKQMTYLAKTSEELLKESSLDKSKEKKERRDNLKNETRSHSEIYMEYEWISDYLVKLKDNHEILSKKNKTEDLKLNRAYELIKNYNVALNKKSKIKIRALTTDHINNNWVNIWGALTHELQEIKGLYLYKVTPKIKKGIYFVLTDKKNYCLNINLYTLKKVPSKDDSEMTEERYLRDISEKYYFTVGEDSIKALKERLQTINDKCIITYVNEIDCKNENIKEKPEFKIAEDYNSWDKKYLSISNEELHHWSVETRRDKKYKENLILNLMEKSSFKLNLHQL

SEQ ID NO: 252 - protein, 849 aa, unidentified
  REGION 1..849: note = Description of Unknown: Cas13c sequence
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 253 - protein, 608 aa, unidentified
  REGION 1..608: note = Description of Unknown: Cas13c sequence
MIKNPANRYSLPKVIISEVDSQNILEFKIKYEKLARLDRFEVKAMHYDDGEIVFDEVLVNGGKLDVEYQDEHKTLLVKIEGKEYSIKGQKIGGKQRLLENRISKSKVQLTIKDNIQTNANGTARQKSTEREFIVPENIKLYSQIVGREITTTKEIYLTKRFLGYRSDLLFYYGFVDNFFQVADNKKELWKIDFQNSQFADYFQYMVNDNLKNSDNYLKDYLNDSSKITDDLEKVKTVFSKLRHALLHFEYDFFEKLFNGEDVGFDLDIGFLNLLIENIDKLNIDAKKEFIADEKIKLFGEELALSKVYALYSSICVNRVGFNKFINSLIMVDGVENETLKSFFDDELKEKNPRLFEALGNRAYYVDIHSNRAYKRIYNQHKELVSKSSALSDGRKIHQANQEITKLKEKMNEITKRNSLARLEHKLRVAFGFLYGEYNDHRAFKDNFDTDIKSKKFETLNSDKSKDYFSSTYQNRKPRTREKLEKVESLNLKTLIEDDRLLKFVLLMFLFMPQEVKGEFIGFIKKYYHDTKGIGEDTKEKELDVVETMPLSLKLKILGNNIRSLTLFKYALSSEVKYNSSSHLFYEEGNRHGRIYKKLGISHNQEEFN

SEQ ID NO: 254 - protein, 832 aa, unidentified
  REGION 1..832: note = Description of Unknown: Cas13c sequence
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 255 - protein, 802 aa, unidentified
  REGION 1..802: note = Description of Unknown: Cas13c sequence
MLKKPSNRYALPKVILSTVDHEKILEFKVKYEKLARLDRLVVERMHFDGESVVFDEVIANSGDLEIAYQDDHRKLLIQAAGKSYTITGKKVGGKKRKLEERISRAKIQLTLTDGQEDQHRRIRATVTEKALLEPKEDRDIYSKISDRKIKTSKEIYLVKRFLSYRSDLLFYYFFVDNFFKVGNNKQELWKIKFQNQPELIEYFRFIINDRFKNAKNDKFDNYLKNDKAIQEDLEKIQKVFEKLRHALMHYDYGFFEKLFGGEDQGFDLDIAFLDNFVKKIDKLNIDTKKEFVDDEKIKIFGEDLNLADLYKLYASISINRVGFNRVVNEMIIKDGIEKSELKRAFEKKLDKTYALDIHSDPSYKKLYNEHKRLVTEVSTYTDGNKIKEGNQKIAKLKYEMKEITKKNALVRLECKMRLAFGLIYGRYDTHEAFKNGFDTDLKRGEFAQIGSEEAIGYFNTTFEKSKPKSKEEIKKIARQIDNLSLSTLIEDDPLMKFIVLMFLFVPRELKGEFLGFWRKYYHDIHSIDSDAKSDEMPDEVSLSLKLKILTRNIRRLNLFEYSLSEKIKYSPKNTQFYTDKSPYQKVYKRLKISHNKEEFDKTLLVPLFRYYSILFKLINDFEIYSLAKANPDASSLSELTKTKHGFRGHYNFTTLMMDAHKVSQGDSKKHFGIRGEIAHINTKDLIYDPLFRKSKMAQQRNDVIDFVLKYEKEIKAVLGYDAINDFRMKVVQLRTKLKVYSDKTQTIEKLLNEVEAPDDFYVLYKVKGVEAINKYLLEIVSVTQAEEEIERKIITGNKRYNT

SEQ ID NO: 256 - protein, 806 aa, unidentified
  REGION 1..806: note = Description of Unknown: Cas13c sequence
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 257 - protein, 835 aa, unidentified
  REGION 1..835: note = Description of Unknown: Cas13c sequence
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 258 - protein, 847 aa, unidentified
  REGION 1..847: note = Description of Unknown: Cas13c sequence
MMTKKPANRHALPKVIISEVDNTNILEFKIKYEKLARLDRVEVKAMHYEDGRIIFDEVVVNGGLIEVEYQDDHKTLFVQVGEKSYSISGQKVGGKQRLLEDRVSKTKVQLELSDGSSERVSRTERELIVADNIKLYSQIVGHEVKTTKEIYLAKRFLGYRSDLLFYYGFVDNFFRESKNLKYGKQPVELWEDKFQVNDKLTAYTKFMFNDDLQNSESYLKEYVKDNHKIKNDLESARDIFATFRHNLMHFNYSFFTRLFNGEDVKIKNLQTKKFESLSDVLRNVEFLNKVIQSIDKLNIDTRKEFIDKEKITLFNEELDLQQLYGFFAYTAINRVAFNKLINSFIIKDGIENEQLKEYFNQRVDGTAYEIDIHQNREYKELYKKHKNLVSKVSTLSDGKEIARGNTEISVLKEQMNKITKANSLKRLEHKLRLAFGFIYTEYGSYKAFVSRFNEDTKRKKIKNVEFEKIGVEKQKEYYESTFTSNNKDKLGELIQEYEKLSLNDLIENDTFLKVILLLFIFMPKEVKGDFLGFIKKYYHDTKHIEEDTKEKDEGFTNTLPIGLKLKIVERNIAKLSVLKHSLSLKVKYNRGQYEEDNTYRKVFKKLNISHNQEEFHKSMFSPLLRYYASLYKLINDFEIYTLSHYITDKYSTLNKVIASEQFHYRYGWNREEKKGELVKTDNYTFSTLLSKKYGHKNSQEISEMRNKISHFDEKILFKFPLEEVSSVPKGKGKYKKDEPIKSLKEKREEIVSLMEKQTDMQKVLGYDAINDFRMKTVQFQTKLKVYSNKEETIKKMIVEAKTPNDYYNIYKVKGVEGINEHLLNVIGETEAEKSIQEQIAEGNKVNV

SEQ ID NO: 259 - protein, 779 aa, unidentified
  REGION 1..779: note = Description of Unknown: Cas13c sequence
ELCKIDFTDARSSSLIEYFKFAINDNLKNDRGYLKAYVNDVEQIRADLKKVGGKQRNLEDRVSRTKVQLTLTNHIEDREGKQRVSRTERELIVPQNIKLYSQIVGREVKTTKEIYLIKRFLEYRSDLLFYYGFVDNFFKVEGNKKELCKIDFTDARSSSLIEYFKFAINDNLKNDRGYLKAYVNDVEQIRTDLQKVKTIFSKLRHALMHFDYDFFEKLFNGEEVGFDFDIKFLNIMIDKVEKLNIETKKEFIEDEVITLFGERLSLKKLYGLFSHIAINRVAFNKFINSFLIKDGIENRALKDFFNDEKGSQAYEIDIHSNAEYKALYVQHKKLVMATSAMSDGNEIAKKNQEISELKEKMNAITKANSLARLEYKLRLAFGFIYTEYGDYTAFKNSFDRDVKSAKYKELSVERLKAYYLATFKASKPQSHEKLEEVAKKIDRLSLKQLIENETLLKFVLLLFTFMPQELKGEFLGFIKKYYHDKKHIEQDTKEKEEEREGLSTGLKLKVLEKNIRSLSILKHALSFQVKYNKKDKNFYEEGNLHGKFYKKLAISHNQEEFNKSVYAPLFRYYVALYKLINDFEIYSLAQHIVNNETLADQVGKAQFRQRGYFNFRKLVNCTYATAQNSSYNVLIFMRNDISHLSYEPLFNCPLEEKASYKQKIRGREKIISVKPLSESRAEIVRFIASQTDMKKLLGYDAVNDFNMKMVQLRRRLSVYANKQETIEKMINKAKTPNDFYNLYKLKGIECINQHLLKVIGVTEAEKRIEKQIEEGNEKY

SEQ ID NO: 260 - protein, 840 aa, unidentified
  REGION 1..840: note = Description of Unknown: Cas13c sequence
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 261 - protein, 840 aa, unidentified
  REGION 1..840: note = Description of Unknown: Cas13c sequence
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 262 - protein, 589 aa, unidentified
  REGION 1..589: note = Description of Unknown: Cas13c sequence
IQKFFDNGLDATKYDISTISLLKTLLEKTEEKIYHEKNNYIEDTDTLSIFDEKEIGFSKLHNFYTKISQKKPAFNKLINSFLSKDGVPNEPFKAYLHGKGYDYFEDIHADKSYKAIYVQHKLLVAQKQKEEAEEKPDGYKLKAYNDKLQELKIQMESITKANSLKRLEVKMRLAFGFITNEYKYDFKKFNAEFTLDVKTKEKLDRFKATSDERLQHYFESTFEEKTFFHFTVSSYDKKQKKSVEKVKTIFNLVENETLQTLVQDSPLLQIITLLYLFIPKELQGDFIGFILRIYHQIKNITSDTKEDEISIEESQNSFALKLKVLAKSLRGLQLFNYSLSHETLYNKNEHFFYEKGNRWKNIYKALGISHNTEEFDIHLVAPIIKYHINLYKLIGDFEIYALLTFAKKSRSHETLSVISKSDALKFKENYNFSTLLSKAFRIDVNNKNNPPYIQTLKQIRNNISHQNIEKMMTAFEQNDISKQRKEIIIYLQTDHQEMQKLLHYNPVNDFTMKTVQYRIMLDKYKTGMADNDERIENRADLIIKQLKKETPNDYYLIYKLKAIELLKQKMIEAIGETEQEKKIRKAIAK

SEQ ID NO: 263 - RNA, 37 nt, synthetic construct
  misc_feature 1..37: note = Description of Artificial Sequence: Synthetic oligonucleotide
  source 1..37: mol_type = other RNA
attgctccttacgaggagacgagctggtggcgtaggc

SEQ ID NO: 264 - RNA, 37 nt, synthetic construct
  misc_feature 1..37: note = Description of Artificial Sequence: Synthetic oligonucleotide
  source 1..37: mol_type = other RNA
attgctccttacgaggagacgagctgatggcgtaggc

SEQ ID NO: 265 - RNA, 53 nt, synthetic construct
  misc_feature 1..53: note = Description of Artificial Sequence: Synthetic oligonucleotide
  source 1..53: mol_type = other RNA
ctttcaagactaatagattgctccttacgaggagacgagctggtggcgtaggc

SEQ ID NO: 266 - RNA, 53 nt, synthetic construct
  misc_feature 1..53: note = Description of Artificial Sequence: Synthetic oligonucleotide
  source 1..53: mol_type = other RNA
ctttcaagactaatagattgctccttacgaggagacgagctgatggcgtaggc

SEQ ID NO: 267 - RNA, 53 nt, synthetic construct
  misc_feature 1..53: note = Description of Artificial Sequence: Synthetic oligonucleotide
  source 1..53: mol_type = other RNA
ctttcaagactaatagattgctccttacgaggagaccctacgccatcagctcc

SEQ ID NO: 268 - RNA, 20 nt, synthetic construct
  modified_base 2: mod_base = OTHER; note = thymine
  modified_base 5..6: mod_base = OTHER; note = thymine
  modified_base 12: mod_base = OTHER; note = thymine
  modified_base 15: mod_base = OTHER; note = thymine
  modified_base 20: mod_base = OTHER; note = thymine
  misc_feature 1..20: note = Description of Artificial Sequence: Synthetic oligonucleotide
  source 1..20: mol_type = other RNA
gtagttggagctggtggcgt

SEQ ID NO: 269 - RNA, 20 nt, synthetic construct
  modified_base 2: mod_base = OTHER; note = thymine
  modified_base 5..6: mod_base = OTHER; note = thymine
  modified_base 12: mod_base = OTHER; note = thymine
  modified_base 15: mod_base = OTHER; note = thymine
  modified_base 20: mod_base = OTHER; note = thymine
  misc_feature 1..20: note = Description of Artificial Sequence: Synthetic oligonucleotide
  source 1..20: mol_type = other RNA
gtagttggagctgatggcgt

SEQ ID NO: 270 - DNA, 36 bp, synthetic construct
  misc_feature 1..36: note = Description of Artificial Sequence: Synthetic oligonucleotide
  source 1..36: mol_type = other DNA
gttagaatataaccctgtttgtaggggtaataaaac

SEQ ID NO: 271 - DNA, 36 bp, synthetic construct
  misc_feature 1..36: note = Description of Artificial Sequence: Synthetic oligonucleotide
  source 1..36: mol_type = other DNA
ttgactacactctctatctcttagggagactgaaac

SEQ ID NO: 272 - DNA, 36 bp, synthetic construct
  misc_feature 1..36: note = Description of Artificial Sequence: Synthetic oligonucleotide
  source 1..36: mol_type = other DNA
actagactatacccccatttgagaggggactaaaac

SEQ ID NO: 273 - DNA, 25 bp, synthetic construct
  misc_feature 1..25: note = Description of Artificial Sequence: Synthetic oligonucleotide
  source 1..25: mol_type = other DNA
ccagctccaactaccacaagtttat

SEQ ID NO: 274 - DNA, 25 bp, synthetic construct
  misc_feature 1..25: note = Description of Artificial Sequence: Synthetic oligonucleotide
  source 1..25: mol_type = other DNA
accagctccaactaccacaagttta

SEQ ID NO: 275 - DNA, 25 bp, synthetic construct
  misc_feature 1..25: note = Description of Artificial Sequence: Synthetic oligonucleotide
  source 1..25: mol_type = other DNA
caccagctccaactaccacaagttt

SEQ ID NO: 276 - DNA, 25 bp, synthetic construct
  misc_feature 1..25: note = Description of Artificial Sequence: Synthetic oligonucleotide
  source 1..25: mol_type = other DNA
ccaccagctccaactaccacaagtt

SEQ ID NO: 277 - DNA, 25 bp, synthetic construct
  misc_feature 1..25: note = Description of Artificial Sequence: Synthetic oligonucleotide
  source 1..25: mol_type = other DNA
gccaccagctccaactaccacaagt

SEQ ID NO: 278 - DNA, 25 bp, synthetic construct
  misc_feature 1..25: note = Description of Artificial Sequence: Synthetic oligonucleotide
  source 1..25: mol_type = other DNA
cgccaccagctccaactaccacaag

SEQ ID NO: 279 - DNA, 25 bp, synthetic construct
  misc_feature 1..25: note = Description of Artificial Sequence: Synthetic oligonucleotide
  source 1..25: mol_type = other DNA
acgccaccagctccaactaccacaa

SEQ ID NO: 280 - DNA, 25 bp, synthetic construct
  misc_feature 1..25: note = Description of Artificial Sequence: Synthetic oligonucleotide
  source 1..25: mol_type = other DNA
tacgccaccagctccaactaccaca

SEQ ID NO: 281 - DNA, 25 bp, synthetic construct
  misc_feature 1..25: note = Description of Artificial Sequence: Synthetic oligonucleotide
  source 1..25: mol_type = other DNA
ctacgccaccagctccaactaccac

SEQ ID NO: 282 - DNA, 25 bp, synthetic construct
  misc_feature 1..25: note = Description of Artificial Sequence: Synthetic oligonucleotide
  source 1..25: mol_type = other DNA
cctacgccaccagctccaactacca

SEQ ID NO: 283 - DNA, 25 bp, synthetic construct
  misc_feature 1..25: note = Description of Artificial Sequence: Synthetic oligonucleotide
  source 1..25: mol_type = other DNA
gcctacgccaccagctccaactacc

SEQ ID NO: 284 - DNA, 25 bp, synthetic construct
  misc_feature 1..25: note = Description of Artificial Sequence: Synthetic oligonucleotide
  source 1..25: mol_type = other DNA
tgcctacgccaccagctccaactac

SEQ ID NO: 285 - RNA, 53 nt, synthetic construct; identical to SEQ ID NO: 265
  misc_feature 1..53: note = Description of Artificial Sequence: Synthetic oligonucleotide
  source 1..53: mol_type = other RNA

(19) United States
(12) Patent Application Publication
(10) Pub. No.: US 2024/0084275 A1
(43) Pub. Date: Mar. 14, 2024

(54) COMPOSITIONS AND METHODS OF USING PROGRAMMABLE NUCLEASES FOR INDUCING CELL DEATH

(71) Applicant: Mammoth Biosciences, Inc., Brisbane, CA (US)

(72) Inventors: Lucas Benjamin HARRINGTON, San Francisco, CA (US); Pei-Qi LIU, Oakland, CA (US); Subhadra JAYARAMAN RUKMINI, Redwood City, CA (US)

(21) Appl. No.: 18/336,718

(22) Filed: Jun. 16, 2023

Related U.S. Application Data
(63) Continuation of application No. PCT/US21/64904, filed on Dec. 22, 2021.
(60) Provisional application No. 63/129,898, filed on Dec. 23, 2020.
(60) Provisional application No. 63/239,338, filed on Aug. 31, 2021.

(51) Int. Cl. C12N 9/22; C12N 15/10

(52) U.S. Cl. CPC C12N 9/22; C12N 15/102; C12N 2310/20

(57) ABSTRACT

Disclosed herein, in certain embodiments, are methods of inducing cell cycle arrest, apoptosis, cell death, or a combination thereof, in a cell or population of cells. Also disclosed herein are methods of treating a disease or condition in an individual in need thereof comprising inducing cell cycle arrest, apoptosis, cell death, or a combination thereof in a population of cells in the individual. The cell or population of cells may comprise a nucleic acid sequence associated with a disease or condition, including an autoimmune disease, cancer, or an infectious disease. The methods described herein generally comprise contacting cells with a CRISPR-associated protein and a guide nucleic acid.

### CROSS REFERENCE

The present application is a continuation of International Patent Application No. PCT/US21/64904, filed Dec. 22, 2021, which claims the benefit of U.S. Provisional Application No. 63/129,898, filed on Dec. 23, 2020, and U.S. Provisional Application No. 63/239,338, filed on Aug. 31, 2021, the entire contents of each of which are herein incorporated by reference.

### INCORPORATION BY REFERENCE OF SEQUENCE LISTING

The instant application contains a Sequence Listing which has been submitted electronically. The Sequence Listing titled 203477-738301_US_SL.xml, which was created on Jun. 15, 2023 and is 518,934 bytes in size, is hereby incorporated by reference in its entirety.

### BACKGROUND

Bacterial adaptive immune systems employ CRISPRs (clustered regularly interspaced short palindromic repeats) and CRISPR-associated (Cas) proteins for RNA-guided nucleic acid cleavage. Various CRISPR-associated proteins (e.g., CRISPR Type VI and V guided nucleases) have been shown to exert cleavage of nucleic acids not only in cis, but in trans. Such CRISPR proteins can become activated after binding of a guide nucleic acid with a target nucleic acid, in which the activated programmable nuclease can cleave the target nucleic acid and can have trans cleavage activity, which can also be referred to as “collateral” or “transcollateral” cleavage. Trans cleavage activity can be non-specific cleavage of nearby single-stranded nucleic acids by the activated programmable nuclease. For example, CRISPR proteins such as Cas12a and Cas13a are capable of nonspecific cleavage of ssDNA (single-stranded DNA) and RNA, respectively, in addition to cis cleavage of a target nucleic acid strand hybridized to an RNA guide. CRISPR systems thus have been leveraged to induce collateral cleavage of, for example, ssDNA reporters, initiated by the recognition and cleavage of a target DNA or RNA, which can then be used for the detection of the target DNA.

### SUMMARY

Provided herein, in some embodiments, is a method of inducing cell cycle arrest, apoptosis, cell death, or a combination thereof, in a cell, the method comprising: contacting a CRISPR-associated protein and a guide nucleic acid molecule to a nucleic acid target site within the cell, wherein the guide nucleic acid molecule is complementary to at least a portion of the nucleic acid target site, and wherein hybridization of the guide nucleic acid molecule to the nucleic acid target site activates non-specific cleavage of DNA, RNA, or a combination thereof in the cell and induces cell cycle arrest, apoptosis, cell death, or a combination thereof, of the cell. Also provided herein, in some embodiments, is a method of inducing cell cycle arrest, apoptosis, cell death, or a combination thereof, in a cell, the method comprising: contacting a CRISPR-associated protein and a guide nucleic acid molecule to a nucleic acid target site within the cell, wherein the guide nucleic acid molecule is complementary to at least a portion of the nucleic acid target site, and wherein hybridization of the guide nucleic acid molecule to the nucleic acid target site activates non-specific cleavage of DNA in the cell and induces cell cycle arrest, apoptosis, cell death, or a combination thereof, of the cell. Also provided herein, in some embodiments, is a method of treating a disease or condition in an individual in need thereof, the method comprising: administering to a population of cells in the individual a CRISPR-associated protein and a guide nucleic acid molecule complementary to at least a portion of a nucleic acid target site, wherein at least a portion of the cell population comprises the nucleic acid target site, wherein hybridization of the guide nucleic acid molecule to the nucleic acid target site activates non-specific cleavage of DNA, RNA, or a combination thereof in the cell population and induces cell cycle arrest, apoptosis, cell death, or a combination thereof in one or more cells within the cell population. In some embodiments, the CRISPR-associated protein induces cell cycle arrest, apoptosis, or cell death of at least 50% of the cells in the cell population as determined by an in vitro viability assay, proliferation assay, apoptosis assay, or cell cycle or DNA damage assay. In some embodiments, the method further comprises administering a second guide nucleic acid molecule complementary to a second nucleic acid target site. In some embodiments, the method further comprises administering a third guide nucleic acid molecule complementary to a third nucleic acid target site. In some embodiments, the nucleic acid target site comprises a DNA molecule. In some embodiments, the nucleic acid target site comprises an RNA molecule. In some embodiments, the hybridization of the guide nucleic acid molecule activates non-specific cleavage of a DNA molecule within the cell or the cell population. In some embodiments, the non-specific cleavage introduces a single-stranded break in the DNA molecule. In some embodiments, the hybridization of the guide nucleic acid molecule activates non-specific cleavage of an RNA molecule within the cell or the cell population. In some embodiments, the non-specific cleavage introduces a single-stranded break in the RNA molecule. In some embodiments, the CRISPR-associated protein comprises an amino acid sequence that is at least 95% identical to any one of SEQ ID NOS: 1-220, 244, and 248-262 herein. In some embodiments, the CRISPR-associated protein comprises a RuvC domain. In some embodiments, the CRISPR-associated protein comprises three partial RuvC domains. In some embodiments, the CRISPR-associated protein comprises at least one HEPN domain. In some embodiments, the CRISPR-associated protein comprises two HEPN domains. In some embodiments, the CRISPR-associated protein comprises a Cas12, Cas13, Cas14, or CasΦ protein, or a catalytically active fragment thereof. In some embodiments, the CRISPR-associated protein comprises a Cas12a, Cas12b, Cas12c, Cas12d, or Cas12e protein. In some embodiments, the CRISPR-associated protein comprises a Cas13a, Cas13b, Cas13c, Cas13d, or Cas13e protein. In some embodiments, the CRISPR-associated protein comprises a Cas14a, Cas14b, Cas14c, Cas14d, Cas14e, Cas14f, Cas14g, Cas14h, Cas14i, Cas14j, or Cas14k protein. In some embodiments, the CRISPR-associated protein comprises a CasΦ protein having an amino acid sequence at least 80% identical to any one of SEQ ID NO: 155-SEQ ID NO: 202 herein. In some embodiments, the CRISPR-associated protein comprises a CasΦ protein having an amino acid sequence comprising any one of SEQ ID NO: 155-SEQ ID NO: 202 herein. In some embodiments, the CRISPR-associated protein is a fusion protein. In some embodiments, the fusion protein comprises an enzymatically inactive CRISPR-associated protein and a polypeptide that exhibits nuclease activity. In some embodiments, the polypeptide that exhibits nuclease activity comprises a restriction enzyme. In some embodiments, the hybridization of the guide nucleic acid molecule to the nucleic acid target site induces a conformational change in the CRISPR-associated protein, and the conformational change releases the restriction enzyme. In some embodiments, the cell is a member of a cell population and wherein at least a portion of the cells within the cell population comprise the nucleic acid target site. In some embodiments, the cell is a cancer cell or the cell population is a cancer cell population. In some embodiments, the cancer cell population is associated with retinoblastoma, glioblastoma, lung cancer, or liver cancer. In some embodiments, the nucleic acid target site comprises a DNA or RNA molecule associated with a cancer. In some embodiments, the nucleic acid target site comprises any of the following cancer-associated genes, or a portion thereof: RB1, KRAS, p53, CDKN2A, EGFR, BRCA1, BRCA2, and HER2. In some embodiments, the nucleic acid target site is located in an oncogene selected from: NRAS, TP53, BRAF, MYC, CTNNB1, CREBBP, EGFR, RB1, PTEN, and JAK1. In some embodiments, the cell population is an autoimmune disease cell population. In some embodiments, the cell population is a causative immune cell population for an autoimmune disease. In some embodiments, the causative immune cell population comprises one or more autoimmune antibodies. In some embodiments, the cell population is an infectious disease cell population. In some embodiments, the infectious disease cell population comprises one or more host cells comprising a viral genome or a portion thereof. In some embodiments, the nucleic acid target site comprises any of the following genes, or a portion thereof: an HBV gene, an HCV gene or an HIV gene. In some embodiments, the method further comprises administering an additional therapeutic agent. In some embodiments, the additional therapeutic agent is an anti-PD1 agent. In some embodiments, the additional therapeutic agent is a PARP inhibitor. In some embodiments, the CRISPR complex is present in one or more nanoparticles. In some embodiments, the CRISPR complex is encoded for by a polynucleotide comprised in one or more delivery vectors. In some embodiments, the CRISPR complex is comprised in a pharmaceutical composition comprising (i) any one of the CRISPR complexes disclosed herein, a delivery vector, any one of the nanoparticles disclosed herein, and (ii) a pharmaceutically acceptable excipient. In some embodiments, contacting the CRISPR-associated protein to the nucleic acid target site within the cell or cell population comprises contacting the cell or cell population with an mRNA encoding the CRISPR-associated protein. In some embodiments, the method comprises contacting the cell with a lipid nanoparticle (LNP) comprising the mRNA, the guide nucleic acid molecule, or a combination thereof. In some embodiments, the CRISPR-associated protein comprises an amino acid sequence that is at least 90% identical to SEQ ID NO: 166. In some embodiments, the CRISPR-associated protein comprises an amino acid sequence that is at least 95% identical to SEQ ID NO: 166. In some embodiments, the CRISPR-associated protein comprises an amino acid sequence that is 100% identical to SEQ ID NO: 166. In some embodiments, the CRISPR-associated protein comprises an amino acid sequence that is 100% identical to any one of SEQ ID NOS: 248-262.

Provided herein, in some embodiments, is the use of the CRISPR-associated protein and any one of the guide nucleic acid molecule or guide nucleic acid molecules disclosed herein for inducing growth arrest, cell death, or a combination thereof in a cell population. In some embodiments, the cell population is a cancer cell population. In some embodiments, the cancer cell population is associated with retinoblastoma, glioblastoma, lung cancer, liver cancer, leukemia, or lymphoma. In some embodiments, the cell population is an autoimmune disease cell population. In some embodiments, the cell population is an infectious disease cell population. In some embodiments, the infectious disease cell population is associated with HBV, HCV, or HIV.

Provided herein, in some embodiments, is the use of the CRISPR-associated protein and the guide nucleic acid molecule or guide nucleic acid molecules in combination with an additional therapeutic agent for inducing growth arrest, cell death, or a combination thereof in a cell population. In some embodiments, the additional therapeutic agent is an anti-PD1 agent. In some embodiments, the additional therapeutic agent is a PARP inhibitor. In some embodiments, the cell population is a cancer cell population. In some embodiments, the cancer cell population is associated with retinoblastoma, glioblastoma, lung cancer, liver cancer, leukemia, or lymphoma. In some embodiments, the cell population is an autoimmune disease cell population. In some embodiments, the cell population is an infectious disease cell population. In some embodiments, the infectious disease cell population is associated with HBV, HCV, or HIV.

Provided herein, in some embodiments, is a composition comprising a CRISPR-associated protein, or a nucleic acid encoding the CRISPR-associated protein, and a guide nucleic acid molecule, wherein a) the CRISPR-associated protein is selected from a Type V guided nuclease or Type VI guided nuclease, and b) the guide nucleic acid molecule comprises a nucleotide sequence that is identical or reverse complementary to an equal length portion of a target nucleic acid that comprises a mutation of at least one nucleotide relative to a corresponding wildtype sequence. In some embodiments, the Type V or Type VI guide nuclease is selected from a Cas12, Cas13, Cas14, or CasΦ protein, or a catalytically active fragment thereof. In some embodiments, the CRISPR-associated protein comprises a Cas12a, Cas12b, Cas12c, Cas12d, or Cas12e protein; a Cas13a, Cas13b, Cas13c, Cas13d, or Cas13e protein; or a Cas14a, Cas14b, Cas14c, Cas14d, Cas14e, Cas14f, Cas14g, Cas14h, Cas14i, Cas14j, or Cas14k protein. In some embodiments, the CRISPR-associated protein comprises an amino acid sequence that is at least 75%, at least 80%, at least 85%, at least 90%, at least 95% or 100% identical to any one of SEQ ID NOs: 1-220, 244, and 248-262. In some embodiments, the amino acid sequence of the CRISPR-associated protein is at least 75%, at least 80%, at least 85%, at least 90%, at least 95% or 100% identical to any one of SEQ ID NO: 1-220, 244, and 248-262. In some embodiments, the mutation is selected from a nucleotide deletion, a nucleotide insertion, and a nucleotide substitution. In some embodiments, the mutation is a single nucleotide polymorphism (SNP). In some embodiments, the nucleotide sequence that is identical or reverse complementary to the equal length portion of the target nucleic acid comprises 4, 5, 6, 7, 8, 9, 10, 11, 12, 13, 14, 15, 16, 17, 18, 19 or 20 nucleobases. In some embodiments, the target nucleic acid is a gene selected from RB1, KRAS, p53, CDKN2A, EGFR, BRCA1, BRCA2, and HER2, or a portion thereof. In some embodiments, the target nucleic acid is located in an oncogene selected from: NRAS, TP53, BRAF, MYC, CTNNB1, CREBBP, EGFR, RB1, PTEN, and JAK1. In some embodiments, the target nucleic acid is KRAS, or a portion thereof. In some embodiments, the mutation is selected from KRAS p.G12C—c.34G>T; KRAS p.G12D—c.35G>A; and KRAS p.G12V—c.35G>T. In some embodiments, the mutation is KRAS p.G12D. In some embodiments, the mutation is KRAS p.G12D—c.35G>A, and the guide nucleic acid molecule comprises a nucleotide sequence selected from SEQ ID NOS: 226, 227, 228, 236, 238, 240, 242, 264, 266, 267, and 269. In some embodiments, the mutation is KRAS p.G12D—c.35G>A, and the guide nucleic acid molecule comprises a nucleotide sequence selected from SEQ ID NOS: 222, 237, 238, 243, 246, 263, 264, 265, 266, 267, 268, and 285. In some embodiments, the mutation is KRAS p.G12V—c.35G>T, and the guide nucleic acid molecule comprises a nucleotide sequence selected from TGGTAGTTGGAGCTGTT (SEQ ID NO: 229); GAGCTGTTGGCGTAGGC (SEQ ID NO: 230); and CCTACGCCAACAGCTCC (SEQ ID NO: 231). In some embodiments, the mutation is KRAS p.G12C—c.34G>T, and the guide nucleic acid molecule comprises a nucleotide sequence selected from TGGTAGTTGGAGCTTGT (SEQ ID NO: 232); GAGCTTGTGGCGTAGGC (SEQ ID NO: 233); and CCTACGCCACAAGCTCC (SEQ ID NO: 234). In some embodiments, the CRISPR-associated protein comprises an amino acid sequence that is at last 95% identical to SEQ ID NO: 166, and wherein the guide nucleic acid comprises a nucleotide sequence that is at least 90% identical to a sequence selected from SEQ ID NOS: 236, 240, 264, 266, and 267. In some embodiments, the CRISPR-associated protein comprises an amino acid sequence that is at last 95% identical to SEQ ID NO: 166, and wherein the guide nucleic acid comprises a nucleotide sequence selected from SEQ ID NOS: 236, 240, 264, 266, and 267. In some embodiments, the target nucleic acid comprises a protospacer adjacent motif of 5′-NTTN′-3,′ optionally wherein the PAM is 5′ of the target sequence of a non-complementary strand of the target nucleic acid. In some embodiments, the nucleic acid encoding the CRISPR-associated protein is a messenger RNA (mRNA). In some embodiments, the nucleic acid encoding the CRISPR-associated protein is an expression vector. In some embodiments, the expression vector is a viral vector.

Provided herein, in some embodiments, is a method of modifying a target nucleic acid in a cell, comprising contacting the cell with any of the compositions disclosed herein. Also provided herein, in some embodiments, is a method of selectively modifying a portion of cells within a population of cells, the method comprising contacting the population of cells with any one of the compositions disclosed herein, wherein the portion of cells comprises the target nucleic acid that comprises the mutation, and the remaining cells comprise the corresponding wildtype sequence. Provided herein, in some embodiments, is a method of modifying expression of a target nucleic acid in a portion of cells within a population of cells, the method comprising contacting the population of cells with any one of the compositions disclosed herein, wherein the portion of cells comprises the target nucleic acid that comprises the mutation, and the remaining cells comprise the corresponding wildtype sequence. Provided herein, in some embodiments, is a method of reducing cell viability, reducing cell proliferation, or increasing cell death of a portion of cells within a population of cells, the method comprising contacting the population of cells with any one of the compositions disclosed herein, wherein the portion of cells comprises the target nucleic acid that comprises the mutation, and the remaining cells comprise the corresponding wildtype sequence. In some embodiments, the cell viability of the portion of the cells is reduced by at least 50%, and cell viability of the remaining cells is reduced by no more than 10%, as measured with a cell viability assay. In some embodiments, proliferation of the portion of the cells is reduced by at least 50%, and proliferation of the remaining cells is reduced by no more than 10%, as measured with a colony forming assay. In some embodiments, cell death of the portion of the cells is increased by at least 50%, and cell death of the remaining cells is increased by no more than 10%, as measured with a cell viability assay or a colony forming assay. In some embodiments, contacting modifies the nucleotide sequence of the target nucleic acid. In some embodiments, modifying expression comprises increasing expression. In some embodiments, modifying expression comprises reducing expression. In some embodiments, the cell or portion of cells comprises a cancer associated mutation. In some embodiments, the cancer associated mutation is a mutation associated with pancreatic cancer. In some embodiments, the cell or portion of cells are pancreatic cancer cells.

Provided herein, in some embodiments, is a cell comprising any one of the compositions disclosed herein. Also provided herein, is a cell or portion of a population of cells modified according to any one of the methods disclosed herein.

Provided herein, in some embodiments, is a method of selectively modifying a first portion of cells within a cell population, the method comprising contacting the cell population with any one of the compositions disclosed herein, wherein modifying the first portion of the cells comprises modifying a first target nucleic acid in the first portion of cells, wherein modification of the first target nucleic acid in the first portion of cells is greater than modification of a second target nucleic acid in a second portion of the cells in the cell population. In some embodiments, modification of the first portion of the cells and the second portion of the cells is quantified by indel formation. In some embodiments, indel formation in the second portion of the cells is less than 10%. In some embodiments, indel formation in the second portion of the cells is less than 5%. In some embodiments, indel formation in the second portion of the cells is less than 1%. In some embodiments, the indel formation in the first portion of cells is at least 30% greater than indel formation in the second portion of the cells. In some embodiments, the indel formation in the first portion of cells is at least about 40% greater than indel formation in the second portion of the cells. In some embodiments, the second target nucleic acid is a wildtype allele of a gene, and the first target nucleic acid is a mutant allele of the gene, and the second portion of cells does not comprise the mutant allele of the gene. In some embodiments, the gene is an oncogene. In some embodiments, the gene is selected from RB1, KRAS, TP53, CDKN2A, EGFR, BRCA1, BRCA2, HER2, NRAS, BRAF, MYC, CTNNB1, CREBBP, EGFR, PTEN, and JAK1. In some embodiments, the gene is KRAS. In some embodiments, the mutant allele of KRAS comprises a mutation selected from: KRAS p.G12C—c.34G>T; KRAS p.G12D—c.35G>A; and KRAS p.G12V—c.35G>T. In some embodiments, the mutant allele of KRAS comprises the mutation, KRAS p.G12D—c.35G>A. In some embodiments, modifying the first target nucleic acid reduces expression of the first target nucleic acid in the first portion of the target nucleic acid. In some embodiments, the cell population comprises pancreatic cells, wherein the first portion of cells are pancreatic cancer cells, and wherein the second portion of cells are not cancer cells. In some embodiments, the method results in cell death of the first portion of the cells. In some embodiments, the seed region of the guide nucleic acid molecule comprises at least 16 nucleotides, and the seed region is 100% complementary to an equal length portion of the first target nucleic acid. In some embodiments, the CRISPR-associated protein comprises an amino acid sequence that is at least 95% identical to SEQ ID NO: 166. In some embodiments, the guide nucleic acid molecule comprises a chemical modification of at least one nucleotide or internucleotide linkage; optionally wherein the chemical modification is selected from: a 2′ O-methyl, a 2′-fluoro, a locked nucleic acid (LNA), a peptide nucleic acid (PNA), a phosphorothioate linkage, and a 5′ cap, and a combination thereof.

Provided herein, in some embodiments, is a method of inducing death of a human cell comprising at least one allele with a genetic mutation, the method comprising: contacting the human cell with a Cas13 protein and a guide nucleic acid molecule that hybridizes to a target sequence of a target mRNA, wherein the target sequence is identical, complementary, or reverse complementary to a portion of the allele comprising the mutation. In some embodiments, the at least one allele is an allele of KRAS. In some embodiments, the genetic mutation is selected from: p.G12D—c.35G>A; p.G12V—c.35G>T; and p.G12C—c.34G>T. In some embodiments, the Cas13 protein cleaves at least one non-target nucleic acid not comprising the target sequence. In some embodiments, a) the Cas13 protein is at least 95% identical to SEQ ID NO: 248; and the guide nucleic acid molecule comprises a repeat sequence that is at least 90% identical to SEQ ID NO: 270 and a spacer sequence that is at least 90% identical to SEQ ID NO: 273; b) the Cas13 protein is at least 95% identical to SEQ ID NO: 249; and the guide nucleic acid molecule comprises a repeat sequence that is at least 90% identical to SEQ ID NO: 270 and a spacer sequence that is at least 90% identical to SEQ ID NO: 274; c) the Cas13 protein is at least 95% identical to SEQ ID NO: 250; and the guide nucleic acid molecule comprises a repeat sequence that is at least 90% identical to SEQ ID NO: 270 and a spacer sequence that is at least 90% identical to SEQ ID NO: 275; d) the Cas13 protein is at least 95% identical to SEQ ID NO: 251; and the guide nucleic acid molecule comprises a repeat sequence that is at least 90% identical to SEQ ID NO: 270 and a spacer sequence that is at least 90% identical to SEQ ID NO: 276; e) the Cas13 protein is at least 95% identical to SEQ ID NO: 252; and the guide nucleic acid molecule comprises a repeat sequence that is at least 90% identical to SEQ ID NO: 270 and a spacer sequence that is at least 90% identical to SEQ ID NO: 277; f) the Cas13 protein is at least 95% identical to SEQ ID NO: 253; and the guide nucleic acid molecule comprises a repeat sequence that is at least 90% identical to SEQ ID NO: 270 and a spacer sequence that is at least 90% identical to SEQ ID NO: 278; g) the Cas13 protein is at least 95% identical to SEQ ID NO: 254; and the guide nucleic acid molecule comprises a repeat sequence that is at least 90% identical to SEQ ID NO: 270 and a spacer sequence that is at least 90% identical to SEQ ID NO: 279; h) the Cas13 protein is at least 95% identical to SEQ ID NO: 255; and the guide nucleic acid molecule comprises a repeat sequence that is at least 90% identical to SEQ ID NO: 270 and a spacer sequence that is at least 90% identical to SEQ ID NO: 280; i) the Cas13 protein is at least 95% identical to SEQ ID NO: 256; and the guide nucleic acid molecule comprises a repeat sequence that is at least 90% identical to SEQ ID NO: 270 and a spacer sequence that is at least 90% identical to SEQ ID NO: 281; j) the Cas13 protein is at least 95% identical to SEQ ID NO: 257; and the guide nucleic acid molecule comprises a repeat sequence that is at least 90% identical to SEQ ID NO: 270 and a spacer sequence that is at least 90% identical to SEQ ID NO: 282; k) the Cas13 protein is at least 95% identical to SEQ ID NO: 258; and the guide nucleic acid molecule comprises a repeat sequence that is at least 90% identical to SEQ ID NO: 270 and a spacer sequence that is at least 90% identical to SEQ ID NO: 283; 1) the Cas13 protein is at least 95% identical to SEQ ID NO: 259; and the guide nucleic acid molecule comprises a repeat sequence that is at least 90% identical to SEQ ID NO: 270 and a spacer sequence that is at least 90% identical to SEQ ID NO: 284; m) the Cas13 protein is at least 95% identical to SEQ ID NO: 260; and the guide nucleic acid molecule comprises a repeat sequence that is at least 90% identical to SEQ ID NO: 271 and a spacer sequence that is at least 90% identical to SEQ ID NO: 273; n) the Cas13 protein is at least 95% identical to SEQ ID NO: 261; and the guide nucleic acid molecule comprises a repeat sequence that is at least 90% identical to SEQ ID NO: 271 and a spacer sequence that is at least 90% identical to SEQ ID NO: 274; or o) the Cas13 protein is at least 95% identical to SEQ ID NO: 262; and the guide nucleic acid molecule comprises a repeat sequence that is at least 90% identical to SEQ ID NO: 272 and a spacer sequence that is at least 90% identical to SEQ ID NO: 273.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a CRISPR protein complexed with a guide RNA inducing cis-cleavage of a target DNA molecule (top) and trans cleavage of an off-target DNA molecule (bottom).

FIG. 2 shows the results of a cell viability assay performed on KRAS mutant pancreatic cells electroporated with Casφ.12 or Cas9 and guide nucleic acids specific for wildtype or mutant KRAS alleles.

FIG. 3 shows Casφ.12 is intolerant of one or two nucleotide mismatches in the first 16 nucleotides of a guide RNA.

FIG. 4A shows indel formation by Casφ.12 in a pancreatic cell line expressing wildtype KRAS.

FIG. 4B shows indel formation by Casφ.12 in a pancreatic cell line expressing a mutant KRAS.

FIG. 5A shows indel formation by Casφ.12 with chemically modified guide RNAs in a pancreatic cell line expressing wildtype KRAS.

FIG. 5B shows indel formation by Casφ.12 with chemically modified guide RNAs in a pancreatic cell line expressing a mutant KRAS.

### DETAILED DESCRIPTION

Provided herein, are methods of using systems comprising a CRISPR protein, also referred to herein as a CRISPR-associated protein or a CRISPR/Cas enzyme, to induce cell death, cell-cycle arrest, apoptosis, or combinations thereof in populations of cells leveraging the trans cleavage activity of said CRISPR proteins. In some examples, the trans cleavage activity of a CRISPR protein (e.g., a CRISPR Type V or Type VI guided nuclease) can be leveraged to induce cell death, cell-cycle arrest, apoptosis, or combinations thereof in a population of cells. The population of cells can be a population of cancer cells, cells infected with a pathogen, or a causative population of cells of an autoimmune disorder. In some examples, inducing cell death of the population of cells treats the cancer, infectious disease, or autoimmune disease in an individual in need thereof. For example, non-specific trans cleavage of nucleic acids in the host cell of a virus can be sufficient to arrest the growth of the host cell and stop the infectious cycle. Similarly, non-specific trans cleavage of nucleic acids in cancer cells can be sufficient to induce cell death of the cancer cell. In some examples, CRISPR proteins induce non-specific cleavage of a plurality of single-stranded DNA molecules within a population of cells. In some examples, non-specifically cleaving single stranded DNA in a disease cell, as compared to single stranded RNA, is preferable as a more efficient manner of inducing cell death, apoptosis, or a combination thereof in the disease cell and/or population of disease cells.

In some aspects, provided herein are CRISPR-associated proteins that are complexed with a guide RNA molecule and can bind to a target DNA molecule (e.g., a nucleic acid target site). The CRISPR-associated protein and guide RNA molecule can form a CRISPR-Cas nucleoprotein complex with trans cleavage activity, which can be activated by binding of a guide nucleic acid with a target nucleic acid. In some examples, when the programmable nuclease is complexed with the guide RNA and the target DNA hybridizes to the guide RNA, trans-cleavage of one or more nucleic acids by the programmable nuclease is activated. In some examples, binding of the guide nucleic acid with the target nucleic acid causes promiscuous cleavage of DNA and RNA molecules within a population of disease cells, e.g., host cells forming a population of cancer cells, infected cells, or cells causative of an autoimmune disorder. In some examples, the promiscuous cleavage is sufficient to induce cell death, apoptosis, cell cycle arrest, or a combination thereof, within the population of disease cells, thereby treating the cancer, infectious disease, or autoimmune disorder.

Described herein, in some instances, are methods of inducing cell cycle arrest, apoptosis, cell death, or a combination thereof in a cell, the method comprising: contacting a CRISPR-associated protein and a guide nucleic acid molecule to a nucleic acid target site within the cell, wherein the guide nucleic acid molecule is complementary to at least a portion of the nucleic acid target site, and wherein hybridization of the guide nucleic acid molecule to the nucleic acid target site activates non-specific cleavage of DNA, RNA, or a combination thereof in the cell and induces cell cycle arrest, apoptosis, cell death, or a combination thereof, of the cell.

Also described herein, are methods of inducing cell cycle arrest, apoptosis, cell death, or a combination thereof in a cell, the method comprising: contacting a CRISPR-associated protein and a guide nucleic acid molecule to a nucleic acid target site within the cell, wherein the guide nucleic acid molecule is complementary to at least a portion of the nucleic acid target site, and wherein hybridization of the guide nucleic acid molecule to the nucleic acid target site activates non-specific cleavage of DNA in the cell and induces cell cycle arrest, apoptosis, cell death, or a combination thereof, of the cell.

Also described herein, are methods of treating a disease or condition in an individual in need thereof, the method comprising: administering to a population of cells in the individual a CRISPR-associated protein and a guide nucleic acid molecule complementary to at least a portion of a nucleic acid target site, wherein at least a portion of the cell population comprises the nucleic acid target site, wherein hybridization of the guide nucleic acid molecule to the nucleic acid target site activates non-specific cleavage of DNA, RNA, or a combination thereof in the cell population and induces cell cycle arrest, apoptosis, cell death, or a combination thereof in one or more cells within the cell population.

Also described herein, are uses of the CRISPR-associated protein and the guide nucleic acid molecule or guide nucleic acid molecules described herein for inducing growth arrest, cell death, or a combination thereof in a cell population.

Also described herein, are uses of the CRISPR-associated proteins and the guide nucleic acid molecule or guide nucleic acid molecules described herein in combination with an additional therapeutic agent for inducing growth arrest, cell death, or a combination thereof in a cell population.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo,” is used to describe an event that takes place in a subject's body.

The term “ex vivo,” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro,” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about,” a number refers to that number plus or minus 10% of that number. The term “about” a range refers to that range minus 10% of its lowest value and plus 10% of its greatest value.

As used herein, the term, “mutation,” refers to a change in the nucleotide sequence of a gene that may be caused by deletion, insertion or substitution of one or more nucleotides in the gene that results in a cellular characteristic or individual phenotype that is not observed in a cell or individual harboring only wildtype alleles of the gene. A cancer-associated mutation refers to a mutation that is present in the cell of an individual who has cancer.

As used herein, the term, “protein coding sequence,” refers to the combined sense strand sequences of all exons in a gene, ordered in a 5′ to 3′ direction. As used herein, the term, “protein coding sequence,” includes the amino acid coding nucleotides of a messenger RNA.

As used herein, the term, “wildtype sequence,” refers to a nucleotide sequence or amino acid sequence that is present in a substantial portion of a species. The substantial portion may be about 30%, about 40%, about 50%, about 60%, about 70%, about 80%, or about 90%.

As used herein, the terms “treatment,” or “treating,” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### A. Programmable Nucleases

In general, compositions and methods described herein comprise a programmable nuclease and uses thereof, respectively. The methods disclosed herein include using a programmable nuclease to effect cell growth arrest, cell death, or a combination thereof, of a population of cells. Any programmable nucleases may be used with the methods of the present disclosure. In some examples, a programmable nuclease used in the methods and systems disclosed herein comprises a CRISPR/Cas enzyme. CRISPR/Cas enzymes can include any of the known classes and types of CRISPR/Cas enzymes. In some examples, the programmable nuclease is a Class 1 CRISPR/Cas enzyme, such as one of the Type I, Type IV, or Type III CRISPR/Cas enzymes. In some examples, the programmable nuclease is a Class 2 CRISPR/Cas enzyme, such as the Type II, Type V, and Type VI CRISPR/Cas enzymes. Preferable programmable nucleases for use in the methods disclosed herein include a Type V or Type VI CRISPR/Cas enzyme.

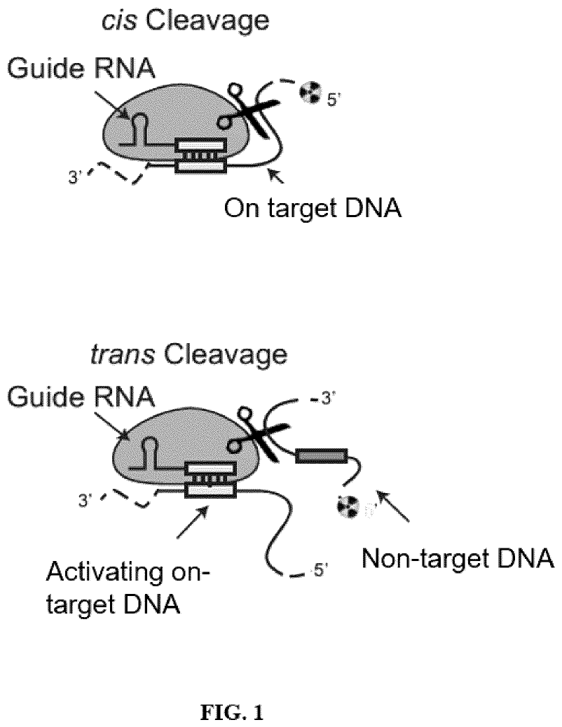

In some examples, a programmable nuclease as disclosed herein is an RNA-activated programmable RNA nuclease. In some embodiments, a programmable nuclease as disclosed herein is a DNA-activated programmable RNA nuclease. In some examples, a programmable nuclease is capable of being activated by a target RNA within a cell to initiate trans cleavage of one or more non-target RNAs within the cell. “Trans” cleavage activity can also be referred to as “collateral” or “transcollateral” cleavage. Trans cleavage activity can be non-specific cleavage of nearby single-stranded nucleic acids by the activated programmable nuclease, such as trans cleavage of detector nucleic acids with a detection moiety. On the other hand, “cis” cleavage activity, can refer to specific on-target cleavage of a DNA or RNA target by a CRISPR-guide RNA complex (FIG. 1 (top)). The trans cleavage activity of the CRISPR enzyme can be activated when the guide RNA is complexed with a nucleic acid target site (FIG. 1 (bottom)). In some examples, the programmable nuclease is capable of being activated by a target DNA in a cell to initiate trans cleavage of one or more non-target DNAs in the cell, such as a Type VI CRISPR/Cas enzyme. In some examples, the programmable nuclease is capable of being activated by a target RNA in a cell to initiate trans cleavage of one or more non-target DNAs in the cell. In some examples, the CRISPR protein can exhibit indiscriminate trans-cleavage of ssDNA in a disease cell.

In some examples, in methods described herein, the trans-cleavage induced by the CRISPR protein is sufficient to induce the death of a disease cell or a population of disease cells. In some instances, “apoptosis” refers to a form of programmed cell death in which a programmed sequence of events leads to the elimination of cells without releasing harmful substances. In some instances, apoptosis can be used to remove toxic or useless cells produced during animal development. In some examples, apoptosis can be induced by a number of external factors, including DNA or RNA degradation within a cell caused by, for example, the cleavage activity of a CRISPR protein. In some examples, “cell cycle arrest” refers to a halting of a series of events that take place in the cell leading to its division and replication. In some examples, cell cycle arrest may be caused by a number of factors, such as, DNA and RNA damage. In some examples, the trans-cleavage induced by the CRISPR protein is sufficient to induce the cell death, cell cycle arrest, or apoptosis, or combinations thereof of at least 5%, at least 10%, at least 20%, at least 30%, at least 40%, at least 50%, at least 60%, at least 70%, at least 80%, at least 90%, or 100% of a population of disease cells.

In some examples, the programmable nuclease is Cas13. In some examples, the programmable nuclease is Cas13a, Cas13b, Cas13c, Cas13d, or Cas13e. In some instances, the programmable nuclease is Mad7 or Mad2. In some cases, the programmable nuclease is Cas12. In some examples, the programmable nuclease comprises Cas12a, Cas12b, Cas12c, Cas12d, or Cas12e. In some examples, the programmable nuclease is Csm1, Cas9, C2c4, C2c8, C2c5, C2c10, C2c9, or CasZ. In some examples, the Csm1 can also be called smCms1, miCms1, obCms1, or suCms1. Sometimes Cas13a can also be called C2c2. Sometimes CasZ can also be called Cas14a, Cas14b, Cas14c, Cas14d, Cas14e, Cas14f, Cas14g, Cas14h, Cas14i, Cas14j, or Cas14k. In some examples, the programmable nuclease is a CasΦ nuclease. In some instances, the programmable nuclease is a type V CRISPR-Cas system. In some instances, the programmable nuclease is a type VI CRISPR-Cas system. In some examples, the programmable nuclease is a type III CRISPR-Cas system.

In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt).

### 1. Cas 12 Proteins

In some examples, the CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. (Murugan et al., Mol Cell. 2017 Oct. 5; 68(1): 15-25). In some instances, the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, or a C2c9 polypeptide.

In some examples, a Cas12 nuclease of the disclosure can exhibit indiscriminate trans-cleavage of ssDNA, enabling its use for inducing cell death, apoptosis, cell cycle arrest, or a combination thereof, in a population cells. In some examples, a Cas12 nuclease of the disclosure can, upon hybridization of a guide nucleic acid molecule to a target DNA or RNA, induce cis-cleavage of the target DNA or RNA.

In some instances, the Cas12 protein has at least 80%, at least 85%, at least 90%, at least 92%, at least 95%, at least 97%, or at least 99% sequence identity to any one of SEQ ID NO: 1-SEQ ID NO: 11. In some instances, the Cas12 protein is selected from SEQ ID NO: 1-SEQ ID NO: 11.

TABLE 1 provides amino acid sequences of illustrative Cas12 polypeptides that can be used in compositions and methods of the disclosure.

TABLE 1
Cas12 Protein Sequences

| # | Sequence | Annotation |
|---|---|---|
| 1 | MSKLEKFTNCYSLSKTLRFKAIPVGKTQENIDNKRLLVEDEKRAEDYKG | Lachnospiraceae |
| | VKKLLDRYYLSFINDVLHSIKLKNLNNYISLFRKKTRTEKENKELENLE | bacterium |
| | INLRKEIAKAFKGNEGYKSLFKKDIIETILPEFLDDKDEIALVNSFNGF | ND2006 |
| | TTAFTGFFDNRENMFSEEAKSTSIAFRCINENLTRYISNMDIFEKVDAI | (LbCas12a) |
| | FDKHEVQEIKEKILNSDYDVEDFFEGEFFNFVLTQEGIDVYNAIIGGFV | |
| | TESGEKIKGLNEYINLYNQKTKQKLPKFKPLYKQVLSDRESLSFYGEGY | |
| | TSDEEVLEVFRNTLNKNSEIFSSIKKLEKLFKNFDEYSSAGIFVKNGPA | |
| | ISTISKDIFGEWNVIRDKWNAEYDDIHLKKKAVVTEKYEDDRRKSFKKI | |
| | GSFSLEQLQEYADADLSVVEKLKEIIIQKVDEIYKVYGSSEKLFDADFV | |
| | LEKSLKKNDAVVAIMKDLLDSVKSFENYIKAFFGEGKETNRDESFYGDF | |
| | VLAYDILLKVDHIYDAIRNYVTQKPYSKDKFKLYFQNPQFMGGWDKDKE | |
| | TDYRATILRYGSKYYLAIMDKKYAKCLQKIDKDDVNGNYEKINYKLLPG | |
| | PNKMLPKVFFSKKWMAYYNPSEDIQKIYKNGTFKKGDMFNLNDCHKLID | |
| | FFKDSISRYPKWSNAYDFNFSETEKYKDIAGFYREVEEQGYKVSFESAS | |
| | KKEVDKLVEEGKLYMFQIYNKDFSDKSHGTPNLHTMYFKLLFDENNHGQ | |
| | IRLSGGAELFMRRASLKKEELVVHPANSPIANKNPDNPKKTTTLSYDVY | |
| | KDKRFSEDQYELHIPIAINKCPKNIFKINTEVRVLLKHDDNPYVIGIDR | |
| | GERNLLYIVVVDGKGNIVEQYSLNEIINNFNGIRIKTDYHSLLDKKEKE | |
| | RFEARQNWTSIENIKELKAGYISQVVHKICELVEKYDAVIALEDLNSGF | |
| | KNSRVKVEKQVYQKFEKMLIDKLNYMVDKKSNPCATGGALKGYQITNKF | |
| | ESFKSMSTQNGFIFYIPAWLTSKIDPSTGFVNLLKTKYTSIADSKKFIS | |
| | SFDRIMYVPEEDLFEFALDYKNFSRTDADYIKKWKLYSYGNRIRIFRNP | |
| | KKNNVFDWEEVCLTSAYKELFNKYGINYQQGDIRALLCEQSDKAFYSSF | |
| | MALMSLMLQMRNSITGRTDVDFLISPVKNSDGIFYDSRNYEAQENAILP | |
| | KNADANGAYNIARKVLWAIGQFKKAEDEKLDKVKIAISNKEWLEYAQTS | |
| | VKH | |
| 2 | MTQFEGFTNLYQVSKTLRFELIPQGKTLKHIQEQGFIEEDKARNDHYKE | Acidaminococcus |
| | LKPIIDRIYKTYADQCLQLVQLDWENLSAAIDSYRKEKTEETRNALIEE | sp. BV316 |
| | QATYRNAIHDYFIGRTDNLTDAINKRHAEIYKGLFKAELFNGKVLKQLG | (AsCas12a) |
| | TVTTTEHENALLRSFDKFTTYFSGFYENRKNVFSAEDISTAIPHRIVQD | |
| | NFPKFKENCHIFTRLITAVPSLREHFENVKKAIGIFVSTSIEEVFSFPF | |
| | YNQLLTQTQIDLYNQLLGGISREAGTEKIKGLNEVLNLAIQKNDETAHI | |
| | IASLPHRFIPLFKQILSDRNTLSFILEEFKSDEEVIQSFCKYKTLLRNE | |
| | NVLETAEALFNELNSIDLTHIFISHKKLETISSALCDHWDTLRNALYER | |
| | RISELTGKITKSAKEKVQRSLKHEDINLQEIISAAGKELSEAFKQKTSE | |
| | ILSHAHAALDQPLPTTLKKQEEKEILKSQLDSLLGLYHLLDWFAVDESN | |
| | EVDPEFSARLTGIKLEMEPSLSFYNKARNYATKKPYSVEKFKLNFQMPT | |
| | LASGWDVNKEKNNGAILFVKNGLYYLGIMPKQKGRYKALSFEPTEKTSE | |
| | GFDKMYYDYFPDAAKMIPKCSTQLKAVTAHFQTHTTPILLSNNFIEPLE | |
| | ITKEIYDLNNPEKEPKKFQTAYAKKTGDQKGYREALCKWIDFTRDFLSK | |
| | YTKTTSIDLSSLRPSSQYKDLGEYYAELNPLLYHISFQRIAEKEIMDAV | |
| | ETGKLYLFQIYNKDFAKGHHGKPNLHTLYWTGLFSPENLAKTSIKLNGQ | |
| | AELFYRPKSRMKRMAHRLGEKMLNKKLKDQKTPIPDTLYQELYDYVNHR | |
| | LSHDLSDEARALLPNVITKEVSHEIIKDRRFTSDKFFFHVPITLNYQAA | |
| | NSPSKFNQRVNAYLKEHPETPIIGIDRGERNLIYITVIDSTGKILEQRS | |
| | LNTIQQFDYQKKLDNREKERVAARQAWSVVGTIKDLKQGYLSQVIHEIV | |
| | DLMIHYQAVVVLENLNFGFKSKRTGIAEKAVYQQFEKMLIDKLNCLVLK | |
| | DYPAEKVGGVLNPYQLTDQFTSFAKMGTQSGFLFYVPAPYTSKIDPLTG | |
| | FVDPFVWKTIKNHESRKHFLEGFDFLHYDVKTGDFILHFKMNRNLSFQR | |
| | GLPGEMPAWDIVFEKNETQFDAKGTPFIAGKRIVPVIENHRFTGRYRDL | |
| | YPANELIALLEEKGIVFRDGSNILPKLLENDDSHAIDTMVALIRSVLQM | |
| | RNSNAATGEDYINSPVRDLNGVCFDSRFQNPEWPMDADANGAYHIALKG | |
| | QLLLNHLKESKDLKLQNGISNQDWLAYIQELRN | |
| 3 | MSIYQEFVNKYSLSKTLRFELIPQGKTLENIKARGLILDDEKRAKDYKK | Francisella |
| | AKQIIDKYHQFFIEEILSSVCISEDLLQNYSDVYFKLKKSDDDNLQKDF | novicida |
| | KSAKDTIKKQISEYIKDSEKFKNLFNQNLIDAKKGQESDLILWLKQSKD | U112 |
| | NGIELFKANSDITDIDEALEIIKSFKGWTTYFKGFHENRKNVYSSNDIP | (FnCas12a) |
| | TSIIYRIVDDNLPKFLENKAKYESLKDKAPEAINYEQIKKDLAEELTFD | |
| | IDYKTSEVNQRVFSLDEVFEIANFNNYLNQSGITKFNTIIGGKFVNGEN | |
| | TKRKGINEYINLYSQQINDKTLKKYKMSVLFKQILSDTESKSFVIDKLE | |
| | DDSDVVTTMQSFYEQIAAFKTVEEKSIKETLSLLFDDLKAQKLDLSKIY | |
| | FKNDKSLTDLSQQVFDDYSVIGTAVLEYITQQIAPKNLDNPSKKEQELI | |
| | AKKTEKAKYLSLETIKLALEEFNKHRDIDKQCRFEEILANFAAIPMIFD | |
| | EIAQNKDNLAQISIKYQNQGKKDLLQASAEDDVKAIKDLLDQTNNLLHK | |
| | LKIFHISQSEDKANILDKDEHFYLVFEECYFELANIVPLYNKIRNYITQ | |
| | KPYSDEKFKLNFENSTLANGWDKNKEPDNTAILFIKDDKYYLGVMNKKN | |
| | NKIFDDKAIKENKGEGYKKIVYKLLPGANKMLPKVFFSAKSIKFYNPSE | |
| | DILRIRNHSTHTKNGSPQKGYEKFEFNIEDCRKFIDFYKQSISKHPEWK | |
| | DFGFRESDTQRYNSIDEFYREVENQGYKLTFENISESYIDSVVNQGKLY | |
| | LFQIYNKDFSAYSKGRPNLHTLYWKALFDERNLQDVVYKLNGEAELFYR | |
| | KQSIPKKITHPAKEAIANKNKDNPKKESVFEYDLIKDKRFTEDKFFFHC | |
| | PITINFKSSGANKFNDEINLLLKEKANDVHILSIDRGERHLAYYTLVDG | |
| | KGNIIKQDTFNIIGNDRMKTNYHDKLAAIEKDRDSARKDWKKINNIKEM | |
| | KEGYLSQVVHEIAKLVIEYNAIVVFEDLNFGFKRGRFKVEKQVYQKLEK | |
| | MLIEKLNYLVFKDNEFDKTGGVLRAYQLTAPFETFKKMGKQTGIIYYVP | |
| | AGFTSKICPVTGFVNQLYPKYESVSKSQEFFSKFDKICYNLDKGYFEFS | |
| | FDYKNFGDKAAKGKWTIASFGSRLINFRNSDKNHNWDTREVYPTKELEK | |
| | LLKDYSIEYGHGECIKAAICGESDKKFFAKLTSVLNTILQMRNSKTGTE | |
| | LDYLISPVADVNGNFFDSRQAPKNMPQDADANGAYHIGLKGLMLLGRIK | |
| | NNQEGKKLNLVIKNEEYFEFVQNRNN | |
| 4 | MKTQHFFEDFTSLYSLSKTIRFELKPIGKTLENIKKNGLIRRDEQRLDD | Porphyromonas |
| | YEKLKKVIDEYHEDFIANILSSFSFSEEILQSYIQNLSESEARAKIEKT | macacae |
| | MRDTLAKAFSEDERYKSIFKKELVKKDIPVWCPAYKSLCKKFDNFTTSL | (PmCas12a) |
| | VPFHENRKNLYTSNEITASIPYRIVHVNLPKFIQNIEALCELQKKMGAD | |
| | LYLEMMENLRNVWPSFVKTPDDLCNLKTYNHLMVQSSISEYNRFVGGYS | |
| | TEDGTKHQGINEWINIYRQRNKEMRLPGLVFLHKQILAKVDSSSFISDT | |
| | LENDDQVFCVLRQFRKLFWNTVSSKEDDAASLKDLFCGLSGYDPEAIYV | |
| | SDAHLATISKNIFDRWNYISDAIRRKTEVLMPRKKESVERYAEKISKQI | |
| | KKRQSYSLAELDDLLAHYSEESLPAGFSLLSYFTSLGGQKYLVSDGEVI | |
| | LYEEGSNIWDEVLIAFRDLQVILDKDFTEKKLGKDEEAVSVIKKALDSA | |
| | LRLRKFFDLLSGTGAEIRRDSSFYALYTDRMDKLKGLLKMYDKVRNYLT | |
| | KKPYSIEKFKLHFDNPSLLSGWDKNKELNNLSVIFRQNGYYYLGIMTPK | |
| | GKNLFKTLPKLGAEEMFYEKMEYKQIAEPMLMLPKVFFPKKTKPAFAPD | |
| | QSVVDIYNKKTFKTGQKGFNKKDLYRLIDFYKEALTVHEWKLFNFSFSP | |
| | TEQYRNIGEFFDEVREQAYKVSMVNVPASYIDEAVENGKLYLFQIYNKD | |
| | FSPYSKGIPNLHTLYWKALFSEQNQSRVYKLCGGGELFYRKASLHMQDT | |
| | TVHPKGISIHKKNLNKKGETSLFNYDLVKDKRFTEDKFFFHVPISINYK | |
| | NKKITNVNQMVRDYIAQNDDLQIIGIDRGERNLLYISRIDTRGNLLEQF | |
| | SLNVIESDKGDLRTDYQKILGDREQERLRRRQEWKSIESIKDLKDGYMS | |
| | QVVHKICNMVVEHKAIVVLENLNLSFMKGRKKVEKSVYEKFERMLVDKL | |
| | NYLVVDKKNLSNEPGGLYAAYQLTNPLFSFEELHRYPQSGILFFVDPWN | |
| | TSLTDPSTGFVNLLGRINYTNVGDARKFFDRFNAIRYDGKGNILFDLDL | |
| | SRFDVRVETQRKLWTLTTFGSRIAKSKKSGKWMVERIENLSLCFLELFE | |
| | QFNIGYRVEKDLKKAILSQDRKEFYVRLIYLFNLMMQIRNSDGEEDYIL | |
| | SPALNEKNLQFDSRLIEAKDLPVDADANGAYNVARKGLMVVQRIKRGDH | |
| | ESIHRIGRAQWLRYVQEGIVE | |
| 5 | MLFQDFTHLYPLSKTVRFELKPIDRTLEHIHAKNFLSQDETMADMHQKV | Moraxella |
| | KVILDDYHRDFIADMMGEVKLTKLAEFYDVYLKFRKNPKDDELQKQLKD | bovoculi |
| | LQAVLRKEIVKPIGNGGKYKAGYDRLFGAKLFKDGKELGDLAKFVIAQE | 237 |
| | GESSPKLAHLAHFEKFSTYFTGFHDNRKNMYSDEDKHTAIAYRLIHENL | (MbCas12a) |
| | PRFIDNLQILTTIKQKHSALYDQIINELTASGLDVSLASHLDGYHKLLT | |
| | QEGITAYNTLLGGISGEAGSPKIQGINELINSHHNQHCHKSERIAKLRP | |
| | LHKQILSDGMSVSFLPSKFADDSEMCQAVNEFYRHYADVFAKVQSLFDG | |
| | FDDHQKDGIYVEHKNLNELSKQAFGDFALLGRVLDGYYVDVVNPEFNER | |
| | FAKAKTDNAKAKLTKEKDKFIKGVHSLASLEQAIEHYTARHDDESVQAG | |
| | KLGQYFKHGLAGVDNPIQKIHNNHSTIKGFLERERPAGERALPKIKSGK | |
| | NPEMTQLRQLKELLDNALNVAHFAKLLTTKTTLDNQDGNFYGEFGVLYD | |
| | ELAKIPTLYNKVRDYLSQKPFSTEKYKLNFGNPTLLNGWDLNKEKDNFG | |
| | VILQKDGCYYLALLDKAHKKVFDNAPNTGKSIYQKMIYKYLEVRKQFPK | |
| | VFFSKEAIAINYHPSKELVEIKDKGRQRSDDERLKLYRFILECLKIHPK | |
| | YDKKFEGAIGDIQLFKKDKKGREVPISEKDLFDKINGIFSSKPKLEMED | |
| | FFIGEFKRYNPSQDLVDQYNIYKKIDSNDNRKKENFYNNHPKFKKDLVR | |
| | YYYESMCKHEEWEESFEFSKKLQDIGCYVDVNELFTEIETRRLNYKISF | |
| | CNINADYIDELVEQGQLYLFQIYNKDFSPKAHGKPNLHTLYFKALFSED | |
| | NLADPIYKLNGEAQIFYRKASLDMNETTIHRAGEVLENKNPDNPKKRQF | |
| | VYDIIKDKRYTQDKFMLHVPITMNFGVQGMTIKEFNKKVNQSIQQYDEV | |
| | NVIGIDRGERHLLYLTVINSKGEILEQCSLNDITTASANGTQMTTPYHK | |
| | ILDKREIERLNARVGWGEIETIKELKSGYLSHVVHQISQLMLKYNAIVV | |
| | LEDLNFGFKRGRFKVEKQIYQNFENALIKKLNHLVLKDKADDEIGSYKN | |
| | ALQLTNNFTDLKSIGKQTGFLFYVPAWNTSKIDPETGFVDLLKPRYENI | |
| | AQSQAFFGKFDKICYNADKDYFEFHIDYAKFTDKAKNSRQIWTICSHGD | |
| | KRYVYDKTANQNKGAAKGINVNDELKSLFARHHINEKQPNLVMDICQNN | |
| | DKEFHKSLMYLLKTLLALRYSNASSDEDFILSPVANDEGVFFNSALADD | |
| | TQPQNADANGAYHIALKGLWLLNELKNSDDLNKVKLAIDNQTWLNFAQN | |
| | R | |
| 6 | MGIHGVPAALFQDFTHLYPLSKTVRFELKPIGRTLEHIHAKNFLSQDET | Moraxella |
| | MADMYQKVKVILDDYHRDFIADMMGEVKLTKLAEFYDVYLKFRKNPKDD | bovoculi |
| | GLQKQLKDLQAVLRKESVKPIGSGGKYKTGYDRLFGAKLFKDGKELGDL | AAX08_00205 |
| | AKFVIAQEGESSPKLAHLAHFEKFSTYFTGFHDNRKNMYSDEDKHTAIA | (Mb2Cas12a) |
| | YRLIHENLPRFIDNLQILTTIKQKHSALYDQIINELTASGLDVSLASHL | |
| | DGYHKLLTQEGITAYNRIIGEVNGYTNKHNQICHKSERIAKLRPLHKQI | |
| | LSDGMGVSFLPSKFADDSEMCQAVNEFYRHYTDVFAKVQSLFDGFDDHQ | |
| | KDGIYVEHKNLNELSKQAFGDFALLGRVLDGYYVDVVNPEFNERFAKAK | |
| | TDNAKAKLTKEKDKFIKGVHSLASLEQAIEHHTARHDDESVQAGKLGQY | |
| | FKHGLAGVDNPIQKIHNNHSTIKGFLERERPAGERALPKIKSGKNPEMT | |
| | QLRQLKELLDNALNVAHFAKLLTTKTTLDNQDGNFYGEFGVLYDELAKI | |
| | PTLYNKVRDYLSQKPFSTEKYKLNFGNPTLLNGWDLNKEKDNFGVILQK | |
| | DGCYYLALLDKAHKKVFDNAPNTGKNVYQKMVYKLLPGPNKMLPKVFFA | |
| | KSNLDYYNPSAELLDKYAKGTHKKGDNFNLKDCHALIDFFKAGINKHPE | |
| | WQHFGFKFSPTSSYRDLSDFYREVEPQGYQVKFVDINADYIDELVEQGK | |
| | LYLFQIYNKDESPKAHGKPNLHTLYFKALFSEDNLADPIYKLNGEAQIF | |
| | YRKASLDMNETTIHRAGEVLENKNPDNPKKRQFVYDIIKDKRYTQDKFM | |
| | LHVPITMNFGVQGMTIKEFNKKVNQSIQQYDEVNVIGIDRGERHLLYLT | |
| | VINSKGEILEQRSLNDITTASANGTQVTTPYHKILDKREIERLNARVGW | |
| | GEIETIKELKSGYLSHVVHQINQLMLKYNAIVVLEDLNFGFKRGRFKVE | |
| | KQIYQNFENALIKKLNHLVLKDKADDEIGSYKNALQLTNNFTDLKSIGK | |
| | QTGFLFYVPAWNTSKIDPETGFVDLLKPRYENIAQSQAFFGKFDKICYN | |
| | TDKGYFEFHIDYAKFTDKAKNSRQKWAICSHGDKRYVYDKTANQNKGAA | |
| | KGINVNDELKSLFARYHINDKQPNLVMDICQNNDKEFHKSLMCLLKTLL | |
| | ALRYSNASSDEDFILSPVANDEGVFFNSALADDTQPQNADANGAYHIAL | |
| | KGLWLLNELKNSDDLNKVKLAIDNQTWINFAQNR | |
| 7 | MGIHGVPAALFQDFTHLYPLSKTVRFELKPIGKTLEHIHAKNFLNQDET | Moraxella |
| | MADMYQKVKAILDDYHRDFIADMMGEVKLTKLAEFYDVYLKFRKNPKDD | bovoculi |
| | GLQKQLKDLQAVLRKEIVKPIGNGGKYKAGYDRLFGAKLFKDGKELGDL | AAX11_00205 |
| | AKFVIAQEGESSPKLAHLAHFEKFSTYFTGFHDNRKNMYSDEDKHTAIA | (Mb3Cas12a) |
| | YRLIHENLPRFIDNLQILATIKQKHSALYDQIINELTASGLDVSLASHL | |
| | DGYHKLLTQEGITAYNTLLGGISGEAGSRKIQGINELINSHHNQHCHKS | |
| | ERIAKLRPLHKQILSDGMGVSFLPSKFADDSEVCQAVNEFYRHYADVFA | |
| | KVQSLFDGEDDYQKDGIYVEYKNLNELSKQAFGDFALLGRVLDGYYVDV | |
| | VNPEFNERFAKAKTDNAKAKLTKEKDKFIKGVHSLASLEQAIEHYTARH | |
| | DDESVQAGKLGQYFKHGLAGVDNPIQKIHNNHSTIKGFLERERPAGERA | |
| | LPKIKSDKSPEIRQLKELLDNALNVAHFAKLLTTKTTLHNQDGNFYGEF | |
| | GALYDELAKIATLYNKVRDYLSQKPFSTEKYKLNFGNPTLLNGWDLNKE | |
| | KDNFGVILQKDGCYYLALLDKAHKKVFDNAPNTGKSVYQKMIYKLLPGP | |
| | NKMLPKVFFAKSNLDYYNPSAELLDKYAQGTHKKGDNFNLKDCHALIDF | |
| | FKAGINKHPEWQHFGFKFSPTSSYQDLSDFYREVEPQGYQVKFVDINAD | |
| | YINELVEQGQLYLFQIYNKDFSPKAHGKPNLHTLYFKALFSEDNLVNPI | |
| | YKLNGEAEIFYRKASLDMNETTIHRAGEVLENKNPDNPKKRQFVYDIIK | |
| | DKRYTQDKFMLHVPITMNFGVQGMTIKEFNKKVNQSIQQYDEVNVIGID | |
| | RGERHLLYLTVINSKGEILEQRSLNDITTASANGTQMTTPYHKILDKRE | |
| | IERLNARVGWGEIETIKELKSGYLSHVVHQISQLMLKYNAIVVLEDLNF | |
| | GFKRGRFKVEKQIYQNFENALIKKLNHLVLKDKADDEIGSYKNALQLTN | |
| | NFTDLKSIGKQTGFLFYVPAWNTSKIDPETGFVDLLKPRYENIAQSQAF | |
| | FGKFDKICYNADRGYFEFHIDYAKFNDKAKNSRQIWKICSHGDKRYVYD | |
| | KTANQNKGATIGVNVNDELKSLFTRYHINDKQPNLVMDICQNNDKEFHK | |
| | SLMYLLKTLLALRYSNASSDEDFILSPVANDEGVFFNSALADDTQPQNA | |
| | DANGAYHIALKGLWLLNELKNSDDLNKVKLAIDNQTWLNFAQNR | |
| 8 | MGIHGVPAATKTFDSEFFNLYSLQKTVRFELKPVGETASFVEDFKNEGL | Thiomicrospira |
| | KRVVSEDERRAVDYQKVKEIIDDYHRDFIEESLNYFPEQVSKDALEQAF | sp. XS5 |
| | HLYQKLKAAKVEEREKALKEWEALQKKLREKVVKCFSDSNKARFSRIDK | (TsCas12a) |
| | KELIKEDLINWLVAQNREDDIPTVETFNNFTTYFTGFHENRKNIYSKDD | |
| | HATAISFRLIHENLPKFFDNVISFNKLKEGFPELKFDKVKEDLEVDYDL | |
| | KHAFEIEYFVNFVTQAGIDQYNYLLGGKTLEDGTKKQGMNEQINLFKQQ | |
| | QTRDKARQIPKLIPLFKQILSERTESQSFIPKQFESDQELFDSLQKLHN | |
| | NCQDKFTVLQQAILGLAEADLKKVFIKTSDLNALSNTIFGNYSVFSDAL | |
| | NLYKESLKTKKAQEAFEKLPAHSIHDLIQYLEQFNSSLDAEKQQSTDTV | |
| | LNYFIKTDELYSRFIKSTSEAFTQVQPLFELEALSSKRRPPESEDEGAK | |
| | GQEGFEQIKRIKAYLDTLMEAVHFAKPLYLVKGRKMIEGLDKDQSFYEA | |
| | FEMAYQELESLIIPIYNKARSYLSRKPFKADKFKINFDNNTLLSGWDAN | |
| | KETANASILFKKDGLYYLGIMPKGKTFLFDYFVSSEDSEKLKQRRQKTA | |
| | EEALAQDGESYFEKIRYKLLPGASKMLPKVFFSNKNIGFYNPSDDILRI | |
| | RNTASHTKNGTPQKGHSKVEFNLNDCHKMIDFFKSSIQKHPEWGSFGFT | |
| | FSDTSDFEDMSAFYREVENQGYVISFDKIKETYIQSQVEQGNLYLFQIY | |
| | NKDFSPYSKGKPNLHTLYWKALFEEANLNNVVAKLNGEAEIFFRRHSIK | |
| | ASDKVVHPANQAIDNKNPHTEKTQSTFEYDLVKDKRYTQDKFFFHVPIS | |
| | LNFKAQGVSKFNDKVNGFLKGNPDVNIIGIDRGERHLLYFTVVNQKGEI | |
| | LVQESLNTLMSDKGHVNDYQQKLDKKEQERDAARKSWTTVENIKELKEG | |
| | YLSHVVHKLAHLIIKYNAIVCLEDLNFGFKRGRFKVEKQVYQKFEKALI | |
| | DKLNYLVFKEKELGEVGHYLTAYQLTAPFESFKKLGKQSGILFYVPADY | |
| | TSKIDPTTGFVNFLDLRYQSVEKAKQLLSDFNAIRFNSVQNYFEFEIDY | |
| | KKLTPKRKVGTQSKWVICTYGDVRYQNRRNQKGHWETEEVNVTEKLKAL | |
| | FASDSKTTTVIDYANDDNLIDVILEQDKASFFKELLWLLKLTMTLRHSK | |
| | IKSEDDFILSPVKNEQGEFYDSRKAGEVWPKDADANGAYHIALKGLWNL | |
| | QQINQWEKGKTLNLAIKNQDWFSFIQEKPYQE | |
| 9 | MGIHGVPAAYYQNLTKKYPVSKTIRNELIPIGKTLENIRKNNILESDVK | Butyrivibrio |
| | RKQDYEHVKGIMDEYHKQLINEALDNYMLPSLNQAAEIYLKKHVDVEDR | sp. |
| | EEFKKTQDLLRREVTGRLKEHENYTKIGKKDILDLLEKLPSISEEDYNA | NC3005 |
| | LESFRNFYTYFTSYNKVRENLYSDEEKSSTVAYRLINENLPKFLDNIKS | (BsCas12a) |
| | YAFVKAAGVLADCIEEEEQDALFMVETFNMTLTQEGIDMYNYQIGKVNS | |
| | AINLYNQKNHKVEEFKKIPKMKVLYKQILSDREEVFIGEFKDDETLLSS | |
| | IGAYGNVLMTYLKSEKINIFFDALRESEGKNVYVKNDLSKTTMSNIVFG | |
| | SWSAFDELLNQEYDLANENKKKDDKYFEKRQKELKKNKSYTLEQMSNLS | |
| | KEDISPIENYIERISEDIEKICIYNGEFEKIVVNEHDSSRKLSKNIKAV | |
| | KVIKDYLDSIKELEHDIKLINGSGQELEKNLVVYVGQEEALEQLRPVDS | |
| | LYNLTRNYLTKKPFSTEKVKLNFNKSTLLNGWDKNKETDNLGILFFKDG | |
| | KYYLGIMNTTANKAFVNPPAAKTENVFKKVDYKLLPGSNKMLPKVFFAK | |
| | SNIGYYNPSTELYSNYKKGTHKKGPSFSIDDCHNLIDFFKESIKKHEDW | |
| | SKFGFEFSDTADYRDISEFYREVEKQGYKLTFTDIDESYINDLIEKNEL | |
| | YLFQIYNKDFSEYSKGKLNLHTLYFMMLFDQRNLDNVVYKLNGEAEVFY | |
| | RPASIAENELVIHKAGEGIKNKNPNRAKVKETSTFSYDIVKDKRYSKYK | |
| | FTLHIPITMNFGVDEVRRFNDVINNALRTDDNVNVIGIDRGERNLLYVV | |
| | VINSEGKILEQISLNSIINKEYDIETNYHALLDEREDDRNKARKDWNTI | |
| | ENIKELKTGYLSQVVNVVAKLVLKYNAIICLEDLNFGFKRGRQKVEKQV | |
| | YQKFEKMLIEKLNYLVIDKSREQVSPEKMGGALNALQLTSKFKSFAELG | |
| | KQSGIIYYVPAYLTSKIDPTTGFVNLFYIKYENIEKAKQFFDGFDFIRF | |
| | NKKDDMFEFSFDYKSFTQKACGIRSKWIVYINGERIIKYPNPEKNNLFD | |
| | EKVINVTDEIKGLFKQYRIPYENGEDIKEIIISKAEADFYKRLFRLLHQ | |
| | TLQMRNSTSDGTRDYIISPVKNDRGEFFCSEFSEGTMPKDADANGAYNI | |
| | ARKGLWVLEQIRQKDEGEKVNLSMTNAEWLKYAQLHLL | |
| 10 | MAVKSIKVKLRLDDMPEIRAGLWKLHKEVNAGVRYYTEWLSLLRQENLY | AacCas12b |
| | RRSPNGDGEQECDKTAEECKAELLERLRARQVENGHRGPAGSDDELLQL | |
| | ARQLYELLVPQAIGAKGDAQQIARKFLSPLADKDAVGGLGIAKAGNKPR | |
| | WVRMREAGEPGWEEEKEKAETRKSADRTADVLRALADFGLKPLMRVYTD | |
| | SEMSSVEWKPLRKGQAVRTWDRDMFQQAIERMMSWESWNQRVGQEYAKL | |
| | VEQKNRFEQKNFVGQEHLVHLVNQLQQDMKEASPGLESKEQTAHYVTGR | |
| | ALRGSDKVFEKWGKLAPDAPFDLYDAEIKNVQRRNTRRFGSHDLFAKLA | |
| | EPEYQALWREDASFLTRYAVYNSILRKLNHAKMFATFTLPDATAHPIWT | |
| | RFDKLGGNLHQYTFLFNEFGERRHAIRFHKLLKVENGVAREVDDVTVPI | |
| | SMSEQLDNLLPRDPNEPIALYFRDYGAEQHFTGEFGGAKIQCRRDQLAH | |
| | MHRRRGARDVYLNVSVRVQSQSEARGERRPPYAAVERLVGDNHRAFVHF | |
| | DKLSDYLAEHPDDGKLGSEGLLSGLRVMSVDLGLRTSASISVFRVARKD | |
| | ELKPNSKGRVPFFFPIKGNDNLVAVHERSQLLKLPGETESKDLRAIREE | |
| | RQRTLRQLRTQLAYLRLLVRCGSEDVGRRERSWAKLIEQPVDAANHMTP | |
| | DWREAFENELQKLKSLHGICSDKEWMDAVYESVRRVWRHMGKQVRDWRK | |
| | DVRSGERPKIRGYAKDVVGGNSIEQIEYLERQYKFLKSWSFFGKVSGQV | |
| | IRAEKGSRFAITLREHIDHAKEDRLKKLADRIIMEALGYVYALDERGKG | |
| | KWVAKYPPCQLILLEELSEYQFNNDRPPSENNQLMQWSHRGVFQELINQ | |
| | AQVHDLLVGTMYAAFSSRFDARTGAPGIRCRRVPARCTQEHNPEPFPWW | |
| | LNKFVVEHTLDACPLRADDLIPTGEGEIFVSPESAEEGDFHQIHADLNA | |
| | AQNLQQRLWSDEDISQIRLRCDWGEVDGELVLIPRLTGKRTADSYSNKV | |
| | FYTNTGVTYYERERGKKRRKVFAQEKLSEEEAELLVEADEAREKSVVLM | |
| | RDPSGIINRGNWTRQKEFWSMVNQRIEGYLVKQIRSRVPLQDSACENTG | |
| | DI | |
| 11 | MKKIDNFVGCYPVSKTLRFKAIPIGKTQENIEKKRLVEEDEVRAKDYKA | Cas12 |
| | VKKLIDRYHREFIEGVLDNVKLDGLEEYYMLFNKSDREESDNKKIEIME | Variant |
| | ERFRRVISKSFKNNEEYKKIFSKKIIEEILPNYIKDEEEKELVKGFKGF | |
| | YTAFVGYAQNRENMYSDEKKSTAISYRIVNENMPRFITNIKVFEKAKSI | |
| | LDVDKINEINEYILNNDYYVDDFFNIDFFNYVLNQKGIDIYNAIIGGIV | |
| | TGDGRKIQGLNECINLYNQENKKIRLPQFKPLYKQILSESESMSFYIDE | |
| | IESDDMLIDMLKESLQIDSTINNAIDDLKVLFNNIFDYDLSGIFINNGL | |
| | PITTISNDVYGQWSTISDGWNERYDVLSNAKDKESEKYFEKRRKEYKKV | |
| | KSFSISDLQELGGKDLSICKKINEIISEMIDDYKSKIEEIQYLFDIKEL | |
| | EKPLVTDLNKIELIKNSLDGLKRIERYVIPFLGTGKEQNRDEVFYGYFI | |
| | KCIDAIKEIDGVYNKTRNYLTKKPYSKDKFKLYFENPQLMGGWDRNKES | |
| | DYRSTLLRKNGKYYVAIIDKSSSNCMMNIEEDENDNYEKINYKLLPGPN | |
| | KMLPKVFFSKKNREYFAPSKEIERIYSTGTFKKDTNFVKKDCENLITFY | |
| | KDSLDRHEDWSKSFDFSFKESSAYRDISEFYRDVEKQGYRVSFDLLSSN | |
| | AVNTLVEEGKLYLFQLYNKDFSEKSHGIPNLHTMYFRSLFDDNNKGNIR | |
| | LNGGAEMFMRRASLNKQDVTVHKANQPIKNKNLLNPKKTTTLPYDVYKD | |
| | KRFTEDQYEVHIPITMNKVPNNPYKINHMVREQLVKDDNPYVIGIDRGE | |
| | RNLIYVVVVDGQGHIVEQLSLNEIINENNGISIRTDYHTLLDAKERERD | |
| | ESRKQWKQIENIKELKEGYISQVVHKICELVEKYDAVIALEDLNSGFKN | |
| | SRVKVEKQVYQKFEKMLITKLNYMVDKKKDYNKPGGVLNGYQLTTQFES | |
| | FSKMGTQNGIMFYIPAWLTSKMDPTTGFVDLLKPKYKNKADAQKFFSQF | |
| | DSIRYDNQEDAFVFKVNYTKFPRTDADYNKEWEIYTNGERIRVFRNPKK | |
| | NNEYDYETVNVSERMKELFDSYDLLYDKGELKETICEMEESKFFEELIK | |
| | LFRLTLQMRNSISGRTDVDYLISPVKNSNGYFYNSNDYKKEGAKYPKDA | |
| | DANGAYNIARKVLWAIEQFKMADEDKLDKTKISIKNQEWLEYAQTHCE | |

### 2. Cas 14 Proteins

In some examples, the Type V CRISPR/Cas enzyme is a programmable Cas14 nuclease. A Cas14 protein of the present disclosure includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. In some examples, the Cas14 protein comprises a Cas14a polypeptide, a Cas14b polypeptide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide. Sometimes any of Cas14a, Cas14b, Cas14c, Cas14d, Cas14e, Cas14f, Cas14g, or Cas14h proteins can be called CasZ.

In some examples, a Cas14 nuclease of the disclosure can exhibit indiscriminate trans-cleavage of ssDNA or ssRNA, enabling its use for inducing cell death, apoptosis, cell cycle arrest, or a combination thereof, in a population cells. In some examples, a Cas14 nuclease of the disclosure can, upon hybridization of a guide nucleic acid molecule to a target DNA or RNA, induce cis-cleavage of the target DNA or RNA.

In some examples, the Cas14 protein has at least 80%, at least 85%, at least 90%, at least 92%, at least 95%, at least 97%, or at least 99% sequence identity to any one of SEQ ID NO: 12-SEQ ID NO: 154, and 244. In some examples, the Cas14 protein is selected from SEQ ID NO: 12-SEQ ID NO: 154 and 244.

TABLE 2 provides amino acid sequences of illustrative Cas14 polypeptides that can be used in compositions and methods of the disclosure.

TABLE 2
Cas14 Protein Sequences

| # | Sequence |
|---|---|
| SEQ ID | MEVQKTVMKTLSLRILRPLYSQEIEKEIKEEKERRKQAGGTGELDGGFYKKLEKKHSEM |
| NO: 12 | FSFDRLNLLLNQLQREIAKVYNHAISELYIATIAQGNKSNKHYISSIVYNRAYGYFYNA |
| | YIALGICSKVEANFRSNELLTQQSALPTAKSDNFPIVLHKQKGAEGEDGGFRISTEGSD |
| | LIFEIPIPFYEYNGENRKEPYKWVKKGGQKPVLKLILSTFRRQRNKGWAKDEGTDAEIR |
| | KVTEGKYQVSQIEINRGKKLGEHQKWFANFSIEQPIYERKPNRSIVGGLDVGIRSPLVC |
| | AINNSFSRYSVDSNDVFKFSKQVFAFRRRLLSKNSLKRKGHGAAHKLEPITEMTEKNDK |
| | FRKKIIERWAKEVTNFFVKNQVGIVQIEDLSTMKDREDHFFNQYLRGFWPYYQMQTLIE |
| | NKLKEYGIEVKRVQAKYTSQLCSNPNCRYWNNYFNFEYRKVNKFPKFKCEKCNLEISAD |
| | YNAARNLSTPDIEKFVAKATKGINLPEK |
| SEQ ID | MEEAKTVSKTLSLRILRPLYSAEIEKEIKEEKERRKQGGKSGELDSGFYKKLEKKHTQM |
| NO: 13 | FGWDKLNLMLSQLQRQIARVFNQSISELYIETVIQGKKSNKHYTSKIVYNRAYSVFYNA |
| | YLALGITSKVEANFRSTELLMQKSSLPTAKSDNFPILLHKQKGVEGEEGGFKISADGND |
| | LIFEIPIPFYEYDSANKKEPFKWIKKGGQKPTIKLILSTFRRQRNKGWAKDEGTDAEIR |
| | KVIEGKYQVSHIEINRGKKLGDHQKWFVNFTIEQPIYERKLDKNIIGGIDVGIKSPLVC |
| | AVNNSFARYSVDSNDVLKFSKQAFAFRRRLLSKNSLKRSGHGSKNKLDPITRMTEKNDR |
| | FRKKIIERWAKEVTNFFIKNQVGTVQIEDLSTMKDRQDNFFNQYLRGFWPYYQMQNLIE |
| | NKLKEYGIETKRIKARYTSQLCSNPSCRHWNSYFSFDHRKTNNFPKFKCEKCALEISAD |
| | YNAARNISTPDIEKFVAKATKGINLPDKNENVILE |
| SEQ ID | MAKNTITKTLKLRIVRPYNSAEVEKIVADEKNNREKIALEKNKDKVKEACSKHLKVAAY |
| NO: 14 | CTTQVERNACLFCKARKLDDKFYQKLRGQFPDAVFWQEISEIFRQLQKQAAEIYNQSLI |
| | ELYYEIFIKGKGIANASSVEHYLSDVCYTRAAELFKNAAIASGLRSKIKSNFRLKELKN |
| | MKSGLPTTKSDNFPIPLVKQKGGQYTGFEISNHNSDFIIKIPFGRWQVKKEIDKYRPWE |
| | KFDFEQVQKSPKPISLLLSTQRRKRNKGWSKDEGTEAEIKKVMNGDYQTSYIEVKRGSK |
| | IGEKSAWMLNLSIDVPKIDKGVDPSIIGGIDVGVKSPLVCAINNAFSRYSISDNDLFHF |
| | NKKMFARRRILLKKNRHKRAGHGAKNKLKPITILTEKSERFRKKLIERWACEIADFFIK |
| | NKVGTVQMENLESMKRKEDSYFNIRLRGFWPYAEMQNKIEFKLKQYGIEIRKVAPNNTS |
| | KTCSKCGHLNNYFNFEYRKKNKFPHFKCEKCNFKENADYNAALNISNPKLKSTKEEP |
| SEQ ID | MERQKVPQIRKIVRVVPLRILRPKYSDVIENALKKFKEKGDDTNTNDFWRAIRDRDTEF |
| NO: 16 | FRKELNFSEDEINQLERDTLFRVGLDNRVLFSYFDFLQEKLMKDYNKIISKLFINRQSK |
| | SSFENDLTDEEVEELIEKDVTPFYGAYIGKGIKSVIKSNLGGKFIKSVKIDRETKKVTK |
| | LTAINIGLMGLPVAKSDTFPIKIIKTNPDYITFQKSTKENLQKIEDYETGIEYGDLLVQ |
| | ITIPWFKNENKDFSLIKTKEAIEYYKLNGVGKKDLLNINLVLTTYHIRKKKSWQIDGSS |
| | QSLVREMANGELEEKWKSFFDTFIKKYGDEGKSALVKRRVNKKSRAKGEKGRELNLDER |
| | IKRLYDSIKAKSFPSEINLIPENYKWKLHFSIEIPPMVNDIDSNLYGGIDFGEQNIATL |
| | CVKNIEKDDYDFLTIYGNDLLKHAQASYARRRIMRVQDEYKARGHGKSRKTKAQEDYSE |
| | RMQKLRQKITERLVKQISDFFLWRNKFHMAVCSLRYEDLNTLYKGESVKAKRMRQFINK |
| | QQLFNGIERKLKDYNSEIYVNSRYPHYTSRLCSKCGKLNLYFDFLKFRTKNIIIRKNPD |
| | GSEIKYMPFFICEFCGWKQAGDKNASANIADKDYQDKLNKEKEFCNIRKPKSKKEDIGE |
| | ENEEERDYSRRFNRNSFIYNSLKKDNKLNQEKLFDEWKNQLKRKIDGRNKFEPKEYKDR |
| | FSYLFAYYQEIIKNESES |
| SEQ ID | MVPTELITKTLQLRVIRPLYFEEIEKELAELKEQKEKEFEETNSLLLESKKIDAKSLKK |
| NO: 17 | LKRKARSSAAVEFWKIAKEKYPDILTKPEMEFIFSEMQKMMARFYNKSMTNIFIEMNND |
| | EKVNPLSLISKASTEANQVIKCSSISSGLNRKIAGSINKTKFKQVRDGLISLPTARTET |
| | FPISFYKSTANKDEIPISKINLPSEEEADLTITLPFPFFEIKKEKKGQKAYSYFNIIEK |
| | SGRSNNKIDLLLSTHRRQRRKGWKEEGGTSAEIRRLMEGEFDKEWEIYLGEAEKSEKAK |
| | NDLIKNMTRGKLSKDIKEQLEDIQVKYFSDNNVESWNDLSKEQKQELSKLRKKKVEELK |
| | DWKHVKEILKTRAKIGWVELKRGKRQRDRNKWFVNITITRPPFINKELDDTKFGGIDLG |
| | VKVPFVCAVHGSPARLIIKENEILQFNKMVSARNRQITKDSEQRKGRGKKNKFIKKEIF |
| | NERNELFRKKIIERWANQIVKFFEDQKCATVQIENLESFDRTSYK |
| SEQ ID | MKSDTKDKKIIIHQTKTLSLRIVKPQSIPMEEFTDLVRYHQMIIFPVYNNGAIDLYKKL |
| NO: 18 | FKAKIQKGNEARAIKYFMNKIVYAPIANTVKNSYIALGYSTKMQSSFSGKRLWDLRFGE |
| | ATPPTIKADFPLPFYNQSGFKVSSENGEFIIGIPFGQYTKKTVSDIEKKTSFAWDKFTL |
| | EDTTKKTLIELLLSTKTRKMNEGWKNNEGTEAEIKRVMDGTYQVTSLEILQRDDSWFVN |
| | FNIAYDSLKKQPDRDKIAGIHMGITRPLTAVIYNNKYRALSIYPNTVMHLTQKQLARIK |
| | EQRTNSKYATGGHGRNAKVTGTDTLSEAYRQRRKKIIEDWIASIVKFAINNEIGTIYLE |
| | DISNTNSFFAAREQKLIYLEDISNTNSFLSTYKYPISAISDTLQHKLEEKAIQVIRKKA |
| | YYVNQICSLCGHYNKGFTYQFRRKNKFPKMKCQGCLEATSTEFNAAANVANPDYEKLLI |
| | KHGLLQLKK |
| SEQ ID | MSTITRQVRLSPTPEQSRLLMAHCQQYISTVNVLVAAFDSEVLTGKVSTKDFRAALPSA |
| NO: 19 | VKNQALRDAQSVFKRSVELGCLPVLKKPHCQWNNQNWRVEGDQLILPICKDGKTQQERF |
| | RCAAVALEGKAGILRIKKKRGKWIADLTVTQEDAPESSGSAIMGVDLGIKVPAVAHIGG |
| | KGTRFFGNGRSQRSMRRRFYARRKTLQKAKKLRAVRKSKGKEARWMKTINHQLSRQIVN |
| | HAHALGVGTIKIEALQGIRKGTTRKSRGAAARKNNRMTNTWSFSQLTLFITYKAQRQGI |
| | TVEQVDPAYTSQDCPACRARNGAQDRTYVCSECGWRGHRDTVGAINISRRAGLSGHRRG |
| | ATGA |
| SEQ ID | MIAQKTIKIKLNPTKEQIIKLNSIIEEYIKVSNFTAKKIAEIQESFTDSGLTQGTCSEC |
| NO: 20 | GKEKTYRKYHLLKKDNKLFCITCYKRKYSQFTLQKVEFQNKTGLRNVAKLPKTYYTNAI |
| | RFASDTFSGFDEIIKKKQNRLNSIQNRLNFWKELLYNPSNRNEIKIKVVKYAPKTDTRE |
| | HPHYYSEAEIKGRIKRLEKQLKKFKMPKYPEFTSETISLQRELYSWKNPDELKISSITD |
| | KNESMNYYGKEYLKRYIDLINSQTPQILLEKENNSFYLCFPITKNIEMPKIDDTFEPVG |
| | IDWGITRNIAVVSILDSKTKKPKFVKFYSAGYILGKRKHYKSLRKHFGQKKRQDKINKL |
| | GTKEDRFIDSNIHKLAFLIVKEIRNHSNKPIILMENITDNREEAEKSMRQNILLHSVKS |
| | RLQNYIAYKALWNNIPTNLVKPEHTSQICNRCGHQDRENRPKGSKLFKCVKCNYMSNAD |
| | FNASINIARKFYIGEYEPFYKDNEKMKSGVNSISM |
| SEQ ID | LKLSEQENITTGVKFKLKLDKETSEGLNDYFDEYGKAINFAIKVIQKELAEDRFAGKVR |
| NO: 21 | LDENKKPLLNEDGKKIWDFPNEFCSCGKQVNRYVNGKSLCQECYKNKFTEYGIRKRMYS |
| | AKGRKAEQDINIKNSTNKISKTHENYAIREAFILDKSIKKQRKERFRRLREMKKKLQEF |
| | IEIRDGNKILCPKIEKQRVERYIHPSWINKEKKLEDFRGYSMSNVLGKIKILDRNIKRE |
| | EKSLKEKGQINFKARRLMLDKSVKFLNDNKISFTISKNLPKEYELDLPEKEKRLNWLKE |
| | KIKIIKNQKPKYAYLLRKDDNFYLQYTLETEFNLKEDYSGIVGIDRGVSHIAVYTEFHN |
| | NGKNERPLFLNSSEILRLKNLQKERDRFLRRKHNKKRKKSNMRNIEKKIQLILHNYSKQ |
| | IVDFAKNKNAFIVFEKLEKPKKNRSKMSKKSQYKLSQFTFKKLSDLVDYKAKREGIKVL |
| | YISPEYTSKECSHCGEKVNTQRPENGNSSLFKCNKCGVELNADYNASINIAKKGLNILN |
| | STN |
| SEQ ID | MEESIITGVKFKLRIDKETTKKLNEYFDEYGKAINFAVKIIQKELADDRFAGKAKLDQN |
| NO: 22 | KNPILDENGKKIYEFPDEFCSCGKQVNKYVNNKPFCQECYKIRFTENGIRKRMYSAKGR |
| | KAEHKINILNSTNKISKTHFNYAIREAFILDKSIKKQRKKRNERLRESKKRLQQFIDMR |
| | DGKREICPTIKGQKVDRFIHPSWITKDKKLEDFRGYTLSIINSKIKILDRNIKREEKSL |
| | KEKGQIIFKAKRLMLDKSIRFVGDRKVLFTISKTLPKEYELDLPSKEKRLNWLKEKIEI |
| | IKNQKPKYAYLLRKNIESEKKPNYEYYLQYTLEIKPELKDFYDGAIGIDRGINHIAVCT |
| | FISNDGKVTPPKFFSSGEILRLKNLQKERDREFLRKHNKNRKKGNMRVIENKINLILHR |
| | YSKQIVDMAKKLNASIVFEELGRIGKSRTKMKKSQRYKLSLFIFKKLSDLVDYKSRREG |
| | IRVTYVPPEYTSKECSHCGEKVNTQRPENGNYSLFKCNKCGIQLNSDYNASINIAKKGL |
| | KIPNST |
| SEQ ID | LWTIVIGDFIEMPKQDLVTTGIKFKLDVDKETRKKLDDYFDEYGKAINFAVKIIQKNLK |
| NO: 23 | EDRFAGKIALGEDKKPLLDKDGKKIYNYPNESCSCGNQVRRYVNAKPFCVDCYKLKFTE |
| | NGIRKRMYSARGRKADSDINIKNSTNKISKTHENYAIREGFILDKSLKKQRSKRIKKLL |
| | ELKRKLQEFIDIRQGQMVLCPKIKNQRVDKFIHPSWLKRDKKLEEFRGYSLSVVEGKIK |
| | IFNRNILREEDSLRQRGHVNFKANRIMLDKSVRFLDGGKVNENLNKGLPKEYLLDLPKK |
| | ENKLSWLNEKISLIKLQKPKYAYLLRREGSFFIQYTIENVPKTEFDYLGAIGIDRGISH |
| | IAVCTFVSKNGVNKAPVFFSSGEILKLKSLQKQRDLFLRGKHNKIRKKSNMRNIDNKIN |
| | LILHKYSRNIVNLAKSEKAFIVFEKLEKIKKSRFKMSKSLQYKLSQFTFKKLSDLVEYK |
| | AKIEGIKVDYVPPEYTSKECSHCGEKVDTQRPFNGNSSLFKCNKCRVQLNADYNASINI |
| | AKKSLNISN |
| SEQ ID | MSKTTISVKLKIIDLSSEKKEFLDNYFNEYAKATTFCQLRIRRLLRNTHWLGKKEKSSK |
| NO: 24 | KWIFESGICDLCGENKELVNEDRNSGEPAKICKRCYNGRYGNQMIRKLFVSTKKREVQE |
| | NMDIRRVAKLNNTHYHRIPEEAFDMIKAADTAEKRRKKNVEYDKKRQMEFIEMENDEKK |
| | RAARPKKPNERETRYVHISKLESPSKGYTLNGIKRKIDGMGKKIERAEKGLSRKKIFGY |
| | QGNRIKLDSNWVRFDLAESEITIPSLFKEMKLRITGPTNVHSKSGQIYFAEWFERINKQ |
| | PNNYCYLIRKTSSNGKYEYYLQYTYEAEVEANKEYAGCLGVDIGCSKLAAAVYYDSKNK |
| | KAQKPIEIFTNPIKKIKMRREKLIKLLSRVKVRHRRRKLMQLSKTEPIIDYTCHKTARK |
| | IVEMANTAKAFISMENLETGIKQKQQARETKKQKFYRNMFLFRKLSKLIEYKALLKGIK |
| | IVYVKPDYTSQTCSSCGADKEKTERPSQAIFRCLNPTCRYYQRDINADFNAAVNIAKKA |
| | LNNTEVVTTLL |
| SEQ ID | MARAKNQPYQKLTTTTGIKFKLDLSEEEGKRFDEYFSEYAKAVNFCAKVIYQLRKNLKF |
| NO: 25 | AGKKELAAKEWKFEISNCDFCNKQKEIYYKNIANGQKVCKGCHRTNFSDNAIRKKMIPV |
| | KGRKVESKFNIHNTTKKISGTHRHWAFEDAADIIESMDKQRKEKQKRLRREKRKLSYFF |
| | ELFGDPAKRYELPKVGKQRVPRYLHKIIDKDSLTKKRGYSLSYIKNKIKISERNIERDE |
| | KSLRKASPIAFGARKIKMSKLDPKRAFDLENNVFKIPGKVIKGQYKFFGTNVANEHGKK |
| | FYKDRISKILAGKPKYFYLLRKKVAESDGNPIFEYYVQWSIDTETPAITSYDNILGIDA |
| | GITNLATTVLIPKNLSAEHCSHCGNNHVKPIFTKFFSGKELKAIKIKSRKQKYFLRGKH |
| | NKLVKIKRIRPIEQKVDGYCHVVSKQIVEMAKERNSCIALEKLEKPKKSKFRQRRREKY |
| | AVSMFVFKKLATFIKYKAAREGIEIIPVEPEGTSYTCSHCKNAQNNQRPYFKPNSKKSW |
| | TSMFKCGKCGIELNSDYNAAFNIAQKALNMTSA |
| SEQ ID | MDEKHFFCSYCNKELKISKNLINKISKGSIREDEAVSKAISIHNKKEHSLILGIKFKLF |
| NO: 26 | IENKLDKKKLNEYFDNYSKAVTFAARIFDKIRSPYKFIGLKDKNTKKWTFPKAKCVFCL |
| | EEKEVAYANEKDNSKICTECYLKEFGENGIRKKIYSTRGRKVEPKYNIFNSTKELSSTH |
| | YNYAIRDAFQLLDALKKQRQKKLKSIFNQKLRLKEFEDIFSDPQKRIELSLKPHQREKR |
| | YIHLSKSGQESINRGYTLRFVRGKIKSLTRNIEREEKSLRKKTPIHFKGNRLMIFPAGI |
| | KFDFASNKVKISISKNLPNEFNFSGTNVKNEHGKSFFKSRIELIKTQKPKYAYVLRKIK |
| | REYSKLRNYEIEKIRLENPNADLCDFYLQYTIETESRNNEEINGIIGIDRGITNLACLV |
| | LLKKGDKKPSGVKFYKGNKILGMKIAYRKHLYLLKGKRNKLRKQRQIRAIEPKINLILH |
| | QISKDIVKIAKEKNFAIALEQLEKPKKARFAQRKKEKYKLALFTFKNLSTLIEYKSKRE |
| | GIPVIYVPPEKTSQMCSHCAINGDEHVDTQRPYKKPNAQKPSYSLFKCNKCGIELNADY |
| | NAAFNIAQKGLKTLMLNHSH |
| SEQ ID | MLQTLLVKLDPSKEQYKMLYETMERFNEACNQIAETVFAIHSANKIEVQKTVYYPIREK |
| NO: 27 | FGLSAQLTILAIRKVCEAYKRDKSIKPEFRLDGALVYDQRVLSWKGLDKVSLVTLQGRQ |
| | IIPIKFGDYQKARMDRIRGQADLILVKGVFYLCVVVEVSEESPYDPKGVLGVDLGIKNL |
| | AVDSDGEVHSGEQTTNTRERLDSLKARLQSKGTKSAKRHLKKLSGRMAKFSKDVNHCIS |
| | KKLVAKAKGTLMSIALEDLQGIRDRVTVRKAQRRNLHTWNFGLLRMFVDYKAKIAGVPL |
| | VFVDPRNTSRTCPSCGHVAKANRPTRDEFRCVSCGFAGAADHIAAMNIAFRAEVSQPIV |
| | TRFFVQSQAPSFRVG |
| SEQ ID | MDEEPDSAEPNLAPISVKLKLVKLDGEKLAALNDYFNEYAKAVNFCELKMQKIRKNLVN |
| NO: 28 | IRGTYLKEKKAWINQTGECCICKKIDELRCEDKNPDINGKICKKCYNGRYGNQMIRKLF |
| | VSTNKRAVPKSLDIRKVARLHNTHYHRIPPEAADIIKAIETAERKRRNRILFDERRYNE |
| | LKDALENEEKRVARPKKPKEREVRYVPISKKDTPSKGYTMNALVRKVSGMAKKIERAKR |
| | NLNKRKKIEYLGRRILLDKNWVREDEDKSEISIPTMKEFFGEMRFEITGPSNVMSPNGR |
| | EYFTKWFDRIKAQPDNYCYLLRKESEDETDFYLQYTWRPDAHPKKDYTGCLGIDIGGSK |
| | LASAVYFDADKNRAKQPIQIFSNPIGKWKTKRQKVIKVLSKAAVRHKTKKLESLRNIEP |
| | RIDVHCHRIARKIVGMALAANAFISMENLEGGIREKQKAKETKKQKFSRNMFVFRKLSK |
| | LIEYKALMEGVKVVYIVPDYTSQLCSSCGTNNTKRPKQAIFMCQNTECRYFGKNINADF |
| | NAAINIAKKALNRKDIVRELS |
| SEQ ID | MEKNNSEQTSITTGIKFKLKLDKETKEKLNNYFDEYGKAINFAVRIIQMQLNDDRLAGK |
| NO: 29 | YKRDEKGKPILGEDGKKILEIPNDFCSCGNQVNHYVNGVSFCQECYKKRFSENGIRKRM |
| | YSAKGRKAEQDINIKNSTNKISKTHFNYAIREAFNLDKSIKKQREKRFKKLKDMKRKLQ |
| | EFLEIRDGKRVICPKIEKQKVERYIHPSWINKEKKLEEFRGYSLSIVNSKIKSFDRNIQ |
| | REEKSLKEKGQINFKAQRLMLDKSVKFLKDNKVSFTISKELPKTFELDLPKKEKKLNWL |
| | NEKLEIIKNQKPKYAYLLRKENNIFLQYTLDSIPEIHSEYSGAVGIDRGVSHIAVYTFL |
| | DKDGKNERPFFLSSSGILRLKNLQKERDKFLRKKHNKIRKKGNMRNIEQKINLILHEYS |
| | KQIVNFAKDKNAFIVFELLEKPKKSRERMSKKIQYKLSQFTFKKLSDLVDYKAKREGIK |
| | VIYVEPAYTSKDCSHCGERVNTQRPFNGNFSLFKCNKCGIVLNSDYNASLNIARKGLNI |
| | SAN |
| SEQ ID | MAEEKFFFCEKCNKDIKIPKNYINKQGAEEKARAKHEHRVHALILGIKFKIYPKKEDIS |
| NO: 30 | KLNDYFDEYAKAVTFTAKIVDKLKAPFLFAGKRDKDTSKKKWVFPVDKCSFCKEKTEIN |
| | YRTKQGKNICNSCYLTEFGEQGLLEKIYATKGRKVSSSFNLFNSTKKLTGTHNNYVVKE |
| | SLQLLDALKKQRSKRLKKLSNTRRKLKQFEEMFEKEDKRFQLPLKEKQRELRFIHVSQK |
| | DRATEFKGYTMNKIKSKIKVLRRNIEREQRSLNRKSPVFFRGTRIRLSPSVQFDDKDNK |
| | IKLTLSKELPKEYSFSGLNVANEHGRKFFAEKLKLIKENKSKYAYLLRRQVNKNNKKPI |
| | YDYYLQYTVEFLPNIITNYNGILGIDRGINTLACIVLLENKKEKPSFVKFFSGKGILNL |
| | KNKRRKQLYFLKGVHNKYRKQQKIRPIEPRIDQILHDISKQIIDLAKEKRVAISLEQLE |
| | KPQKPKFRQSRKAKYKLSQFNFKTLSNYIDYKAKKEGIRVIYIAPEMTSQNCSRCAMKN |
| | DLHVNTQRPYKNTSSLFKCNKCGVELNADYNAAFNIAQKGLKILNS |
| SEQ ID | MISLKLKLLPDEEQKKLLDEMFWKWASICTRVGFGRADKEDLKPPKDAEGVWFSLTQLN |
| NO: 31 | QANTDINDLREAMKHQKHRLEYEKNRLEAQRDDTQDALKNPDRREISTKRKDLFRPKAS |
| | VEKGFLKLKYHQERYWVRRLKEINKLIERKTKTLIKIEKGRIKFKATRITLHQGSFKIR |
| | FGDKPAFLIKALSGKNQIDAPFVVVPEQPICGSVVNSKKYLDEITTNFLAYSVNAMLFG |
| | LSRSEEMLLKAKRPEKIKKKEEKLAKKQSAFENKKKELQKLLGRELTQQEEAIIEETRN |
| | QFFQDFEVKITKQYSELLSKIANELKQKNDFLKVNKYPILLRKPLKKAKSKKINNLSPS |
| | EWKYYLQFGVKPLLKQKSRRKSRNVLGIDRGLKHLLAVTVLEPDKKTFVWNKLYPNPIT |
| | GWKWRRRKLLRSLKRLKRRIKSQKHETIHENQTRKKLKSLQGRIDDLLHNISRKIVETA |
| | KEYDAVIVVEDLQSMRQHGRSKGNRLKTLNYALSLFDYANVMQLIKYKAGIEGIQIYDV |
| | KPAGTSQNCAYCLLAQRDSHEYKRSQENSKIGVCLNPNCQNHKKQIDADLNAARVIASC |
| | YALKINDSQPFGTRKRFKKRTTN |
| SEQ ID | METLSLKLKLNPSKEQLLVLDKMFWKWASICTRLGLKKAEMSDLEPPKDAEGVWFSKTQ |
| NO: 32 | LNQANTDVNDLRKAMQHQGKRIEYELDKVENRRNEIQEMLEKPDRRDISPNRKDLFRPK |
| | AAVEKGYLKLKYHKLGYWSKELKTANKLIERKRKTLAKIDAGKMKFKPTRISLHTNSFR |
| | IKFGEEPKIALSTTSKHEKIELPLITSLQRPLKTSCAKKSKTYLDAAILNFLAYSTNAA |
| | LFGLSRSEEMLLKAKKPEKIEKRDRKLATKRESFDKKLKTLEKLLERKLSEKEKSVFKR |
| | KQTEFFDKFCITLDETYVEALHRIAEELVSKNKYLEIKKYPVLLRKPESRLRSKKLKNL |
| | KPEDWTYYIQFGFQPLLDTPKPIKTKTVLGIDRGVRHLLAVSIFDPRTKTFTFNRLYSN |
| | PIVDWKWRRRKLLRSIKRLKRRLKSEKHVHLHENQFKAKLRSLEGRIEDHFHNLSKEIV |
| | DLAKENNSVIVVENLGGMRQHGRGRGKWLKALNYALSHFDYAKVMQLIKYKAELAGVFV |
| | YDVAPAGTSINCAYCLLNDKDASNYTRGKVINGKKNTKIGECKTCKKEFDADLNAARVI |
| | ALCYEKRLNDPQPFGTRKQFKPKKP |
| SEQ ID | MKALKLQLIPTRKQYKILDEMFWKWASLANRVSQKGESKETLAPKKDIQKIQFNATQLN |
| NO: 33 | QIEKDIKDLRGAMKEQQKQKERLLLQIQERRSTISEMLNDDNNKERDPHRPLNFRPKGW |
| | RKFHTSKHWVGELSKILRQEDRVKKTIERIVAGKISFKPKRIGIWSSNYKINFFKRKIS |
| | INPLNSKGFELTLMTEPTQDLIGKNGGKSVLNNKRYLDDSIKSLLMFALHSRFFGLNNT |
| | DTYLLGGKINPSLVKYYKKNQDMGEFGREIVEKFERKLKQEINEQQKKIIMSQIKEQYS |
| | NRDSAFNKDYLGLINEFSEVFNQRKSERAEYLLDSFEDKIKQIKQEIGESLNISDWDFL |
| | IDEAKKAYGYEEGFTEYVYSKRYLEILNKIVKAVLITDIYFDLRKYPILLRKPLDKIKK |
| | ISNLKPDEWSYYIQFGYDSINPVQLMSTDKFLGIDRGLTHLLAYSVFDKEKKEFIINQL |
| | EPNPIMGWKWKLRKVKRSLQHLERRIRAQKMVKLPENQMKKKLKSIEPKIEVHYHNISR |
| | KIVNLAKDYNASIVVESLEGGGLKQHGRKKNARNRSLNYALSLFDYGKIASLIKYKADL |
| | EGVPMYEVLPAYTSQQCAKCVLEKGSFVDPEIIGYVEDIGIKGSLLDSLFEGTELSSIQ |
| | VLKKIKNKIELSARDNHNKEINLILKYNFKGLVIVRGQDKEEIAEHPIKEINGKFAILD |
| | FVYKRGKEKVGKKGNQKVRYTGNKKVGYCSKHGQVDADLNASRVIALCKYLDINDPILF |
| | GEQRKSFK |
| SEQ ID | MVTRAIKLKLDPTKNQYKLLNEMFWKWASLANRFSQKGASKETLAPKDGTQKIQFNATQ |
| NO: 34 | LNQIKKDVDDLRGAMEKQGKQKERLLIQIQERLLTISEILRDDSKKEKDPHRPQNFRPF |
| | GWRRFHTSAYWSSEASKLTRQVDRVRRTIERIKAGKINFKPKRIGLWSSTYKINFLKKK |
| | INISPLKSKSFELDLITEPQQKIIGKEGGKSVANSKKYLDDSIKSLLIFAIKSRLFGLN |
| | NKDKPLFENIITPNLVRYHKKGQEQENFKKEVIKKFENKLKKEISQKQKEIIFSQIERQ |
| | YENRDATFSEDYLRAISEFSEIFNQRKKERAKELLNSFNEKIRQLKKEVNGNISEEDLK |
| | ILEVEAEKAYNYENGFIEWEYSEQFLGVLEKIARAVLISDNYFDLKKYPILIRKPTNKS |
| | KKITNLKPEEWDYYIQFGYGLINSPMKIETKNFMGIDRGLTHLLAYSIFDRDSEKFTIN |
| | QLELNPIKGWKWKLRKVKRSLQHLERRMRAQKGVKLPENQMKKRLKSIEPKIESYYHNL |
| | SRKIVNLAKANNASIVVESLEGGGLKQHGRKKNSRHRALNYALSLFDYGKIASLIKYKS |
| | DLEGVPMYEVLPAYTSQQCAKCVLKKGSFVEPEIIGYIEEIGFKENLLTLLFEDTGLSS |
| | VQVLKKSKNKMTLSARDKEGKMVDLVLKYNFKGLVISQEKKKEEIVEFPIKEIDGKFAV |
| | LDSAYKRGKERISKKGNQKLVYTGNKKVGYCSVHGQVDADLNASRVIALCKYLGINEPI |
| | VFGEQRKSFK |
| SEQ ID | LDLITEPIQPHKSSSLRSKEFLEYQISDFLNFSLHSLFFGLASNEGPLVDFKIYDKIVI |
| NO: 35 | PKPEERFPKKESEEGKKLDSFDKRVEEYYSDKLEKKIERKLNTEEKNVIDREKTRIWGE |
| | VNKLEEIRSIIDEINEIKKQKHISEKSKLLGEKWKKVNNIQETLLSQEYVSLISNLSDE |
| | LTNKKKELLAKKYSKFDDKIKKIKEDYGLEFDENTIKKEGEKAFLNPDKFSKYQFSSSY |
| | LKLIGEIARSLITYKGFLDLNKYPIIFRKPINKVKKIHNLEPDEWKYYIQFGYEQINNP |
| | KLETENILGIDRGLTHILAYSVFEPRSSKFILNKLEPNPIEGWKWKLRKLRRSIQNLER |
| | RWRAQDNVKLPENQMKKNLRSIEDKVENLYHNLSRKIVDLAKEKNACIVFEKLEGQGMK |
| | QHGRKKSDRLRGLNYKLSLFDYGKIAKLIKYKAEIEGIPIYRIDSAYTSQNCAKCVLES |
| | RRFAQPEEISCLDDFKEGDNLDKRILEGTGLVEAKIYKKLLKEKKEDFEIEEDIAMFDT |
| | KKVIKENKEKTVILDYVYTRRKEIIGTNHKKNIKGIAKYTGNTKIGYCMKHGQVDADLN |
| | ASRTIALCKNFDINNPEIWK |
| SEQ ID | MSDESLVSSEDKLAIKIKIVPNAEQAKMLDEMFKKWSSICNRISRGKEDIETLRPDEGK |
| NO: 36 | ELQFNSTQLNSATMDVSDLKKAMARQGERLEAEVSKLRGRYETIDASLRDPSRRHTNPQ |
| | KPSSFYPSDWDISGRLTPRFHTARHYSTELRKLKAKEDKMLKTINKIKNGKIVFKPKRI |
| | TLWPSSVNMAFKGSRLLLKPFANGFEMELPIVISPQKTADGKSQKASAEYMRNALLGLA |
| | GYSINQLLFGMNRSQKMLANAKKPEKVEKFLEQMKNKDANFDKKIKALEGKWLLDRKLK |
| | ESEKSSIAVVRTKFFKSGKVELNEDYLKLLKHMANEILERDGFVNLNKYPILSRKPMKR |
| | YKQKNIDNLKPNMWKYYIQFGYEPIFERKASGKPKNIMGIDRGLTHLLAVAVFSPDQQK |
| | FLFNHLESNPIMHWKWKLRKIRRSIQHMERRIRAEKNKHIHEAQLKKRLGSIEEKTEQH |
| | YHIVSSKIINWAIEYEAAIVLESLSHMKQRGGKKSVRTRALNYALSLFDYEKVARLITY |
| | KARIRGIPVYDVLPGMTSKTCATCLLNGSQGAYVRGLETTKAAGKATKRKNMKIGKCMV |
| | CNSSENSMIDADLNAARVIAICKYKNLNDPQPAGSRKVFKRF |
| SEQ ID | MLALKLKIMPTEKQAEILDAMFWKWASICSRIAKMKKKVSVKENKKELSKKIPSNSDIW |
| NO: 37 | FSKTQLCQAEVDVGDHKKALKNFEKRQESLLDELKYKVKAINEVINDESKREIDPNNPS |
| | KFRIKDSTKKGNLNSPKFFTLKKWQKILQENEKRIKKKESTIEKLKRGNIFFNPTKISL |
| | HEEEYSINFGSSKLLLNCFYKYNKKSGINSDQLENKFNEFQNGLNIICSPLQPIRGSSK |
| | RSFEFIRNSIINFLMYSLYAKLFGIPRSVKALMKSNKDENKLKLEEKLKKKKSSFNKTV |
| | KEFEKMIGRKLSDNESKILNDESKKFFEIIKSNNKYIPSEEYLKLLKDISEEIYNSNID |
| | FKPYKYSILIRKPLSKFKSKKLYNLKPTDYKYYLQLSYEPFSKQLIATKTILGIDRGLK |
| | HLLAVSVFDPSQNKFVYNKLIKNPVFKWKKRYHDLKRSIRNRERRIRALTGVHIHENQL |
| | IKKLKSMKNKINVLYHNVSKNIVDLAKKYESTIVLERLENLKQHGRSKGKRYKKLNYVL |
| | SNFDYKKIESLISYKAKKEGVPVSNINPKYTSKTCAKCLLEVNQLSELKNEYNRDSKNS |
| | KIGICNIHGQIDADLNAARVIALCYSKNLNEPHFK |
| SEQ ID | VINLFGYKFALYPNKTQEELLNKHLGECGWLYNKAIEQNEYYKADSNIEEAQKKFELLP |
| NO: 38 | DKNSDEAKVLRGNISKDNYVYRTLVKKKKSEINVQIRKAVVLRPAETIRNLAKVKKKGL |
| | SVGRLKFIPIREWDVLPFKQSDQIRLEENYLILEPYGRLKFKMHRPLLGKPKTFCIKRT |
| | ATDRWTISFSTEYDDSNMRKNDGGQVGIDVGLKTHLRLSNENPDEDPRYPNPKIWKRYD |
| | RRLTILQRRISKSKKLGKNRTRLRLRLSRLWEKIRNSRADLIQNETYEILSENKLIAIE |
| | DLNVKGMQEKKDKKGRKGRTRAQEKGLHRSISDAAFSEFRRVLEYKAKRFGSEVKPVSA |
| | IDSSKECHNCGNKKGMPLESRIYECPKCGLKIDRDLNSAKVILARATGVRPGSNARADT |
| | KISATAGASVQTEGTVSEDFRQQMETSDQKPMQGEGSKEPPMNPEHKSSGRGSKHVNIG |
| | CKNKVGLYNEDENSRSTEKQIMDENRSTTEDMVEIGALHSPVLTT |
| SEQ ID | MIASIDYEAVSQALIVFEFKAKGKDSQYQAIDEAIRSYRFIRNSCLRYWMDNKKVGKYD |
| NO: 39 | LNKYCKVLAKQYPFANKLNSQARQSAAECSWSAISRFYDNCKRKVSGKKGFPKFKKHAR |
| | SVEYKTSGWKLSENRKAITFTDKNGIGKLKLKGTYDLHFSQLEDMKRVRLVRRADGYYV |
| | QFCISVDVKVETEPTGKAIGLDVGIKYFLADSSGNTIENPQFYRKAEKKLNRANRRKSK |
| | KYIRGVKPQSKNYHKARCRYARKHLRVSRQRKEYCKRVAYCVIHSNDVVAYEDLNVKGM |
| | VKNRHLAKSISDVAWSTFRHWLEYFAIKYGKLTIPVAPHNTSQNCSNCDKKVPKSLSTR |
| | THICHHCGYSEDRDVNAAKNILKKALSTVGQTGSLKLGEIEPLLVLEQSCTRKEFL |
| SEQ ID | LAEENTLHLTLAMSLPLNDLPENRTRSELWRRQWLPQKKLSLLLGVNQSVRKAAADCLR |
| NO: 40 | WFEPYQELLWWEPTDPDGKKLLDKEGRPIKRTAGHMRVLRKLEEIAPFRGYQLGSAVKN |
| | GLRHKVADLLLSYAKRKLDPQFTDKTSYPSIGDQFPIVWTGAFVCYEQSITGQLYLYLP |
| | LFPRGSHQEDITNNYDPDRGPALQVFGEKEIARLSRSTSGLLLPLQFDKWGEATFIRGE |
| | NNPPTWKATHRRSDKKWLSEVLLREKDFQPKRVELLVRNGRIFVNVACEIPTKPLLEVE |
| | NFMGVSFGLEHLVTVVVINRDGNVVHQRQEPARRYEKTYFARLERLRRRGGPFSQELET |
| | FHYRQVAQIVEEALRFKSVPAVEQVGNIPKGRYNPRLNLRLSYWPFGKLADLTSYKAVK |
| | EGLPKPYSVYSATAKMLCSTCGAANKEGDQPISLKGPTVYCGNCGTRHNTGFNTALNLA |
| | RRAQELFVKGVVAR |
| SEQ ID | MSQSLLKWHDMAGRDKDASRSLQKSAVEGVLLHLTASHRVALEMLEKSVSQTVAVTMEA |
| NO: 41 | AQQRLVIVLEDDPTKATSRKRVISADLQFTREEFGSLPNWAQKLASTCPEIATKYADKH |
| | INSIRIAWGVAKESTNGDAVEQKLQWQIRLLDVTMFLQQLVLQLADKALLEQIPSSIRG |
| | GIGQEVAQQVTSHIQLLDSGTVLKAELPTISDRNSELARKQWEDAIQTVCTYALPFSRE |
| | RARILDPGKYAAEDPRGDRLINIDPMWARVLKGPTVKSLPLLFVSGSSIRIVKLTLPRK |
| | HAAGHKHTFTATYLVLPVSREWINSLPGTVQEKVQWWKKPDVLATQELLVGKGALKKSA |
| | NTLVIPISAGKKRFFNHILPALQRGFPLQWQRIVGRSYRRPATHRKWFAQLTIGYTNPS |
| | SLPEMALGIHFGMKDILWWALADKQGNILKDGSIPGNSILDFSLQEKGKIERQQKAGKN |
| | VAGKKYGKSLLNATYRVVNGVLEFSKGISAEHASQPIGLGLETIRFVDKASGSSPVNAR |
| | HSNWNYGQLSGIFANKAGPAGFSVTEITLKKAQRDLSDAEQARVLAIEATKRFASRIKR |
| | LATKRKDDTLFV |
| SEQ ID | VEPVEKERFYYRTYTFRLDGQPRTQNLTTQSGWGLLTKAVLDNTKHYWEIVHHARIANQ |
| NO: 42 | PIVFENPVIDEQGNPKLNKLGQPRFWKRPISDIVNQLRALFENQNPYQLGSSLIQGTYW |
| | DVAENLASWYALNKEYLAGTATWGEPSFPEPHPLTEINQWMPLTFSSGKVVRLLKNASG |
| | RYFIGLPILGENNPCYRMRTIEKLIPCDGKGRVTSGSLILFPLVGIYAQQHRRMTDICE |
| | SIRTEKGKLAWAQVSIDYVREVDKRRRMRRTRKSQGWIQGPWQEVFILRLVLAHKAPKL |
| | YKPRCFAGISLGPKTLASCVILDQDERVVEKQQWSGSELLSLIHQGEERLRSLREQSKP |
| | TWNAAYRKQLKSLINTQVFTIVTFLRERGAAVRLESIARVRKSTPAPPVNFLLSHWAYR |
| | QITERLKDLAIRNGMPLTHSNGSYGVRFTCSQCGATNQGIKDPTKYKVDIESETFLCSI |
| | CSHREIAAVNTATNLAKQLLDE |
| SEQ ID | MNDTETSETLTSHRTVCAHLHVVGETGSLPRLVEAALAELITLNGRATQALLSLAKNGL |
| NO: 43 | VLRRDKEENLIAAELTLPCRKNKYADVAAKAGEPILATRINNKGKLVTKKWYGEGNSYH |
| | IVRFTPETGMFTVRVFDRYAFDEELLHLHSEVVFGSDLPKGIKAKTDSLPANFLQAVFT |
| | SFLELPFQGFPDIVVKPAMKQAAEQLLSYVQLEAGENQQAEYPDTNERDPELRLVEWQK |
| | SLHELSVRTEPFEFVRARDIDYYAETDRRGNRFVNITPEWTKFAESPFARRLPLKIPPE |
| | FCILLRRKTEGHAKIPNRIYLGLQIFDGVTPDSTLGVLATAEDGKLFWWHDHLDEFSNL |
| | EGKPEPKLKNKPQLLMVSLEYDREQRFEESVGGDRKICLVILKETRNFRRGWNGRILGI |
| | HFQHNPVITWALMDHDAEVLEKGFIEGNAFLGKALDKQALNEYLQKGGKWVGDRSFGNK |
| | LKGITHTLASLIVRLAREKDAWIALEEISWVQKQSADSVANHEIVEQPHHSLTR |
| SEQ ID | MNDTETSETLTSHRTVCAHLHVVGETGSLPRLVEAALAELITLNGRATQALLSLAKNGL |
| NO: 44 | VLRRDKEENLIAAELTLPCRKNKYADVAAKAGEPILATRINNKGKLVTKKWYGEGNSYH |
| | IVRFTPETGMFTVRVFDRYAFDEELLHLHSEVVFGSDLPKGIKAKTDSLPANFLQAVFT |
| | SFLELPFQGFPDIVVKPAMKQAAEQLLSYVQLEAGENQQAEYPDTNERDPELRLVEWQK |
| | SLHELSVRTEPFEFVRARDIDYYAETDRRGNRFVNITPEWTKFAESPFARRLPLKIPPE |
| | FCILLRRKTEGHAKIPNRIYLGLQIFDGVTPDSTLGVLATAEDGKLFWWHDHLDEFSNL |
| | EGKPEPKLKNKPQLLMVSLEYDREQRFEESVGGDRKICLVTLKETRNFRRGRHGHTRTD |
| | RLPAGNTLWRADFATSAEVAAPKWNGRILGIHFQHNPVITWALMDHDAEVLEKGFIEGN |
| | AFLGKALDKQALNEYLQKGGKWVGDRSFGNKLKGITHTLASLIVRLAREKDAWIALEEI |
| | SWVQKQSADSVANRRFSMWNYSRLATLIEWLGTDIATRDCGTAAPLAHKVSDYLTHFTC |
| | PECGACRKAGQKKEIADTVRAGDILTCRKCGFSGPIPDNFIAEFVAKKALERMLKKKPV |
| SEQ ID | MAKRNFGEKSEALYRAVRFEVRPSKEELSILLAVSEVLRMLFNSALAERQQVETEFIAS |
| NO: 45 | LYAELKSASVPEEISEIRKKLREAYKEHSISLFDQINALTARRVEDEAFASVTRNWQEE |
| | TLDALDGAYKSFLSLRRKGDYDAHSPRSRDSGFFQKIPGRSGFKIGEGRIALSCGAGRK |
| | LSFPIPDYQQGRLAETTKLKKFELYRDQPNLAKSGRFWISVVYELPKPEATTCQSEQVA |
| | FVALGASSIGVVSQRGEEVIALWRSDKHWVPKIEAVEERMKRRVKGSRGWLRLLNSGKR |
| | RMHMISSRQHVQDEREIVDYLVRNHGSHFVVTELVVRSKEGKLADSSKPERGGSLGLNW |
| | AAQNTGSLSRLVRQLEEKVKEHGGSVRKHKLTLTEAPPARGAENKLWMARKLRESFLKE |
| | V |
| SEQ ID | LAKNDEKELLYQSVKFEIYPDESKIRVLTRVSNILVLVWNSALGERRARFELYIAPLYE |
| NO: 46 | ELKKFPRKSAESNALRQKIREGYKEHIPTFFDQLKKLLTPMRKEDPALLGSVPRAYQEE |
| | TLNTLNGSFVSFMTLRRNNDMDAKPPKGRAEDRFHEISGRSGFKIDGSEFVLSTKEQKL |
| | RFPIPNYQLEKLKEAKQIKKFTLYQSRDRRFWISIAYEIELPDQRPFNPEEVIYIAFGA |
| | SSIGVISPEGEKVIDFWRPDKHWKPKIKEVENRMRSCKKGSRAWKKRAAARRKMYAMTQ |
| | RQQKLNHREIVASLLRLGFHFVVTEYTVRSKPGKLADGSNPKRGGAPQGFNWSAQNTGS |
| | FGEFILWLKQKVKEQGGTVQTFRLVLGQSERPEKRGRDNKIEMVRLLREKYLESQTIVV |
| SEQ ID | MAKGKKKEGKPLYRAVRFEIFPTSDQITLFLRVSKNLQQVWNEAWQERQSCYEQFFGSI |
| NO: 47 | YERIGQAKKRAQEAGFSEVWENEAKKGLNKKLRQQEISMQLVSEKESLLQELSIAFQEH |
| | GVTLYDQINGLTARRIIGEFALIPRNWQEETLDSLDGSFKSFLALRKNGDPDAKPPRQR |
| | VSENSFYKIPGRSGFKVSNGQIYLSFGKIGQTLTSVIPEFQLKRLETAIKLKKFELCRD |
| | ERDMAKPGRFWISVAYEIPKPEKVPVVSKQITYLAIGASRLGVVSPKGEFCLNLPRSDY |
| | HWKPQINALQERLEGVVKGSRKWKKRMAACTRMFAKLGHQQKQHGQYEVVKKLLRHGVH |
| | FVVTELKVRSKPGALADASKSDRKGSPTGPNWSAQNTGNIARLIQKLTDKASEHGGTVI |
| | KRNPPLLSLEERQLPDAQRKIFIAKKLREEFLADQK |
| SEQ ID | MAKREKKDDVVLRGTKMRIYPTDRQVTLMDMWRRRCISLWNLLLNLETAAYGAKNTRSK |
| NO: 48 | LGWRSIWARVVEENHAKALIVYQHGKCKKDGSFVLKRDGTVKHPPRERFPGDRKILLGL |
| | FDALRHTLDKGAKCKCNVNQPYALTRAWLDETGHGARTADIIAWLKDFKGECDCTAIST |
| | AAKYCPAPPTAELLTKIKRAAPADDLPVDQAILLDLFGALRGGLKQKECDHTHARTVAY |
| | FEKHELAGRAEDILAWLIAHGGTCDCKIVEEAANHCPGPRLFIWEHELAMIMARLKAEP |
| | RTEWIGDLPSHAAQTVVKDLVKALQTMLKERAKAAAGDESARKTGFPKFKKQAYAAGSV |
| | YFPNTTMFFDVAAGRVQLPNGCGSMRCEIPRQLVAELLERNLKPGLVIGAQLGLLGGRI |
| | WRQGDRWYLSCQWERPQPTLLPKTGRTAGVKIAASIVFTTYDNRGQTKEYPMPPADKKL |
| | TAVHLVAGKQNSRALEAQKEKEKKLKARKERLRLGKLEKGHDPNALKPLKRPRVRRSKL |
| | FYKSAARLAACEAIERDRRDGFLHRVTNEIVHKFDAVSVQKMSVAPMMRRQKQKEKQIE |
| | SKKNEAKKEDNGAAKKPRNLKPVRKLLRHVAMARGRQFLEYKYNDLRGPGSVLIADRLE |
| | PEVQECSRCGTKNPQMKDGRRLLRCIGVLPDGTDCDAVLPRNRNAARNAEKRLRKHREA |
| | HNA |
| SEQ ID | MNEVLPIPAVGEDAADTIMRGSKMRIYPSVRQAATMDLWRRRCIQLWNLLLELEQAAYS |
| NO: 49 | GENRRTQIGWRSIWATVVEDSHAEAVRVAREGKKRKDGTFRKAPSGKEIPPLDPAMLAK |
| | IQRQMNGAVDVDPKTGEVTPAQPRLFMWEHELQKIMARLKQAPRTHWIDDLPSHAAQSV |
| | VKDLIKALQAMLRERKKRASGIGGRDTGFPKFKKNRYAAGSVYFANTQLRFEAKRGKAG |
| | DPDAVRGEFARVKLPNGVGWMECRMPRHINAAHAYAQATLMGGRIWRQGENWYLSCQWK |
| | MPKPAPLPRAGRTAAIKIAAAIPITTVDNRGQTREYAMPPIDRERIAAHAAAGRAQSRA |
| | LEARKRRAKKREAYAKKRHAKKLERGIAAKPPGRARIKLSPGFYAAAAKLAKLEAEDAN |
| | AREAWLHEITTQIVRNFDVIAVPRMEVAKLMKKPEPPEEKEEQVKAPWQGKRRSLKAAR |
| | VMMRRTAMALIQTTLKYKAVDLRGPQAYEEIAPLDVTAAACSGCGVLKPEWKMARAKGR |
| | EIMRCQEPLPGGKTCNTVLTYTRNSARVIGRELAVRLAERQKA |
| SEQ ID | MTTQKTYNFCFYDQRFFELSKEAGEVYSRSLEEFWKIYDETGVWLSKFDLQKHMRNKLE |
| NO: 50 | RKLLHSDSFLGAMQQVHANLASWKQAKKVVPDACPPRKPKFLQAILFKKSQIKYKNGFL |
| | RLTLGTEKEFLYLKWDINIPLPIYGSVTYSKTRGWKINLCLETEVEQKNLSENKYLSID |
| | LGVKRVATIFDGENTITLSGKKFMGLMHYRNKLNGKTQSRLSHKKKGSNNYKKIQRAKR |
| | KTTDRLLNIQKEMLHKYSSFIVNYAIRNDIGNIIIGDNSSTHDSPNMRGKTNQKISQNP |
| | EQKLKNYIKYKFESISGRVDIVPEPYTSRKCPHCKNIKKSSPKGRTYKCKKCGFIFDRD |
| | GVGAINIYNENVSFGQIISPGRIRSLTEPIGMKFHNEIYFKSYVAA |
| SEQ ID | MSVRSFQARVECDKQTMEHLWRTHKVFNERLPEIIKILFKMKRGECGQNDKQKSLYKSI |
| NO: 51 | SQSILEANAQNADYLLNSVSIKGWKPGTAKKYRNASFTWADDAAKLSSQGIHVYDKKQV |
| | LGDLPGMMSQMVCRQSVEAISGHIELTKKWEKEHNEWLKEKEKWESEDEHKKYLDLREK |
| | FEQFEQSIGGKITKRRGRWHLYLKWLSDNPDFAAWRGNKAVINPLSEKAQIRINKAKPN |
| | KKNSVERDEFFKANPEMKALDNLHGYYERNFVRRRKTKKNPDGFDHKPTFTLPHPTIHP |
| | RWFVFNKPKTNPEGYRKLILPKKAGDLGSLEMRLLTGEKNKGNYPDDWISVKFKADPRL |
| | SLIRPVKGRRVVRKGKEQGQTKETDSYEFFDKHLKKWRPAKLSGVKLIFPDKTPKAAYL |
| | YFTCDIPDEPLTETAKKIQWLETGDVTKKGKKRKKKVLPHGLVSCAVDLSMRRGTTGFA |
| | TLCRYENGKIHILRSRNLWVGYKEGKGCHPYRWTEGPDLGHIAKHKREIRILRSKRGKP |
| | VKGEESHIDLQKHIDYMGEDRFKKAARTIVNFALNTENAASKNGFYPRADVLLLENLEG |
| | LIPDAEKERGINRALAGWNRRHLVERVIEMAKDAGFKRRVFEIPPYGTSQVCSKCGALG |
| | RRYSIIRENNRREIRFGYVEKLFACPNCGYCANADHNASVNLNRRFLIEDSFKSYYDWK |
| | RLSEKKQKEEIETIESKLMDKLCAMHKISRGSISK |
| SEQ ID | MHLWRTHCVFNQRLPALLKRLFAMRRGEVGGNEAQRQVYQRVAQFVLARDAKDSVDLLN |
| NO: 52 | AVSLRKRSANSAFKKKATISCNGQAREVTGEEVFAEAVALASKGVFAYDKDDMRAGLPD |
| | SLFQPLTRDAVACMRSHEELVATWKKEYREWRDRKSEWEAEPEHALYLNLRPKFEEGEA |
| | ARGGRFRKRAERDHAYLDWLEANPQLAAWRRKAPPAVVPIDEAGKRRIARAKAWKQASV |
| | RAEEFWKRNPELHALHKIHVQYLREFVRPRRTRRNKRREGFKQRPTFTMPDPVRHPRWC |
| | LFNAPQTSPQGYRLLRLPQSRRTVGSVELRLLTGPSDGAGFPDAWVNVRFKADPRLAQL |
| | RPVKVPRTVTRGKNKGAKVEADGFRYYDDQLLIERDAQVSGVKLLFRDIRMAPFADKPI |
| | EDRLLSATPYLVFAVEIKDEARTERAKAIRFDETSELTKSGKKRKTLPAGLVSVAVDLD |
| | TRGVGFLTRAVIGVPEIQQTHHGVRLLQSRYVAVGQVEARASGEAEWSPGPDLAHIARH |
| | KREIRRLRQLRGKPVKGERSHVRLQAHIDRMGEDRFKKAARKIVNEALRGSNPAAGDPY |
| | TRADVLLYESLETLLPDAERERGINRALLRWNRAKLIEHLKRMCDDAGIRHFPVSPFGT |
| | SQVCSKCGALGRRYSLARENGRAVIRFGWVERLFACPNPECPGRRPDRPDRPFTCNSDH |
| | NASVNLHRVFALGDQAVAAFRALAPRDSPARTLAVKRVEDTLRPQLMRVHKLADAGVDS |
| | PF |
| SEQ ID | MATLVYRYGVRAHGSARQQDAVVSDPAMLEQLRLGHELRNALVGVQHRYEDGKRAVWSG |
| NO: 53 | FASVAAADHRVTTGETAVAELEKQARAEHSADRTAATRQGTAESLKAARAAVKQARADR |
| | KAAMAAVAEQAKPKIQALGDDRDAEIKDLYRRFCQDGVLLPRCGRCAGDLRSDGDCTDC |
| | GAAHEPRKLYWATYNAIREDHQTAVKLVEAKRKAGQPARLRFRRWTGDGTLTVQLQRMH |
| | GPACRCVTCAEKLTRRARKTDPQAPAVAADPAYPPTDPPRDPALLASGQGKWRNVLQLG |
| | TWIPPGEWSAMSRAERRRVGRSHIGWQLGGGRQLTLPVQLHRQMPADADVAMAQLTRVR |
| | VGGRHRMSVALTAKLPDPPQVQGLPPVALHLGWRQRPDGSLRVATWACPQPLDLPPAVA |
| | DVVVSHGGRWGEVIMPARWLADAEVPPRLLGRRDKAMEPVLEALADWLEAHTEACTARM |
| | TPALVRRWRSQGRLAGLTNRWRGQPPTGSAEILTYLEAWRIQDKLLWERESHLRRRLAA |
| | RRDDAWRRVASWLARHAGVLVVDDADIAELRRRDDPADTDPTMPASAAQAARARAALAA |
| | PGRLRHLATITATRDGLGVHTVASAGLTRLHRKCGHQAQPDPRYAASAVVTCPGCGNGY |
| | DQDYNAAMLMLDRQQQP |
| SEQ ID | MSRVELHRAYKFRLYPTPAQVAELAEWERQLRRLYNLAHSQRLAAMQRHVRPKSPGVLK |
| NO: 54 | SECLSCGAVAVAEIGTDGKAKKTVKHAVGCSVLECRSCGGSPDAEGRTAHTAACSFVDY |
| | YRQGREMTQLLEEDDQLARVVCSARQETLRDLEKAWQRWHKMPGFGKPHFKKRIDSCRI |
| | YFSTPKSWAVDLGYLSFTGVASSVGRIKIRQDRVWPGDAKFSSCHVVRDVDEWYAVFPL |
| | TFTKEIEKPKGGAVGINRGAVHAIADSTGRVVDSPKFYARSLGVIRHRARLLDRKVPFG |
| | RAVKPSPTKYHGLPKADIDAAAARVNASPGRLVYEARARGSIAAAEAHLAALVLPAPRQ |
| | TSQLPSEGRNRERARRFLALAHQRVRRQREWFLHNESAHYAQSYTKIAIEDWSTKEMTS |
| | SEPRDAEEMKRVTRARNRSILDVGWYELGRQIAYKSEATGAEFAKVDPGLRETETHVPE |
| | AIVRERDVDVSGMLRGEAGISGTCSRCGGLLRASASGHADAECEVCLHVEVGDVNAAVN |
| | VLKRAMFPGAAPPSKEKAKVTIGIKGRKKKRAA |
| SEQ ID | MSRVELHRAYKFRLYPTPVQVAELSEWERQLRRLYNLGHEQRLLTLTRHLRPKSPGVLK |
| NO: 55 | GECLSCDSTQVQEVGADGRPKTTVRHAEQCPTLACRSCGALRDAEGRTAHTVACAFVDY |
| | YRQGREMTELLAADDQLARVVCSARQEVLRDLDKAWQRWRKMPGFGKPRFKRRTDSCRI |
| | YFSTPKAWKLEGGHLSFTGAATTVGAIKMRQDRNWPASVQFSSCHVVRDVDEWYAVFPL |
| | TFVAEVARPKGGAVGINRGAVHAIADSTGRVVDSPRYYARALGVIRHRARLFDRKVPSG |
| | HAVKPSPTKYRGLSAIEVDRVARATGFTPGRVVTEALNRGGVAYAECALAAIAVLGHGP |
| | ERPLTSDGRNREKARKFLALAHQRVRRQREWFLHNESAHYARTYSKIAIEDWSTKEMTA |
| | SEPQGEETRRVTRSRNRSILDVGWYELGRQLAYKTEATGAEFAQVDPGLKETETNVPKA |
| | IADARDVDVSGMLRGEAGISGTCSKCGGLLRAPASGHADAECEICLNVEVGDVNAAVNV |
| | LKRAMFPGDAPPASGEKPKVSIGIKGRQKKKKAA |
| SEQ ID | MEAIATGMSPERRVELGILPGSVELKRAYKERLYPMKVQQAELSEWERQLRRLYNLAHE |
| NO: 56 | QRLAALLRYRDWDFQKGACPSCRVAVPGVHTAACDHVDYFRQAREMTQLLEVDAQLSRV |
| | ICCARQEVLRDLDKAWQRWRKKLGGRPRFKRRTDSCRIYLSTPKHWEIAGRYLRLSGLA |
| | SSVGEIRIEQDRAFPEGALLSSCSIVRDVDEWYACLPLTFTQPIERAPHRSVGLNRGVV |
| | HALADSDGRVVDSPKFFERALATVQKRSRDLARKVSGSRNAHKARIKLAKAHQRVRRQR |
| | AAFLHQESAYYSKGFDLVALEDMSVRKMTATAGEAPEMGRGAQRDLNRGILDVGWYELA |
| | RQIDYKRLAHGGELLRVDPGQTTPLACVTEEQPARGISSACAVCGIPLARPASGNARMR |
| | CTACGSSQVGDVNAAENVLTRALSSAPSGPKSPKASIKIKGRQKRLGTPANRAGEASGG |
| | DPPVRGPVEGGTLAYVVEPVSESQSDT |
| SEQ ID | MTVRTYKYRAYPTPEQAEALTSWLRFASQLYNAALEHRKNAWGRHDAHGRGFRFWDGDA |
| NO: 57 | APRKKSDPPGRWVYRGGGGAHISKNDQGKLLTEFRREHAELLPPGMPALVQHEVLARLE |
| | RSMAAFFQRATKGQKAGYPRWRSEHRYDSLTFGLTSPSKERFDPETGESLGRGKTVGAG |
| | TYHNGDLRLTGLGELRILEHRRIPMGAIPKSVIVRRSGKRWFVSIAMEMPSVEPAASGR |
| | PAVGLDMGVVTWGTAFTADTSAAAALVADLRRMATDPSDCRRLEELEREAAQLSEVLAH |
| | CRARGLDPARPRRCPKELTKLYRRSLHRLGELDRACARIRRRLQAAHDIAEPVPDEAGS |
| | AVLIEGSNAGMRHARRVARTQRRVARRTRAGHAHSNRRKKAVQAYARAKERERSARGDH |
| | RHKVSRALVRQFEEISVEALDIKQLTVAPEHNPDPQPDLPAHVQRRRNRGELDAAWGAF |
| | FAALDYKAADAGGRVARKPAPHTTQECARCGTLVPKPISLRVHRCPACGYTAPRTVNSA |
| | RNVLQRPLEEPGRAGPSGANGRGVPHAVA |
| SEQ ID | MNCRYRYRIYPTPGQRQSLARLFGCVRVVWNDALFLCRQSEKLPKNSELQKLCITQAKK |
| NO: 58 | TEARGWLGQVSAIPLQQSVADLGVAFKNFFQSRSGKRKGKKVNPPRVKRRNNRQGARFT |
| | RGGFKVKTSKVYLARIGDIKIKWSRPLPSEPSSVTVIKDCAGQYFLSFVVEVKPEIKPP |
| | KNPSIGIDLGLKTFASCSNGEKIDSPDYSRLYRKLKRCQRRLAKRQRGSKRRERMRVKV |
| | AKLNAQIRDKRKDFLHKLSTKVVNENQVIALEDLNVGGMLKNRKLSRAISQAGWYEFRS |
| | LCEGKAEKHNRDFRVISRWEPTSQVCSECGYRWGKIDLSVRSIVCINCGVEHDRDDNAS |
| | VNIEQAGLKVGVGHTHDSKRTGSACKTSNGAVCVEPSTHREYVQLTLFDW |
| SEQ ID | MKSRWTFRCYPTPEQEQHLARTFGCVRFVWNWALRARTDAFRAGERIGYPATDKALTLL |
| NO: 59 | KQQPETVWLNEVSSVCLQQALRDLQVAFSNFFDKRAAHPSFKRKEARQSANYTERGFSF |
| | DHERRILKLAKIGAIKVKWSRKAIPHPSSIRLIRTASGKYFVSLVVETQPAPMPETGES |
| | VGVDFGVARLATLSNGERISNPKHGAKWQRRLAFYQKRLARATKGSKRRMRIKRHVARI |
| | HEKIGNSRSDTLHKLSTDLVTRFDLICVEDLNLRGMVKNHSLARSLHDASIGSAIRMIE |
| | EKAERYGKNVVKIDRWFPSSKTCSDCGHIVEQLPLNVREWTCPECGTTHDRDANAAANI |
| | LAVGQTVSAHGGTVRRSRAKASERKSQRSANRQGVNRA |
| SEQ ID | KEPLNIGKTAKAVFKEIDPTSLNRAANYDASIELNCKECKFKPFKNVKRYEFNFYNNWY |
| NO: 60 | RCNPNSCLQSTYKAQVRKVEIGYEKLKNEILTQMQYYPWFGRLYQNFFHDERDKMTSLD |
| | EIQVIGVQNKVFFNTVEKAWREIIKKRFKDNKETMETIPELKHAAGHGKRKLSNKSLLR |
| | RRFAFVQKSFKFVDNSDVSYRSFSNNIACVLPSRIGVDLGGVISRNPKREYIPQEISFN |
| | AFWKQHEGLKKGRNIEIQSVQYKGETVKRIEADTGEDKAWGKNRQRRFTSLILKLVPKQ |
| | GGKKVWKYPEKRNEGNYEYFPIPIEFILDSGETSIRFGGDEGEAGKQKHLVIPFNDSKA |
| | TPLASQQTLLENSRFNAEVKSCIGLAIYANYFYGYARNYVISSIYHKNSKNGQAITAIY |
| | LESIAHNYVKAIERQLQNLLLNLRDFSFMESHKKELKKYFGGDLEGTGGAQKRREKEEK |
| | IEKEIEQSYLPRLIRLSLTKMVTKQVEM |
| SEQ ID | ELIVNENKDPLNIGKTAKAVFKEIDPTSINRAANYDASIELACKECKFKPFNNTKRHDF |
| NO: 62 | SFYSNWHRCSPNSCLQSTYRAKIRKTEIGYEKLKNEILNQMQYYPWFGRLYQNFFNDQR |
| | DKMTSLDEIQVTGVQNKIFFNTVEKAWREIIKKRFRDNKETMRTIPDLKNKSGHGSRKL |
| | SNKSLLRRRFAFAQKSFKLVDNSDVSYRAFSNNVACVLPSKIGVDIGGIINKDLKREYI |
| | PQEITFNVFWKQHDGLKKGRNIEIHSVQYKGEIVKRIEADTGEDKAWGKNRQRRFTSLI |
| | LKITPKQGGKKIWKFPEKKNASDYEYFPIPIEFILDNGDASIKFGGEEGEVGKQKHLLI |
| | PFNDSKATPLSSKQMLLETSRFNAEVKSTIGLALYANYFVSYARNYVIKSTYHKNSKKG |
| | QIVTEIYLESISQNFVRAIQRQLQSLMLNLKDWGFMQTHKKELKKYFGSDLEGSKGGQK |
| | RREKEEKIEKEIEASYLPRLIRLSLTKSVTKAEEM |
| SEQ ID | PEEKTSKLKPNSINLAANYDANEKFNCKECKFHPFKNKKRYEFNFYNNLHGCKSCTKST |
| NO: 63 | NNPAVKRIEIGYQKLKFEIKNQMEAYPWFGRLRINFYSDEKRKMSELNEMQVTGVKNKI |
| | FFDAIECAWREILKKRFRESKETLITIPKLKNKAGHGARKHRNKKLLIRRRAFMKKNFH |
| | FLDNDSISYRSFANNIACVLPSKVGVDIGGIISPDVGKDIKPVDISLNLMWASKEGIKS |
| | GRKVEIYSTQYDGNMVKKIEAETGEDKSWGKNRKRRQTSLLLSIPKPSKQVQEFDFKEW |
| | PRYKDIEKKVQWRGFPIKIIFDSNHNSIEFGTYQGGKQKVLPIPFNDSKTTPLGSKMNK |
| | LEKLRFNSKIKSRLGSAIAANKFLEAARTYCVDSLYHEVSSANAIGKGKIFIEYYLEIL |
| | SQNYIEAAQKQLQRFIESIEQWFVADPFQGRLKQYFKDDLKRAKCFLCANREVQTTCYA |
| | AVKLHKSCAEKVKDKNKELAIKERNNKEDAVIKEVEASNYPRVIRLKLTKTITNKAM |
| SEQ ID | SESENKIIEQYYAFLYSFRDKYEKPEFKNRGDIKRKLQNKWEDFLKEQNLKNDKKLSNY |
| NO: 64 | IFSNRNFRRSYDREEENEEGIDEKKSKPKRINCFEKEKNLKDQYDKDAINASANKDGAQ |
| | KWGCFECIFFPMYKIESGDPNKRIIINKTRFKLFDFYLNLKGCKSCLRSTYHPYRSNVY |
| | IESNYDKLKREIGNFLQQKNIFQRMRKAKVSEGKYLTNLDEYRLSCVAMHEKNRWLFFD |
| | SIQKVLRETIKQRLKQMRESYDEQAKTKRSKGHGRAKYEDQVRMIRRRAYSAQAHKLLD |
| | NGYITLFDYDDKEINKVCLTAINQEGFDIGGYLNSDIDNVMPPIEISFHLKWKYNEPIL |
| | NIESPFSKAKISDYLRKIREDLNLERGKEGKARSKKNVRRKVLASKGEDGYKKIFTDFF |
| | SKWKEELEGNAMERVLSQSSGDIQWSKKKRIHYTTLVLNINLLDKKGVGNLKYYEIAEK |
| | TKILSFDKNENKFWPITIQVLLDGYEIGTEYDEIKQLNEKTSKQFTIYDPNTKIIKIPF |
| | TDSKAVPLGMLGINIATLKTVKKTERDIKVSKIFKGGLNSKIVSKIGKGIYAGYFPTVD |
| | KEILEEVEEDTLDNEFSSKSQRNIFLKSIIKNYDKMLKEQLFDFYSFLVRNDLGVRFLT |
| | DRELQNIEDESFNLEKRFFETDRDRIARWFDNTNTDDGKEKFKKLANEIVDSYKPRLIR |
| | LPVVRVIKRIQPVKQREM |
| SEQ ID | KYSTRDFSELNEIQVTACKQDEFFKVIQNAWREIIKKRFLENRENFIEKKIFKNKKGRG |
| NO: 65 | KRQESDKTIQRNRASVMKNFQLIENEKIILRAPSGHVACVFPVKVGLDIGGFKTDDLEK |
| | NIFPPRTITINVFWKNRDRQRKGRKLEVWGIKARTKLIEKVHKWDKLEEVKKKRLKSLE |
| | QKQEKSLDNWSEVNNDSFYKVQIDELQEKIDKSLKGRTMNKILDNKAKESKEAEGLYIE |
| | WEKDFEGEMLRRIEASTGGEEKWGKRRQRRHTSLLLDIKNNSRGSKEIINFYSYAKQGK |
| | KEKKIEFFPFPLTITLDAEEESPLNIKSIPIEDKNATSKYFSIPFTETRATPLSILGDR |
| | VQKFKTKNISGAIKRNLGSSISSCKIVQNAETSAKSILSLPNVKEDNNMEIFINTMSKN |
| | YFRAMMKQMESFIFEMEPKTLIDPYKEKAIKWFEVAASSRAKRKLKKLSKADIKKSELL |
| | LSNTEEFEKEKQEKLEALEKEIEEFYLPRIVRLQLTKTILETPVM |
| SEQ ID | KKLQLLGHKILLKEYDPNAVNAAANFETSTAELCGQCKMKPFKNKRRFQYTFGKNYHGC |
| NO: 66 | LSCIQNVYYAKKRIVQIAKEELKHQLTDSIASIPYKYTSLFSNINSIDELYILKQERAA |
| | FFSNTNSIDELYITGIENNIAFKVISAIWDEIIKKRRQRYAESLTDTGTVKANRGHGGT |
| | AYKSNTRQEKIRALQKQTLHMVTNPYISLARYKNNYIVATLPRTIGMHIGAIKDRDPQK |
| | KLSDYAINFNVFWSDDRQLIELSTVQYTGDMVRKIEAETGENNKWGENMKRTKTSLLLE |
| | ILTKKTTDELTFKDWAFSTKKEIDSVTKKTYQGFPIGIIFEGNESSVKFGSQNYFPLPF |
| | DAKITPPTAEGFRLDWLRKGSFSSQMKTSYGLAIYSNKVTNAIPAYVIKNMFYKIARAE |
| | NGKQIKAKFLKKYLDIAGNNYVPFIIMQHYRVLDTFEEMPISQPKVIRLSLTKTQHIII |
| | KKDKTDSKM |
| SEQ ID | NTSNLINLGKKAINISANYDANLEVGCKNCKELSSNGNFPRQTNVKEGCHSCEKSTYEP |
| NO: 67 | SIYLVKIGERKAKYDVLDSLKKFTFQSLKYQSKKSMKSRNKKPKELKEFVIFANKNKAF |
| | DVIQKSYNHLILQIKKEINRMNSKKRKKNHKRRLFRDREKQLNKLRLIESSNLFLPREN |
| | KGNNHVFTYVAIHSVGRDIGVIGSYDEKLNFETELTYQLYFNDDKRLLYAYKPKQNKII |
| | KIKEKLWNLRKEKEPLDLEYEKPLNKSITFSIKNDNLFKVSKDLMLRRAKFNIQGKEKL |
| | SKEERKINRDLIKIKGLVNSMSYGRFDELKKEKNIWSPHIYREVRQKEIKPCLIKNGDR |
| | IEIFEQLKKKMERLRRFREKRQKKISKDLIFAERIAYNFHTKSIKNTSNKINIDQEAKR |
| | GKASYMRKRIGYETFKNKYCEQCLSKGNVYRNVQKGCSCFENPFDWIKKGDENLLPKKN |
| | EDLRVKGAFRDEALEKQIVKIAFNIAKGYEDFYDNLGESTEKDLKLKFKVGTTINEQES |
| | LKL |
| SEQ ID | TSNPIKLGKKAINISANYDSNLQIGCKNCKFLSYNGNFPRQTNVKEGCHSCEKSTYEPP |
| NO: 68 | VYTVRIGERRSKYDVLDSLKKFIFLSLKYRQSKKMKTRSKGIRGLEEFVISANLKKAMD |
| | VIQKSYRHLILNIKNEIVRMNGKKRNKNHKRLLFRDREKQLNKLRLIEGSSFFKPPTVK |
| | GDNSIFTCVAIHNIGRDIGIAGDYFDKLEPKIELTYQLYYEYNPKKESEINKRLLYAYK |
| | PKQNKIIEIKEKLWNLRKEKSPLDLEYEKPLTKSITFLVKRDGVFRISKDLMLRKAKFI |
| | IQGKEKLSKEERKINRDLIKIKSNIISLTYGRFDELKKDKTIWSPHIFRDVKQGKITPC |
| | IERKGDRMDIFQQLRKKSERLRENRKKRQKKISKDLIFAERIAYNFHTKSIKNTSNLIN |
| | IKHEAKRGKASYMRKRIGNETFRIKYCEQCFPKNNVYKNVQKGCSCFEDPFEYIKKGNE |
| | DLIPNKNQDLKAKGAFRDDALEKQIIKVAFNIAKGYEDFYENLKKTTEKDIRLKFKVGT |
| | IISEEM |
| SEQ ID | NNSINLSKKAINISANYDANLQVRCKNCKFLSSNGNFPRQTDVKEGCHSCEKSTYEPPV |
| NO: 69 | YDVKIGEIKAKYEVLDSLKKFTFQSLKYQLSKSMKFRSKKIKELKEFVIFAKESKALNV |
| | INRSYKHLILNIKNDINRMNSKKRIKNHKGRLFLDRQKQLSKLKLIEGSSFFVPAKNVG |
| | NKSVFTCVAIHSIGRDIGIAGLYDSFTKPVNEITYQIFFSGERRLLYAYKPKQLKILSI |
| | KENLWSLKNEKKPLDLLYEKPLGKNLNFNVKGGDLFRVSKDLMIRNAKFNVHGRQRLSD |
| | EERLINRNFIKIKGEVVSLSYGRFEELKKDRKLWSPHIFKDVRQNKIKPCLVMQGQRID |
| | IFEQLKRKLELLKKIRKSRQKKLSKDLIFGERIAYNFHTKSIKNTSNKINIDSDAKRGR |
| | ASYMRKRIGNETFKLKYCDVCFPKANVYRRVQNGCSCSENPYNYIKKGDKDLLPKKDEG |
| | LAIKGAFRDEKLNKQIIKVAFNIAKGYEDFYDDLKKRTEKDVDLKFKIGTTVLDQKPME |
| | IFDGIVITWL |
| SEQ ID | LLTTVVETNNLAKKAINVAANFDANIDRQYYRCTPNLCRFIAQSPRETKEKDAGCSSCT |
| NO: 70 | QSTYDPKVYVIKIGKLLAKYEILKSLKRFLFMNRYFKQKKTERAQQKQKIGTELNEMSI |
| | FAKATNAMEVIKRATKHCTYDIIPETKSLQMLKRRRHRVKVRSLLKILKERRMKIKKIP |
| | NTFIEIPKQAKKNKSDYYVAAALKSCGIDVGLCGAYEKNAEVEAEYTYQLYYEYKGNSS |
| | TKRILYCYNNPQKNIREFWEAFYIQGSKSHVNTPGTIRLKMEKFLSPITIESEALDERV |
| | WNSDLKIRNGQYGFIKKRSLGKEAREIKKGMGDIKRKIGNLTYGKSPSELKSIHVYRTE |
| | RENPKKPRAARKKEDNFMEIFEMQRKKDYEVNKKRRKEATDAAKIMDFAEEPIRHYHTN |
| | NLKAVRRIDMNEQVERKKTSVFLKRIMQNGYRGNYCRKCIKAPEGSNRDENVLEKNEGC |
| | LDCIGSEFIWKKSSKEKKGLWHTNRLLRRIRLQCFTTAKAYENFYNDLFEKKESSLDII |
| | KLKVSITTKSM |
| SEQ ID | ASTMNLAKQAINFAANYDSNLEIGCKGCKFMSTWSKKSNPKFYPRQNNQANKCHSCTYS |
| NO: 71 | TGEPEVPIIEIGERAAKYKIFTALKKFVFMSVAYKERRRQRFKSKKPKELKELAICSNR |
| | EKAMEVIQKSVVHCYGDVKQEIPRIRKIKVLKNHKGRLFYKQKRSKIKIAKLEKGSFFK |
| | TFIPKVHNNGCHSCHEASLNKPILVTTALNTIGADIGLINDYSTIAPTETDISWQVYYE |
| | FIPNGDSEAVKKRLLYFYKPKGALIKSIRDKYFKKGHENAVNTGFFKYQGKIVKGPIKF |
| | VNNELDFARKPDLKSMKIKRAGFAIPSAKRLSKEDREINRESIKIKNKIYSLSYGRKKT |
| | LSDKDIIKHLYRPVRQKGVKPLEYRKAPDGFLEFFYSLKRKERRLRKQKEKRQKDMSEI |
| | IDAADEFAWHRHTGSIKKTTNHINFKSEVKRGKVPIMKKRIANDSFNTRHCGKCVKQGN |
| | AINKYYIEKQKNCFDCNSIEFKWEKAALEKKGAFKLNKRLQYIVKACFNVAKAYESFYE |
| | DFRKGEEESLDLKFKIGTTTTLKQYPQNKARAM |
| SEQ ID | HSHNLMLTKLGKQAINFAANYDANLEIGCKNCKFLSYSPKQANPKKYPRQTDVHEDGNI |
| NO: 72 | ACHSCMQSTKEPPVYIVPIGERKSKYEILTSLNKFTFLALKYKEKKRQAFRAKKPKELQ |
| | ELAIAFNKEKAIKVIDKSIQHLILNIKPEIARIQRQKRLKNRKGKLLYLHKRYAIKMGL |
| | IKNGKYFKVGSPKKDGKKLLVLCALNTIGRDIGIIGNIEENNRSETEITYQLYFDCLDA |
| | NPNELRIKEIEYNRLKSYERKIKRLVYAYKPKQTKILEIRSKFFSKGHENKVNTGSFNF |
| | ENPLNKSISIKVKNSAFDFKIGAPFIMLRNGKFHIPTKKRLSKEEREINRTLSKIKGRV |
| | FRLTYGRNISEQGSKSLHIYRKERQHPKLSLEIRKQPDSFIDEFEKLRLKQNFISKLKK |
| | QRQKKLADLLQFADRIAYNYHTSSLEKTSNFINYKPEVKRGRTSYIKKRIGNEGFEKLY |
| | CETCIKSNDKENAYAVEKEELCFVCKAKPFTWKKTNKDKLGIFKYPSRIKDFIRAAFTV |
| | AKSYNDFYENLKKKDLKNEIFLKFKIGLILSHEKKNHISIAKSVAEDERISGKSIKNIL |
| | NKSIKLEKNCYSCFFHKEDM |
| SEQ ID | SLERVIDKRNLAKKAINIAANFDANINKGFYRCETNQCMFIAQKPRKTNNTGCSSCLQS |
| NO: 73 | TYDPVIYVVKVGEMLAKYEILKSLKRFVFMNRSFKQKKTEKAKQKERIGGELNEMSIFA |
| | NAALAMGVIKRAIRHCHVDIRPEINRLSELKKTKHRVAAKSLVKIVKQRKTKWKGIPNS |
| | FIQIPQKARNKDADFYVASALKSGGIDIGLCGTYDKKPHADPRWTYQLYFDTEDESEKR |
| | LLYCYNDPQAKIRDFWKTFYERGNPSMVNSPGTIEFRMEGFFEKMTPISIESKDFDFRV |
| | WNKDLLIRRGLYEIKKRKNLNRKAREIKKAMGSVKRVLANMTYGKSPTDKKSIPVYRVE |
| | REKPKKPRAVRKEENELADKLENYRREDFLIRNRRKREATEIAKIIDAAEPPIRHYHTN |
| | HLRAVKRIDLSKPVARKNTSVFLKRIMQNGYRGNYCKKCIKGNIDPNKDECRLEDIKKC |
| | ICCEGTQNIWAKKEKLYTGRINVLNKRIKQMKLECFNVAKAYENFYDNLAALKEGDLKV |
| | LKLKVSIPALNPEASDPEEDM |
| SEQ ID | NASINLGKRAINLSANYDSNLVIGCKNCKFLSFNGNFPRQTNVREGCHSCDKSTYAPEV |
| NO: 74 | YIVKIGERKAKYDVLDSLKKFTFQSLKYQIKKSMRERSKKPKELLEFVIFANKDKAFNV |
| | IQKSYEHLILNIKQEINRMNGKKRIKNHKKRLFKDREKQLNKLRLIGSSSLFFPRENKG |
| | DKDLFTYVAIHSVGRDIGVAGSYESHIEPISDLTYQLFINNEKRLLYAYKPKQNKIIEL |
| | KENLWNLKKEKKPLDLEFTKPLEKSITFSVKNDKLFKVSKDLMLRQAKFNIQGKEKLSK |
| | EERQINRDFSKIKSNVISLSYGRFEELKKEKNIWSPHIYREVKQKEIKPCIVRKGDRIE |
| | LFEQLKRKMDKLKKFRKERQKKISKDLNFAERIAYNFHTKSIKNTSNKINIDQEAKRGK |
| | ASYMRKRIGNESFRKKYCEQCFSVGNVYHNVQNGCSCFDNPIELIKKGDEGLIPKGKED |
| | RKYKGALRDDNLQMQIIRVAFNIAKGYEDFYNNLKEKTEKDLKLKFKIGTTISTQESNN |
| | KEM |
| SEQ ID | SNLIKLGKQAINFAANYDANLEVGCKNCKFLSSTNKYPRQTNVHLDNKMACRSCNQSTM |
| NO: 75 | EPAIYIVRIGEKKAKYDIYNSLTKFNFQSLKYKAKRSQRFKPKQPKELQELSIAVRKEK |
| | ALDIIQKSIDHLIQDIRPEIPRIKQQKRYKNHVGKLFYLQKRRKNKLNLIGKGSFFKVF |
| | SPKEKKNELLVICALTNIGRDIGLIGNYNTIINPLFEVTYQLYYDYIPKKNNKNVQRRL |
| | LYAYKSKNEKILKLKEAFFKRGHENAVNLGSFSYEKPLEKSLTLKIKNDKDDFQVSPSL |
| | RIRTGRFFVPSKRNLSRQEREINRRLVKIKSKIKNMTYGKFETARDKQSVHIFRLERQK |
| | EKLPLQFRKDEKEFMEEFQKLKRRTNSLKKLRKSRQKKLADLLQLSEKVVYNNHTGTLK |
| | KTSNFLNFSSSVKRGKTAYIKELLGQEGFETLYCSNCINKGQKTRYNIETKEKCFSCKD |
| | VPFVWKKKSTDKDRKGAFLFPAKLKDVIKATFTVAKAYEDFYDNLKSIDEKKPYIKFKI |
| | GLILAHVRHEHKARAKEEAGQKNIYNKPIKIDKNCKECFFFKEEAM |
| SEQ ID | NTTRKKFRKRTGFPQSDNIKLAYCSAIVRAANLDADIQKKHNQCNPNLCVGIKSNEQSR |
| NO: 76 | KYEHSDRQALLCYACNQSTGAPKVDYIQIGEIGAKYKILQMVNAYDFLSLAYNLTKLRN |
| | GKSRGHQRMSQLDEVVIVADYEKATEVIKRSINHLLDDIRGQLSKLKKRTQNEHITEHK |
| | QSKIRRKLRKLSRLLKRRRWKWGTIPNPYLKNWVFTKKDPELVTVALLHKLGRDIGLVN |
| | RSKRRSKQKLLPKVGFQLYYKWESPSLNNIKKSKAKKLPKRLLIPYKNVKLFDNKQKLE |
| | NAIKSLLESYQKTIKVEFDQFFQNRTEEIIAEEQQTLERGLLKQLEKKKNEFASQKKAL |
| | KEEKKKIKEPRKAKLLMEESRSLGFLMANVSYALFNTTIEDLYKKSNVVSGCIPQEPVV |
| | VFPADIQNKGSLAKILFAPKDGFRIKFSGQHLTIRTAKFKIRGKEIKILTKTKREILKN |
| | IEKLRRVWYREQHYKLKLFGKEVSAKPRFLDKRKTSIERRDPNKLADQTDDRQAELRNK |
| | EYELRHKQHKMAERLDNIDTNAQNLQTLSFWVGEADKPPKLDEKDARGFGVRTCISAWK |
| | WEMEDLLKKQEEDPLLKLKLSIM |
| SEQ ID | PKKPKFQKRTGFPQPDNLRKEYCLAIVRAANLDADFEKKCTKCEGIKTNKKGNIVKGRT |
| NO: 77 | YNSADKDNLLCYACNISTGAPAVDYVFVGALEAKYKILQMVKAYDFHSLAYNLAKLWKG |
| | RGRGHQRMGGLNEVVIVSNNEKALDVIEKSLNHFHDEIRGELSRLKAKFQNEHLHVHKE |
| | SKLRRKLRKISRLLKRRRWKWDVIPNSYLRNFTFTKTRPDFISVALLHRVGRDIGLVTK |
| | TKIPKPTDLLPQFGFQIYYTWDEPKLNKLKKSRLRSEPKRLLVPYKKIELYKNKSVLEE |
| | AIRHLAEVYTEDLTICFKDFFETQKRKFVSKEKESLKRELLKELTKLKKDFSERKTALK |
| | RDRKEIKEPKKAKLLMEESRSLGFLAANTSYALFNLIAADLYTKSKKACSTKLPRQLST |
| | ILPLEIKEHKSTTSLAIKPEEGFKIRFSNTHLSIRTPKFKMKGADIKALTKRKREILKN |
| | ATKLEKSWYGLKHYKLKLYGKEVAAKPRFLDKRNPSIDRRDPKELMEQIENRRNEVKDL |
| | EYEIRKGQHQMAKRLDNVDTNAQNLQTKSFWVGEADKPPELDSMEAKKLGLRTCISAWK |
| | WFMKDLVLLQEKSPNLKLKLSLTEM |
| SEQ ID | KFSKRQEGFLIPDNIDLYKCLAIVRSANLDADVQGHKSCYGVKKNGTYRVKQNGKKGVK |
| NO: 78 | EKGRKYVFDLIAFKGNIEKIPHEAIEEKDQGRVIVLGKFNYKLILNIEKNHNDRASLEI |
| | KNKIKKLVQISSLETGEFLSDLLSGKIGIDEVYGIIEPDVFSGKELVCKACQQSTYAPL |
| | VEYMPVGELDAKYKILSAIKGYDFLSLAYNLSRNRANKKRGHQKLGGGELSEVVISANY |
| | DKALNVIKRSINHYHVEIKPEISKLKKKMQNEPLKVMKQARIRRELHQLSRKVKRLKWK |
| | WGMIPNPELQNIIFEKKEKDFVSYALLHTLGRDIGLFKDTSMLQVPNISDYGFQIYYSW |
| | EDPKLNSIKKIKDLPKRLLIPYKRLDFYIDTILVAKVIKNLIELYRKSYVYETFGEEYG |
| | YAKKAEDILFDWDSINLSEGIEQKIQKIKDEFSDLLYEARESKRQNFVESFENILGLYD |
| | KNFASDRNSYQEKIQSMIIKKQQENIEQKLKREFKEVIERGFEGMDQNKKYYKVLSPNI |
| | KGGLLYTDTNNLGFFRSHLAFMLLSKISDDLYRKNNLVSKGGNKGILDQTPETMLTLEF |
| | GKSNLPNISIKRKFFNIKYNSSWIGIRKPKFSIKGAVIREITKKVRDEQRLIKSLEGVW |
| | HKSTHFKRWGKPRFNLPRHPDREKNNDDNLMESITSRREQIQLLLREKQKQQEKMAGRL |
| | DKIDKEIQNLQTANFQIKQIDKKPALTEKSEGKQSVRNALSAWKWFMEDLIKYQKRTPI |
| | LQLKLAKM |
| SEQ ID | KFSKRQEGFVIPENIGLYKCLAIVRSANLDADVQGHVSCYGVKKNGTYVLKQNGKKSIR |
| NO: 79 | EKGRKYASDLVAFKGDIEKIPFEVIEEKKKEQSIVLGKFNYKLVLDVMKGEKDRASLTM |
| | KNKSKKLVQVSSLGTDEFLLTLLNEKFGIEEIYGIIEPEVFSGKKLVCKACQQSTYAPL |
| | VEYMPVGELDSKYKILSAIKGYDELSLAYNLARHRSNKKRGHQKLGGGELSEVVISANN |
| | AKALNVIKRSLNHYYSEIKPEISKLRKKMQNEPLKVGKQARMRRELHQLSRKVKRLKWK |
| | WGKIPNLELQNITFKESDRDFISYALLHTLGRDIGMFNKTEIKMPSNILGYGFQIYYDW |
| | EEPKLNTIKKSKNTPKRILIPYKKLDFYNDSILVARAIKELVGLFQESYEWEIFGNEYN |
| | YAKEAEVELIKLDEESINGNVEKKLQRIKENFSNLLEKAREKKRQNFIESFESIARLYD |
| | ESFTADRNEYQREIQSFIIEKQKQSIEKKLKNEFKKIVEKKFNEQEQGKKHYRVLNPTI |
| | INEFLPKDKNNLGFLRSKIAFILLSKISDDLYKKSNAVSKGGEKGIIKQQPETILDLEF |
| | SKSKLPSINIKKKLFNIKYTSSWLGIRKPKFNIKGAKIREITRRVRDVQRTLKSAESSW |
| | YASTHFRRWGFPRENQPRHPDKEKKSDDRLIESITLLREQIQILLREKQKGQKEMAGRL |
| | DDVDKKIQNLQTANFQIKQTGDKPALTEKSAGKQSFRNALSAWKWFMENLLKYQNKTPD |
| | LKLKIARTVM |
| SEQ ID | KWIEPNNIDFNKCLAITRSANLDADVQGHKMCYGIKTNGTYKAIGKINKKHNTGIIEKR |
| NO: 80 | RTYVYDLIVTKEKNEKIVKKTDFMAIDEEIEFDEKKEKLLKKYIKAEVLGTGELIRKDL |
| | NDGEKFDDLCSIEEPQAFRRSELVCKACNQSTYASDIRYIPIGEIEAKYKILKAIKGYD |
| | FLSLKYNLGRLRDSKKRGHQKMGQGELKEFVICANKEKALDVIKRSLNHYLNEVKDEIS |
| | RLNKKMQNEPLKVNDQARWRRELNQISRRLKRLKWKWGEIPNPELKNLIFKSSRPEFVS |
| | YALIHTLGRDIGLINETELKPNNIQEYGFQIYYKWEDPELNHIKKVKNIPKRFIIPYKN |
| | LDLFGKYTILSRAIEGILKLYSSSFQYKSFKDPNLFAKEGEKKITNEDFELGYDEKIKK |
| | IKDDFKSYKKALLEKKKNTLEDSLNSILSVYEQSLLTEQINNVKKWKEGLLKSKESIHK |
| | QKKIENIEDIISRIEELKNVEGWIRTKERDIVNKEETNLKREIKKELKDSYYEEVRKDF |
| | SDLKKGEESEKKPFREEPKPIVIKDYIKFDVLPGENSALGFFLSHLSFNLFDSIQYELF |
| | EKSRLSSSKHPQIPETILDL |
| SEQ ID | FRKFVKRSGAPQPDNLNKYKCIAIVRAANLDADIMSNESSNCVMCKGIKMNKRKTAKGA |
| NO: 81 | AKTTELGRVYAGQSGNLLCTACTKSTMGPLVDYVPIGRIRAKYTILRAVKEYDELSLAY |
| | NLARTRVSKKGGRQKMHSLSELVIAAEYEIAWNIIKSSVIHYHQETKEEISGLRKKLQA |
| | EHIHKNKEARIRREMHQISRRIKRLKWKWHMIPNSELHNFLFKQQDPSFVAVALLHTLG |
| | RDIGMINKPKGSAKREFIPEYGFQIYYKWMNPKLNDINKQKYRKMPKRSLIPYKNLNVF |
| | GDRELIENAMHKLLKLYDENLEVKGSKFFKTRVVAISSKESEKLKRDLLWKGELAKIKK |
| | DFNADKNKMQELFKEVKEPKKANALMKQSRNMGFLLQNISYGALGLLANRMYEASAKQS |
| | KGDATKQPSIVIPLEMEFGNAFPKLLLRSGKFAMNVSSPWLTIRKPKFVIKGNKIKNIT |
| | KLMKDEKAKLKRLETSYHRATHFRPTLRGSIDWDSPYFSSPKQPNTHRRSPDRLSADIT |
| | EYRGRLKSVEAELREGQRAMAKKLDSVDMTASNLQTSNFQLEKGEDPRLTEIDEKGRSI |
| | RNCISSWKKFMEDLMKAQEANPVIKIKIALKDESSVLSEDSM |
| SEQ ID | KFHPENLNKSYCLAIVRAANLDADIQGHINCIGIKSNKSDRNYENKLESLQNVELLCKA |
| NO: 82 | CTKSTYKPNINSVPVGEKKAKYSILSEIKKYDFNSLVYNLKKYRKGKSRGHQKLNELRE |
| | LVITSEYKKALDVINKSVNHYLVNIKNKMSKLKKILQNEHIHVGTLARIRRERNRISRK |
| | LDHYRKKWKFVPNKILKNYVFKNQSPDFVSVALLHKLGRDIGLITKTAILQKSFPEYSL |
| | QLYYKYDTPKLNYLKKSKFKSLPKRILISYKYPKFDINSNYIEESIDKLLKLYEESPIY |
| | KNNSKIIEFFKKSEDNLIKSENDSLKRGIMKEFEKVTKNFSSKKKKLKEELKLKNEDKN |
| | SKMLAKVSRPIGFLKAYLSYMLFNIISNRIFEFSRKSSGRIPQLPSCIINLGNQFENFK |
| | NELQDSNIGSKKNYKYFCNLLLKSSGFNISYEEEHLSIKTPNFFINGRKLKEITSEKKK |
| | IRKENEQLIKQWKKLTFFKPSNLNGKKTSDKIRFKSPNNPDIERKSEDNIVENIAKVKY |
| | KLEDLLSEQRKEFNKLAKKHDGVDVEAQCLQTKSFWIDSNSPIKKSLEKKNEKVSVKKK |
| | MKAIRSCISAWKWFMADLIEAQKETPMIKLKLALM |
| SEQ ID | TTLVPSHLAGIEVMDETTSRNEDMIQKETSRSNEDENYLGVKNKCGINVHKSGRGSSKH |
| NO: 83 | EPNMPPEKSGEGQMPKQDSTEMQQRFDESVTGETQVSAGATASIKTDARANSGPRVGTA |
| | RALIVKASNLDRDIKLGCKPCEYIRSELPMGKKNGCNHCEKSSDIASVPKVESGFRKAK |
| | YELVRRFESFAADSISRHLGKEQARTRGKRGKKDKKEQMGKVNLDEIAILKNESLIEYT |
| | ENQILDARSNRIKEWLRSLRLRLRTRNKGLKKSKSIRRQLITLRRDYRKWIKPNPYRPD |
| | EDPNENSLRLHTKLGVDIGVQGGDNKRMNSDDYETSFSITWRDTATRKICFTKPKGLLP |
| | RHMKFKLRGYPELILYNEELRIQDSQKFPLVDWERIPIFKLRGVSLGKKKVKALNRITE |
| | APRLVVAKRIQVNIESKKKKVLTRYVYNDKSINGRLVKAEDSNKDPLLEFKKQAEEINS |
| | DAKYYENQEIAKNYLWGCEGLHKNLLEEQTKNPYLAFKYGFLNIV |
| SEQ ID | LDFKRTCSQELVLLPEIEGLKLSGTQGVTSLAKKLINKAANVDRDESYGCHHCIHTRTS |
| NO: 84 | LSKPVKKDCNSCNQSTNHPAVPITLKGYKIAFYELWHRFTSWAVDSISKALHRNKVMGK |
| | VNLDEYAVVDNSHIVCYAVRKCYEKRQRSVRLHKRAYRCRAKHYNKSQPKVGRIYKKSK |
| | RRNARNLKKEAKRYFQPNEITNGSSDALFYKIGVDLGIAKGTPETEVKVDVSICFQVYY |
| | GDARRVLRVRKMDELQSFHLDYTGKLKLKGIGNKDTFTIAKRNESLKWGSTKYEVSRAH |
| | KKFKPFGKKGSVKRKCNDYFRSIASWSCEAASQRAQSNLKNAFPYQKALVKCYKNLDYK |
| | GVKKNDMWYRLCSNRIFRYSRIAEDIAQYQSDKGKAKFEFVILAQSVAEYDISAIM |
| SEQ ID | VFLTDDKRKTALRKIRSAFRKTAEIALVRAQEADSLDRQAKKLTIETVSFGAPGAKNAF |
| NO: 85 | IGSLQGYNWNSHRANVPSSGSAKDVFRITELGLGIPQSAHEASIGKSFELVGNVVRYTA |
| | NLLSKGYKKGAVNKGAKQQREIKGKEQLSFDLISNGPISGDKLINGQKDALAWWLIDKM |
| | GFHIGLAMEPLSSPNTYGITLQAFWKRHTAPRRYSRGVIRQWQLPFGRQLAPLIHNFFR |
| | KKGASIPIVLTNASKKLAGKGVLLEQTALVDPKKWWQVKEQVTGPLSNIWERSVPLVLY |
| | TATFTHKHGAAHKRPLTLKVIRISSGSVFLLPLSKVTPGKLVRAWMPDINILRDGRPDE |
| | AAYKGPDLIRARERSFPLAYTCVTQIADEWQKRALESNRDSITPLEAKLVTGSDLLQIH |
| | STVQQAVEQGIGGRISSPIQELLAKDALQLVLQQLFMTVDLLRIQWQLKQEVADGNTSE |
| | KAVGWAIRISNIHKDAYKTAIEPCTSALKQAWNPLSGFEERTFQLDASIVRKRSTAKTP |
| | DDELVIVLRQQAAEMTVAVTQSVSKELMELAVRHSATLHLLVGEVASKQLSRSADKDRG |
| | AMDHWKLLSQSM |
| SEQ ID | EDLLQKALNTATNVAAIERHSCISCLFTESEIDVKYKTPDKIGQNTAGCQSCTFRVGYS |
| NO: 86 | GNSHTLPMGNRIALDKLRETIQRYAWHSLLFNVPPAPTSKRVRAISELRVAAGRERLFT |
| | VITFVQTNILSKLQKRYAANWTPKSQERLSRLREEGQHILSLLESGSWQQKEVVREDQD |
| | LIVCSALTKPGLSIGAFCRPKYLKPAKHALVLRLIFVEQWPGQIWGQSKRTRRMRRRKD |
| | VERVYDISVQAWALKGKETRISECIDTMRRHQQAYIGVLPFLILSGSTVRGKGDCPILK |
| | EITRMRYCPNNEGLIPLGIFYRGSANKLLRVVKGSSFTLPMWQNIETLPHPEPFSPEGW |
| | TATGALYEKNLAYWSALNEAVDWYTGQILSSGLQYPNQNEFLARLQNVIDSIPRKWFRP |
| | QGLKNLKPNGQEDIVPNEFVIPQNAIRAHHVIEWYHKTNDLVAKTLLGWGSQTTLNQTR |
| | PQGDLRFTYTRYYFREKEVPEV |
| SEQ ID | VPKKKLMRELAKKAVFEAIFNDPIPGSFGCKRCTLIDGARVTDAIEKKQGAKRCAGCEP |
| NO: 87 | CTFHTLYDSVKHALPAATGCDRTAIDTGLWEILTALRSYNWMSFRRNAVSDASQKQVWS |
| | IEELAIWADKERALRVILSALTHTIGKLKNGFSRDGVWKGGKQLYENLAQKDLAKGLFA |
| | NGEIFGKELVEADHDMLAWTIVPNHQFHIGLIRGNWKPAAVEASTAFDARWLTNGAPLR |
| | DTRTHGHRGRRFNRTEKLTVLCIKRDGGVSEEFRQERDYELSVMLLQPKNKLKPEPKGE |
| | LNSFEDLHDHWWFLKGDEATALVGLTSDPTVGDFIQLGLYIRNPIKAHGETKRRLLICF |
| | EPPIKLPLRRAFPSEAFKTWEPTINVFRNGRRDTEAYYDIDRARVFEFPETRVSLEHLS |
| | KQWEVLRLEPDRENTDPYEAQQNEGAELQVYSLLQEAAQKMAPKVVIDPFGQFPLELFS |
| | TFVAQLFNAPLSDTKAKIGKPLDSGFVVESHLHLLEEDFAYRDFVRVTEMGTEPTFRVI |
| | HYSNGEGYWKKTVLKGKNNIRTALIPEGAKAAVDAYKNKRCPLTLEAAILNEEKDRRLV |
| | LGNKALSLLAQTARGNLTILEALAAEVLRPLSGTEGVVHLHACVTRHSTLTESTETDNM |
| SEQ ID | VEKLFSERLKRAMWLKNEAGRAPPAETLTLKHKRVSGGHEKVKEELQRVLRSLSGTNQA |
| NO: 88 | AWNLGLSGGREPKSSDALKGEKSRVVLETVVFHSGHNRVLYDVIEREDQVHQRSSIMHM |
| | RRKGSNLLRLWGRSGKVRRKMREEVAEIKPVWHKDSRWLAIVEEGRQSVVGISSAGLAV |
| | FAVQESQCTTAEPKPLEYVVSIWFRGSKALNPQDRYLEFKKLKTTEALRGQQYDPIPFS |
| | LKRGAGCSLAIRGEGIKFGSRGPIKQFFGSDRSRPSHADYDGKRRLSLFSKYAGDLADL |
| | TEEQWNRTVSAFAEDEVRRATLANIQDFLSISHEKYAERLKKRIESIEEPVSASKLEAY |
| | LSAIFETFVQQREALASNFLMRLVESVALLISLEEKSPRVEFRVARYLAESKEGFNRKA |
| | M |
| SEQ ID | VVITQSELYKERLLRVMEIKNDRGRKEPRESQGLVLRFTQVTGGQEKVKQKLWLIFEGF |
| NO: 89 | SGTNQASWNFGQPAGGRKPNSGDALKGPKSRVTYETVVFHFGLRLLSAVIERHNLKQQR |
| | QTMAYMKRRAAARKKWARSGKKCSRMRNEVEKIKPKWHKDPRWFDIVKEGEPSIVGISS |
| | AGFAIYIVEEPNFPRQDPLEIEYAISIWFRRDRSQYLTFKKIQKAEKLKELQYNPIPFR |
| | LKQEKTSLVFESGDIKFGSRGSIEHFRDEARGKPPKADMDNNRRLTMFSVFSGNLTNLT |
| | EEQYARPVSGLLAPDEKRMPTLLKKLQDFFTPIHEKYGERIKQRLANSEASKRPFKKLE |
| | EYLPAIYLEFRARREGLASNWVLVLINSVRTLVRIKSEDPYIEFKVSQYLLEKEDNKAL |
| SEQ ID | KQDALFEERLKKAIFIKRQADPLQREELSLLPPNRKIVTGGHESAKDTLKQILRAINGT |
| NO: 90 | NQASWNPGTPSGKRDSKSADALAGPKSRVKLETVVFHVGHRLLKKVVEYQGHQKQQHGL |
| | KAFMRTCAAMRKKWKRSGKVVGELREQLANIQPKWHYDSRPLNLCFEGKPSVVGLRSAG |
| | IALYTIQKSVVPVKEPKPIEYAVSIWFRGPKAMDREDRCLEFKKLKIATELRKLQFEPI |
| | VSTLTQGIKGFSLYIQGNSVKFGSRGPIKYFSNESVRQRPPKADPDGNKRLALFSKFSG |
| | DLSDLTEEQWNRPILAFEGIIRRATLGNIQDYLTVGHEQFAISLEQLLSEKESVLQMSI |
| | EQQRLKKNLGKKAENEWVESFGAEQARKKAQGIREYISGFFQEYCSQREQWAENWVQQL |
| | NKSVRLFLTIQDSTPFIEFRVARYLPKGEKKKGKAM |
| SEQ ID | ANHAERHKRLRKEANRAANRNRPLVADCDTGDPLVGICRLLRRGDKMQPNKTGCRSCEQ |
| NO: 91 | VEPELRDAILVSGPGRLDNYKYELFQRGRAMAVHRLLKRVPKLNRPKKAAGNDEKKAEN |
| | KKSEIQKEKQKQRRMMPAVSMKQVSVADFKHVIENTVRHLFGDRRDREIAECAALRAAS |
| | KYFLKSRRVRPRKLPKLANPDHGKELKGLRLREKRAKLKKEKEKQAELARSNQKGAVLH |
| | VATLKKDAPPMPYEKTQGRNDYTTFVISAAIKVGATRGTKPLLTPQPREWQCSLYWRDG |
| | QRWIRGGLLGLQAGIVLGPKLNRELLEAVLQRPIECRMSGCGNPLQVRGAAVDFFMTTN |
| | PFYVSGAAYAQKKFKPFGTKRASEDGAAAKAREKLMTQLAKVLDKVVTQAAHSPLDGIW |
| | ETRPEAKLRAMIMALEHEWIFLRPGPCHNAAEEVIKCDCTGGHAILWALIDEARGALEH |
| | KEFYAVTRAHTHDCEKQKLGGRLAGFLDLLIAQDVPLDDAPAARKIKTLLEATPPAPCY |
| | KAATSIATCDCEGKFDKLWAIIDATRAGHGTEDLWARTLAYPQNVNCKCKAGKDLTHRL |
| | ADFLGLLIKRDGPFRERPPHKVTGDRKLVFSGDKKCKGHQYVILAKAHNEEVVRAWISR |
| | WGLKSRTNKAGYAATELNLLLNWLSICRRRWMDMLTVQRDTPYIRMKTGRLVVDDKKER |
| | KAM |
| SEQ ID | AKQREALRVALERGIVRASNRTYTLVTNCTKGGPLPEQCRMIERGKARAMKWEPKLVGC |
| NO: 92 | GSCAAATVDLPAIEEYAQPGRLDVAKYKLTTQILAMATRRMMVRAAKLSRRKGQWPAKV |
| | QEEKEEPPEPKKMLKAVEMRPVAIVDENRVIQTTIEHLWAERANADEAELKALKAAAAY |
| | FGPSLKIRARGPPKAAIGRELKKAHRKKAYAERKKARRKRAELARSQARGAAAHAAIRE |
| | RDIPPMAYERTQGRNDVTTIPIAAAIKIAATRGARPLPAPKPMKWQCSLYWNEGQRWIR |
| | GGMLTAQAYAHAANIHRPMRCEMWGVGNPLKVRAFEGRVADPDGAKGRKAEFRLQTNAF |
| | YVSGAAYRNKKFKPFGTDRGGIGSARKKRERLMAQLAKILDKVVSQAAHSPLDDIWHTR |
| | PAQKLRAMIKQLEHEWMFLRPQAPTVEGTKPDVDVAGNMQRQIKALMAPDLPPIEKGSP |
| | AKRFTGDKRKKGERAVRVAEAHSDEVVTAWISRWGIQTRRNEGSYAAQELELLLNWLQI |
| | CRRRWLDMTAAQRVSPYIRMKSGRMITDAADEGVAPIPLVENM |
| SEQ ID | KSISGRSIKHMACLKDMLKSEITEIEEKQKKESLRKWDYYSKFSDEILFRRNLNVSANH |
| NO: 93 | DANACYGCNPCAFLKEVYGFRIERRNNERIISYRRGLAGCKSCVQSTGYPPIEFVRRKF |
| | GADKAMEIVREVLHRRNWGALARNIGREKEADPILGELNELLLVDARPYFGNKSAANET |
| | NLAFNVITRAAKKFRDEGMYDIHKQLDIHSEEGKVPKGRKSRLIRIERKHKAIHGLDPG |
| | ETWRYPHCGKGEKYGVWLNRSRLIHIKGNEYRCLTAFGTTGRRMSLDVACSVLGHPLVK |
| | KKRKKGKKTVDGTELWQIKKATETLPEDPIDCTFYLYAAKPTKDPFILKVGSLKAPRWK |
| | KLHKDFFEYSDTEKTQGQEKGKRVVRRGKVPRILSLRPDAKFKVSIWDDPYNGKNKEGT |
| | LLRMELSGLDGAKKPLILKRYGEPNTKPKNFVFWRPHITPHPLTFTPKHDFGDPNKKTK |
| | RRRVFNREYYGHLNDLAKMEPNAKFFEDREVSNKKNPKAKNIRIQAKESLPNIVAKNGR |
| | WAAFDPNDSLWKLYLHWRGRRKTIKGGISQEFQEFKERLDLYKKHEDESEWKEKEKLWE |
| | NHEKEWKKTLEIHGSIAEVSQRCVMQSMMGPLDGLVQKKDYVHIGQSSLKAADDAWTFS |
| | ANRYKKATGPKWGKISVSNLLYDANQANAELISQSISKYLSKQKDNQGCEGRKMKFLIK |
| | IIEPLRENFVKHTRWLHEMTQKDCEVRAQFSRVSM |
| SEQ ID | FPSDVGADALKHVRMLQPRLTDEVRKVALTRAPSDRPALARFAAVAQDGLAFVRHLNVS |
| NO: 94 | ANHDSNCTFPRDPRDPRRGPCEPNPCAFLREVWGFRIVARGNERALSYRRGLAGCKSCV |
| | QSTGFPSVPFHRIGADDCMRKLHEILKARNWRLLARNIGREREADPLLTELSEYLLVDA |
| | RTYPDGAAPNSGRLAENVIKRAAKKFRDEGMRDIHAQLRVHSREGKVPKGRLQRLRRIE |
| | RKHRAIHALDPGPSWEAEGSARAEVQGVAVYRSQLLRVGHHTQQIEPVGIVARTLFGVG |
| | RTDLDVAVSVLGAPLTKRKKGSKTLESTEDFRIAKARETRAEDKIEVAFVLYPTASLLR |
| | DEIPKDAFPAMRIDRFLLKVGSVQADREILLQDDYYRFGDAEVKAGKNKGRTVTRPVKV |
| | PRLQALRPDAKFRVNVWADPFGAGDSPGTLLRLEVSGVTRRSQPLRLLRYGQPSTQPAN |
| | FLCWRPHRVPDPMTFTPRQKFGERRKNRRTRRPRVFERLYQVHIKHLAHLEPNRKWFEE |
| | ARVSAQKWAKARAIRRKGAEDIPVVAPPAKRRWAALQPNAELWDLYAHDREARKRFRGG |
| | RAAEGEEFKPRLNLYLAHEPEAEWESKRDRWERYEKKWTAVLEEHSRMCAVADRTLPQF |
| | LSDPLGARMDDKDYAFVGKSALAVAEAFVEEGTVERAQGNCSITAKKKFASNASRKRLS |
| | VANLLDVSDKADRALVFQAVRQYVQRQAENGGVEGRRMAFLRKLLAPLRQNFVCHTRWL |
| | HM |
| SEQ ID | AARKKKRGKIGITVKAKEKSPPAAGPFMARKLVNVAANVDGVEVHLCVECEADAHGSAS |
| NO: 95 | ARLLGGCRSCTGSIGAEGRLMGSVDVDRERVIAEPVHTETERLGPDVKAFEAGTAESKY |
| | AIQRGLEYWGVDLISRNRARTVRKMEEADRPESSTMEKTSWDEIAIKTYSQAYHASENH |
| | LFWERQRRVRQHALALFRRARERNRGESPLQSTQRPAPLVLAALHAEAAAISGRARAEY |
| | VLRGPSANVRAAAADIDAKPLGHYKTPSPKVARGFPVKRDLLRARHRIVGLSRAYFKPS |
| | DVVRGTSDAIAHVAGRNIGVAGGKPKEIEKTFTLPFVAYWEDVDRVVHCSSFKADGPWV |
| | RDQRIKIRGVSSAVGTFSLYGLDVAWSKPTSFYIRCSDIRKKFHPKGFGPMKHWRQWAK |
| | ELDRLTEQRASCVVRALQDDEELLQTMERGQRYYDVFSCAATHATRGEADPSGGCSRCE |
| | LVSCGVAHKVTKKAKGDTGIEAVAVAGCSLCESKLVGPSKPRVHRQMAALRQSHALNYL |
| | RRLQREWEALEAVQAPTPYLRFKYARHLEVRSM |
| SEQ ID | AAKKKKQRGKIGISVKPKEGSAPPADGPFMARKLVNVAANVDGVEVNLCIECEADAHGS |
| NO: 96 | APARLLGGCKSCTGSIGAEGRLMGSVDVDRADAIAKPVNTETEKLGPDVQAFEAGTAET |
| | KYALQRGLEYWGVDLISRNRSRTVRRTEEGQPESATMEKTSWDEIAIKSYTRAYHASEN |
| | HLFWERQRRVRQHALALFKRAKERNRGDSTLPREPGHGLVAIAALACEAYAVGGRNLAE |
| | TVVRGPTFGTARAVRDVEIASLGRYKTPSPKVAHGSPVKRDFLRARHRIVGLARAYYRP |
| | SDVVRGTSDAIAHVAGRNIGVAGGKPRAVEAVFTLPFVAYWEDVDRVVHCSSFQVSAPW |
| | NRDQRMKIAGVTTAAGTFSLHGGELKWAKPTSFYIRCSDTRRKFRPKGFGPMKRWRQWA |
| | KDLDRIVEQRASCVVRALQDDAALLETMERGQRYYDVFACAVTHATRGEADRLAGCSRC |
| | ALTPCQEAHRVTTKPRGDAGVEQVQTSDCSLCEGKLVGPSKPRLHRTLTLLRQEHGLNY |
| | LRRLQREWESLEAVQVPTPYLRFKYARHLEVRSM |
| SEQ ID | TDSQSESVPEVVYALTGGEVPGRVPPDGGSAEGARNAPTGLRKQRGKIKISAKPSKPGS |
| NO: 97 | PASSLARTLVNEAANVDGVQSSGCATCRMRANGSAPRALPIGCVACASSIGRAPQEETV |
| | CALPTTQGPDVRLLEGGHALRKYDIQRALEYWGVDLIGRNLDRQAGRGMEPAEGATATM |
| | KRVSMDELAVLDFGKSYYASEQHLFAARQRRVRQHAKALKIRAKHANRSGSVKRALDRS |
| | RKQVTALAREFFKPSDVVRGDSDALAHVVGRNLGVSRHPAREIPQTFTLPLCAYWEDVD |
| | RVISCSSLLAGEPFARDQEIRIEGVSSALGSLRLYRGAIEWHKPTSLYIRCSDTRRKFR |
| | PRGGLKKRWRQWAKDLDRLVEQRACCIVRSLQADVELLQTMERAQRFYDVHDCAATHVG |
| | PVAVRCSPCAGKQFDWDRYRLLAALRQEHALNYLRRLQREWESLEAQQVKMPYLRFKYA |
| | RKLEVSGPLIGLEVRREPSMGTAIAEM |
| SEQ ID | AGTAGRRHGSLGARRSINIAGVTDRHGRWGCESCVYTRDQAGNRARCAPCDQSTYAPDV |
| NO: 98 | QEVTIGQRQAKYTIFLTLQSFSWTNTMRNNKRAAAGRSKRTTGKRIGQLAEIKITGVGL |
| | AHAHNVIQRSLQHNITKMWRAEKGKSKRVARLKKAKQLTKRRAYFRRRMSRQSRGNGFF |
| | RTGKGGIHAVAPVKIGLDVGMIASGSSEPADEQTVTLDAIWKGRKKKIRLIGAKGELAV |
| | AACRFREQQTKGDKCIPLILQDGEVRWNQNNWQCHPKKLVPLCGLEVSRKFVSQADRLA |
| | QNKVASPLAARFDKTSVKGTLVESDFAAVLVNVTSIYQQCHAMLLRSQEPTPSLRVQRT |
| | ITSM |
| SEQ ID | GVRFSPAQSQVFFRTVIPQSVEARFAINMAAIHDAAGAFGCSVCRFEDRTPRNAKAVHG |
| NO: 99 | CSPCTRSTNRPDVFVLPVGAIKAKYDVFMRLLGFNWTHLNRRQAKRVTVRDRIGQLDEL |
| | AISMLTGKAKAVLKKSICHNVDKSFKAMRGSLKKLHRKASKTGKSQLRAKLSDLRERTN |
| | TTQEGSHVEGDSDVALNKIGLDVGLVGKPDYPSEESVEVVVCLYFVGKVLILDAQGRIR |
| | DMRAKQYDGFKIPIIQRGQLTVLSVKDLGKWSLVRQDYVLAGDLRFEPKISKDRKYAEC |
| | VKRIALITLQASLGFKERIPYYVTKQVEIKNASHIAFVTEAIQNCAENFREMTEYLMKY |
| | QEKSPDLKVLLTQLM |
| SEQ ID | RAVVGKVFLEQARRALNLATNFGTNHRTGCNGCYVTPGKLSIPQDGEKNAAGCTSCLMK |
| NO: 100 | ATASYVSYPKPLGEKVAKYSTLDALKGFPWYSLRLNLRPNYRGKPINGVQEVAPVSKFR |
| | LAEEVIQAVQRYHFTELEQSFPGGRRRLRELRAFYTKEYRRAPEQRQHVVNGDRNIVVV |
| | TVLHELGESVGMFNEVELLPKTPIECAVNVFIRGNRVLLEVRKPQFDKERLLVESLWKK |
| | DSRRHTAKWTPPNNEGRIFTAEGWKDFQLPLLLGSTSRSLRAIEKEGFVQLAPGRDPDY |
| | NNTIDEQHSGRPFLPLYLYLQGTISQEYCVFAGTWVIPFQDGISPYSTKDTFQPDLKRK |
| | AYSLLLDAVKHRLGNKVASGLQYGRFPAIEELKRLVRMHGATRKIPRGEKDLLKKGDPD |
| | TPEWWLLEQYPEFWRLCDAAAKRVSQNVGLLLSLKKQPLWQRRWLESRTRNEPLDNLPL |
| | SMALTLHLTNEEAL |
| SEQ ID | AAVYSKFYIENHFKMGIPETLSRIRGPSIIQGFSVNENYINIAGVGDRDFIFGCKKCKY |
| NO: 101 | TRGKPSSKKINKCHPCKRSTYPEPVIDVRGSISEFKYKIYNKLKQEPNQSIKQNTKGRM |
| | NPSDHTSSNDGIIINGIDNRIAYNVIFSSYKHLMEKQINLLRDTTKRKARQIKKYNNSG |
| | KKKHSLRSQTKGNLKNRYHMLGMFKKGSLTITNEGDFITAVRKVGLDISLYKNESLNKQ |
| | EVETELCLNIKWGRTKSYTVSGYIPLPINIDWKLYLFEKETGLTLRLFGNKYKIQSKKF |
| | LIAQLFKPKRPPCADPVVKKAQKWSALNAHVQQMAGLFSDSHLLKRELKNRMHKQLDFK |
| | SLWVGTEDYIKWFEELSRSYVEGAEKSLEFFRQDYFCFNYTKQTTM |
| SEQ ID | PQQQRDLMLMAANYDQDYGNGCGPCTVVASAAYRPDPQAQHGCKRHLRTLGASAVTHVG |
| NO: 102 | LGDRTATITALHRLRGPAALAARARAAQAASAPMTPDTDAPDDRRRLEAIDADDVVLVG |
| | AHRALWSAVRRWADDRRAALRRRLHSEREWLLKDQIRWAELYTLIEASGTPPQGRWRNT |
| | LGALRGQSRWRRVLAPTMRATCAETHAELWDALAELVPEMAKDRRGLLRPPVEADALWR |
| | APMIVEGWRGGHSVVVDAVAPPLDLPQPCAWTAVRLSGDPRQRWGLHLAVPPLGQVQPP |
| | DPLKATLAVSMRHRGGVRVRTLQAMAVDADAPMQRHLQVPLTLQRGGGLQWGIHSRGVR |
| | RREARSMASWEGPPIWTGLQLVNRWKGQGSALLAPDRPPDTPPYAPDAAVAPAQPDTKR |
| | ARRTLKEACTVCRCAPGHMRQLQVTLTGDGTWRRFRLRAPQGAKRKAEVLKVATQHDER |
| | IANYTAWYLKRPEHAAGCDTCDGDSRLDGACRGCRPLLVGDQCFRRYLDKIEADRDDGL |
| | AQIKPKAQEAVAAMAAKRDARAQKVAARAAKLSEATGQRTAATRDASHEARAQKELEAV |
| | ATEGTTVRHDAAAVSAFGSWVARKGDEYRHQVGVLANRLEHGLRLQELMAPDSVVADQQ |
| | RASGHARVGYRYVLTAM |
| SEQ ID | AVAHPVGRGNAGSPGARGPEELPRQLVNRASNVTRPATYGCAPCRHVRLSIPKPVLTGC |
| NO: 103 | RACEQTTHPAPKRAVRGGADAAKYDLAAFFAGWAADLEGRNRRRQVHAPLDPQPDPNHE |
| | PAVTLQKIDLAEVSIEEFQRVLARSVKHRHDGRASREREKARAYAQVAKKRRNSHAHGA |
| | RTRRAVRRQTRAVRRAHRMGANSGEILVASGAEDPVPEAIDHAAQLRRRIRACARDLEG |
| | LRHLSRRYLKTLEKPCRRPRAPDLGRARCHALVESLQAAERELEELRRCDSPDTAMRRL |
| | DAVLAAAASTDATFATGWTVVGMDLGVAPRGSAAPEVSPMEMAISVFWRKGSRRVIVSK |
| | PIAGMPIRRHELIRLEGLGTLRLDGNHYTGAGVTKGRGLSEGTEPDFREKSPSTLGFTL |
| | SDYRHESRWRPYGAKQGKTARQFFAAMSRELRALVEHQVLAPMGPPLLEAHERRFETLL |
| | KGQDNKSIHAGGGGRYVWRGPPDSKKRPAADGDWFRFGRGHADHRGWANKRHELAANYL |
| | QSAFRLWSTLAEAQEPTPYARYKYTRVTM |
| SEQ ID | WDFLTLQVYERHTSPEVCVAGNSTKCASGTRKSDHTHGVGVKLGAQEINVSANDDRDHE |
| NO: 104 | VGCNICVISRVSLDIKGWRYGCESCVQSTPEWRSIVREDRNHKEAKGECLSRFEYWGAQ |
| | SIARSLKRNKLMGGVNLDELAIVQNENVVKTSLKHLFDKRKDRIQANLKAVKVRMRERR |
| | KSGRQRKALRRQCRKLKRYLRSYDPSDIKEGNSCSAFTKLGLDIGISPNKPPKIEPKVE |
| | VVFSLFYQGACDKIVTVSSPESPLPRSWKIKIDGIRALYVKSTKVKFGGRTFRAGQRNN |
| | RRKVRPPNVKKGKRKGSRSQFFNKFAVGLDAVSQQLPIASVQGLWGRAETKKAQTICLK |
| | QLESNKPLKESQRCLFLADNWVVRVCGFLRALSQRQGPTPYIRYRYRCNM |
| SEQ ID | ARNVGQRNASRQSKRESAKARSRRVTGGHASVTQGVALINAAANADRDHTTGCEPCTWE |
| NO: 105 | RVNLPLQEVIHGCDSCTKSSPFWRDIKVVNKGYREAKEEIMRIASGISADHLSRALSHN |
| | KVMGRLNLDEVCILDFRTVLDTSLKHLTDSRSNGIKEHIRAVHRKIRMRRKSGKTARAL |
| | RKQYFALRRQWKAGHKPNSIREGNSLTALRAVGFDVGVSEGTEPMPAPQTEVVLSVFYK |
| | GSATRILRISSPHPIAKRSWKVKIAGIKALKLIRREHDFSFGRETYNASQRAEKRKFSP |
| | HAARKDFFNSFAVQLDRLAQQLCVSSVENLWVTEPQQKLLTLAKDTAPYGIREGARFAD |
| | TRARLAWNWVFRVCGFTRALHQEQEPTPYCRFTWRSKM |
| SEQ ID | MAGKKKDKDVINKTLSVRIIRPRYSDDIEKEISDEKAKRKQDGKTGELDRAFFSELKSR |
| NO: 106 | NPDIITNDELFPLFTEIQKNLTEIYNKSISLLYMKLIVEEEGGSTASALSAGPYKECKA |
| | RFNSYISLGLRQKIQSNFRRKELKGFQVSLPTAKSDRFPIPFCHQVENGKGGFKVYETG |
| | DDFIFEVPLIKYTATNKKSTSGKNYTKVQLNNPPVPMNVPLLLSTMRRRQTKKGMQWNK |
| | DEGTNAELRRVMSGEYKVSYAEIIRRTRFGKHDDWFVNESIKFKNKTDELNQNVRGGID |
| | IGVSNPLVCAVINGLDRYIVANNDIMAFNERAMARRRTLLRKNRFKRSGHGAKNKLEPI |
| | TVLTEKNERFRKSILQRWAREVAEFFKRTSASVVNMEDLSGITEREDFFSTKLRTTWNY |
| | RLMQTTIENKLKEYGIAVNYISPKYTSQTCHSCGKRNDYFTFSYRSENNYPPFECKECN |
| | KVKCNADFNAAKNIALKVVL |
| SEQ ID | MRISKTLSLRIVRPFYTPEVEAGIKAEKDKREAQGQTRSLDAKFFNELKKKHSEIILSS |
| NO: 107 | EFYSLLSEVQRQLTSIYNHAMSNLYHKIIVEGEKTSTSKALSNIGYDECKAIFPSYMAL |
| | GLRQKIQSNFRRRDLKNFRMAVPTAKSDKFPIPIYRQVDGSKGGFKISENDGKDFIVEL |
| | PLVDYVAEEVKTAKGRFTKINISKPPKIKNIPVILSTLRRRQSGQWFSDDGTNAEIRRV |
| | ISGEYKVSWIEIVRRTRFGKHDDWFVNMVIKYDKPEEGLDSKVVGGIDVGVSSPLVCAL |
| | NNSLDRYFVKSSDIIAFNKRAMARRRTLLRQNKYKRSGHGSKNKLEPITVLTEKNERFK |
| | KSIMQRWAKEVAEFFRGKGASVVRMEELSGLKEKDNFFSSYLRMYWNYGQLQQIIENKL |
| | KEYGIKVNYVSPKDTSKKCHSCTHINEFFTFEYRQKNNFPLFKCEKCGVECSADYNAAK |
| | NMAIA |
| SEQ ID | MKDYIRKTLSLRILRPYYGEEIEKEIAAAKKKSQAEGGDGALDNKFWDRLKAEHPEIIS |
| NO: 244 | SREFYDLLDAIQRETTLYYNRAISKLYHSLIVEREQVSTAKALSAGPYHEFREKFNAYI |
| | SLGLREKIQSNFRRKELARYQVALPTAKSDTFPIPIYKGFDKNGKGGFKVREIENGDFV |
| | IDLPLMAYHRVGGKAGREYIELDRPPAVLNVPVILSTSRRRANKTWFRDEGTDAEIRRV |
| | MAGEYKVSWVEILQRKRFGKPYGGWYVNFTIKYQPRDYGLDPKVKGGIDIGLSSPLVCA |
| | VTNSLARLTIRDNDLVAFNRKAMARRRTLLRQNRYKRSGHGSANKLKPIEALTEKNELY |
| | RKAIMRRWAREAADFFRQHRAATVNMEDLTGIKDREDYFSQMLRCYWNYSQLQTMLENK |
| | LKEYGIAVKYIEPKDTSKTCHSCGHVNEYFDFNYRSAHKFPMFKCEKCGVECGADYNAA |
| | RNIAQA |
| SEQ ID | VKISKTLSLRIIRPYYTPEVESAIKAEKDKREAQGQTRNLDAKFFNELKKKHPQIILSG |
| NO: 108 | EFYSLLFEMQRQLTSIYNRAMSSLYHKIIVEGEKTSTSKALSDIGYDECKSVFPSYIAL |
| | GLRQKIQSNFRRKELKGFRMAVPTAKSDKFPIPIYKQVDDGKGGFKISENKEGDFIVEL |
| | PLVEYTAEDVKTAKGKFTKINISKPPKIKNIPVILSTLRRKQSGQWFSDEGTNAEIRRV |
| | ISGEYKVSWIEVVRRTRFGKHDDWFLNIVIKYDKTEDGLDPEVVGGIDVGVSTPLVCAV |
| | NNSLDRYFVKSSDIIAFKKRAMARRRTLLRQNRFKRSGHGSKSKLEPITILTEKNERFK |
| | KSIMQRWAKEVAEFFKGERASVVQMEELSGLKEKDNFFGSYLRMYWNYGQLQQIIENKL |
| | KEYGIKVNYVSPKDTSKKCHSCGYINEFFTFEFRQKNNFPLFKCKKCGVECNADYNAAK |
| | NIAIA |
| SEQ ID | VPITKTISLRILRPYYPPEIEAKIKAEKEKRKENGDTGSLNSSYYRELKKEYPSIIIND |
| NO: 109 | EFFPLLSEMQRNITSIYNRTISHLYHRLIIKKESISTAKALSEGPYRDFKSTFNSYIAL |
| | GLRQKVQSNFRKKDLMAFKIALPTAKSDKFPIPIYMQTNFKIKESPDSDFIIELPLVEY |
| | IAKETKGKNKMFTKVEILSPPKVKNIPVILSTRRRKESGQWFSDEGTNAEIRRIISGEY |
| | KVSWIEIVKRTRFGKHDWFVNMVISFEESQEGLDPDVIGGIDIGVSKPLICAINNSLDR |
| | YIVKGDDIIAFNRRALSRRRSLLRRNRLKRSGHGSRNKLEPITVLTEKNERFKKSIMQR |
| | WAKEVAEFFKSKRASIVQMEELTGIKEREDFFSKTLRMYWNYGQLQKTVENKLREYGIE |
| | VRYASPKDTSRRCHSCGHINDYFTFEFRQQNNFPLFKCMNCGIECSADYNAARNIAIAR |
| SEQ ID | MAKKGTNRKKMIVKVMKYELKYESGCADFNEMQNELWKLQRQTREVMNRTIQLCYHWSY |
| NO: 110 | VQADYCKQHGCARRDVKPCDVYETNATSLDGYIYQLFKDEYPNFLMANLIATLRKAHQK |
| | YDALLFDIQEGNSSIPSFKKDQPLIFSKEAIRLPECLSDKRQITLFCFSKPYKSAHPTL |
| | DKITFAVRARSASEKSIFDHIISGKYALGESQLVYEKKKWFFLLSYKFTPESVDVNPEK |
| | VLGVDLGVVNALCAGSVENPHDSLFIKGTEAIEQIRRLEARKRDLQKQARYPGDGRIGH |
| | GTKTRVSPVYQTRDAIARMQDTLNHRWSRALIDFACKKGYGTIQMEDLSGIKALESEKP |
| | YLKHWTYFDLQSKIIYKAEEKGIRVVKVNPKCTSRRCSACGYISKENRKNQVEFLCVNC |
| | GYHHNADYNAAQNLSIPQIDRLIEKQLKEQESEENEAGANPK |
| SEQ ID | MAKGTLSKVMKYELRYLDGCGDFQNMQKELWTLQRQSREILNRTIQIAYHWDYTDREQF |
| NO: 111 | KKTGQHLDIKAETGYKRLDGYIYDSLKEDVQNFASVNVNATIQKAWAKYKSSKIDVLRG |
| | DMSLPSYKSDQPLVLHAQSMKIFSSDDDDVLQVTLFSNAYKKACNYSNIRFIIGLHDAT |
| | QRTIIKKVLSGDWGIGQSQIVYKRPKWFLYLTYNFSPEQHEVNPDKILGVDLGESIAIY |
| | ASSIGEYGSLRIEGGEISAFAKQLEARKRSLQKQAAYCGKGRIGHGTKSRVSDVYKMED |
| | KIANFRNTVNHRYSKMLIDYALKHMYGTIQMEDLSGIKKETGFPKFLQHWTYYDLQQKI |
| | EAKAKEHGINFIKVDPAFTSQRCSKCGNIDSENRPSQAVFCCKKCGYKTNADFNAS |
| SEQ ID | MNVTKVMRYQLIYQGGGGDFESLQNQLWEFQRQTRAILNKTIQTMYLATANQEKFSEKA |
| NO: 112 | LYHDLCAEYPDMISSTVNATLREATKKYRSSVREILAGRMSLPSYKRDHPILLHNQSVA |
| | LKQGNQGSYFATISVFSRKYQQGTPGVKQPSFQLIAKDNTQRTILQRLLSGEYKLGQCQ |
| | LIYIRPKWFLNVAYSFTPSEKALDQEKVLGVDLGCVYAIYASSYGNHGIFKISGDEITS |
| | FERKQAAIQNRAFKNDLTRIREIEERRKQKLEQARYCGEGRIGHGVKTRVAPAYQDEGK |
| | ISRFRETINHRYSKALVDYAEKNGYGTIQMEDLSGIKSSTGFPKRLQHWTYFDLQQKIK |
| | YKAEEQGIKVVKIKPAYTSQRCSRCGHIDPANRKSQSEFKCIACGFSSNADYNASQNIS |
| | MRNIEKIIQGKAN |
| SEQ ID | MAKGTITKVMKYELRYLGGFSDFHEMQKEVWQLQRQYREILNKTIQIALHWDYVSAQQF |
| NO: 113 | GESGTYLDIREETGYKTLDGYIYNCLKGAYSEMASANLNAAVQKAWKKYKNSKTQVLQG |
| | VMSLPSYKSDQPILIDKGNVKLSAEENNGRAVLTLFSRNYRDTRGLKGNVEFSVLLHDG |
| | TQKSIFRNLIDKTYALGQCQLVYERKKWFLLLTYSFTPAGHALDPEKILGVDLGECYAL |
| | YASSCYAPGILKIEGGEIAEYALRLEKRKRSLQQQARYCGEGRIGHGTKTRVGVVYKAE |
| | DRIASFRETINHRYSKELVDYAVSNGYGTIQMEDLSAIQKDLGFPKRLRHWTYYDLQMK |
| | ITNKAKEHGIAVVKIDPRYTSQRCSKCGHIDPANRPRQEEFCCTACGYACNADYNASQN |
| | ISIKGIEKIIQKMLSAKAD |
| SEQ ID | MSKGMLTKVMKYTLRYVGGCGDFHEMQSILWELQKQTRAVLNKTIQIAFEWDYRSREAF |
| NO: 114 | QETGEYLDVHAETGYKRLDGYIYNCLKNEYADFAGKNLNAAIQTAWKKYNQSKRDIQTG |
| | KMSLPSYRSNQPLIIHNDNVMISQDMQAAPSVRFTLLSLEYKKAHDLNTNPTFEVLIND |
| | GTQRAIFEKVRSGEYKLGQCMIQYDKKKWFLLLTYSFQPEKLTLDKNKILGVDLGETIV |
| | ICASSVSERGRFVIDGGEITRFATQIEARKRSQQHQAAYCGEGRIGHGTKTRVDAVYKT |
| | EDRIANFRDTINHRYSRALVNYAVKHGFGTIQMEDLSGIKSSDDFPKFLRHWTYYDLQS |
| | KIESKAKERGIAVVKVNPRFTSRRCSKCGYIDEGNRKDQAHFCCLSCGFRANADFNASQ |
| | NLSIKGIDKIIEKEYNANSKQT |
| SEQ ID | MGKPITKTMKYQIHYIDGCGDFHNMQKELWDLQRIVRQILNKTINESYLWFVRSEQYYR |
| NO: 115 | DTGENLSVEEQTGYKTLDGHIYNLLKQEYTQKLVSNSLNASIQAAYKKMKDSRRDVMIG |
| | TMSLPSYRSDQPIIIYNKNIKFSSHPEHGFVVDCSLFSDAYKKSQGYEKSVKFQVSVDD |
| | NTQRSIFENILTGNYKHGQCSIVYEKKKWFLLLTYSFVPEETKLDPDKILGVDVGVVYA |
| | LYASSKGNHGTFKIKGDEAITFIQRVEARKHSRQLQGTYCGDGRIGHGTKTRVQPVYNE |
| | RALISNFQDTINHRYSKALIDYAKKNGYGTIQMEDLSGIKEVQQYPKYLQHWTYYDLQL |
| | KIQYKAKEAGIGFVKVTPKYTSQRCSHCGNIDEANRPKQDVFRCTVCGYERNADYNASQ |
| | NLSIKGIDRIIDDQLKQMNKANPKKTENA |
| SEQ ID | MSGGAITKVMKYDLTYKDGYGNFKDMQEAVWKLIRDTRTILNETIKIAYHWDYLNEKSK |
| NO: 116 | RETGEHLDLLEETGYKRLDGYIYDDLKDRFPDFASSNLNAAIQTAWKKYKQSQKDVYIG |
| | KMTLPSYKSDQPLPINKQSIKIYDEEREHIVELNLFSTKHKKEHGLASNVRFRINLHDN |
| | TQHAIYERVLSGEYTLGQCQLLYDRPKWFFILTYSFKPAQNKLDPDKILGVDMGETCAL |
| | YASTFGEQGSFVINGGEVSEYAKREEARKRSLQKQAAVCGEGRIGHGTKTRVSSVYKEQ |
| | ERISNFRDTINHRYSKALIEYAVKNGCGTIQMEDLSGIRQSTDFPKFLRHWTYYDLQQK |
| | IKTKAKETGIAVSMIDPRYTSQRCSRCGHIDKANRKDQAHFHCLKCGYSCNADFNASQN |
| | ISIRGIDKIIQKELGAKAKQTD |
| SEQ ID | MKEIAKVMKYQLIYLDGGGDFYELQQTLWDLQRQTREILNKTIQSMYLATATNTAFEEN |
| NO: 117 | ALYHRFGAEYPMMAALNVNATLRTAKKRYTSTIKETLRGTMSLPSYKRDQPILLHNQTI |
| | HLALEDGQYSALFSVYSEKFQKAHEGVARPRFALMARDGTQRAILDRLLDGSYRLGQSQ |
| | MTYEQKKWFLSLTYKFVPEVRELDKSKILGVDLGCVYAIYASSMQQKGIFKISGDEITE |
| | FEKRQAAMQNREPVSTLERVEQLEQRRWQKQQQARYCGEGRVGHGTGTRVAPAYRDADK |
| | IARFRDTINHRYSKALVEYAEKNGFGTIQMEDLSGIKEDTGFPKRLRHWTYFDLQTKIQ |
| | YKAAERGITVVKIDPQYTSQRCSRCGYIDKANRASQEKFLCQSCGFEANADYNASQNIS |
| | VEKIDKLIAKDKKKLART |
| SEQ ID | MGQVTKVMRYQLIYQDGGGDFYTVQQELWELQRQTREILNKTIQTMYLADANKEKFDNA |
| NO: 118 | AERTLNRRFCVDHPDMYTKTVTATLRKAKAKYNASQKEILAGRMSLPSYKRDQPILLNP |
| | QGFKIEEESDSFFAAIAVFSDKYKNKHPDVDVKRLRFRLVVKDGTQRAIIRRVISGEYK |
| | LGRSQLLYSKKKWFLNVTYSFEPAEKKVDPDKILGVDLGCVYAIYASSFGSPGVFKISG |
| | DEVSSFERKQAAIQNRSPKSTLERVEKIEERHKQKQQQARYCGEGRIGHGTKTRIAPVY |
| | QDEDKIARFRDTVNHRYSKALIDYAEKNGYGTIQMEDLSGIKSATGFPKRLKHWTYYDL |
| | QTKIEYKAEERGIKVVKIDPRYTSQRCSRCGYIDSGNRKSQAEFCCMACGFSCNADYNA |
| | SQNISIGGIAKIIADKRKEADAK |
| SEQ ID | YLDIREETGYKTLDGYIYNCLKGAYSEMASANLNAAVQKAWKKYKNSKTQVLQGVMSLP |
| NO: 119 | SYKSDQPILIDKGNVKLSAEENNGRAVLTLFSRNYRDTRGLKGNVEFSVLLHDGTQKSI |
| | FRNLIDKTYALGQCQLVYERKKWFLLLTYSFTPAGHALDPEKILGVDLGECYALYASSC |
| | YAPGILKIEGGEIAEYALRLEKRKRSLQQQARYCGEGRIGHGTKTRVGVVYKAEDRIAS |
| | FRETINHRYSKELVDYAVSNGYGTIQMEDLSAIQKDLGFPKRLRHWTYYDLQMKITNKA |
| | KEHGIAVVKIDPRYTSQRCSKCGHIDPANRPRQEEFCCTACGYACNADYNASQNISIKG |
| | IEKIIQKMLSAKAD |
| SEQ ID | MAEKTIVKVMKFELRYIDGAGEFSEMQKHLWELQKQTREVLNKTIQMGYALECKRFAHH |
| NO: 120 | DKTGQWLDDKELTGSKYKAVADYINAELKEDYNIFYSDCRNSTVRKAYKKFKDAKNKIF |
| | SGEMSLPSYRSNQPIIIHNRNVIIRGNAESALVGLKVFSDGFKALHGFPAAVNFKLCVK |
| | DGTQRAIIENVISEIYKISESQLIYDNKKWFLILAYRFTQKKNDLNPDKILGVDLGVKF |
| | AVYASSIGEYGSFRIKGGEVTEFIKRLEKRKKSLQNQATVCGDGRIGHGTKTRVADVYK |
| | ARDKISNFQDTINHRYSRAIVDYARKNGYGTIQLEKLDNSIEKKGDYSPVLVHWTYYDL |
| | RTKMEYKAAEYGIKVIAVEPKYTSQRCSKCGYISSENRKTQESFECIKCGYKCNADFNA |
| | SQNLSVRDIDRIIDEYLGANPELT |
| SEQ ID | VVNVAKGALSKVMKFELSYLDGCGDFQNMQKELWTLQRQTREILNRTIQIAYHWDYTDR |
| NO: 121 | EHFKKTGQHLDVKSETGYKRLDGYIYDELKETVQNFASVNVNATIQKAWAKYKSSKTDV |
| | LRGDMSLPSYKSDQPLVLHAQSIKLSEDKDGPVLQVTLFSNAHKKACDYSNVRFAFRLH |
| | DATQRAIFKNVLSGEYGLGQSQIVYKRPKWFLYLTYNFSPEQHGLDPDKILGVDLGESI |
| | ALYASSLGDYGSLRIEGGEVTAFAKQLEARKRSLQKQAAHCGEGRVGHGTRARVSDVYK |
| | AEDKIANFRNTVNHRYSKKLIEYAIQNRYGTIQMEDLSGIKQDTGFPKFLQHWTYYDLQ |
| | QKIEAKAKENGINFIKVDPSYTSQRCSKCGNIDSDNRPSQAVFCCTKCGFRANADFNAS |
| | QNLSIPEIDKIIKKERGANTK |
| SEQ ID | MAKKGTNRKKMIVKVMKYELKYEKGCADFNEMQNELWKLQRQTREVMNRTVQLCYHWNY |
| NO: 122 | VQADYCKQHGCAHRDVKPCDVYETNATSLDGYIYQLFKDEYPNFLMANLIATLRKAHQK |
| | YDALLPDIQEGNSSIPSFKKDQPLIFSKEAIHLPECLSDKRQITLFCFSKPYKSAHPTL |
| | DKITFAVRAHSASEKSIFDNIINGKYALGTSQLVYEKKKWFFLLSYKFTPESVDVNPEK |
| | VLGVDLGVVNALCAGSVENPHDSLFIKGTEAIEQIRRLEARKRDLQKQARYPGDGRIGH |
| | GTKTRVSPVYQTRDAIARMQDTLNHRWSRALIDFACKKGYGTIQMEDLSGIKAMESEKP |
| | YLKHWTYFDLQSKIIYKAEEKGIRVVKVNPKCTSRRCSACGYISKENRKNQAEFLCVNC |
| | GYHHNADYNAAQNLSIPQIDRLIEKQLKEQESEESEAGANPK |
| SEQ ID | MTERHDNESSKIKAEVSLLNSSVPDFEKKRHVKVLKLHILKPAGDMKWDELGALLRDAR |
| NO: 123 | YRVFRLANLAISEAYLDFHKWRSGGNEQPKLKISQLNRNLRSMLEDEVTGKQTKMIKSD |
| | RYSKSGALPDSIVSPLSMYKLGGLTSKSKWSEVLRGKSSLPTFKLNMAIPVRCDKPGDR |
| | RIERTKNGDAEVELRICLQPYPRVIIATGRNSLGDGQRAILDRLLDNTKYSEQGYRQRC |
| | FEIKEDQRSGKWHLFVTYDFPAIEPAKNLSRERIVGVDLGAACPLYAAINTGHARLGWK |
| | HFSPLAARVRALQNQTIRRRRQILRGGKVSLSEDSARSGHGRKRKLKPISKLEGKIDRA |
| | YTTLNHQLSATVIKFAKDNGAGVVQMEDLKGLRETLTGTFLGERWRYEELQRFIRYKAD |
| | EAGIEIRLVNPQYTSRRCSECGHIHKDFTREFRDKSREGNKSVRFLCPDCGFTADPDYN |
| | AARNLASLDIAAIIERQLEIQGLRKHDP |
| SEQ ID | MKEKSKTLVKVARLRILKPAGDMKWSELGEMLRTVRYRVFRLANLAVSEAYLGFHMYRT |
| NO: 124 | NRATEFKAETIGKLSRRLREMLIEEGVDEKDLSRYSQTGAVPDTVAGALGQYKIRGITS |
| | PTKWRQVVRGQAALPTFRNDMAIPIRCDKQYQRRLEKTEAGEIEVELMICRKPYPRIVL |
| | GTADLGPGQRAILERLLQNTDNSADGYRQRLFEAKQDTQTKKWWLYVTYDEPRLKEGKL |
| | NQEIVVGVDLGFSIPLYVALNIGHARLGRRHFQALGNRIRSLQRQVLARRRSIQRGGRV |
| | NISHSTARSGHGRKRKLLPTEKLRGRIEKSYSTLNHQLSASVIDFAKNHHAGTIQIEDL |
| | ANLKEELAGTFIGARWRYHQLQQFLKYKAEEAGITLNQVNPRYTSRRCSECGFINIDED |
| | RAFRDAGRTEGRVTKFLCPECGYEADPDYNAARNISILDIDKLIRVQCKKQGLTYDAH |
| SEQ ID | MPERPKTVNKVIWFQIHKPAGDMTWKELGNLLREARYRVFRLANLAVSEKYLSFHMWRT |
| NO: 125 | GQEYKSETIGKLNRRLREMLIEEGVEEESQKRFSATGALPDTVVSTLAKGKLAAITSKS |
| | KWKDVVNGKTSLPTFKLNMAIPVRCDKAEQRRLRRTESGDVELELMICKQPYPRVVLKT |
| | GKLKSGQRAILDRLVENNDNSKEGYSQRVFEIKQVENNDGSKEWRLYISYTFPKKAVEA |
| | NADVAVGVDIGFSVPLVAAVNNGLERLGYNDFRALNERIRSLQRQVLVRRRSMQSGGRD |
| | YVSTPTARSGHGRKRKLLPIQTLRKRWDNAYTTLNHQLSHAVVSFAENHGAATIQIENV |
| | KSLKDELRGTFLGQRWRYFELQQFLKYKADEVGIELREVNARYTSRRCSECGYINMAFT |
| | RQARDKGRVDGKPMEFVCPECGYKAHPDYNAARNIAMLDIEQKMQVQCKQQGITYADDS |
| | EVL |
| SEQ ID | MTWPELGNMLRTVRYRVFRLANLAVSEAYLGFHMERTKRAEEFKAETMGKLSRRLREML |
| NO: 126 | IEEGVDEKDLSRYSQTGAVPDTVAGALSQYKIRGITSPTKWRQIVRGQVALPTFRNTMS |
| | IPVRCDKLYQRRLEQGDSGEVEVELMICRNPYPRVVLGTGDLNPGQQAILERLLQNTDN |
| | SADGYRQRLFEIKEDVQTRKWWLYVTYDFPKTTGKLNPEIVVGVDLGFSIPLYVALNSG |
| | HARLGYLHFKALGERIKSLQKQVMARRRAIQRGGRVSISHSTARTGHGVKRKLQPTEKL |
| | RGRIEKSYSTLNHQLSASVIDFAKNHHAGVIQIEDLSGLKEQLTGTFIGARWRYHQLQQ |
| | FLKYKAEEAGITLKQINPRYTSRRCSECGFINMDFDRAFRDAGRTYGKVTKFLCPECGY |
| | EADPDYNAARNIATLDIEKLIRVQCEKHGLKFDAH |
| SEQ ID | VGKEGKRNVKVMKIRILKPCDGMTWNELGQLLRDARYRVFRLANLTVSEAYLNFHLWRT |
| NO: 127 | GRSQEFKKQTIGQLNRQLRNILQQEKYDDEKLNRYSKTGALPDTVCSALWQYKLMAVMK |
| | KSKWSEVIRGKSSLPTFRNDMAIPVRCDKPEQKRIEKTEQGQVEAALQVCVQPYPRVIL |
| | GTHTLGDGQDAILKRLLDNQNQAIGGYRQRSFEIKYDEQKRWWLFITYDFPATEVATDK |
| | TIAVGVDLGVSVPLYAAVNNGPARLGRREFGGLGRRIRDLRNQTDARRRSIQRSGREGQ |
| | SDDTARAGHGRKRKLLPIHILEGRLDKAYTTLNHQMSAAVIKFAAEQGAGIIQIENLAG |
| | LQDELRGTFIGGRWRYRQLQDFLKYKTQEMGIELRQVNPKYTSRRCSKCGFIHKDFDRD |
| | YRNRHSENGKPAQFVCPNPDCKYESDPDYNAARNLATLDIEEQIRVQCQKQGLEYDSKK |
| | DKNAL |
| SEQ ID | MKEKSKTLVKVARLRILKPAGDMTWSELGEMLRTVRYRVFRLANLAVSEAYLGFHMFRT |
| NO: 128 | QRAAEFKAETMGKLSRRLREMLIEEGVDEKELNCYSLTGAVPDTVAGALHQYKIRGITS |
| | PTKWRQVVRGQAALPTFRNDMSIPIRCDKPYQRRLEKTEAGEVEVELMICRKPYPRIVL |
| | GTADVGPGQEVILERLLQNKDNSSDGYRQRLFEAKQDRQTGKWWLYVTYDFPRPEEGEL |
| | NPEIVVGVDLGFSVPLYVAINNGYARLGRRHFQALGNRIRSLQRQVLARRRSIQRGGRV |
| | NISHDTARSGHGIKRKLLPTEKLRGRIEKSYSTLNHQLSASVIDFTKNHHAGTIQIEDL |
| | ANLKEVLAGTFIGARWRYHQLQQFLKYKADEAGITLKEVNPRYTSRRCSECGFIHKDFD |
| | RAFRDSGRTDGKVARFVCPECGYGPVDPDYNAAKNISTLDIEKHIRVQCKKQGLEYEVH |
| SEQ ID | MKEKAKTLVKVARLRILKPAGDMTWPELGNMLRTVRYRVFRLANLAVSEAYLGFHMFRT |
| NO: 129 | KRAEEFKAETMGKLSRRLREMLIEEGVDEKDLSRYSQTGAVPDTVAGALSQYKIRGITS |
| | PTKWRQIVRGQVALPTFRNTMSIPVRCDKLYQRRLEQGDSGEVEVELMICRNPYPRVVL |
| | GTGDLNPGQQAILERLLQNTDNSADGYRQRLFEIKEDVQTRKWWLYVTYDFPKTTGKLN |
| | PEIVVGVDLGFSIPLYVALNSGHARLGYLHFKALGERIKSLQKQVMARRRAIQRGGRVS |
| | ISHSTARTGHGVKRKLQPTEKLRGRIEKSYSTLNHQLSASVIDFAKNHHAGVIQIEDLS |
| | GLKEQLTGTFIGARWRYHQLQQFLKYKAEEAGITLKQINPRYTSRRCSECGFINMDFDR |
| | AFRDAGRTYGKVTKFLCPECGYEADPDYNAARNIATLDIEKLIRVQCEKHGLKFDAH |
| SEQ ID | MAKKAKTMFKVTNFRILKPAGDMTWKELGQLLRDARYRTFRMANLALSEAYLNFYLLKK |
| NO: 130 | GDLKEYKNVKIGQIAKRLRDMLIEEGVDEEVQNRFSPKVALPAYVYSALDQFKLRGLTS |
| | KSNWKKVLRGQASLPTFRLNMSVPIRCDKPEHRRLEKTENGNVEVDLMICRKPYPRVVL |
| | ETLKLDGSSKAILDRLLENEDNSPGNYRQRCFEVKQNPRSNDWWLYVTYEMPVDKDKKL |
| | DPKVIVGVDLGFSVPLYVAINNGHARLGRRHFQALGKRIHNLQNQVLARRRSIQRGGQV |
| | NLSHSTSRSGHGRKRKLQPTEKLQQKINSAYSTLNHQLSSSVIDFANNHKAGTIQIEDL |
| | ETLKEQLTGTYIGRQWRYYQLQQFIEYKAKENSITVKKINPKYTSRRCSMCGHIHADFD |
| | RTFRDRSSNKGFVTKFICPECNFEADPDYNAAKNISTLDIENKIKLQCKKQKIDY |
| SEQ ID | MPKITRKIELLFDRSGLSEEECKEKWRFIYQINDNLYRVANRLVNQLYLADEIDDILRL |
| NO: 131 | SDQEYIALRKKLANKKLDEATRISLEEQMSQVMKRVNERRSAILQRPQQSFAYSVVTDS |
| | DTEGLTAKILDVLKQDVLSHYKADTKEVLKGEKSISNYKKGMPIPFAFNDSLRLYKEDG |
| | FFYLKWYNGIRFLLNFGRDASNNQLIVERCLGISKDEISYKACSSSIQIKKKGNHSKIF |
| | LLLVVDVPVEQYAQKPNMVVGVDLGLNVPIYAASNSTLERKAIGSREAFLNQRGAFQRR |
| | FRALQRLQTTKGGRGRLHKLEPLERVREAERNWVRTQNHLFSREVINFAIDVGASTIQM |
| | EKLANFGRDAQGEVREDKKYVLRNWSYFELQNLIEYKAKRAGIKVKYINPAFTSQTCSE |
| | CGQLGERDSIHFKCTNPDCPNCGKDIHADYNGARNIAKSKDYIK |
| SEQ ID | MPTITRKIELTLLTEGLSEEQRKEQWGLLYHINDNLYKAANNISSKLYLDDHVSSMVRM |
| NO: 132 | KHAEYLSLLKELARAEKQKTPDADAIAELRKKVAAAEKEMTDQEHAICKYATEMSTQSL |
| | SYRFATELETNIFAKILDCLKQGVFATFNSDARDVKRGERAIRNYKKGMPIPFAWDKSL |
| | RIEKDNKDFYLRWYNGLRFLFNFGKDRSNNRLIVERCLKMDADYDGEYKLCNSSIQIAK |
| | REGKTKLFLLLVVKIPQEHVELNKKVVVGVDLGINVPAYVATNITEERKAIGDREHFLN |
| | SRMAFQRRYKSLQRLRGTAGGKGRAKKLEPLERLRKAEHNWVHTQNHLFSREVVDFAVK |
| | SHAATIHMEDLSGFGKDNDGNADERKEFVLRNWSYYELQNMIAYKAAKYGIKVEKIHPA |
| | YTSKTCSWCGQLGFREGVTFICENPECKQCGEKVHADYNAARNIANSKDIIKKNE |
| SEQ ID | MPTITRKIELTLCTDGLSEEQRKEQWGLLYHINDNLYKAANNISSKLYLDDHVSSMVRM |
| NO: 133 | KHAEYLSLLKELARAEKQKTPDADAIAELKKKVAATEKEMTDQEHAICKYATEMSTQSL |
| | SYRFSTEFETKIFAKILDCLKQGVFATFNSDAKDVKRGERAIRNYKKGMPIPFAWTDSL |
| | RIKKDNKDFYLLWYNGLRFLFNFGKDRSNNRLIVERCLKMDADYDGEYKLCNSSIQIAK |
| | REGKVKLFLLLVVSIPKEHVELNKKVVVSVDLGINVPAYVATNITEERKAIGDREHFLN |
| | SRMAFQRRYKSLQRLKGTTGGKGRTKKLEPLERLRKAEHNWVHTQNHLFSREVVDFAVK |
| | THAATIHMEDLSGFGKDNDGNADERKEFVLRNWSYYELQNMISYKAAKYGIKVEKIRPA |
| | YTSKTCSWCGQHGFREGVTFICENPACKQCGEKVHADYNAARNIANSKEIIKKNE |
| SEQ ID | MPTITRKIELTLCTEGLSDQERKDQWNLLYHINDNLYRAANNISSKLYLDDHVGSMVRL |
| NO: 134 | KHAEYLSLLRALEKAKKQKAPDEEVIAELSQQVATAEQEMDEQAKAICQYATEMSTQTL |
| | SYRFATELETNIFGQILTCLRQGVESTENSDARDVKRGERSIRTYKKGMPIPFPWNDSL |
| | RIGFEDGEFYLRWYNGLRFRFDFGKDRSNNCLIVQRCMKMDKDYEGDYKLCNSSIQMVK |
| | REGKPKFFLLLVVNIPQERVELNKNIVVGVDLGINAPAYVATNTTPERKQIGDREHFLN |
| | ERMAFQRRFKSLQRLKGTTGGRGRAKKLEPLERLRKAEQNWVHTQNHLFSREVIDFAVK |
| | ARAATIHMEDLSGFGKDNDGNADERKEFVLRNWSYYELQNMITYKAAKYGIKVEKIRPA |
| | YTSKTCSWCGHQGFREGITFICENPECKKFGEKEHADYNAARNIANSKEIIKNNEE |
| SEQ ID | MPTITRKIELTLLTEGLSEEQRKEQWGLLYHINDNLYKAANNISSKLYLDDHVSTMVRM |
| NO: 135 | KHAEYLSLLRELARAEKQKKPDVDAIAELREKVTAAEKEMSDQERAICTYATEMSTQSL |
| | SYRFATEIETNIFAKILDCLKQGVFATFNSDARDVKRGERAIRNYKKGMPIPFAWDKSL |
| | RIEKDNKDFYLRWYNGLRFLFNFGKDRSNNRLIVERCLKMDADYDGEYKLCNSSIQIVK |
| | REGKVKLFLLLVVSIPQEHVELNKKIVVGVDLGINVPAYVATNITEERKAIGDREHFLN |
| | SRMAFQRRYKSLQRLKGTAGGKGRTKKLEPLERLRKAEHNWVHTQNHLFSREVVDFAVK |
| | SHAATIHMEDLSGFGKDNDGNADERKEFVLRNWSYYELQNMIAYKAAKYGIKVERIRPA |
| | YTSKTCSWCGQLGFREGVTFICENPECKQCGEKVHADYNAARNIANSKDIIKKNE |
| SEQ ID | MPTMTRKIELKLCTEGLSDEERKAQLGLLYHINDNLYKAANNISSKLYLDDHVSSMVRL |
| NO: 136 | KHAEYLSLLNEFEKAKKKGDEEQIVELSLRVAAAEKELTDQELAICKYATEMSTDTLAY |
| | RFANEIEINVFGQILACLKQGIHSTFKKDAADVKRGERAIRNFKKGMPIPFPWSKSIRI |
| | ENEGSDFYLRWYNGLRFREDEGKDRSNNRLIVSRCLNLDPDFEDEYKLSNSSLQMVKRD |
| | GRPKLFLLLVVNIPQENVELNKKIVVGVDLGINSPAYVATNITMERQRIGSRDTFLNAR |
| | MAIQRRFQSLQKLQNTAGGRGRKKKLEPLERLKETERNWVRTQNHLESRDVVQFAVKTR |
| | AATIHMEDLSGFGKDDDGNADEKKEFVLRNWSYYELQTMIKYKAAKYGIKVEKIRPAYT |
| | SRTCSWCGHEGDRKGETFICENPECEKYGKKENADYNAARNIANSTDIIK |
| SEQ ID | MPTITRKIELTLCTEGLSDEQRKEQWGLLYHINDNLYKAANNISSKLYLDEHVSSMVRM |
| NO: 137 | KHAEYLSLLKELARAEKQQTPDEGLIAELSRKLSAAEKEMADQELAICKYATEMSTQTL |
| | SYNFAKEIETNIFGQILTCLRQGVYATFNSDAKDVKRGERAIRNYKKGMPIPFPWNNSL |
| | KIESDSGEFYLRWYNGLRFLLTFGKDRSNNRMIVNRCMKMDEDFEGEYKLCNSSIQLAK |
| | RDGKPKLFLLLVVNIPQEHVKLNKKIVVGVDLGVNVPAYVATNITEERKAIGDREHFLN |
| | TRMAFQRRYKSLQRLKGTAGGKGRTKKLEPLERLRDAERNWVHTQNHLFSREVVNFAVQ |
| | ARAATIHMEDLSGFGKDKDGNADEKKEFVLRNWSFYELQNMIAYKSAKYGIKVVKIRPA |
| | YTSKTCSWCGQQGDRKSTTFICENPKCKHYGESIHADYNAARNIANSNDIVKENE |
| SEQ ID | MPKITRKIEMTLCTEGLSDEQRKEQWGLLYHINDNLYKAANNISTKLYLDEHVSSMVRM |
| NO: 138 | KHADYLSLLKELAKAEKKSPDEDLIAELREKLAAAEQEMTDQELAICKYATEMSTQTLA |
| | YKFATEIEINVFGQILACLKQAAQSNEKSDAKDVKRGERAIRNYKKGMPIPFPWNDNIR |
| | IDADGDEFYLRWYNGLRFHLTFGKDKSNNRMIVKRCLKMDKDFEGEYKLCNSSIQMVKR |
| | DGKPKLFLLLVVNIPQEHVELNKNVVVGVDLGVNVPAYVATNITEERKAIGEREHFLNT |
| | RMQIQRRYKSLQRLKATAGGKGRTKKLEPLERLRKAEHNWVHTQNHLFSREVVNFAVQT |
| | HAATIHMEDLSGFGKDDDGNADEQKEFVLRNWSFYELQNMIAYKAAKYGIKVEKVKPAY |
| | TSKTCSWCGQLGFRQGVTFICENPACKQCGEKVHADYNAARNIANSKDIIKKNE |
| SEQ ID | MPTITRKIELHLCTDGLTDEQQKAQRLLLYHINDNLYKAANNVSSKLYLDEHVSSMVRL |
| NO: 139 | KHDEYLSLSRELARAEKKHDDELTTELRGKLAAAEREMTDQELAICKYATEMSTQSLSY |
| | RLVTELETKIFAKILDCLKQGVYATFNSDARDVKRGERAIRNYKKGMPIPFAWNDSVRI |
| | EYDEKEKDFYLRWYNDIRFKFHFGRDRSNNRLIVSRCLKLDKDYEGDYQLCNSSIQIVK |
| | RDGSTKFFLLLVVKIPQEHVELNKRIVVGVDLGINYPAYVATNCTEERMYIGDREHFLN |
| | TRMQFQRRYKSLQKLKGTAGGKGRSKKLEPLERLRNAERNWVHTQNHLFSLKVVNFAVQ |
| | THAATIHLEDLSGFGKDDDGNADERKEFVLRNWSYYELQSMIEYKAKKYGIKVEKIRPA |
| | YTSQTCSWCGQRGFRQGVTFICENPECKKCGEKENADYNAARNIANSKDVIKDKNE |
| SEQ ID | TPFVLYFQNYSLSLRQHITLYSMPTITRKIELTLCTEGLSDQERKDQWNLLYHINDNLY |
| NO: 140 | RAANNISSKLYLDDHVGSMVRLKHAEYLSLLRAMEKAKKQKAPDEEVIAELSQQVAAAE |
| | QEMDEQAKAICQYATEMSTQTLSYRFATELETNIFGQILTCLRQGVESTENSDARDVKR |
| | GERSIRTYKKGMPIPFPWNDSLRIGFEDGEFYLRWYNGLRFRFDFGKDRSNNRLIVQRC |
| | MKMDKDYEGDYKLCNSSIQMVKREGKPKFFLLLVVNIPQERVELNKNIVVGVDLGINAP |
| | AYVATNTTPERKQIGDREHFLNERMAFQRRFKSLQRLKGTTGGRGRAKKLEPLERLRKA |
| | EQNWVHTQNHLFSREVIDFAVKARAATIHMEDLSGFGKDRDGNADERKEFVLRNWSYYE |
| | LQNMITYKAAKYGIKVEKIRPAYTSKTCSWCGHQGFREGITFICENPECKKFGEKEHAD |
| | YNAARNIANSKEIIKNNEE |
| SEQ ID | MPTITRKIELHLCTEELSDEQQKAQRLLLYHINDNLYKAANNVSSKLYLDEHVSSMVRL |
| NO: 141 | KHDEYLSLLRELARAEKKADDELATQLREKLVAAEREMTDQELAICKYATEMSTQSLSY |
| | RFVTELETKIFAKILDCLKQGVYATFNSDSRDVKRGERAIRNYKKGMPIPFAWDKSVRI |
| | EYEEKEKDFFLRWYNDIRFKFHFGRDRSNNRLIVSRCMKLDKDYEGDYQLCNSSIQIVK |
| | RDGSTKYFLLLVVKIPQEHVELNKKIVVGVDLGINYPAFAATNCTEERMSIGDREHFLN |
| | TRMQFQRRFKSLQRLKGTTGGKGRNKKLEPLERLRKAEHNWVHTQNHLFSLKVVNFAVQ |
| | AHAATIHLEDLSGFGKDDDGNADERKEFVLRNWSYYELQNMIKYKAKKFGIQVEKIRPA |
| | YTSQTCSWCGQRGFRQGITFICENPECKKCGEKENADYNAARNIANSKDIIKDKDE |
| SEQ ID | MPIITRKIELHISKEGLSAEDYKAQWQYLRQINDNLYMAANRVSSHCFLNDEYKYRLCL |
| NO: 142 | QIPDYIDIEKQLKDSKRARLSKEELGQLKKRKKELENTVKGRFQDEFEKNSLYTIISNE |
| | FGEIIPGQILTCLRQCVQSKYNRAKEELEKGERAISTYKKGMPIPFPINKSIRLQKQGE |
| | DFVLKWYNKIVFKLHFGRDRSNNRVIVERLIQSALNDKQKGEDYVMNNSSIQLVEKDKM |
| | TKIFLLLSMDIPTQKRKLDSELVLGVDLGLNFPLYYATNQSANIHDHIGDKDIFLKERM |
| | VFQRRFKELQRLQCTQGGRGRKKKLEPLEKLRDKERNWVRTKNHIFSREVIKVALHLGA |
| | GTIHLENLHNFGKDGNGELKNSKKFVFRNWSYFELQSMIEYKAKMEGITVKYVNPAYTS |
| | QTCSVCGMIGERKEQAVFRCMNSSCLEYGKEVNADFNAARNIAKAKM |
| SEQ ID | MPTITRKIELTLCTDGLSDDLRKDQWQLLYHINDNLYKAANNISSKLYLDEHVASMVRL |
| NO: 143 | KHAEYLGLIKELAKARKRADDEAVRDLCSKLAVAEQEMNEQAKAICDYATEMSTQTLSY |
| | NFAKEIETNIFGQILTCLRQGVLLNFNSDARDVKRGERAIRNYKKGMPIPFPWNDTIKI |
| | VSEGDEFYLRWFSGLRFHLNFGKDRSNNRMIVRRCLKMEQDFDEEYKISNSSIQVAKRD |
| | GKQKLFLLLVVQIPQEQVVLNKKIVVGVDLGVNVPAYVATNITEERKAIGDREHFLNTR |
| | MQFQRRYKSLQRLKTTEGGRGRAKKLEPLERLRKAEHNWVHTQNHLFSREVVNFALQTQ |
| | AATINMEDLSGFGKDNDGNADECKEFVLRNWSYYELQNMIVYKASKYGIRVQKIRPAYT |
| | SKTCSWCGHMGFREGVTFICENPDCKQFGEKVHADYNAARNIANSKEIIKNDE |
| SEQ ID | MSKTVTKTVKIALICEHTNKYGEKVDYKDINKLLWKLQKQTRELKNKTIQLCWEYNNFS |
| NO: 144 | CDYYKEHHEYPNMEDILKYKRINGFVENKLKTVNDLYSSNCSTTILSTCNEFQNYRSEF |
| | LKGTRSINSYKSDQPLDLHKGAIKLEHDGKDFYVSLKLLKRSAFNAMEFKGSDIRFKLN |
| | VKDKDKSTLKILESCYDKIYSISASKMTYDRKAGKWFLLLAYSFTPAKTENLDPEKILG |
| | VDLGIKIPICASVYGDLDRLTIEGGKIEEFRRRVEARKRSLQKQGKQCGDGRIGHGTKK |
| | RIKPITDIGDKIARFRDTENHIYSRYLIEYAVKKGCGTIQMEKLEGITREKDIFLKNWT |
| | YFDLQKKIEYKAKEKGIKVVYIEPAYTSKRCSSCGFIDTDNRLDQAHFKCLKCGFNENA |
| | DYNASQNIGIKNIDKIIKEEHKSASDKLTSE |
| SEQ ID | VIILTKVVKLYLISEQINKEGQKIDYQRINSILWDLQKQTRDIKNRTVQLCWEWMNFSS |
| NO: 145 | DYCKTQEEYPKERDILGYTLEGYVYDYFKTGYDLYTGNISTSSREVCSSFKNVKKEILK |
| | GERSILSYKANQPLDLHKKAISLEYDNFNFFVKLKLLNRTGKKKYDITEDINFKIQVND |
| | KSTRTILERCYDKEYKISGSKLIYEKKKKLWRLNLCYSFENSQVETLEKDKILGIDLGI |
| | VYPLMASIYGEYDRFSIKGGEIEEFRRRTEARKRSILQQTKYCGDGRIGHGRNKRTQPA |
| | YKINDKIARFRDTANHKYSRALIEYAVKKNCGIIQMENLTGISDNTDCFLKDWSYYDLQ |
| | TKIENKAKEMGIKVVYIKAQYTSQRCSRCGYIDVNNRIRQALFKCQNCGYETNADYNAS |
| | QNIGMYDIENIIEETLKIQSANVKQS |
| SEQ ID | MTKVTKVYLISEQIDKDGNKIDFKKISELLWNLQMQTRDIKNKCVQLCWEWLNFSSDYY |
| NO: 146 | KKSEEYPKEKDTLGYTLSGFVYDRIKNGSDLYSSNLSTSSRDTCTAFSNYKKEMLKGER |
| | SVLSFKANQPLDIHNKAIKLSYENGNFFVALKMLNRAGKEKYGIKDDLRFRMQVRDKSV |
| | RTILERLMNDEYKVSASKLMYDKKKKLWKLNLCYSFDNHVISTLDTEKIMGVDLGVVYP |
| | IMASVNGDYARFSIKGGEIEAFRSRVEARRRSLLNQSRYCGDGRIGHGRKKRTEPATQI |
| | ADKIARFRDTTNHKYSRALIDYAIKNGCGTIQMEKLTGITSSAEHFLKEWSYFDLQTKI |
| | ESKAKEAGIKVVYINPKFTSQRCNKCGYIHTDNRPVQARFCCQKCGYEENADYNASQNI |
| | GTKHIDVIIEETLKMQCEPETPTE |
| SEQ ID | MNKVVKLALICEQSDKDNSPVDYKKINEILWELQKQTREIKNKAIQYCWEYNNFSSDYY |
| NO: 147 | KKFNEYPKEKDILSYTLVGFVNDKFKTGNDLYSGNCSTTVRNACTEFKNSKKELIKGSR |
| | SIINYRSNQPLDIHNKCIRIEFENNCFYTYLKLLNRPAFKKYNFANTEIKFKILVRDNS |
| | TKTILERCISNEYEIAASKLLYDQKKKCWFLNLVYAFEIKSNNSLDPNKILGVDLGIHY |
| | PICASVYGSLDRFTIDGGEIDEFRRRVESRKISMLKQGKNCGDGRIGHGIKARNKPVYN |
| | IEDKIARFRDTANHKYSRALIEYAVKHTCGTIQMEDLTGITDIANRFLKNWSYYDLQTK |
| | IEYKAKEAGINIVYIDPKNTSRRCSKCGYIDKENRETQSRFICLKCGFKENADYNASQN |
| | IGIKDIDKLIKEDVH |
| SEQ ID | VTLLVKVVKIYLISEQFDKAGNQIDYKEVNKILWELQKQTREAKNKTVQLLWEWNNFSS |
| NO: 148 | DYVKASGIYPKAKDIFGYSSVHGQANKELRTKLALNSSNLSTTTMDVCKIFNTYKKEVW |
| | EGKRSVPSYKSDQPLDLHKESIKLIYENNEFYVRLALLKKAEFAKYGFKDGFRFKMQVK |
| | DNSTKTILERCFDEVYKINASKLLYDQKKKKWKLNLSYSFDNKNISELDKEKILGVDVG |
| | VNCPLVASVEGDRDRFIIKGGEIEKERKSVEARRRSMLEQTKYCGDGRIGHGRKKRTEP |
| | ALNIGDKIARFRDTTNHKYSRALIEYAVKKGCGTIQMEKLTGITSKSDRFLKDWTYYDL |
| | QTKIENKAKEVGINVVYIAPKYTSQRCSKCGYIHKDNRPNQAKFRCLECDFESNADYNA |
| | SQNIGIKNIDKIIEKDLQKQESEVQVNENK |
| SEQ ID | MNKVVKLALICEQSDKNNSPVDYKKVNEILWELQKQTREIKNKTIQYCWEYYNFSSDYY |
| NO: 149 | KKFNKYPKEKDILSYTLWGFINDKFKTGNDLYSGNCSATTKKVIKEFKNSKKELIRGSR |
| | SIINYKSNQPLNIHNKCIHLQFKNNNFYVSINLLNRRSFKKYNFANTAIKFKILVRDNS |
| | TKAILERCISNEYKISESQLIYNKKKKCWFLNLSYAFEIKSNNSLDPNKILGVDLGIHY |
| | PICASVYGSLDRFTIDGGEIDEFRRRVESRKISMLKQGKNCGDGRIGHGIKARNKPVYN |
| | IEDKIARFRDTANHKYSRALIEYAVKNNCGTIQMEDLTGITDNANRFLKNWSYYDLQTK |
| | IEYKAKEASINVVYINPENTSRRCSKCGYIDKENRKTQSSFICLKCGFKENADYNASQN |
| | ISIKDIDKLIKEDVH |
| SEQ ID | VTLLVKVVKIHLISEQFDKAGNRIDYEEVNKILWELQKQTREAKNKTVQLLWEWNNFSS |
| NO: 150 | DYVKASGIYPKAKDIFGYSSVHGQANKELRTKLALNSSNLSTTTMDVCKNFNTYKKEVW |
| | KGKRSVPSYKSDQPLDLHKDSIKLIYENNQFYVRLALLKKAEFAKYGFKDGFHFKMQVK |
| | DNSTKTILERCFDEVYKINASKLLYDQKKKKWKLNLSYSFDNKNISELDKEKILGVDVG |
| | VSYPLVASVFGDRDRFKIKGGEIEKFRKSVEARRRSMLEQTKYCGDGRIGHGRKKRTEP |
| | ALNIGDKIARFRDTTNHKYSRALIEYAVKKGCGTIQMEKLTGITSKADRFLKDWTYYDL |
| | QTKIENKAKEVGINVVYIAPKYTSQRCSKCGYIHKDNRPNQAKERCLECDFESNADYNA |
| | SQNIGIKNIDKIIEKDLQKQESEVQVNENK |
| SEQ ID | LIWKDALGGIILTKIVKLYLISEQIDKDGNRVDYKEINSILWNLQKQTRDIKNKTVQLC |
| NO: 151 | WEWMNFSSDYYKKNELYPNEKEILNLTLRGYAYDHFKQGYDLYSSNISVLTEAVCGAFK |
| | NAKKEMLNGEKSVLSYKAEQPLDIHKKCIKLEYDKNFYVKLKMLNKAGKKKYGIEDDLN |
| | FKIQVEDKSTRTILERCIDGEYVVSGSKLIYDKKKKLWKLNLCYSFKANEIESLDKNKI |
| | LGIDLGIACPLMASVNGEFDRFSIKGGEIETFRKRIEARKRSVLHQTKYCGDGRIGHGR |
| | NKRTEPAYKINDKIARFRDTANHKYSRALIDYAIRKNCGMIQMENLTGISDKKEHFLKE |
| | WSYYDLQTKIENKAKEKGIKIVYINPEYTSQRCSKCGYIDANNRELRAVFKCQKCGFEA |
| | DADYNASQNIGIKNIEDIIENTLKISSANEKQTKNT |
| SEQ ID | VFYSTFLCYILTKYIDFSANECYNINTSSEVKQLMNKVVKLALICEQSDKDNSPVDYKK |
| NO: 152 | INEILWELQKQTREIKNKAIQYCWEYNNFSSDYYKKENFYPKEKDILSYTLVGFVNDKF |
| | KTGNDLYSGNCSTTVRNACTEFKNSKKELIKGSRSIINYRSNQPLDIHNKCIRIEFENN |
| | CFYTYLKLLNRPAFKKYNFANTEIKFKILVRDNSTKTILERCISNEYEIAASKLLYDQK |
| | KKCWFLNLVYAFEIKSNNSLDPNKILGVDLGIHYPICASVYGSLDRFTIDGGEIDEFRR |
| | RVESRKISMLKQGKNCGDGRIGHGIKARNKPVYNIEDKIARFRDTANHKYSRALIEYAV |
| | KHTCGTIQMEDLTGITDIANRFLKNWSYYDLQTKIEYKAKEAGINIVYIDPKNTSRRCS |
| | KCGYIDKENRETQSRFICLKCGFKENADYNASQNIGIKDIDKLIKEDVH |
| SEQ ID | LISEQIDKDGNRVDYKEINSILWNLQKQTRDIKNKTVQLCWEWMNFSSDYYKKNELYPN |
| NO: 153 | EKEILNLTLRGYAYDHFKQGYDLYSSNISVLTEAVCGAFKNAKKEMLNGEKSVLSYKAE |
| | QPLDIHKKCIKLEYDKNFYVKLKMLNKAGKKKYGIEDDLNFKIQVEDKSTRTILERCID |
| | GEYVVSGSKLIYDKKKKLWKLNLCYSFKANEIESLDKNKILGIDLGIACPLMASVNGEF |
| | DRFSIKGGEIETFRKRIEARKRSVLHQTKYCGDGRIGHGRNKRTEPAYKINDKIARFRD |
| | TANHKYSRALIDYAIRKNCGMIQMENLTGISDNKEHFLKEWSYYDLQTKIENKAKEKGI |
| | KIVYINPEYTSQRCSKCGYIDANNRELRAVFKCQNCGFEADADYNASQNIGIKNIEDII |
| | ENTLKISSANEKQTKNT |
| SEQ ID | LVKVVKIYLISEQVDEQGKDVDYNTICGVLWDLQWETREIKNKTVQLCWEWSGFSSDYY |
| NO: 154 | KKYGEYPKEKNLLDYTMGGFVYDKLKSKYHLYTANLSTTSQNTCGIFRTYKVDFVKGNR |
| | SVLSFKADQPLDVHKKSISIDRIDDNYFVKLKLLNKSGIQKYGIRDDFHFRMLVKDNST |
| | KTILERCVGGDYKAAASKIIYDKKKKMWCLNLSYEFDVNTAKDLNKNRILGIDIGIVYP |
| | VVASVNGELDRFVIQGGEIETFRRRVENRKKSLLKQTKYCGDGRIGHGRNKRTEPVDII |
| | SDQIARFRNTANHKYSRAVIDYAVRKQCGTIQMENLKGITDKSDRFLKNWSYYDLQQKI |
| | EYKAKEKGINVVFINPKYTSQRCSRCGYIDSANRPKLPNQSKFLCIKCGFTENADYNAS |
| | QNIALYNIEKLIDAEA |

### 3. CasΦ Proteins

In some examples, the Type V CRISPR/Cas enzyme is a CasΦ nuclease. A CasΦ polypeptide can function as an endonuclease that catalyzes cleavage at a specific sequence in a target nucleic acid. A programmable CasΦ nuclease of the present disclosure may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

In some embodiments, the RuvC domain is a RuvC-like domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons, as described in review articles such as Shmakov et al. (Nature Reviews Microbiology volume 15, pages 169-182 (2017)) and Koonin E. V. and Makarova K. S. (2019, Phil. Trans. R. Soc., B 374:20180087). In some embodiments, the RuvC-like domain shares homology with the transposase IS605, OrfB, C-terminal. A transposase IS605, OrfB, C-terminal is easily identified by the skilled person using bioinformatics tools, such as PFAM (Finn et al. (Nucleic Acids Res. 2014 Jan. 1; 42 (Database issue): D222-D230); El-Gebali et al. (2019) Nucleic Acids Res. doi:10.1093/nar/gky995). PFAM is a database of protein families in which each entry is composed of a seed alignment which forms the basis to build a profile hidden Markov model (HMM) using the HMMER software (hmmer.org). It is readily accessible via pfam.xfam.org, maintained by EMBL-EBI, which easily allows an amino acid sequence to be analyzed against the current release of PFAM (e.g. version 33.1 from May 2020), but local builds can also be implemented using publicly- and freely-available database files and tools. A transposase IS605, OrfB, C-terminal is easily identified by the skilled person using the HMM PF07282 (accession number PF07282.12). The skilled person would also be able to identify a RuvC domain, for example with the HMM PF18516 (accession number PF18516.2), using the PFAM tool. In some embodiments, the programmable CasΦ nuclease comprises a RuvC-like domain which matches PFAM family PF07282 but does not match PFAM family PF18516, as assessed using the PFAM tool (e.g. using PFAM version 33.1, and the HMM accession numbers PF07282.12 and PF18516.2). PFAM searches should ideally be performed using an E-value cut off set at 1.0.

In some examples, a CasΦ nuclease of the disclosure can exhibit indiscriminate trans-cleavage of ssDNA, enabling its use for inducing cell death, apoptosis, cell cycle arrest, or a combination thereof, in a population cells. In some examples, a CasΦ nuclease of the disclosure can, upon hybridization of a guide nucleic acid molecule to a target DNA or RNA, induce cis-cleavage of the target DNA or RNA.

In some instances, the CasΦ protein has at least 80%, at least 85%, at least 90%, at least 92%, at least 95%, at least 97%, or at least 99% sequence identity to any one of SEQ ID NO: 155-SEQ ID NO: 202. In some examples, the CasΦ protein is selected from SEQ ID NO: 155-SEQ ID NO: 202.

TABLE 3 provides amino acid sequences of illustrative CasΦ polypeptides that can be used in compositions and methods of the disclosure.

TABLE 3
CasΦ Protein Sequences

| # | Sequence | Annotation |
|---|---|---|
| SEQ | MADTPTLFTQFLRHHLPGQRFRKDILKQAGRILANKGEDATIAFLRGKSEES | CasΦ.1 |
| ID | PPDFQPPVKCPIIACSRPLTEWPIYQASVAIQGYVYGQSLAEFEASDPGCSK | |
| NO: | DGLLGWFDKTGVCTDYFSVQGLNLIFQNARKRYIGVQTKVTNRNEKRHKKLK | |
| 155 | RINAKRIAEGLPELTSDEPESALDETGHLIDPPGLNTNIYCYQQVSPKPLAL | |
| | SEVNQLPTAYAGYSTSGDDPIQPMVTKDRLSISKGQPGYIPEHQRALLSQKK | |
| | HRRMRGYGLKARALLVIVRIQDDWAVIDLRSLLRNAYWRRIVQTKEPSTITK | |
| | LLKLVTGDPVLDATRMVATFTYKPGIVQVRSAKCLKNKQGSKLESERYLNET | |
| | VSVTSIDLGSNNLVAVATYRLVNGNTPELLQRFTLPSHLVKDFERYKQAHDT | |
| | LEDSIQKTAVASLPQGQQTEIRMWSMYGFREAQERVCQELGLADGSIPWNVM | |
| | TATSTILTDLFLARGGDPKKCMFTSEPKKKKNSKQVLYKIRDRAWAKMYRTL | |
| | LSKETREAWNKALWGLKRGSPDYARLSKRKEELARRCVNYTISTAEKRAQCG | |
| | RTIVALEDLNIGFFHGRGKQEPGWVGLFTRKKENRWLMQALHKAFLELAHHR | |
| | GYHVIEVNPAYTSQTCPVCRHCDPDNRDQHNREAFHCIGCGERGNADLDVAT | |
| | HNIAMVAITGESLKRARGSVASKTPQPLAAE | |
| SEQ | MPKPAVESEFSKVLKKHFPGERFRSSYMKRGGKILAAQGEEAVVAYLQGKSE | CasΦ.2 |
| ID | EEPPNFQPPAKCHVVTKSRDFAEWPIMKASEAIQRYIYALSTTERAACKPGK | |
| NO: | SSESHAAWFAATGVSNHGYSHVQGLNLIFDHTLGRYDGVLKKVQLRNEKARA | |
| 156 | RLESINASRADEGLPEIKAEEEEVATNETGHLLQPPGINPSFYVYQTISPQA | |
| | YRPRDEIVLPPEYAGYVRDPNAPIPLGVVRNRCDIQKGCPGYIPEWQREAGT | |
| | AISPKTGKAVTVPGLSPKKNKRMRRYWRSEKEKAQDALLVTVRIGTDWVVID | |
| | VRGLLRNARWRTIAPKDISLNALLDLFTGDPVIDVRRNIVTFTYTLDACGTY | |
| | ARKWTLKGKQTKATLDKLTATQTVALVAIDLGQTNPISAGISRVTQENGALQ | |
| | CEPLDRFTLPDDLLKDISAYRIAWDRNEEELRARSVEALPEAQQAEVRALDG | |
| | VSKETARTQLCADFGLDPKRLPWDKMSSNTTFISEALLSNSVSRDQVEFTPA | |
| | PKKGAKKKAPVEVMRKDRTWARAYKPRLSVEAQKLKNEALWALKRTSPEYLK | |
| | LSRRKEELCRRSINYVIEKTRRRTQCQIVIPVIEDLNVRFFHGSGKRLPGWD | |
| | NFFTAKKENRWFIQGLHKAFSDLRTHRSFYVFEVRPERTSITCPKCGHCEVG | |
| | NRDGEAFQCLSCGKTCNADLDVATHNLTQVALTGKTMPKREEPRDAQGTAPA | |
| | RKTKKASKSKAPPAEREDQTPAQEPSQTS | |
| SEQ | MYILEMADLKSEPSLLAKLLRDRFPGKYWLPKYWKLAEKKRLTGGEEAACEY | CasΦ.3 |
| ID | MADKQLDSPPPNERPPARCVILAKSRPFEDWPVHRVASKAQSEVIGLSEQGE | |
| NO: | AALRAAPPSTADARRDWLRSHGASEDDLMALEAQLLETIMGNAISLHGGVLK | |
| 157 | KIDNANVKAAKRLSGRNEARLNKGLQELPPEQEGSAYGADGLLVNPPGLNLN | |
| | IYCRKSCCPKPVKNTARFVGHYPGYLRDSDSILISGTMDRLTIIEGMPGHIP | |
| | AWQREQGLVKPGGRRRRLSGSESNMRQKVDPSTGPRRSTRSGTVNRSNQRTG | |
| | RNGDPLLVEIRMKEDWVLLDARGLLRNLRWRESKRGLSCDHEDLSLSGLLAL | |
| | FSGDPVIDPVRNEVVFLYGEGIIPVRSTKPVGTRQSKKLLERQASMGPLTLI | |
| | SCDLGQTNLIAGRASAISLTHGSLGVRSSVRIELDPEIIKSFERLRKDADRL | |
| | ETEILTAAKETLSDEQRGEVNSHEKDSPQTAKASLCRELGLHPPSLPWGQMG | |
| | PSTTFIADMLISHGRDDDAFLSHGEFPTLEKRKKEDKRFCLESRPLLSSETR | |
| | KALNESLWEVKRTSSEYARLSQRKKEMARRAVNFVVEISRRKTGLSNVIVNI | |
| | EDLNVRIFHGGGKQAPGWDGFFRPKSENRWFIQAIHKAFSDLAAHHGIPVIE | |
| | SDPQRTSMTCPECGHCDSKNRNGVRFLCKGCGASMDADFDAACRNLERVALT | |
| | GKPMPKPSTSCERLLSATTGKVCSDHSLSHDAIEKAS | |
| SEQ | MEKEITELTKIRREFPNKKESSTDMKKAGKLLKAEGPDAVRDELNSCQEIIG | CasΦ. 4 |
| ID | DFKPPVKTNIVSISRPFEEWPVSMVGRAIQEYYFSLTKEELESVHPGTSSED | |
| NO: | HKSFFNITGLSNYNYTSVQGLNLIFKNAKAIYDGTLVKANNKNKKLEKKENE | |
| 158 | INHKRSLEGLPIITPDFEEPFDENGHLNNPPGINRNIYGYQGCAAKVFVPSK | |
| | HKMVSLPKEYEGYNRDPNLSLAGERNRLEIPEGEPGHVPWFQRMDIPEGQIG | |
| | HVNKIQRFNFVHGKNSGKVKFSDKTGRVKRYHHSKYKDATKPYKFLEESKKV | |
| | SALDSILAIITIGDDWVVFDIRGLYRNVFYRELAQKGLTAVQLLDLFTGDPV | |
| | IDPKKGVVTFSYKEGVVPVFSQKIVPRFKSRDTLEKLTSQGPVALLSVDLGQ | |
| | NEPVAARVCSLKNINDKITLDNSCRISFLDDYKKQIKDYRDSLDELEIKIRL | |
| | EAINSLETNQQVEIRDLDVESADRAKANTVDMFDIDPNLISWDSMSDARVST | |
| | QISDLYLKNGGDESRVYFEINNKRIKRSDYNISQLVRPKLSDSTRKNLNDSI | |
| | WKLKRTSEEYLKLSKRKLELSRAVVNYTIRQSKLLSGINDIVIILEDLDVKK | |
| | KENGRGIRDIGWDNFFSSRKENRWFIPAFHKAFSELSSNRGLCVIEVNPAWT | |
| | SATCPDCGFCSKENRDGINFTCRKCGVSYHADIDVATLNIARVAVLGKPMSG | |
| | PADRERLGDTKKPRVARSRKTMKRKDISNSTVEAMVTA | |
| SEQ | MDMLDTETNYATETPAQQQDYSPKPPKKAQRAPKGFSKKARPEKKPPKPITL | CasΦ.5 |
| ID | FTQKHFSGVRFLKRVIRDASKILKLSESRTITFLEQAIERDGSAPPDVTPPV | |
| NO: | HNTIMAVTRPFEEWPEVILSKALQKHCYALTKKIKIKTWPKKGPGKKCLAAW | |
| 159 | SARTKIPLIPGQVQATNGLEDRIGSIYDGVEKKVTNRNANKKLEYDEAIKEG | |
| | RNPAVPEYETAYNIDGTLINKPGYNPNLYITQSRTPRLITEADRPLVEKILW | |
| | QMVEKKTQSRNQARRARLEKAAHLQGLPVPKFVPEKVDRSQKIEIRIIDPLD | |
| | KIEPYMPQDRMAIKASQDGHVPYWQRPFLSKRRNRRVRAGWGKQVSSIQAWL | |
| | TGALLVIVRLGNEAFLADIRGALRNAQWRKLLKPDATYQSLFNLFTGDPVVN | |
| | TRTNHLTMAYREGVVNIVKSRSFKGRQTREHLLTLLGQGKTVAGVSFDLGQK | |
| | HAAGLLAAHFGLGEDGNPVFTPIQACFLPQRYLDSLTNYRNRYDALTLDMRR | |
| | QSLLALTPAQQQEFADAQRDPGGQAKRACCLKLNLNPDEIRWDLVSGISTMI | |
| | SDLYIERGGDPRDVHQQVETKPKGKRKSEIRILKIRDGKWAYDERPKIADET | |
| | RKAQREQLWKLQKASSEFERLSRYKINIARAIANWALQWGRELSGCDIVIPV | |
| | LEDLNVGSKFFDGKGKWLLGWDNRFTPKKENRWFIKVLHKAVAELAPHRGVP | |
| | VYEVMPHRTSMTCPACHYCHPTNREGDRFECQSCHVVKNTDRDVAPYNILRV | |
| | AVEGKTLDRWQAEKKPQAEPDRPMILIDNQES | |
| SEQ | MDMLDTETNYATETPAQQQDYSPKPPKKAQRAPKGFSKKARPEKKPPKPITL | CasΦ.6 |
| ID | FTQKHFSGVRFLKRVIRDASKILKLSESRTITFLEQAIERDGSAPPDVTPPV | |
| NO: | HNTIMAVTRPFEEWPEVILSKALQKHCYALTKKIKIKTWPKKGPGKKCLAAW | |
| 160 | SARTKIPLIPGQVQATNGLEDRIGSIYDGVEKKVTNRNANKKLEYDEAIKEG | |
| | RNPAVPEYETAYNIDGTLINKPGYNPNLYITQSRTPRLITEADRPLVEKILW | |
| | QMVEKKTQSRNQARRARLEKAAHLQGLPVPKFVPEKVDRSQKIEIRIIDPLD | |
| | KIEPYMPQDRMAIKASQDGHVPYWQRPFLSKRRNRRVRAGWGKQVSSIQAWL | |
| | TGALLVIVRLGNEAFLADIRGALRNAQWRKLLKPDATYQSLENLFTGDPVVN | |
| | TRTNHLTMAYREGVVDIVKSRSFKGRQTREHLLTLLGQGKTVAGVSFDLGQK | |
| | HAAGLLAAHFGLGEDGNPVFTPIQACFLPQRYLDSLTNYRNRYDALTLDMRR | |
| | QSLLALTPAQQQEFADAQRDPGGQAKRACCLKLNLNPDEIRWDLVSGISTMI | |
| | SDLYIERGGDPRDVHQQVETKPKGKRKSEIRILKIRDGKWAYDERPKIADET | |
| | RKAQREQLWKLQKASSEFERLSRYKINIARAIANWALQWGRELSGCDIVIPV | |
| | LEDLNVGSKFFDGKGKWLLGWDNRFTPKKENRWFIKVLHKAVAELAPHKGVP | |
| | VYEVMPHRTSMTCPACHYCHPTNREGDRFECQSCHVVKNTDRDVAPYNILRV | |
| | AVEGKTLDRWQAEKKPQAEPDRPMILIDNQES | |
| SEQ | MSSLPTPLELLKQKHADLFKGLQFSSKDNKMAGKVLKKDGEEAALAFLSERG | CasΦ. 7 |
| ID | VSRGELPNFRPPAKTLVVAQSRPFEEFPIYRVSEAIQLYVYSLSVKELETVP | |
| NO: | SGSSTKKEHQRFFQDSSVPDFGYTSVQGLNKIFGLARGIYLGVITRGENQLQ | |
| 161 | KAKSKHEALNKKRRASGEAETEFDPTPYEYMTPERKLAKPPGVNHSIMCYVD | |
| | ISVDEFDERNPDGIVLPSEYAGYCREINTAIEKGTVDRLGHLKGGPGYIPGH | |
| | QRKESTTEGPKINFRKGRIRRSYTALYAKRDSRRVRQGKLALPSYRHHMMRL | |
| | NSNAESAILAVIFFGKDWVVFDLRGLLRNVRWRNLFVDGSTPSTLLGMEGDP | |
| | VIDPKRGVVAFCYKEQIVPVVSKSITKMVKAPELLNKLYLKSEDPLVLVAID | |
| | LGQTNPVGVGVYRVMNASLDYEVVTRFALESELLREIESYRQRTNAFEAQIR | |
| | AETFDAMTSEEQEEITRVRAFSASKAKENVCHRFGMPVDAVDWATMGSNTIH | |
| | IAKWVMRHGDPSLVEVLEYRKDNEIKLDKNGVPKKVKLTDKRIANLTSIRLR | |
| | FSQETSKHYNDTMWELRRKHPVYQKLSKSKADFSRRVVNSIIRRVNHLVPRA | |
| | RIVFIIEDLKNLGKVFHGSGKRELGWDSYFEPKSENRWFIQVLHKAFSETGK | |
| | HKGYYIIECWPNWTSCTCPKCSCCDSENRHGEVERCLACGYTCNTDFGTAPD | |
| | NLVKIATTGKGLPGPKKRCKGSSKGKNPKIARSSETGVSVTESGAPKVKKSS | |
| | PTQTSQSSSQSAP | |
| SEQ | MNKIEKEKTPLAKLMNENFAGLRFPFAIIKQAGKKLLKEGELKTIEYMTGKG | CasΦ.8 |
| ID | SIEPLPNFKPPVKCLIVAKRRDLKYFPICKASCEIQSYVYSLNYKDEMDYES | |
| NO: | TPMTSQKQHEEFFKKSGLNIEYQNVAGLNLIFNNVKNTYNGVILKVKNRNEK | |
| 162 | LKKKAIKNNYEFEEIKTENDDGCLINKPGINNVIYCFQSISPKILKNITHLP | |
| | KEYNDYDCSVDRNIIQKYVSRLDIPESQPGHVPEWQRKLPEFNNTNNPRRRR | |
| | KWYSNGRNISKGYSVDQVNQAKIEDSLLAQIKIGEDWIILDIRGLLRDLNRR | |
| | ELISYKNKLTIKDVLGFFSDYPIIDIKKNLVTFCYKEGVIQVVSQKSIGNKK | |
| | SKQLLEKLIENKPIALVSIDLGQTNPVSVKISKLNKINNKISIESFTYRELN | |
| | EEILKEIEKYRKDYDKLELKLINEA | |
| SEQ | MDMLDTETNYATETPSQQQDYSPKPPKKDRRAPKGFSKKARPEKKPPKPITL | CasΦ.9 |
| ID | FTQKHFSGVRFLKRVIRDASKILKLSESRTITFLEQAIERDGSAPPDVTPPV | |
| NO: | HNTIMAVTRPFEEWPEVILSKALQKHCYALTKKIKIKTWPKKGPGKKCLAAW | |
| 163 | SARTKIPLIPGQVQATNGLEDRIGSIYDGVEKKVTNRNANKKLEYDEAIKEG | |
| | RNPAVPEYETAYNIDGTLINKPGYNPNLYITQSRTPRLITEADRPLVEKILW | |
| | QMVEKKTQSRNQARRARLEKAAHLQGLPVPKFVPEKVDRSQKIEIRIIDPLD | |
| | KIEPYMPQDRMAIKASQDGHVPYWQRPFLSKRRNRRVRAGWGKQVSSIQAWL | |
| | TGALLVIVRLGNEAFLADIRGALRNAQWRKLLKPDATYQSLENLFTGDPVVN | |
| | TRTNHLTMAYREGVVDIVKSRSFKGRQTREHLLTLLGQGKTVAGVSEDLGQK | |
| | HAAGLLAAHFGLGEDGNPVFTPIQACELPQRYLDSLTNYRNRYDALTLDMRR | |
| | QSLLALTPAQQQEFADAQRDPGGQAKRACCLKLNLNPDEIRWDLVSGISTMI | |
| | SDLYIERGGDPRDVHQQVETKPKGKRKSEIRILKIRDGKWAYDFRPKIADET | |
| | RKAQREQLWKLQKASSEFERLSRYKINIARAIANWALQWGRELSGCDIVIPV | |
| | LEDLNVGSKFFDGKGKWLLGWDNRFTPKKENRWFIKVLHKAVAELAPHRGVP | |
| | VYEVMPHRTSMTCPACHYCHPTNREGDRFECQSCHVVKNTDRDVAPYNILRV | |
| | AVEGKTLDRWQAEKKPQAEPDRPMILIDNQES | |
| SEQ | MDMLDTETNYATETPSQQQDYSPKPPKKDRRAPKGFSKKARPEKKPPKPITL | CasΦ.10 |
| ID | FTQKHFSGVRFLKRVIRDASKILKLSESRTITFLEQAIERDGSAPPDVTPPV | |
| NO: | HNTIMAVTRPFEEWPEVILSKALQKHCYALTKKIKIKTWPKKGPGKKCLAAW | |
| 164 | SARTKIPLIPGQVQATNGLEDRIGSIYDGVEKKVTNRNANKKLEYDEAIKEG | |
| | RNPAVPEYETAYNIDGTLINKPGYNPNLYITQSRTPRLITEADRPLVEKILW | |
| | QMVEKKTQSRNQARRARLEKAAHLQGLPVPKFVPEKVDRSQKIEIRIIDPLD | |
| | KIEPYMPQDRMAIKASQDGHVPYWQRPFLSKRRNRRVRAGWGKQVSSIQAWL | |
| | TGALLVIVRLGNEAFLADIRGALRNAQWRKLLKPDATYQSLENLFTGDPVVN | |
| | TRTNHLTMAYREGVVNIVKSRSFKGRQTREHLLTLLGQGKTVAGVSEDLGQK | |
| | HAAGLLAAHFGLGEDGNPVFTPIQACELPQRYLDSLTNYRNRYDALTLDMRR | |
| | QSLLALTPAQQQEFADAQRDPGGQAKRACCLKLNLNPDEIRWDLVSGISTMI | |
| | SDLYIERGGDPRDVHQQVETKPKGKRKSEIRILKIRDGKWAYDERPKIADET | |
| | RKAQREQLWKLQKASSEFERLSRYKINIARAIANWALQWGRELSGCDIVIPV | |
| | LEDLNVGSKFFDGKGKWLLGWDNRFTPKKENRWFIKVLHKAVAELAPHRGVP | |
| | VYEVMPHRTSMTCPACHYCHPTNREGDRFECQSCHVVKNTDRDVAPYNILRV | |
| | AVEGKTLDRWQAEKKPQAEPDRPMILIDNQES | |
| SEQ | MSNKTTPPSPLSLLLRAHFPGLKFESQDYKIAGKKLRDGGPEAVISYLTGKG | CasΦ.11 |
| ID | QAKLKDVKPPAKAFVIAQSRPFIEWDLVRVSRQIQEKIFGIPATKGRPKQDG | |
| NO: | LSETAFNEAVASLEVDGKSKLNEETRAAFYEVLGLDAPSLHAQAQNALIKSA | |
| 165 | ISIREGVLKKVENRNEKNLSKTKRRKEAGEEATFVEEKAHDERGYLIHPPGV | |
| | NQTIPGYQAVVIKSCPSDFIGLPSGCLAKESAEALTDYLPHDRMTIPKGQPG | |
| | YVPEWQHPLLNRRKNRRRRDWYSASLNKPKATCSKRSGTPNRKNSRTDQIQS | |
| | GRFKGAIPVLMRFQDEWVIIDIRGLLRNARYRKLLKEKSTIPDLLSLFTGDP | |
| | SIDMRQGVCTFIYKAGQACSAKMVKTKNAPEILSELTKSGPVVLVSIDLGQT | |
| | NPIAAKVSRVTQLSDGQLSHETLLRELLSNDSSDGKEIARYRVASDRLRDKL | |
| | ANLAVERLSPEHKSEILRAKNDTPALCKARVCAALGLNPEMIAWDKMTPYTE | |
| | FLATAYLEKGGDRKVATLKPKNRPEMLRRDIKEKGTEGVRIEVSPEAAEAYR | |
| | EAQWDLQRTSPEYLRLSTWKQELTKRILNQLRHKAAKSSQCEVVVMAFEDLN | |
| | IKMMHGNGKWADGGWDAFFIKKRENRWEMQAFHKSLTELGAHKGVPTIEVTP | |
| | HRTSITCTKCGHCDKANRDGERFACQKCGFVAHADLEIATDNIERVALTGKP | |
| | MPKPESERSGDAKKSVGARKAAFKPEEDAEAAE | |
| SEQ | MIKPTVSQFLTPGFKLIRNHSRTAGLKLKNEGEEACKKFVRENEIPKDECPN | CasΦ.12 |
| ID | FQGGPAIANIIAKSREFTEWEIYQSSLAIQEVIFTLPKDKLPEPILKEEWRA | |
| NO: | QWLSEHGLDTVPYKEAAGLNLIIKNAVNTYKGVQVKVDNKNKNNLAKINRKN | |
| 166 | EIAKLNGEQEISFEEIKAFDDKGYLLQKPSPNKSIYCYQSVSPKPFITSKYH | |
| | NVNLPEEYIGYYRKSNEPIVSPYQFDRLRIPIGEPGYVPKWQYTELSKKENK | |
| | RRKLSKRIKNVSPILGIICIKKDWCVEDMRGLLRTNHWKKYHKPTDSINDLF | |
| | DYFTGDPVIDTKANVVRFRYKMENGIVNYKPVREKKGKELLENICDQNGSCK | |
| | LATVDVGQNNPVAIGLFELKKVNGELTKTLISRHPTPIDFCNKITAYRERYD | |
| | KLESSIKLDAIKQLTSEQKIEVDNYNNNFTPQNTKQIVCSKLNINPNDLPWD | |
| | KMISGTHFISEKAQVSNKSEIYFTSTDKGKTKDVMKSDYKWFQDYKPKLSKE | |
| | VRDALSDIEWRLRRESLEFNKLSKSREQDARQLANWISSMCDVIGIENLVKK | |
| | NNFFGGSGKREPGWDNFYKPKKENRWWINAIHKALTELSQNKGKRVILLPAM | |
| | RTSITCPKCKYCDSKNRNGEKENCLKCGIELNADIDVATENLATVAITAQSM | |
| | PKPTCERSGDAKKPVRARKAKAPEFHDKLAPSYTVVLREAV | |
| SEQ | MRQPAEKTAFQVFRQEVIGTQKLSGGDAKTAGRLYKQGKMEAAREWLLKGAR | CasΦ.13 |
| ID | DDVPPNFQPPAKCLVVAVSHPFEEWDISKTNHDVQAYIYAQPLQAEGHLNGL | |
| NO: | SEKWEDTSADQHKLWFEKTGVPDRGLPVQAINKIAKAAVNRAFGVVRKVENR | |
| 167 | NEKRRSRDNRIAEHNRENGLTEVVREAPEVATNADGELLHPPGIDPSILSYA | |
| | SVSPVPYNSSKHSFVRLPEEYQAYNVEPDAPIPQFVVEDRFAIPPGQPGYVP | |
| | EWQRLKCSTNKHRRMRQWSNQDYKPKAGRRAKPLEFQAHLTRERAKGALLVV | |
| | MRIKEDWVVFDVRGLLRNVEWRKVLSEEAREKLTLKGLLDLFTGDPVIDTKR | |
| | GIVTFLYKAEITKILSKRTVKTKNARDLLLRLTEPGEDGLRREVGLVAVDLG | |
| | QTHPIAAAIYRIGRTSAGALESTVLHRQGLREDQKEKLKEYRKRHTALDSRL | |
| | RKEAFETLSVEQQKEIVTVSGSGAQITKDKVCNYLGVDPSTLPWEKMGSYTH | |
| | FISDDFLRRGGDPNIVHFDRQPKKGKVSKKSQRIKRSDSQWVGRMRPRLSQE | |
| | TAKARMEADWAAQNENEEYKRLARSKQELARWCVNTLLQNTRCITQCDEIVV | |
| | VIEDLNVKSLHGKGAREPGWDNFFTPKTENRWFIQILHKTFSELPKHRGEHV | |
| | IEGCPLRTSITCPACSYCDKNSRNGEKFVCVACGATFHADFEVATYNLVRLA | |
| | TTGMPMPKSLERQGGGEKAGGARKARKKAKQVEKIVVQANANVTMNGASLHS | |
| | P | |
| SEQ | MSSLPTPLELLKQKHADLFKGLQFSSKDNKMAGKVLKKDGEEAALAFLSERG | CasΦ.14 |
| ID | VSRGELPNFRPPAKTLVVAQSRPFEEFPIYRVSEAIQLYVYSLSVKELETVP | |
| NO: | SGSSTKKEHQRFFQDSSVPDFGYTSVQGLNKIFGLARGIYLGVITRGENQLQ | |
| 168 | KAKSKHEALNKKRRASGEAETEFDPTPYEYMTPERKLAKPPGVNHSIMCYVD | |
| | ISVDEFDERNPDGIVLPSEYAGYCREINTAIEKGTVDRLGHLKGGPGYIPGH | |
| | QRKESTTEGPKINFRKGRIRRSYTALYAKRDSRRVRQGKLALPSYRHHMMRL | |
| | NSNAESAILAVIFFGKDWVVFDLRGLLRNVRWRNLFVDGSTPSTLLGMFGDP | |
| | VIDPKRGVVAFCYKEQIVPVVSKSITKMVKAPELLNKLYLKSEDPLVLVAID | |
| | LGQTNPVGVGVYRVMNASLDYEVVTRFALESELLREIESYRQRTNAFEAQIR | |
| | AETFDAMTSEEQEEITRVRAFSASKAKENVCHRFGMPVDAVDWATMGSNTIH | |
| | IAKWVMRHGDPSLVEVLEYRKDNEIKLDKNGVPKKVKLTDKRIANLTSIRLR | |
| | FSQETSKHYNDTMWELRRKHPVYQKLSKSKADESRRVVNSIIRRVNHLVPRA | |
| | RIVFIIEDLKNLGKVFHGSGKRELGWDSYFEPKSENRWFIQVLHKAFSETGK | |
| | HKGYYIIECWPNWTSCTCPKCSCCDSENRHGEVERCLACGYTCNTDFGTAPD | |
| | NLVKIATTGKGLPGPKKRCKGSSKGKNPKIARSSETGVSVTESGAPKVKKSS | |
| | PTQTSQSSSQSAP | |
| SEQ | MIKPTVSQFLTPGFKLIRNHSRTAGLKLKNEGEEACKKFVRENEIPKDECPN | CasΦ.15 |
| ID | FQGGPAIANIIAKSREFTEWEIYQSSLAIQEVIFTLPKDKLPEPILKEEWRA | |
| NO: | QWLSEHGLDTVPYKEAAGLNLIIKNAVNTYKGVQVKVDNKNKNNLAKINRKN | |
| 169 | EIAKLNGEQEISFEEIKAFDDKGYLLQKPSPNKSIYCYQSVSPKPFITSKYH | |
| | NVNLPEEYIGYYRKSNEPIVSPYQFDRLRIPIGEPGYVPKWQYTELSKKENK | |
| | RRKLSKRIKNVSPILGIICIKKDWCVFDMRGLLRTNHWKKYHKPTDSINDLF | |
| | DYFTGDPVIDTKANVVRFRYKMENGIVNYKPVREKKGKELLENICDQNGSCK | |
| | LATVDVGQNNPVAIGLFELKKVNGELTKTLISRHPTPIDFCNKITAYRERYD | |
| | KLESSIKLDAIKQLTSEQKIEVDNYNNNFTPQNTKQIVCSKLNINPNDLPWD | |
| | KMISGTHFISEKAQVSNKSEIYFTSTDKGKTKDVMKSDYKWFQDYKPKLSKE | |
| | VRDALSDIEWRLRRESLEFNKLSKSREQDARQLANWISSMCDVIGIENLVKK | |
| | NNFFGGSGKREPGWDNFYKPKKENRWWINAIHKALTELSQNKGKRVILLPAM | |
| | RTSITCPKCKYCDSKNRNGEKENCLKCGIELNADIDVATENLATVAITAQSM | |
| | PKPTCERSGDAKKPVRARKAKAPEFHDKLAPSYTVVLREAV | |
| SEQ | MSNKTTPPSPLSLLLRAHFPGLKFESQDYKIAGKKLRDGGPEAVISYLTGKG | CasΦ.16 |
| ID | QAKLKDVKPPAKAFVIAQSRPFIEWDLVRVSRQIQEKIFGIPATKGRPKQDG | |
| NO: | LSETAFNEAVASLEVDGKSKLNEETRAAFYEVLGLDAPSLHAQAQNALIKSA | |
| 170 | ISIREGVLKKVENRNEKNLSKTKRRKEAGEEATFVEEKAHDERGYLIHPPGV | |
| | NQTIPGYQAVVIKSCPSDFIGLPSGCLAKESAEALTDYLPHDRMTIPKGQPG | |
| | YVPEWQHPLLNRRKNRRRRDWYSASLNKPKATCSKRSGTPNRKNSRTDQIQS | |
| | GRFKGAIPVLMRFQDEWVIIDIRGLLRNARYRKLLKEKSTIPDLLSLETGDP | |
| | SIDMRQGVCTFIYKAGQACSAKMVKTKNAPEILSELTKSGPVVLVSIDLGQT | |
| | NPIAAKVSRVTQLSDGQLSHETLLRELLSNDSSDGKEIARYRVASDRLRDKL | |
| | ANLAVERLSPEHKSEILRAKNDTPALCKARVCAALGLNPEMIAWDKMTPYTE | |
| | FLATAYLEKGGDRKVATLKPKNRPEMLRRDIKEKGTEGVRIEVSPEAAEAYR | |
| | EAQWDLQRTSPEYLRLSTWKQELTKRILNQLRHKAAKSSQCEVVVMAFEDLN | |
| | IKMMHGNGKWADGGWDAFFIKKRENRWEMQAFHKSLTELGAHKGVPTIEVTP | |
| | HRTSITCTKCGHCDKANRDGERFACQKCGEVAHADLEIATDNIERVALTGKP | |
| | MPKPESERSGDAKKSVGARKAAFKPEEDAEAAE | |
| SEQ | MYSLEMADLKSEPSLLAKLLRDRFPGKYWLPKYWKLAEKKRLTGGEEAACEY | CasΦ.17 |
| ID | MADKQLDSPPPNERPPARCVILAKSRPFEDWPVHRVASKAQSFVIGLSEQGE | |
| NO: | AALRAAPPSTADARRDWLRSHGASEDDLMALEAQLLETIMGNAISLHGGVLK | |
| 1171 | KIDNANVKAAKRLSGRNEARLNKGLQELPPEQEGSAYGADGLLVNPPGLNLN | |
| | IYCRKSCCPKPVKNTARFVGHYPGYLRDSDSILISGTMDRLTIIEGMPGHIP | |
| | AWQREQGLVKPGGRRRRLSGSESNMRQKVDPSTGPRRSTRSGTVNRSNQRTG | |
| | RNGDPLLVEIRMKEDWVLLDARGLLRNLRWRESKRGLSCDHEDLSLSGLLAL | |
| | FSGDPVIDPVRNEVVFLYGEGIIPVRSTKPVGTRQSKKLLERQASMGPLTLI | |
| | SCDLGQTNLIAGRASAISLTHGSLGVRSSVRIELDPEIIKSFERLRKDADRL | |
| | ETEILTAAKETLSDEQRGEVNSHEKDSPQTAKASLCRELGLHPPSLPWGQMG | |
| | PSTTFIADMLISHGRDDDAFLSHGEFPTLEKRKKEDKRFCLESRPLLSSETR | |
| | KALNESLWEVKRTSSEYARLSQRKKEMARRAVNFVVEISRRKTGLSNVIVNI | |
| | EDLNVRIFHGGGKQAPGWDGFFRPKSENRWFIQAIHKAFSDLAAHHGIPVIE | |
| | SDPQRTSMTCPECGHCDSKNRNGVRFLCKGCGASMDADEDAACRNLERVALT | |
| | GKPMPKPSTSCERLLSATTGKVCSDHSLSHDAIEKAS | |
| SEQ | MEKEITELTKIRREFPNKKESSTDMKKAGKLLKAEGPDAVRDELNSCQEIIG | CasΦ.18 |
| ID | DFKPPVKTNIVSISRPFEEWPVSMVGRAIQEYYFSLTKEELESVHPGTSSED | |
| NO: | HKSFFNITGLSNYNYTSVQGLNLIFKNAKAIYDGTLVKANNKNKKLEKKENE | |
| 172 | INHKRSLEGLPIITPDFEEPFDENGHLNNPPGINRNIYGYQGCAAKVFVPSK | |
| | HKMVSLPKEYEGYNRDPNLSLAGERNRLEIPEGEPGHVPWFQRMDIPEGQIG | |
| | HVNKIQRFNFVHGKNSGKVKFSDKTGRVKRYHHSKYKDATKPYKFLEESKKV | |
| | SALDSILAIITIGDDWVVFDIRGLYRNVFYRELAQKGLTAVQLLDLFTGDPV | |
| | IDPKKGVVTFSYKEGVVPVFSQKIVPREKSRDTLEKLTSQGPVALLSVDLGQ | |
| | NEPVAARVCSLKNINDKITLDNSCRISFLDDYKKQIKDYRDSLDELEIKIRL | |
| | EAINSLETNQQVEIRDLDVESADRAKANTVDMFDIDPNLISWDSMSDARVST | |
| | QISDLYLKNGGDESRVYFEINNKRIKRSDYNISQLVRPKLSDSTRKNLNDSI | |
| | WKLKRTSEEYLKLSKRKLELSRAVVNYTIRQSKLLSGINDIVIILEDLDVKK | |
| | KFNGRGIRDIGWDNFFSSRKENRWFIPAFHKTFSELSSNRGLCVIEVNPAWT | |
| | SATCPDCGFCSKENRDGINFTCRKCGVSYHADIDVATLNIARVAVLGKPMSG | |
| | PADRERLGDTKKPRVARSRKTMKRKDISNSTVEAMVTA | |
| SEQ | MLVRTSTLVQDNKNSRSASRAFLKKPKMPKNKHIKEPTELAKLIRELFPGQR | CasΦ.19 |
| ID | FTRAINTQAGKILKHKGRDEVVEFLKNKGIDKEQFMDERPPTKARIVATSGA | |
| NO: | IEEFSYLRVSMAIQECCFGKYKFPKEKVNGKLVLETVGLTKEELDDELPKKY | |
| 173 | YENKKSRDRFFLKTGICDYGYTYAQGLNEIERNTRAIYEGVFTKVNNRNEKR | |
| | REKKDKYNEERRSKGLSEEPYDEDESATDESGHLINPPGVNLNIWTCEGFCK | |
| | GPYVTKLSGTPGYEVILPKVEDGYNRDPNEIISCGITDRFAIPEGEPGHIPW | |
| | HQRLEIPEGQPGYVPGHQRFADTGQNNSGKANPNKKGRMRKYYGHGTKYTQP | |
| | GEYQEVERKGHREGNKRRYWEEDFRSEAHDCILYVIHIGDDWVVCDLRGPLR | |
| | DAYRRGLVPKEGITTQELCNLFSGDPVIDPKHGVVTFCYKNGLVRAQKTISA | |
| | GKKSRELLGALTSQGPIALIGVDLGQTEPVGARAFIVNQARGSLSLPTLKGS | |
| | FLLTAENSSSWNVEKGEIKAYREAIDDLAIRLKKEAVATLSVEQQTEIESYE | |
| | AFSAEDAKQLACEKFGVDSSFILWEDMTPYHTGPATYYFAKQFLKKNGGNKS | |
| | LIEYIPYQKKKSKKTPKAVLRSDYNIACCVRPKLLPETRKALNEAIRIVQKN | |
| | SDEYQRLSKRKLEFCRRVVNYLVRKAKKLTGLERVIIAIEDLKSLEKFFTGS | |
| | GKRDNGWSNFFRPKKENRWFIPAFHKAFSELAPNRGFYVIECNPARTSITDP | |
| | DCGYCDGDNRDGIKFECKKCGAKHHTDLDVAPLNIAIVAVTGRPMPKTVSNK | |
| | SKRERSGGEKSVGASRKRNHRKSKANQEMLDATSSAAE | |
| SEQ | MPKIKKPTEISLLRKEVFPDLHFAKDRMRAASLVLKNEGREAAIEYLRVNHE | CasΦ.20 |
| ID | DKPPNEMPPAKTPYVALSRPLEQWPIAQASIAIQKYIFGLTKDEFSATKKLL | |
| NO: | YGDKSTPNTESRKRWFEVTGVPNFGYMSAQGLNAIFSGALARYEGVVQKVEN | |
| 174 | RNKKRFEKLSEKNQLLIEEGQPVKDYVPDTAYHTPETLQKLAENNHVRVEDL | |
| | GDMIDRLVHPPGIHRSIYGYQQVPPFAYDPDNPKGIILPKAYAGYTRKPHDI | |
| | IEAMPNRLNIPEGQAGYIPEHQRDKLKKGGRVKRLRTTRVRVDATETVRAKA | |
| | EALNAEKARLRGKEAILAVEQIEEDWALIDMRGLLRNVYMRKLIAAGELTPT | |
| | TLLGYFTETLTLDPRRTEATFCYHLRSEGALHAEYVRHGKNTRELLLDLTKD | |
| | NEKIALVTIDLGQRNPLAAAIFRVGRDASGDLTENSLEPVSRMLLPQAYLDQ | |
| | IKAYRDAYDSFRQNIWDTALASLTPEQQRQILAYEAYTPDDSKENVLRLLLG | |
| | GNVMPDDLPWEDMTKNTHYISDRYLADGGDPSKVWFVPGPRKRKKNAPPLKK | |
| | PPKPRELVKRSDHNISHLSEFRPQLLKETRDAFEKAKIDTERGHVGYQKLST | |
| | RKDQLCKEILNWLEAEAVRLTRCKTMVLGLEDLNGPFFNQGKGKVRGWVSFF | |
| | RQKQENRWIVNGFRKNALARAHDKGKYILELWPSWTSQTCPKCKHVHADNRH | |
| | GDDFVCLQCGARLHADAEVATWNLAVVAIQGHSLPGPVREKSNDRKKSGSAR | |
| | KSKKANESGKVVGAWAAQATPKRATSKKETGTARNPVYNPLETQASCPAP | |
| SEQ | MTPSPQIARLVETPLAAALKAHHPGKKFRSDYLKKAGKILKDQGVEAAMAHL | CasΦ.21 |
| ID | DGKDQAEPPNFKPPAKCRIVARSREFSEWPIVKASVEIQKYIYGLTLEERKA | |
| NO: | CDPGKSSASHKAWFAKTGVNTEGYSSVQGENLIFGHTLGRYDGVLVKTENLN | |
| 175 | KKRAEKNERFRAKALAEGRAEPVCPPLVTATNDTGQDVTLEDGRVVRPGQLL | |
| | QPPGINPNIYAYQQVSPKAYVPGIIELPEEFQGYSRDPNAVILPLVPRDRLS | |
| | IPKGQPGYVPEPHREGLTGRKDRRMRRYYETERGTKLKRPPLTAKGRADKAN | |
| | EALLVVVRIDSDWVVMDVRGLLRNARWRRLVSKEGITLNGLLDLFTGDPVLN | |
| | PKDCSVSRDTGDPVNDPRHGVVTFCYKLGVVDVCSKDRPIKGERTKEVLERL | |
| | TSSGTVGMVSIDLGQTNPVAAAVSRVTKGLQAETLETFTLPDDLLGKVRAYR | |
| | AKTDRMEEGFRRNALRKLTAEQQAEITRYNDATEQQAKALVCSTYGIGPEEV | |
| | PWERMTSNTTYISDHILDHGGDPDTVFFMATKRGQNKPTLHKRKDKAWGQKF | |
| | RPAISVETRLARQAAEWELRRASLEFQKLSVWKTELCRQAVNYVMERTKKRT | |
| | QCDVIIPVIEDLPVPLFHGSGKRDPGWANFFVHKRENRWFIDGLHKAFSELG | |
| | KHRGIYVFEVCPQRTSITCPKCGHCDPDNRDGEKFVCLSCQATLNADLDVAT | |
| | TNLVRVALTGKVMPRSERSGDAQTPGPARKARTGKIKGSKPTSAPQGATQTD | |
| | AKAHLSQTGV | |
| SEQ | MTPSPQIARLVETPLAAALKAHHPGKKERSDYLKKAGKILKDQGVEAAMAHL | CasΦ.22 |
| ID | DGKDQAEPPNEKPPAKCRIVARSREFSEWPIVKASVEIQKYIYGLTLEERKA | |
| NO: | CDPGKSSASHKAWFAKTGVNTFGYSSVQGENLIFGHTLGRYDGVLVKTENLN | |
| 176 | KKRAEKNERFRAKALAEGRAEPVCPPLVTATNDTGQDVTLEDGRVVRPGQLL | |
| | QPPGINPNIYAYQQVSPKAYVPGIIELPEEFQGYSRDPNAVILPLVPRDRLS | |
| | IPKGQPGYVPEPHREGLTGRKDRRMRRYYETERGTKLKRPPLTAKGRADKAN | |
| | EALLVVVRIDSDWVVMDVRGLLRNARWRRLVSKEGITLNGLLDLFTGDPVLN | |
| | PKDCSVSRDTGDPVNDPRHGVVTFCYKLGVVDVCSKDRPIKGERTKEVLERL | |
| | TSSGTVGMVSIDLGQTNPVAAAVSRVTKGLQAETLETFTLPDDLLGKVRAYR | |
| | AKTDRMEEGFRRNALRKLTAEQQAEITRYNDATEQQAKALVCSTYGIGPEEV | |
| | PWERMTSNTTYISDHILDHGGDPDTVFFMATKRGQNKPTLHKRKDKAWGQKF | |
| | RPAISVETRLARQAAEWELRRASLEFQKLSVWKTELCRQAVNYVMERTKKRT | |
| | QCDVIIPVIEDLPVPLFHGSGKRDPGWANFFVHKRENRWFIDGLHKAFSELG | |
| | KHRGIYVFEVCPQRTSITCPKCGHCDPDNRDGEKFVCLSCQATLHADLDVAT | |
| | TNLVRVALTGKVMPRSERSGDAQTPGPARKARTGKIKGSKPTSAPQGATQTD | |
| | AKAHLSQTGV | |
| SEQ | MKTEKPKTALTLLREEVFPGKKYRLDVLKEAGKKLSTKGREATIEFLTGKDE | CasΦ.23 |
| ID | ERPQNFQPPAKTSIVAQSRPFDQWPIVQVSLAVQKYIYGLTQSEFEANKKAL | |
| NO: | YGETGKAISTESRRAWFEATGVDNFGFTAAQGINPIFSQAVARYEGVIKKVE | |
| 177 | NRNEKKLKKLTKKNLLRLESGEEIEDFEPEATFNEEGRLLQPPGANPNIYCY | |
| | QQISPRIYDPSDPKGVILPQIYAGYDRKPEDIISAGVPNRLAIPEGQPGYIP | |
| | EHQRAGLKTQGRIRCRASVEAKARAAILAVVHLGEDWVVLDLRGLLRNVYWR | |
| | KLASPGTLTLKGLLDFFTGGPVLDARRGIATFSYTLKSAAAVHAENTYKGKG | |
| | TREVLLKLTENNSVALVTVDLGQRNPLAAMIARVSRTSQGDLTYPESVEPLT | |
| | RLFLPDPFLEEVRKYRSSYDALRLSIREAAIASLTPEQQAEIRYIEKFSAGD | |
| | AKKNVAEVFGIDPTQLPWDAMTPRTTYISDLFLRMGGDRSRVFFEVPPKKAK | |
| | KAPKKPPKKPAGPRIVKRTDGMIARLREIRPRLSAETNKAFQEARWEGERSN | |
| | VAFQKLSVRRKQFARTVVNHLVQTAQKMSRCDTVVLGIEDLNVPFFHGRGKY | |
| | QPGWEGFFRQKKENRWLINDMHKALSERGPHRGGYVLELTPFWTSLRCPKCG | |
| | HTDSANRDGDDFVCVKCGAKLHSDLEVATANLALVAITGQSIPRPPREQSSG | |
| | KKSTGTARMKKTSGETQGKGSKACVSEALNKIEQGTARDPVYNPLNSQVSCP | |
| | AP | |
| SEQ | VYNPDMKKPNNIRRIREEHFEGLCFGKDVLTKAGKIYEKDGEEAAIDELMGK | CasΦ.24 |
| ID | DEEDPPNFKPPAKTTIVAQSRPFDQWPIYQVSQAVQERVFAYTEEEFNASKE | |
| NO: | ALFSGDISSKSRDFWFKTNNISDQGIGAQGLNTILSHAFSRYSGVIKKVENR | |
| 178 | NKKRLKKLSKKNQLKIEEGLEILEFKPDSAFNENGLLAQPPGINPNIYGYQA | |
| | VTPFVEDPDNPGDVILPKQYEGYSRKPDDIIEKGPSRLDIPKGQPGYVPEHQ | |
| | RKNLKKKGRVRLYRRTPPKTKALASILAVLQIGKDWVLEDMRGLLRSVYMRE | |
| | AATPGQISAKDLLDTFTGCPVLNTRTGEFTFCYKLRSEGALHARKIYTKGET | |
| | RTLLTSLTSENNTIALVTVDLGQRNPAAIMISRLSRKEELSEKDIQPVSRRL | |
| | LPDRYLNELKRYRDAYDAFRQEVRDEAFTSLCPEHQEQVQQYEALTPEKAKN | |
| | LVLKHFFGTHDPDLPWDDMTSNTHYIANLYLERGGDPSKVFFTRPLKKDSKS | |
| | KKPRKPTKRTDASISRLPEIRPKMPEDARKAFEKAKWEIYTGHEKFPKLAKR | |
| | VNQLCREIANWIEKEAKRLTLCDTVVVGIEDLSLPPKRGKGKFQETWQGFER | |
| | QKFENRWVIDTLKKAIQNRAHDKGKYVLGLAPYWTSQRCPACGFIHKSNRNG | |
| | DHFKCLKCEALFHADSEVATWNLALVAVLGKGITNPDSKKPSGQKKTGTTRK | |
| | KQIKGKNKGKETVNVPPTTQEVEDIIAFFEKDDETVRNPVYKPTGT | |
| SEQ | MKKPNNIRRIREEHFEGLCFGKDVLTKAGKIYEKDGEEAAIDFLMGKDEEDP | CasΦ.25 |
| ID | PNFKPPAKTTIVAQSRPFDQWPIYQVSQAVQERVFAYTEEEFNASKEALESG | |
| NO: | DISSKSRDFWFKTNNISDQGIGAQGLNTILSHAFSRYSGVIKKVENRNKKRL | |
| 179 | KKLSKKNQLKIEEGLEILEFKPDSAFNENGLLAQPPGINPNIYGYQAVTPFV | |
| | FDPDNPGDVILPKQYEGYSRKPDDIIEKGPSRLDIPKGQPGYVPEHQRKNLK | |
| | KKGRVRLYRRTPPKTKALASILAVLQIGKDWVLEDMRGLLRSVYMREAATPG | |
| | QISAKDLLDTFTGCPVLNTRTGEFTFCYKLRSEGALHARKIYTKGETRTLLT | |
| | SLTSENNTIALVTVDLGQRNPAAIMISRLSRKEELSEKDIQPVSRRLLPDRY | |
| | LNELKRYRDAYDAFRQEVRDEAFTSLCPEHQEQVQQYEALTPEKAKNLVLKH | |
| | FFGTHDPDLPWDDMTSNTHYIANLYLERGGDPSKVFFTRPLKKDSKSKKPRK | |
| | PTKRTDASISRLPEIRPKMPEDARKAFEKAKWEIYTGHEKFPKLAKRVNQLC | |
| | REIANWIEKEAKRLTLCDTVVVGIEDLSLPPKRGKGKFQETWQGFFRQKFEN | |
| | RWVIDTLKKAIQNRAHDKGKYVLGLAPYWTSQRCPACGFIHKSNRNGDHFKC | |
| | LKCEALFHADSEVATWNLALVAVLGKGITNPDSKKPSGQKKTGTTRKKQIKG | |
| | KNKGKETVNVPPTTQEVEDIIAFFEKDDETVRNPVYKPTGT | |
| SEQ | VIKTHFPAGRFRKDHQKTAGKKLKHEGEEACVEYLRNKVSDYPPNEKPPAKG | CasΦ.26 |
| ID | TIVAQSRPFSEWPIVRASEAIQKYVYGLTVAELDVFSPGTSKPSHAEWFAKT | |
| NO: | GVENYGYRQVQGLNTIFQNTVNRFKGVLKKVENRNKKSLKRQEGANRRRVEE | |
| 180 | GLPEVPVTVESATDDEGRLLQPPGVNPSIYGYQGVAPRVCTDLQGFSGMSVD | |
| | FAGYRRDPDAVLVESLPEGRLSIPKGERGYVPEWQRDPERNKFPLREGSRRQ | |
| | RKWYSNACHKPKPGRTSKYDPEALKKASAKDALLVSISIGEDWAIIDVRGLL | |
| | RDARRRGFTPEEGLSLNSLLGLFTEYPVEDVQRGLITFTYKLGQVDVHSRKT | |
| | VPTFRSRALLESLVAKEEIALVSVDLGQTNPASMKVSRVRAQEGALVAEPVH | |
| | RMFLSDVLLGELSSYRKRMDAFEDAIRAQAFETMTPEQQAEITRVCDVSVEV | |
| | ARRRVCEKYSISPQDVPWGEMTGHSTFIVDAVLRKGGDESLVYFKNKEGETL | |
| | KFRDLRISRMEGVRPRLTKDTRDALNKAVLDLKRAHPTFAKLAKQKLELARR | |
| | CVNFIEREAKRYTQCERVVFVIEDLNVGFFHGKGKRDRGWDAFFTAKKENRW | |
| | VIQALHKAFSDLGLHRGSYVIEVTPQRTSMTCPRCGHCDKGNRNGEKFVCLQ | |
| | CGATLHADLEVATDNIERVALTGKAMPKPPVRERSGDVQKAGTARKARKPLK | |
| | PKQKTEPSVQEGSSDDGVDKSPGDASRNPVYNPSDTLSI | |
| SEQ | MAKAKTLAALLRELLPGQHLAPHHRWVANKLLMTSGDAAAFVIGKSVSDPVR | CasΦ.27 |
| ID | GSFRKDVITKAGRIFKKDGPDAAAAFLDGKWEDRPPNFQPPAKAAIVAISRS | |
| NO: | FDEWPIVKVSCAIQQYLYALPVQEFESSVPEARAQAHAAWFQDTGVDDCNEK | |
| 181 | STQGLNAIFNHGKRTYEGVLKKAQNRNDKKNLRLERINAKRAEAGQAPLVAG | |
| | PDESPTDDAGCLLHPPGINANIYCYQQVSPRPYEQSCGIQLPPEYAGYNRLS | |
| | NVAIPPMPNRLDIPQGQPGYVPEHHRHGIKKFGRVRKRYGVVPGRNRDADGK | |
| | RTRQVLTEAGAAAKARDSVLAVIRIGDDWTVVDLRGLLRNAQWRKLVPDGGI | |
| | TVQGLLDLFTGDPVIDPRRGVVTFIYKADSVGIHSEKVCRGKQSKNLLERLC | |
| | AMPEKSSTRLDCARQAVALVSVDLGQRNPVAARFSRVSLAEGQLQAQLVSAQ | |
| | FLDDAMVAMIRSYREEYDRFESLVREQAKAALSPEQLSEIVRHEADSAESVK | |
| | SCVCAKFGIDPAGLSWDKMTSGTWRIADHVQAAGGDVEWFFFKTCGKGKEIK | |
| | TVRRSDENVAKQFRLRLSPETRKDWNDAIWELKRGNPAYVSFSKRKSEFARR | |
| | VVNDLVHRARRAVRCDEVVFAIEDLNISFFHGKGQRQMGWDAFFEVKQENRW | |
| | FIQALHKAFVERATHKGGYVLEVAPARTSTTCPECRHCDPESRRGEQFCCIK | |
| | CRHTCHADLEVATENIEQVALTGVSLPKRLSSTLL | |
| SEQ | MSKEKTPPSAYAILKAKHFPDLDFEKKHKMMAGRMEKNGASEQEVVQYLQGK | CasΦ.28 |
| ID | GSESLMDVKPPAKSPILAQSRPEDEWEMVRTSRLIQETIFGIPKRGSIPKRD | |
| NO: | GLSETQFNELVASLEVGGKPMLNKQTRAIFYGLLGIKPPTFHAMAQNILIDL | |
| 182 | AINIRKGVLKKVDNLNEKNRKKVKRIRDAGEQDVMVPAEVTAHDDRGYLNHP | |
| | PGVNPTIPGYQGVVIPFPEGFEGLPSGMTPVDWSHVLVDYLPHDRLSIPKGS | |
| | PGYIPEWQRPLLNRHKGRRHRSWYANSLNKPRKSRTEEAKDRQNAGKRTALI | |
| | EAERLKGVLPVLMRFKEDWLIIDARGLLRNARYRGVLPEGSTLGNLIDLESD | |
| | SPRVDTRRGICTFLYRKGRAYSTKPVKRKESKETLLKLTEKSTIALVSIDLG | |
| | QTNPLTAKLSKVRQVDGCLVAEPVLRKLIDNASEDGKEIARYRVAHDLLRAR | |
| | ILEDAIDLLGIYKDEVVRARSDTPDLCKERVCRFLGLDSQAIDWDRMTPYTD | |
| | FIAQAFVAKGGDPKVVTIKPNGKPKMERKDRSIKNMKGIRLDISKEASSAYR | |
| | EAQWAIQRESPDFQRLAVWQSQLTKRIVNQLVAWAKKCTQCDTVVLAFEDLN | |
| | IGMMHGSGKWANGGWNALFLHKQENRWEMQAFHKALTELSAHKGIPTIEVLP | |
| | HRTSITCTQCGHCHPGNRDGEREKCLKCEFLANTDLEIATDNIERVALTGLP | |
| | MPKGERSSAKRKPGGTRKTKKSKHSGNSPLAAE | |
| SEQ | MEKAGPTSPLSVLIHKNFEGCRFQIDHLKIAGRKLAREGEAAAIEYLLDKKC | CasΦ.29 |
| ID | EGLPPNFQPPAKGNVIAQSRPFTEWAPYRASVAIQKYIYSLSVDERKVCDPG | |
| NO: | SSSDSHEKWFKQTGVQNYGYTHVQGLNLIFKHALARYDGVLKKVDNRNEKNR | |
| 183 | KKAERVNSFRREEGLPEEVFEEEKATDETGHLLQPPGVNHSIYCYQSVRPKP | |
| | FNPRKPGGISLPEAYSGYSLKPQDELPIGSLDRLSIPPGQPGYVPEWQRSQL | |
| | TTQKHRRKRSWYSAQKWKPRTGRTSTEDPDRLNCARAQGAILAVVRIHEDWV | |
| | VEDVRGLLRNALWRELAGKGLTVRDLLDFFTGDPVVDTKRGVVTFTYKLGKV | |
| | DVHSLRTVRGKRSKKVLEDLTLSSDVGLVTIDLGQTNVLAADYSKVTRSENG | |
| | ELLAVPLSKSFLPKHLLHEVTAYRTSYDQMEEGERRKALLTLTEDQQVEVTL | |
| | VRDFSVESSKTKLLQLGVDVTSLPWEKMSSNTTYISDQLLQQGADPASLFFD | |
| | GERDGKPCRHKKKDRTWAYLVRPKVSPETRKALNEALWALKNTSPEFESLSK | |
| | RKIQFSRRCMNYLLNEAKRISGCGQVVFVIEDLNVRVHHGRGKRAIGWDNFF | |
| | KPKRENRWEMQALHKAASELAIHRGMHIIEACPARSSITCPKCGHCDPENRC | |
| | SSDREKFLCVKCGAAFHADLEVATENLRKVALTGTALPKSIDHSRDGLIPKG | |
| | ARNRKLKEPQANDEKACA | |
| SEQ | MKEQSPLSSVLKSNFPGKKELSADIRVAGRKLAQLGEAAAVEYLSPRQRDSV | CasΦ.30 |
| ID | PNFRPPAFCTVVAKSRPFEEWPIYKASVLLQEQIYGMTGQEFEERCGSIPTS | |
| NO: | LSGLRQWASSVGLGAAMEGLHVQGMNLMVKNAINRYKGVLVKVENRNKKLVE | |
| 184 | ANEAKNSSREERGLPPLRPPELGSAFGPDGRLVNPPGIDKSIRLYQGVSPVP | |
| | VVKTTGRPTVHRLDIPAGEKGHVPLWQREAGLVKEGPRRRRMWYSNSNLKRS | |
| | RKDRSAEASEARKADSVVVRVSVKEDWVDIDVRGLLRNVAWRGIERAGESTE | |
| | DLLSLFSGDPVVDPSRDSVVELYKEGVVDVLSKKVVGAGKSRKQLEKMVSEG | |
| | PVALVSCDLGQTNYVAARVSVLDESLSPVRSFRVDPREFPSADGSQGVVGSL | |
| | DRIRADSDRLEAKLLSEAEASLPEPVRAEIEFLRSERPSAVAGRLCLKLGID | |
| | PRSIPWEKMGSTTSFISEALSAKGSPLALHDGAPIKDSRFAHAARGRLSPES | |
| | RKALNEALWERKSSSREYGVISRRKSEASRRMANAVLSESRRLTGLAVVAVN | |
| | LEDLNMVSKFFHGRGKRAPGWAGFFTPKMENRWFIRSIHKAMCDLSKHRGIT | |
| | VIESRPERTSISCPECGHCDPENRSGERFSCKSCGVSLHADFEVATRNLERV | |
| | ALTGKPMPRRENLHSPEGATASRKTRKKPREATASTELDLRSVLSSAENEGS | |
| | GPAARAG | |
| SEQ | MLPPSNKIGKSMSLKEFINKRNFKSSIIKQAGKILKKEGEEAVKKYLDDNYV | CasΦ.31 |
| ID | EGYKKRDFPITAKCNIVASNRKIEDFDISKFSSFIQNYVENLNKDNFEEFSK | |
| NO: | IKYNRKSFDELYKKIANEIGLEKPNYENIQGEIAVIRNAINIYNGVLKKVEN | |
| 185 | RNKKIQEKNQSKDPPKLLSAFDDNGFLAERPGINETIYGYQSVRLRHLDVEK | |
| | DKDIIVQLPDIYQKYNKKSTDKISVKKRLNKYNVDEYGKLISKRRKERINKD | |
| | DAILCVSNEGDDWIIFDARGLLRQTYRYKLKKKGLCIKDLLNLFTGDPIINP | |
| | TKTDLKEALSLSFKDGIINNRTLKVKNYKKCPELISELIRDKGKVAMISIDL | |
| | GQTNPISYRLSKFTANNVAYIENGVISEDDIVKMKKWREKSDKLENLIKEEA | |
| | IASLSDDEQREVRLYENDIADNTKKKILEKFNIREEDLDESKMSNNTYFIRD | |
| | CLKNKNIDESEFTFEKNGKKLDPTDACFAREYKNKLSELTRKKINEKIWEIK | |
| | KNSKEYHKISIYKKETIRYIVNKLIKQSKEKSECDDIIVNIEKLQIGGNFFG | |
| | GRGKRDPGWNNFFLPKEENRWFINACHKAFSELAPHKGIIVIESDPAYTSQT | |
| | CPKCENCDKENRNGEKFKCKKCNYEANADIDVATENLEKIAKNGRRLIKNED | |
| | QLGERLPGAEMPGGARKRKPSKSLPKNGRGAGVGSEPELINQSPSQVIA | |
| SEQ | VPDKKETPLVALCKKSFPGLRFKKHDSRQAGRILKSKGEGAAVAFLEGKGGT | CasΦ.32 |
| ID | TQPNFKPPVKCNIVAMSRPLEEWPIYKASVVIQKYVYAQSYEEFKATDPGKS | |
| NO: | EAGLRAWLKATRVDTDGYFNVQGLNLIFQNARATYEGVLKKVENRNSKKVAK | |
| 186 | IEQRNEHRAERGLPLLTLDEPETALDETGHLRHRPGINCSVEGYQHMKLKPY | |
| | VPGSIPGVTGYSRDPSTPIAACGVDRLEIPEGQPGYVPPWDRENLSVKKHRR | |
| | KRASWARSRGGAIDDNMLLAVVRVADDWALLDLRGLLRNTQYRKLLDRSVPV | |
| | TIESLLNLVINDPTLSVVKKPGKPVRYTATLIYKQGVVPVVKAKVVKGSYVS | |
| | KMLDDTTETFSLVGVDLGVNNLIAANALRIRPGKCVERLQAFTLPEQTVEDE | |
| | FRFRKAYDKHQENLRLAAVRSLTAEQQAEVLALDTFGPEQAKMQVCGHLGLS | |
| | VDEVPWDKVNSRSSILSDLAKERGVDDTLYMFPFFKGKGKKRKTEIRKRWDV | |
| | NWAQHERPQLTSETRKALNEAKWEAERNSSKYHQLSIRKKELSRHCVNYVIR | |
| | TAEKRAQCGKVIVAVEDLHHSFRRGGKGSRKSGWGGFFAAKQEGRWLMDALF | |
| | GAFCDLAVHRGYRVIKVDPYNTSRTCPECGHCDKANRDRVNREAFICVCCGY | |
| | RGNADIDVAAYNIAMVAITGVSLRKAARASVASTPLESLAAE | |
| SEQ | MSKTKELNDYQEALARRLPGVRHQKSVRRAARLVYDRQGEDAMVAFLDGKEV | CasΦ.33 |
| ID | DEPYTLQPPAKCHILAVSRPIEEWPIARVTMAVQEHVYALPVHEVEKSRPET | |
| NO: | TEGSRSAWFKNSGVSNHGVTHAQTLNAILKNAYNVYNGVIKKVENRNAKKRD | |
| 187 | SLAAKNKSRERKGLPHFKADPPELATDEQGYLLQPPSPNSSVYLVQQHLRTP | |
| | QIDLPSGYTGPVVDPRSPIPSLIPIDRLAIPPGQPGYVPLHDREKLTSNKHR | |
| | RMKLPKSLRAQGALPVCFRVEDDWAVVDGRGLLRHAQYRRLAPKNVSIAELL | |
| | ELYTGDPVIDIKRNLMTFRFAEAVVEVTARKIVEKYHNKYLLKLTEPKGKPV | |
| | REIGLVSIDLNVQRLIALAIYRVHQTGESQLALSPCLHREILPAKGLGDEDK | |
| | YKSKENQLTEEILTAAVQTLTSAQQEEYQRYVEESSHEAKADLCLKYSITPH | |
| | ELAWDKMTSSTQYISRWLRDHGWNASDETQITKGRKKVERLWSDSRWAQELK | |
| | PKLSNETRRKLEDAKHDLQRANPEWQRLAKRKQEYSRHLANTVLSMAREYTA | |
| | CETVVIAIENLPMKGGFVDGNGSRESGWDNFFTHKKENRWMIKDIHKALSDL | |
| | APNRGVHVLEVNPQYTSQTCPECGHRDKANRDPIQRERFCCTHCGAQRHADL | |
| | EVATHNIAMVATTGKSLTGKSLAPQRLQEAAE | |
| SEQ | VLLSDRIQYTDPSAPIPAMTVVDRRKIKKGEPGYVPPFMRKNLSTNKHRRMR | CasΦ.41 |
| ID | LSRGQKEACALPVGLRLPDGKDGWDFIIFDGRALLRACRRLRLEVTSMDDVL | |
| NO: | DKFTGDPRIQLSPAGETIVTCMLKPQHTGVIQQKLITGKMKDRLVQLTAEAP | |
| 188 | IAMLTVDLGEHNLVACGAYTVGQRRGKLQSERLEAFLLPEKVLADFEGYRRD | |
| | SDEHSETLRHEALKALSKRQQREVLDMLRTGADQARESLCYKYGLDLQALPW | |
| | DKMSSNSTFIAQHLMSLGFGESATHVRYRPKRKASERTILKYDSRFAAEEKI | |
| | KLTDETRRAWNEAIWECQRASQEFRCLSVRKLQLARAAVNWTLTQAKQRSRC | |
| | PRVVVVVEDLNVRFMHGGGKRQEGWAGFFKARSEKRWFIQALHKAYTELPTN | |
| | RGIHVMEVNPARTSITCTKCGYCDPENRYGEDFHCRNPKCKVRGGHVANADL | |
| | DIATENLARVALSGPMPKAPKLK | |
| SEQ | MTPSFGYQMIIVTPIHHASGAWATLRLLFLNPKTSGVMLGMTKTKSAFALMR | CasΦ.34 |
| ID | EEVFPGLLFKSADLKMAGRKFAKEGREAAIEYLRGKDEERPANFKPPAKGDI | |
| NO: | IAQSRPFDQWPIVQVSQAIQKYIFGLTKAEFDATKTLLYGEGNHPTTESRRR | |
| 189 | WFEATGVPDFGFTSAQGLNAIFSSALARYEGVIQKVENRNEKRLKKLSEKNQ | |
| | RLVEEGHAVEAYVPETAFHTLESLKALSEKSLVPLDDLMDKIDRLAQPPGIN | |
| | PCLYGYQQVAPYIYDPENPRGVVLPDLYLGYCRKPDDPITACPNRLDIPKGQ | |
| | PGYIPEHQRGQLKKHGRVRRFRYTNPQAKARAKAQTAILAVLRIDEDWVVMD | |
| | LRGLLRNVYFREVAAPGELTARTLLDTFTGCPVLNLRSNVVTFCYDIESKGA | |
| | LHAEYVRKGWATRNKLLDLTKDGQSVALLSVDLGQRHPVAVMISRLKRDDKG | |
| | DLSEKSIQVVSRTFADQYVDKLKRYRVQYDALRKEIYDAALVSLPPEQQAEI | |
| | RAYEAFAPGDAKANVLSVMFQGEVSPDELPWDKMNINTHYISDLYLRRGGDP | |
| | SRVFFVPQPSTPKKNAKKPPAPRKPVKRTDENVSHMPEFRPHLSNETREAFQ | |
| | KAKWTMERGNVRYAQLSRFLNQIVREANNWLVSEAKKLTQCQTVVWAIEDLH | |
| | VPFFHGKGKYHETWDGFFRQKKEDRWFVNVFHKAISERAPNKGEYVMEVAPY | |
| | RTSQRCPVCGFVDADNRHGDHFKCLRCGVELHADLEVATWNIALVAVQGHGI | |
| | AGPPREQSCGGETAGTARKGKNIKKNKGLADAVTVEAQDSEGGSKKDAGTAR | |
| | NPVYIPSESQVNCPAP | |
| SEQ | MKPKTPKPPKTPVAALIDKHFPGKRFRASYLKSVGKKLKNQGEDVAVRELTG | CasΦ.35 |
| ID | KDEERPPNFQPPAKSNIVAQSRPIEEWPIHKVSVAVQEYVYGLTVAEKEACS | |
| NO: | DAGESSSSHAAWFAKTGVENFGYTSVQGLNKIFPPTENREDGVIKKVENRNE | |
| 190 | KKRQKATRINEAKRNKGQSEDPPEAEVKATDDAGYLLQPPGINHSVYGYQSI | |
| | TLCPYTAEKFPTIKLPEEYAGYHSNPDAPIPAGVPDRLAIPEGQPGHVPEEH | |
| | RAGLSTKKHRRVRQWYAMANWKPKPKRTSKPDYDRLAKARAQGALLIVIRID | |
| | EDWVVVDARGLLRNVRWRSLGKREITPNELLDLFTGDPVLDLKRGVVTFTYA | |
| | EGVVNVCSRSTTKGKQTKVLLDAMTAPRDGKKRQIGMVAVDLGQTNPIAAEY | |
| | SRVGKNAAGTLEATPLSRSTLPDELLREIALYRKAHDRLEAQLREEAVLKLT | |
| | AEQQAENARYVETSEEGAKLALANLGVDTSTLPWDAMTGWSTCISDHLINHG | |
| | GDTSAVFFQTIRKGTKKLETIKRKDSSWADIVRPRLTKETREALNDELWELK | |
| | RSHEGYEKLSKRLEELARRAVNHVVQEVKWLTQCQDIVIVIEDLNVRNFHGG | |
| | GKRGGGWSNFFTVKKENRWEMQALHKAFSDLAAHRGIPVLEVYPARTSITCL | |
| | GCGHCDPENRDGEAFVCQQCGATFHADLEVATRNIARVALTGEAMPKAPARE | |
| | QPGGAKKRGTSRRRKLTEVAVKSAEPTIHQAKNQQLNGTSRDPVYKGSELPA | |
| | L | |
| SEQ | MSEITDLLKANFKGKTFKSADMRMAGRILKKSGAQAVIKYLSDKGAVDPPDE | CasΦ.43 |
| ID | RPPAKCNIIAQSRPEDEWPICKASMAIQQHIYGLTKNEFDESSPGTSSASHE | |
| NO: | QWFAKTGVDTHGFTHVQGLNLIFQHAKKRYEGVIKKVENYNEKERKKFEGIN | |
| 191 | ERRSKEGMPLLEPRLRTAFGDDGKFAEKPGVNPSIYLYQQTSPRPYDKTKHP | |
| | YVHAPFELKEITTIPTQDDRLKIPFGAPGHVPEKHRSQLSMAKHKRRRAWYA | |
| | LSQNKPRPPKDGSKGRRSVRDLADLKAASLADAIPLVSRVGEDWVVIDGRGL | |
| | LRNLRWRKLAHEGMTVEEMLGFFSGDPVIDPRRNVATFIYKAEHATVKSRKP | |
| | IGGAKRAREELLKATASSDGVIRQVGLISVDLGQTNPVAYEISRMHQANGEL | |
| | VAEHLEYGLLNDEQVNSIQRYRAAWDSMNESFRQKAIESLSMEAQDEIMQAS | |
| | TGAAKRTREAVLTMFGPNATLPWSRMSSNTTCISDALIEVGKEEETNFVTSN | |
| | GPRKRTDAQWAAYLRPRVNPETRALLNQAVWDLMKRSDEYERLSKRKLEMAR | |
| | QCVNFVVARAEKLTQCNNIGIVLENLVVRNFHGSGRRESGWEGFFEPKRENR | |
| | WFMQVLHKAFSDLAQHRGVMVFEVHPAYSSQTCPACRYVDPKNRSSEDRERF | |
| | KCLKCGRSFNADREVATFNIREIARTGVGLPKPDCERSRGVQTTGTARNPGR | |
| | SLKSNKNPSEPKRVLQSKTRKKITSTETQNEPLATDLKT | |
| SEQ | MTPKTESPLSALCKKHFPGKRFRTNYLKDAGKILKKHGEDAVVAFLSDKQED | CasΦ.44 |
| ID | EPANFCPPAKVHILAQSRPFEDWPINLASKAIQTYVYGLTADERKTCEPGTS | |
| NO: | KESHDRWFKETGVDHHGFTSVQGLNLIFKHTLNRYDGVIKKVETRNEKRRSS | |
| 192 | VVRINEKKAAEGLPLIAAEAEETAFGEDGRLLQPPGVNHSIYCFQQVSPQPY | |
| | SSKKHPQVVLPHAVQGVDPDAPIPVGRPNRLDIPKGQPGYVPEWQRPHLSMK | |
| | CKRVRMWYARANWRRKPGRRSVLNEARLKEASAKGALPIVLVIGDDWLVMDA | |
| | RGLLRSVFWRRVAKPGLSLSELLNVTPTGLESGDPVIDPKRGLVTFTSKLGV | |
| | VAVHSRKPTRGKKSKDLLLKMTKPTDDGMPRHVGMVAIDLGQTNPVAAEYSR | |
| | VVQSDAGTLKQEPVSRGVLPDDLLKDVARYRRAYDLTEESIRQEAIALLSEG | |
| | HRAEVTKLDQTTANETKRLLVDRGVSESLPWEKMSSNTTYISDCLVALGKTD | |
| | DVFFVPKAKKGKKETGIAVKRKDHGWSKLLRPRTSPEARKALNENQWAVKRA | |
| | SPEYERLSRRKLELGRRCVNHIIQETKRWTQCEDIVVVLEDLNVGFFHGSGK | |
| | RPDGWDNFFVSKRENRWFIQVLHKAFGDLATHRGTHVIEVHPARTSITCIKC | |
| | GHCDAGNRDGESFVCLASACGDRRHADLEVATRNVARVAITGERMPPSEQAR | |
| | DVQKAGGARKRKPSARNVKSSYPAVEPAPASP | |
| SEQ | MSDNKMKKLSKEEKPLTPLQILIRKYIDKSQYPSGFKTTIIKQAGVRIKSVK | CasΦ.36 |
| ID | SEQDEINLANWIISKYDPTYIKRDENPSAKCQIIATSRSVADEDIVKMSNKV | |
| NO: | QEIFFASSHLDKNVEDIGKSKSDHDSWFERNNVDRGIYTYSNVQGMNLIFSN | |
| 193 | TKNTYLGVAVKAQNKFSSKMKRIQDINNFRITNHQSPLPIPDEIKIYDDAGE | |
| | LLNPPGVNPNIFGYQSCLLKPLENKEIISKTSFPEYSRLPADMIEVNYKISN | |
| | RLKFSNDQKGFIQFKDKLNLFKINSQELFSKRRRLSGQPILLVASFGDDWVV | |
| | LDGRGLLRQVYYRGIAKPGSITISELLGFFTGDPIVDPIRGVVSLGFKPGVL | |
| | SQETLKTTSARIFAEKLPNLVLNNNVGLMSIDLGQTNPVSYRLSEITSNMSV | |
| | EHICSDELSQDQISSIEKAKTSLDNLEEEIAIKAVDHLSDEDKINFANESKL | |
| | NLPEDTRQSLFEKYPELIGSKLDFGSMGSGTSYIADELIKFENKDAFYPSGK | |
| | KKFDLSFSRDLRKKLSDETRKSYNDALFLEKRTNDKYLKNAKRRKQIVRTVA | |
| | NSLVSKIEELGLTPVINIENLAMSGGFFDGRGKREKGWDNFFKVKKENRWVM | |
| | KDFHKAFSELSPHHGVIVIESPPYCTSVTCTKCNFCDKKNRNGHKFTCQRCG | |
| | LDANADLDIATENLEKVAISGKRMPGSERSSDERKVAVARKAKSPKGKAIKG | |
| | VKCTITDEPALLSANSQDCSQSTS | |
| SEQ | MALSLAEVRERHFKGLRFRSSYLKRAGKILKKEGEAACVAYLTGKDEESPPN | CasΦ.37 |
| ID | FKPPAKCDVVAQSRPFEEWPIVQASVAVQSYVYGLTKEAFEAFNPGTTKQSH | |
| NO: | EACLAATGIDTCGYSNVQGLNLIFRQAKNRYEGVITKVENRNKKAKKKLTRK | |
| 194 | NEWRQKNGHSELPEAPEELTENDEGRLLQPPGINPSLYTYQQISPTPWSPKD | |
| | SSILPPQYAGYERDPNAPIPFGVAKDRLTIASGCPGYIPEWMRTAGEKTNPR | |
| | TQKKFMHPGLSTRKNKRMRLPRSVRSAPLGALLVTIHLGEDWLVLDVRGLLR | |
| | NARWRGVAPKDISTQGLLNLFTGDPVIDTRRGVVTFTYKPETVGIHSRTWLY | |
| | KGKQTKEVLEKLTQDQTVALVAIDLGQTNPVSAAASRVSRSGENLSIETVDR | |
| | FFLPDELIKELRLYRMAHDRLEERIREESTLALTEAQQAEVRALEHVVRDDA | |
| | KNKVCAAFNLDAASLPWDQMTSNTTYLSEAILAQGVSRDQVFFTPNPKKGSK | |
| | EPVEVMRKDRAWVYAFKAKLSEETRKAKNEALWALKRASPDYARLSKRREEL | |
| | CRRSVNMVINRAKKRTQCQVVIPVLEDLNIGFFHGSGKRLPGWDNFFVAKKE | |
| | NRWLMNGLHKSFSDLAVHRGFYVFEVMPHRTSITCPACGHCDSENRDGEAFV | |
| | CLSCKRTYHADLDVATHNLTQVAGTGLPMPEREHPGGTKKPGGSRKPESPQT | |
| | HAPILHRTDYSESADRLGS | |
| SEQ | QAVIKYLSDKGAVDPPDERPPAKCNIIAQSRPEDEWPICKASMAIQQHIYGL | Cas.45 |
| ID | TKNEFDESSPGTSSASHEQWFAKTGVDTHGFTHVQGLNLIFQHAKKRYEGVI | |
| NO: | KKVENYNEKERKKFEGINERRSKEGMPLLEPRLRTAFGDDGKFAEKPGVNPS | |
| 195 | IYLYQQTSPRPYDKTKHPYVHAPFELKEITTIPTQDDRLKIPFGAPGHVPEK | |
| | HRSQLSMAKHKRRRAWYALSQNKPRPPKDGSKGRRSVRDLADLKAASLADAI | |
| | PLVSRVGFDWVVIDGRGLLRNLRWRKLAHEGMTVEEMLGFFSGDPVIDPRRN | |
| | VATFIYKAEHATVKSRKPIGGAKRAREELLKATASSDGVIRQVGLISVDLGQ | |
| | TNPVAYEISRMHQANGELVAEHLEYGLINDEQVNSIQRYRAAWDSMNESFRQ | |
| | KAIESLSMEAQDEIMQASTGAAKRTREAVLTMFGPNATLPWSRMSSNTTCIS | |
| | DALIEVGKEEETNFVTSNGPRKRTDAQWAAYLRPRVNPETRALLNQAVWDLM | |
| | KRSDEYERLSKRKLEMARQCVNFVVARAEKLTQCNNIGIVLENLVVRNFHGS | |
| | GRRESGWEGFFEPKRENRWFMQVLHKAFSDLAQHRGVMVFEVHPAYSSQTCP | |
| | ACRYVDPKNRSSEDRERFKCLKCGRSENADREVATFNIREIARTGVGLPKPD | |
| | CERSRDVQTPGTARKSGRSLKSQDNLSEPKRVLQSKTRKKITSTETQNEPLA | |
| | TDLKT | |
| SEQ | MIKEQSELSKLIEKYYPGKKFYSNDLKQAGKHLKKSEHLTAKESEELTVEFL | Cas@.38 |
| ID | KSCKEKLYDFRPPAKALIISTSRPFEEWPIYKASESIQKYIYSLTKEELEKY | |
| NO: | NISTDKTSQENFFKESLIDNYGFANVSGLNLIFQHTKAIYDGVLKKVNNRNN | |
| 196 | KILKKYKRKIEEGIEIDSPELEKAIDESGHFINPPGINKNIYCYQQVSPTIF | |
| | NSFKETKIICPFNYKRNPNDIIQKGVIDRLAIPFGEPGYIPDHQRDKVNKHK | |
| | KRIRKYYKNNENKNKDAILAKINIGEDWVLEDLRGLLRNAYWRKLIPKQGIT | |
| | PQQLLDMESGDPVIDPIKNNITFIYKESIIPIHSESIIKTKKSKELLEKLTK | |
| | DEQIALVSIDLGQTNPVAARFSRLSSDLKPEHVSSSFLPDELKNEICRYREK | |
| | SDLLEIEIKNKAIKMLSQEQQDEIKLVNDISSEELKNSVCKKYNIDNSKIPW | |
| | DKMNGFTTFIADEFINNGGDKSLVYFTAKDKKSKKEKLVKLSDKKIANSFKP | |
| | KISKETREILNKITWDEKISSNEYKKLSKRKLEFARRATNYLINQAKKATRL | |
| | NNVVLVVEDLNSKFFHGSGKREDGWDNFFIPKKENRWFIQALHKSLTDVSIH | |
| | RGINVIEVRPERTSITCPKCGCCDKENRKGEDFKCIKCDSVYHADLEVATEN | |
| | IEKVAITGESMPKPDCERLGGEESIG | |
| SEQ | VAFLDGKEVDEPYTLQPPAKCHILAVSRPIEEWPIARVTMAVQEHVYALPVH | Caso.39 |
| ID | EVEKSRPETTEGSRSAWFKNSGVSNHGVTHAQTLNAILKNAYNVYNGVIKKV | |
| NO: | ENRNAKKRDSLAAKNKSRERKGLPHFKADPPELATDEQGYLLQPPSPNSSVY | |
| 197 | LVQQHLRTPQIDLPSGYTGPVVDPRSPIPSLIPIDRLAIPPGQPGYVPLHDR | |
| | EKLTSNKHRRMKLPKSLRAQGALPVCFRVEDDWAVVDGRGLLRHAQYRRLAP | |
| | KNVSIAELLELYTGDPVIDIKRNLMTFRFAEAVVEVTARKIVEKYHNKYLLK | |
| | LTEPKGKPVREIGLVSIDLNVQRLIALAIYRVHQTGESQLALSPCLHREILP | |
| | AKGLGDFDKYKSKFNQLTEEILTAAVQTLTSAQQEEYQRYVEESSHEAKADL | |
| | CLKYSITPHELAWDKMTSSTQYISRWLRDHGWNASDFTQITKGRKKVERLWS | |
| | DSRWAQELKPKLSNETRRKLEDAKHDLQRANPEWQRLAKRKQEYSRHLANTV | |
| | LSMAREYTACETVVIAIENLPMKGGFVDGNGSRESGWDNFFTHKKENRWMIK | |
| | DIHKALSDLAPNRGVHVLEVNPQYTSQTCPECGHRDKANRDPIQRERFCCTH | |
| | CGAQRHADLEVATHNIAMVATTGKSLTGKSLAPQRLQ | |
| SEQ | LEIPEGEPGHVPWFQRMDIPEGQIGHVNKIQRFNFVHGKNSGKVKFSDKTGR | CasΦ.42 |
| ID | VKRYHHSKYKDATKPYKFLEESKKVSALDSILAIITIGDDWVVFDIRGLYRN | |
| NO: | VFYRELAQKGLTAVQLLDLFTGDPVIDPKKGIITFSYKEGVVPVESQKIVSR | |
| 198 | FKSRDTLEKLTSQGPVALLSVDLGQNEPVAARVCSLKNINDKIALDNSCRIP | |
| | FLDDYKKQIKDYRDSLDELEIKIRLEAINSLDVNQQVEIRDLDVESADRAKA | |
| | STVDMEDIDPNLISWDSMSDARFSTQISDLYLKNGGDESRVYFEINNKRIKR | |
| | SDYNISQLVRPKLSDSTRKNLNDSIWKLKRTSEEYLKLSKRKLELSRAVVNY | |
| | TIRQSKLLSGINDIVIILEDLDVKKKENGRGIRDIGWDNFFSSRKENRWFIP | |
| | AFHKSFSELSSNRGLCVIEVNPAWTSATCPDCGFCSKENRDGINFTCRKCGV | |
| | SYHADIDVATLNIARVAVLGKPMSGPADRERLGGTKKPRVARSRKDMKRKDI | |
| | SNGTVEVMVTA | |
| SEQ | IPSFGYLDRLKIAKGQPGYIPEWQRETINPSKKVRRYWATNHEKIRNAIPLV | CasΦ.46 |
| ID | VFIGDDWVIIDGRGLLRDARRRKLADKNTTIEQLLEMVSNDPVIDSTRGIAT | |
| NO: | LSYVEGVVPVRSFIPIGEKKGREYLEKSTQKESVTLLSVDIGQINPVSCGVY | |
| 199 | KVSNGCSKIDFLDKFELDKKHLDAIQKYRTLQDSLEASIVNEALDEIDPSEK | |
| | KEYQNINSQTSNDVKKSLCTEYNIDPEAISWQDITAHSTLISDYLIDNNITN | |
| | DVYRTVNKAKYKTNDFGWYKKESAKLSKEAREALNEKIWELKIASSKYKKLS | |
| | VRKKEIARTIANDCVKRAETYGDNVVVAMESLTKNNKVMSGRGKRDPGWHNL | |
| | GQAKVENRWFIQAISSAFEDKATHHGTPVLKVNPAYTSQTCPSCGHCSKDNR | |
| | SSKDRTIFVCKSCGEKFNADLDVATYNIAHVAFSGKKLSPPSEKSSATKKPR | |
| | SARKSKKSRKS | |
| SEQ | SPIEKLLNGLLVKITFGNDWIICDARGLLDNVQKGIIHKSYFTNKSSLVDLI | CasΦ.47 |
| ID | DLFTCNPIVNYKNNVVTFCYKEGVVDVKSFTPIKSGPKTQENLIKKLKYSRF | |
| NO: | QNEKDACVLGVGVDVGVTNPFAINGFKMPVDESSEWVMLNEPLFTIETSQAF | |
| 200 | REEIMAYQQRTDEMNDQFNQQSIDLLPPEYKVEFDNLPEDINEVAKYNLLHT | |
| | LNIPNNFLWDKMSNTTQFISDYLIQIGRGTETEKTITTKKGKEKILTIRDVN | |
| | WENTFKPKISEETGKARTEIKRDLQKNSDQFQKLAKSREQSCRTWVNNVTEE | |
| | AKIKSGCPLIIFVIEALVKDNRVESGKGHRAIGWHNFGKQKNERRWWVQAIH | |
| | KAFQEQGVNHGYPVILCPPQYTSQTCPKCNHVDRDNRSGEKFKCLKYGWIGN | |
| | ADLDVGAYNIARVAITGKALSKPLEQKKIKKAKNKT | |
| SEQ | LLDNVQKGIIHKSYFTNKSSLVDLIDLFTCNPIVNYKNNVVTFCYKEGVVDV | CasΦ.48 |
| ID | KSFTPIKSGPKTQENLIKKLKYSRFQNEKDACVLGVGVDVGVTNPFAINGEK | |
| NO: | MPVDESSEWVMLNEPLFTIETSQAFREEIMAYQQRTDEMNDQFNQQSIDLLP | |
| 201 | PEYKVEFDNLPEDINEVAKYNLLHTLNIPNNFLWDKMSNTTQFISDYLIQIG | |
| | RGTETEKTITTKKGKEKILTIRDVNWENTFKPKISEETGKARTEIKRDLQKN | |
| | SDQFQKLAKSREQSCRTWVNNVTEEAKIKSGCPLIIFVIEALVKDNRVESGK | |
| | GHRAIGWHNFGKQKNERRWWVQAIHKAFQEQGVNHGYPVILCPPQYTSQTCP | |
| | KCNHVDRDNRSGEKFKCLKYGWIGNADLDVGAYNIARVAITGKALSKPLEQK | |
| | KIKKAKNKT | |
| SEQ | MIKPTVSQFLTPGFKLIRNHSRTAGLKLKNEGEEACKKFVRENEIPKDECPN | CasΦ.49 |
| ID | FQGGPAIANIIAKSREFTEWEIYQSSLAIQEVIFTLPKDKLPEPILKEEWRA | |
| NO: | QWLSEHGLDTVPYKEAAGLNLIIKNAVNTYKGVQVKVDNKNKNNLAKINRKN | |
| 202 | EIAKLNGEQEISFEEIKAFDDKGYLLQKPSPNKSIYCYQSVSPKPFITSKYH | |
| | NVNLPEEYIGYYRKSNEPIVSPYQFDRLRIPIGEPGYVPKWQYTELSKKENK | |
| | RRKLSKRIKNVSPILGIICIKKDWCVEDMRGLLRTNHWKKYHKPTDSINDLF | |
| | DYFTGDPVIDTKANVVRFRYKMENGIVNYKPVREKKGKELLENICDQNGSCK | |
| | LATVDVGQNNPVAIGLFELKKVNGELTKTLISRHPTPIDFCNKITAYRERYD | |
| | KLESSIKLDAIKQLTSEQKIEVDNYNNNFTPQNTKQIVCSKLNINPNDLPWD | |
| | KMISGTHFISEKAQVSNKSEIYFTSTDKGKTKDVMKSDYKWFQDYKPKLSKE | |
| | VRDALSDIEWRLRRESLEENKLSKSREQDARQLANWISSMCDVIGIENLVKK | |
| | NNFFGGSGKREPGWDNFYKPKKENRWWINAIHKALTELSQNKGKRVILLPAM | |
| | RTSITCPKCKYCDSKNRNGEKFNCLKCGIELNADIDVATENLATVAITAQSM | |
| | PKPTCERSGDAKKPVRARKAKAPEFHDKLAPSYTVVLREAVKRPAATKKAGQ | |
| | AKKKKEF | |
| | (Bold sequence is Nuclear Localization Signal) | |

### 4. Cas 13 Proteins

In some examples, the CRISPR/Cas effector protein is a Cas13 protein. The general architecture of a Cas13 protein includes an N-terminal domain and two HEPN (higher eukaryotes and prokaryotes nucleotide-binding) domains separated by two helical domains (Liu et al., Cell 2017 Jan. 12; 168(1-2):121-134.e12). The HEPN domains each comprise aR-X4-H motif. Shared features across Cas13 proteins include that upon binding of the crRNA of the guide nucleic acid to a target nucleic acid, the protein undergoes a conformational change to bring together the HEPN domains and form a catalytically active RNase. (Tambe et al., Cell Rep. 2018 Jul. 24; 24(4): 1025-1036.). Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains.

In some examples, the Cas13 is at least one of LbuCas13a, LwaCas13a, LbaCas13a, HheCas13a, PprCas13a, EreCas13a, CamCas13a, or LshCas13a. In some examples, the trans cleavage activity of the CRISPR enzyme can be activated when the crRNA is complexed with the target nucleic acid. In some instances, the trans cleavage activity of the CRISPR enzyme is activated when the guide nucleic acid comprising a tracrRNA and crRNA are complexed with the target nucleic acid. In some examples, the target nucleic acid is RNA or DNA.

In some examples, a Cas13 nuclease of the disclosure can exhibit indiscriminate trans-cleavage of ssRNA, enabling its use for inducing cell death, apoptosis, cell cycle arrest, or a combination thereof, in a population cells. In some examples, a Cas13 nuclease of the disclosure can, upon hybridization of a guide nucleic acid molecule to a target DNA or RNA, induce cis-cleavage of the target DNA or RNA.

In some examples, the Cas13 protein comprises a Cas13a polypeptide, a Cas13b polypeptide, a Cas13c polypeptide, a Cas13c polypeptide, a Cas13d polypeptide, or a Cas13e polypeptide. Sometimes Cas13a can also be also called C2c2. In some examples, the Cas13 protein has at least 80%, at least 85%, at least 90%, at least 92%, at least 95%, at least 97%, or at least 99% sequence identity to any one of SEQ ID NOs: 203-220, and 248-262. In some examples, the Cas13 protein is selected from SEQ ID NOs: 203-220, and 248-262.

TABLE 4 provides amino acid sequences of illustrative Cas13 polypeptides that can be used in compositions and methods of the disclosure.

TABLE 4
Cas13 Protein Sequences

| # | Sequence | Annotation |
|---|---|---|
| SEQ | MWISIKTLIHHLGVLFFCDYMYNRREKKIIEVKTMRITKVEVDRKKVLISRDK | Listeria |
| ID | NGGKLVYENEMQDNTEQIMHHKKSSFYKSVVNKTICRPEQKQMKKLVHGLLQE | seeligeri |
| NO: | NSQEKIKVSDVTKLNISNELNHREKKSLYYFPENSPDKSEEYRIEINLSQLLE | C2c2 |
| 203 | DSLKKQQGTFICWESFSKDMELYINWAENYISSKTKLIKKSIRNNRIQSTESR | amino |
| | SGQLMDRYMKDILNKNKPFDIQSVSEKYQLEKLTSALKATFKEAKKNDKEINY | acid |
| | KLKSTLQNHERQIIEELKENSELNQFNIEIRKHLETYFPIKKTNRKVGDIRNL | sequence |
| | EIGEIQKIVNHRLKNKIVQRILQEGKLASYEIESTVNSNSLQKIKIEEAFALK | |
| | FINACLFASNNLRNMVYPVCKKDILMIGEFKNSFKEIKHKKFIRQWSQFFSQE | |
| | ITVDDIELASWGLRGAIAPIRNEIIHLKKHSWKKFFNNPTFKVKKSKIINGKT | |
| | KDVTSEFLYKETLFKDYFYSELDSVPELIINKMESSKILDYYSSDQLNQVETI | |
| | PNFELSLLTSAVPFAPSFKRVYLKGFDYQNQDEAQPDYNLKLNIYNEKAENSE | |
| | AFQAQYSLFKMVYYQVFLPQFTTNNDLFKSSVDFILTLNKERKGYAKAFQDIR | |
| | KMNKDEKPSEYMSYIQSQLMLYQKKQEEKEKINHFEKFINQVFIKGENSFIEK | |
| | NRLTYICHPTKNTVPENDNIEIPFHTDMDDSNIAFWLMCKLLDAKQLSELRNE | |
| | MIKFSCSLQSTEEISTFTKAREVIGLALLNGEKGCNDWKELEDDKEAWKKNMS | |
| | LYVSEELLQSLPYTQEDGQTPVINRSIDLVKKYGTETILEKLESSSDDYKVSA | |
| | KDIAKLHEYDVTEKIAQQESLHKQWIEKPGLARDSAWTKKYQNVINDISNYQW | |
| | AKTKVELTQVRHLHQLTIDLLSRLAGYMSIADRDFQFSSNYILERENSEYRVT | |
| | SWILLSENKNKNKYNDYELYNLKNASIKVSSKNDPQLKVDLKQLRLTLEYLEL | |
| | FDNRLKEKRNNISHFNYLNGQLGNSILELFDDARDVLSYDRKLKNAVSKSLKE | |
| | ILSSHGMEVTFKPLYQTNHHLKIDKLQPKKIHHLGEKSTVSSNQVSNEYCQLV | |
| | RTLLTMK | |
| SEQ | MKVTKVGGISHKKYTSEGRLVKSESEENRTDERLSALLNMRLDMYIKNPSSTE | Leptotrichia |
| ID | TKENQKRIGKLKKFFSNKMVYLKDNTLSLKNGKKENIDREYSETDILESDVRD | buccalis |
| NO: | KKNFAVLKKIYLNENVNSEELEVFRNDIKKKLNKINSLKYSFEKNKANYQKIN | (Lbu) |
| 204 | ENNIEKVEGKSKRNIIYDYYRESAKRDAYVSNVKEAFDKLYKEEDIAKLVLEI | C2c2 |
| | ENLTKLEKYKIREFYHEIIGRKNDKENFAKIIYEEIQNVNNMKELIEKVPDMS | amino |
| | ELKKSQVFYKYYLDKEELNDKNIKYAFCHEVEIEMSQLLKNYVYKRLSNISND | acid |
| | KIKRIFEYQNLKKLIENKLLNKLDTYVRNCGKYNYYLODGEIATSDFIARNRQ | sequence |
| | NEAFLRNIIGVSSVAYFSLRNILETENENDITGRMRGKTVKNNKGEEKYVSGE | |
| | VDKIYNENKKNEVKENLKMFYSYDENMDNKNEIEDFFANIDEAISSIRHGIVH | |
| | FNLELEGKDIFAFKNIAPSEISKKMFQNEINEKKLKLKIFRQLNSANVERYLE | |
| | KYKILNYLKRTRFEFVNKNIPFVPSFTKLYSRIDDLKNSLGIYWKTPKINDDN | |
| | KTKEIIDAQIYLLKNIYYGEFLNYFMSNNGNFFEISKEIIELNKNDKRNLKTG | |
| | FYKLQKFEDIQEKIPKEYLANIQSLYMINAGNQDEEEKDTYIDFIQKIFLKGF | |
| | MTYLANNGRLSLIYIGSDEETNTSLAEKKQEFDKELKKYEQNNNIKIPYEINE | |
| | FLREIKLGNILKYTERLNMFYLILKLLNHKELTNLKGSLEKYQSANKEEAFSD | |
| | QLELINLLNLDNNRVTEDFELEADEIGKELDENGNKVKDNKELKKEDTNKIYF | |
| | DGENIIKHRAFYNIKKYGMLNLLEKIADKAGYKISIEELKKYSNKKNEIEKNH | |
| | KMQENLHRKYARPRKDEKFTDEDYESYKQAIENIEEYTHLKNKVEFNELNLLQ | |
| | GLLLRILHRLVGYTSIWERDLRFRLKGEFPENQYIEEIFNFENKKNVKYKGGQ | |
| | IVEKYIKFYKELHQNDEVKINKYSSANIKVLKQEKKDLYIRNYIAHFNYIPHA | |
| | EISLLEVLENLRKLLSYDRKLKNAVMKSVVDILKEYGFVATFKIGADKKIGIQ | |
| | TLESEKIVHLKNLKKKKLMTDRNSEELCKLVKIMFEYKMEEKKSEN | |
| SEQ | MGNLFGHKRWYEVRDKKDFKIKRKVKVKRNYDGNKYILNINENNNKEKIDNNK | Leptotrichia |
| ID | FIRKYINYKKNDNILKEFTRKFHAGNILFKLKGKEGIIRIENNDDFLETEEVV | shahii |
| NO: | LYIEAYGKSEKLKALGITKKKIIDEAIRQGITKDDKKIEIKRQENEEEIEIDI | (Lsh) |
| 205 | RDEYTNKTLNDCSIILRIIENDELETKKSIYEIFKNINMSLYKIIEKIIENET | C2c2 |
| | EKVFENRYYEEHLREKLLKDDKIDVILTNEMEIREKIKSNLEILGFVKFYLNV | protein |
| | GGDKKKSKNKKMLVEKILNINVDLTVEDIADEVIKELEFWNITKRIEKVKKVN | |
| | NEFLEKRRNRTYIKSYVLLDKHEKFKIERENKKDKIVKFFVENIKNNSIKEKI | |
| | EKILAEFKIDELIKKLEKELKKGNCDTEIFGIFKKHYKVNEDSKKESKKSDEE | |
| | KELYKIIYRYLKGRIEKILVNEQKVRLKKMEKIEIEKILNESILSEKILKRVK | |
| | QYTLEHIMYLGKLRHNDIDMTTVNTDDESRLHAKEELDLELITFFASTNMELN | |
| | KIFSRENINNDENIDFFGGDREKNYVLDKKILNSKIKIIRDLDFIDNKNNITN | |
| | NFIRKFTKIGTNERNRILHAISKERDLQGTQDDYNKVINIIQNLKISDEEVSK | |
| | ALNLDVVEKDKKNIITKINDIKISEENNNDIKYLPSFSKVLPEILNLYRNNPK | |
| | NEPFDTIETEKIVLNALIYVNKELYKKLILEDDLEENESKNIFLQELKKTLGN | |
| | IDEIDENIIENYYKNAQISASKGNNKAIKKYQKKVIECYIGYLRKNYEELFDF | |
| | SDFKMNIQEIKKQIKDINDNKTYERITVKTSDKTIVINDDFEYIISIFALLNS | |
| | NAVINKIRNRFFATSVWLNTSEYQNIIDILDEIMQLNTLRNECITENWNLNLE | |
| | EFIQKMKEIEKDEDDEKIQTKKEIFNNYYEDIKNNILTEFKDDINGCDVLEKK | |
| | LEKIVIFDDETKFEIDKKSNILQDEQRKLSNINKKDLKKKVDQYIKDKDQEIK | |
| | SKILCRIIFNSDFLKKYKKEIDNLIEDMESENENKFQEIYYPKERKNELYIYK | |
| | KNLFLNIGNPNEDKIYGLISNDIKMADAKELENIDGKNIRKNKISEIDAILKN | |
| | LNDKLNGYSKEYKEKYIKKLKENDDEFAKNIQNKNYKSFEKDYNRVSEYKKIR | |
| | DLVEFNYLNKIESYLIDINWKLAIQMARFERDMHYIVNGLRELGIIKLSGYNT | |
| | GISRAYPKRNGSDGFYTTTAYYKFFDEESYKKFEKICYGFGIDLSENSEINKP | |
| | ENESIRNYISHFYIVRNPFADYSIAEQIDRVSNLLSYSTRYNNSTYASVFEVF | |
| | KKDVNLDYDELKKKFKLIGNNDILERLMKPKKVSVLELESYNSDYIKNLIIEL | |
| | LTKIENTNDTL | |
| SEQ | MQIGKVQGRTISEFGDPAGGLKRKISTDGKNRKELPAHLSSDPKALIGQWISG | Rhodobacter |
| ID | IDKIYRKPDSRKSDGKAIHSPTPSKMQFDARDDLGEAFWKLVSEAGLAQDSDY | capsulatus |
| NO: | DQFKRRLHPYGDKFQPADSGAKLKFEADPPEPQAFHGRWYGAMSKRGNDAKEL | C2c2 |
| 206 | AAALYEHLHVDEKRIDGQPKRNPKTDKFAPGLVVARALGIESSVLPRGMARLA | amino |
| | RNWGEEEIQTYFVVDVAASVKEVAKAAVSAAQAFDPPRQVSGRSLSPKVGFAL | acid |
| | AEHLERVTGSKRCSFDPAAGPSVLALHDEVKKTYKRLCARGKNAARAFPADKT | sequence |
| | ELLALMRHTHENRVRNQMVRMGRVSEYRGQQAGDLAQSHYWTSAGQTEIKESE | |
| | IFVRLWVGAFALAGRSMKAWIDPMGKIVNTEKNDRDLTAAVNIRQVISNKEMV | |
| | AEAMARRGIYFGETPELDRLGAEGNEGFVFALLRYLRGCRNQTFHLGARAGEL | |
| | KEIRKELEKTRWGKAKEAEHVVLTDKTVAAIRAIIDNDAKALGARLLADLSGA | |
| | FVAHYASKEHFSTLYSEIVKAVKDAPEVSSGLPRLKLLLKRADGVRGYVHGLR | |
| | DTRKHAFATKLPPPPAPRELDDPATKARYIALLRLYDGPFRAYASGITGTALA | |
| | GPAARAKEAATALAQSVNVTKAYSDVMEGRSSRLRPPNDGETLREYLSALTGE | |
| | TATEFRVQIGYESDSENARKQAEFIENYRRDMLAFMFEDYIRAKGEDWILKIE | |
| | PGATAMTRAPVLPEPIDTRGQYEHWQAALYLVMHFVPASDVSNLLHQLRKWEA | |
| | LQGKYELVQDGDATDQADARREALDLVKRFRDVLVLFLKTGEARFEGRAAPED | |
| | LKPFRALFANPATFDRLFMATPTTARPAEDDPEGDGASEPELRVARTLRGLRQ | |
| | IARYNHMAVLSDLFAKHKVRDEEVARLAEIEDETQEKSQIVAAQELRTDLHDK | |
| | VMKCHPKTISPEERQSYAAAIKTIEEHRFLVGRVYLGDHLRLHRLMMDVIGRL | |
| | IDYAGAYERDTGTELINASKQLGAGADWAVTIAGAANTDARTQTRKDLAHFNV | |
| | LDRADGTPDLTALVNRAREMMAYDRKRKNAVPRSILDMLARLGLTLKWQMKDH | |
| | LLQDATITQAAIKHLDKVRLTVGGPAAVTEARFSQDYLQMVAAVFNGSVQNPK | |
| | PRRRDDGDAWHKPPKPATAQSQPDQKPPNKAPSAGSRLPPPQVGEVYEGVVVK | |
| | VIDTGSLGFLAVEGVAGNIGLHISRLRRIREDAIIVGRRYRFRVEIYVPPKSN | |
| | TSKLNAADLVRID | |
| SEQ | MRITKVKIKLDNKLYQVTMQKEEKYGTLKLNEESRKSTAEILRLKKASFNKSF | Carnobacterium |
| ID | HSKTINSQKENKNATIKKNGDYISQIFEKLVGVDTNKNIRKPKMSLTDLKDLP | gallinarum |
| NO: | KKDLALFIKRKFKNDDIVEIKNLDLISLFYNALQKVPGEHFTDESWADFCQEM | C2c2 |
| 207 | MPYREYKNKFIERKIILLANSIEQNKGFSINPETFSKRKRVLHQWAIEVQERG | amino |
| | DFSILDEKLSKLAEIYNFKKMCKRVQDELNDLEKSMKKGKNPEKEKEAYKKQK | acid |
| | NEKIKTIWKDYPYKTHIGLIEKIKENEELNQFNIEIGKYFEHYFPIKKERCTE | sequence |
| | DEPYYLNSETIATTVNYQLKNALISYLMQIGKYKQFGLENQVLDSKKLQEIGI | |
| | YEGFQTKFMDACVFATSSLKNIIEPMRSGDILGKREFKEAIATSSFVNYHHFF | |
| | PYFPFELKGMKDRESELIPFGEQTEAKQMQNIWALRGSVQQIRNEIFHSFDKN | |
| | QKFNLPQLDKSNFEFDASENSTGKSQSYIETDYKFLFEAEKNQLEQFFIERIK | |
| | SSGALEYYPLKSLEKLFAKKEMKESLGSQVVAFAPSYKKLVKKGHSYQTATEG | |
| | TANYLGLSYYNRYELKEESFQAQYYLLKLIYQYVELPNFSQGNSPAFRETVKA | |
| | ILRINKDEARKKMKKNKKELRKYAFEQVREMEFKETPDQYMSYLQSEMREEKV | |
| | RKAEKNDKGFEKNITMNFEKLLMQIFVKGEDVELTTFAGKELLLSSEEKVIKE | |
| | TEISLSKKINEREKTLKASIQVEHQLVATNSAISYWLFCKLLDSRHLNELRNE | |
| | MIKFKQSRIKENHTQHAELIQNLLPIVELTILSNDYDEKNDSQNVDVSAYFED | |
| | KSLYETAPYVQTDDRTRVSFRPILKLEKYHTKSLIEALLKDNPQFRVAATDIQ | |
| | EWMHKREEIGELVEKRKNLHTEWAEGQQTLGAEKREEYRDYCKKIDRFNWKAN | |
| | KVTLTYLSQLHYLITDLLGRMVGESALFERDLVYFSRSESELGGETYHISDYK | |
| | NLSGVLRLNAEVKPIKIKNIKVIDNEENPYKGNEPEVKPFLDRLHAYLENVIG | |
| | IKAVHGKIRNQTAHLSVLQLELSMIESMNNLRDLMAYDRKLKNAVTKSMIKIL | |
| | DKHGMILKLKIDENHKNFEIESLIPKEIIHLKDKAIKTNQVSEEYCQLVLALL | |
| | TTNPGNQLN | |
| SEQ | MKLTRRRISGNSVDQKITAAFYRDMSQGLLYYDSEDNDCTDKVIESMDFERSW | Herbinix |
| ID | RGRILKNGEDDKNPFYMFVKGLVGSNDKIVCEPIDVDSDPDNLDILINKNLTG | hemicellulosilytica |
| NO: | FGRNLKAPDSNDTLENLIRKIQAGIPEEEVLPELKKIKEMIQKDIVNRKEQLL | C2c2 |
| 208 | KSIKNNRIPFSLEGSKLVPSTKKMKWLFKLIDVPNKTENEKMLEKYWEIYDYD | amino |
| | KLKANITNRLDKTDKKARSISRAVSEELREYHKNLRTNYNRFVSGDRPAAGLD | acid |
| | NGGSAKYNPDKEEFLLELKEVEQYFKKYFPVKSKHSNKSKDKSLVDKYKNYCS | sequence |
| | YKVVKKEVNRSIINQLVAGLIQQGKLLYYFYYNDTWQEDELNSYGLSYIQVEE | |
| | AFKKSVMTSLSWGINRLTSFFIDDSNTVKEDDITTKKAKEAIESNYENKLRTC | |
| | SRMQDHFKEKLAFFYPVYVKDKKDRPDDDIENLIVLVKNAIESVSYLRNRTFH | |
| | FKESSLLELLKELDDKNSGQNKIDYSVAAEFIKRDIENLYDVFREQIRSLGIA | |
| | EYYKADMISDCFKTCGLEFALYSPKNSLMPAFKNVYKRGANLNKAYIRDKGPK | |
| | ETGDQGQNSYKALEEYRELTWYIEVKNNDQSYNAYKNLLQLIYYHAFLPEVRE | |
| | NEALITDFINRTKEWNRKETEERLNTKNNKKHKNFDENDDITVNTYRYESIPD | |
| | YQGESLDDYLKVLQRKQMARAKEVNEKEEGNNNYIQFIRDVVVWAFGAYLENK | |
| | LKNYKNELQPPLSKENIGLNDTLKELFPEEKVKSPFNIKCRESISTFIDNKGK | |
| | STDNTSAEAVKTDGKEDEKDKKNIKRKDLLCFYLFLRLLDENEICKLQHQFIK | |
| | YRCSLKERRFPGNRTKLEKETELLAELEELMELVRFTMPSIPEISAKAESGYD | |
| | TMIKKYFKDFIEKKVFKNPKTSNLYYHSDSKTPVTRKYMALLMRSAPLHLYKD | |
| | IFKGYYLITKKECLEYIKLSNIIKDYQNSLNELHEQLERIKLKSEKQNGKDSL | |
| | YLDKKDFYKVKEYVENLEQVARYKHLQHKINFESLYRIFRIHVDIAARMVGYT | |
| | QDWERDMHFLFKALVYNGVLEERRFEAIFNNNDDNNDGRIVKKIQNNLNNKNR | |
| | ELVSMLCWNKKLNKNEFGAIIWKRNPIAHLNHFTQTEQNSKSSLESLINSLRI | |
| | LLAYDRKRQNAVTKTINDLLLNDYHIRIKWEGRVDEGQIYFNIKEKEDIENEP | |
| | IIHLKHLHKKDCYIYKNSYMFDKQKEWICNGIKEEVYDKSILKCIGNLFKFDY | |
| | EDKNKSSANPKHT | |
| SEQ | MRVSKVKVKDGGKDKMVLVHRKTTGAQLVYSGQPVSNETSNILPEKKRQSFDL | Paludibacter |
| ID | STLNKTIIKFDTAKKQKLNVDQYKIVEKIFKYPKQELPKQIKAEEILPFLNHK | propionicigenes |
| NO: | FQEPVKYWKNGKEESFNLTLLIVEAVQAQDKRKLQPYYDWKTWYIQTKSDLLK | C2c2 |
| 209 | KSIENNRIDLTENLSKRKKALLAWETEFTASGSIDLTHYHKVYMTDVLCKMLQ | amino |
| | DVKPLTDDKGKINTNAYHRGLKKALQNHQPAIFGTREVPNEANRADNQLSIYH | acid |
| | LEVVKYLEHYFPIKTSKRRNTADDIAHYLKAQTLKTTIEKQLVNAIRANIIQQ | sequence |
| | GKTNHHELKADTTSNDLIRIKTNEAFVLNLTGTCAFAANNIRNMVDNEQTNDI | |
| | LGKGDFIKSLLKDNTNSQLYSFFFGEGLSTNKAEKETQLWGIRGAVQQIRNNV | |
| | NHYKKDALKTVFNISNFENPTITDPKQQTNYADTIYKARFINELEKIPEAFAQ | |
| | QLKTGGAVSYYTIENLKSLLTTFQFSLCRSTIPFAPGFKKVFNGGINYQNAKQ | |
| | DESFYELMLEQYLRKENFAEESYNARYFMLKLIYNNLFLPGFTTDRKAFADSV | |
| | GFVQMQNKKQAEKVNPRKKEAYAFEAVRPMTAADSIADYMAYVQSELMQEQNK | |
| | KEEKVAEETRINFEKFVLQVFIKGEDSFLRAKEFDFVQMPQPQLTATASNQQK | |
| | ADKLNQLEASITADCKLTPQYAKADDATHIAFYVFCKLLDAAHLSNLRNELIK | |
| | FRESVNEFKFHHLLEIIEICLLSADVVPTDYRDLYSSEADCLARLRPFIEQGA | |
| | DITNWSDLFVQSDKHSPVIHANIELSVKYGTTKLLEQIINKDTQFKTTEANFT | |
| | AWNTAQKSIEQLIKQREDHHEQWVKAKNADDKEKQERKREKSNFAQKFIEKHG | |
| | DDYLDICDYINTYNWLDNKMHFVHLNRLHGLTIELLGRMAGEVALFDRDFQFF | |
| | DEQQIADEFKLHGFVNLHSIDKKLNEVPTKKIKEIYDIRNKIIQINGNKINES | |
| | VRANLIQFISSKRNYYNNAFLHVSNDEIKEKQMYDIRNHIAHFNYLTKDAADE | |
| | SLIDLINELRELLHYDRKLKNAVSKAFIDLFDKHGMILKLKLNADHKLKVESL | |
| | EPKKIYHLGSSAKDKPEYQYCTNQVMMAYCNMCRSLLEMKK | |
| SEQ | MYMKITKIDGVSHYKKQDKGILKKKWKDLDERKQREKIEARYNKQIESKIYKE | Leptotrichia |
| ID | FFRLKNKKRIEKEEDQNIKSLYFFIKELYLNEKNEEWELKNINLEILDDKERV | wadei |
| NO: | IKGYKFKEDVYFFKEGYKEYYLRILENNLIEKVQNENREKVRKNKEFLDLKEI | (Lwa) |
| 210 | FKKYKNRKIDLLLKSINNNKINLEYKKENVNEEIYGINPTNDREMTFYELLKE | C2c2 |
| | IIEKKDEQKSILEEKLDNFDITNFLENIEKIFNEETEINIIKGKVLNELREYI | amino |
| | KEKEENNSDNKLKQIYNLELKKYIENNFSYKKQKSKSKNGKNDYLYLNFLKKI | acid |
| | MFIEEVDEKKEINKEKFKNKINSNFKNLFVQHILDYGKLLYYKENDEYIKNTG | sequence |
| | QLETKDLEYIKTKETLIRKMAVLVSFAANSYYNLFGRVSGDILGTEVVKSSKT | |
| | NVIKVGSHIFKEKMLNYFFDFEIFDANKIVEILESISYSIYNVRNGVGHENKL | |
| | ILGKYKKKDINTNKRIEEDLNNNEEIKGYFIKKRGEIERKVKEKELSNNLQYY | |
| | YSKEKIENYFEVYEFEILKRKIPFAPNFKRIIKKGEDLENNKNNKKYEYFKNF | |
| | DKNSAEEKKEFLKTRNELLKELYYNNFYKEFLSKKEEFEKIVLEVKEEKKSRG | |
| | NINNKKSGVSFQSIDDYDTKINISDYIASIHKKEMERVEKYNEEKQKDTAKYI | |
| | RDFVEEIFLTGFINYLEKDKRLHELKEEFSILCNNNNNVVDENININEEKIKE | |
| | FLKENDSKTLNLYLFENMIDSKRISEFRNELVKYKQFTKKRLDEEKEFLGIKI | |
| | ELYETLIEFVILTREKLDTKKSEEIDAWLVDKLYVKDSNEYKEYEEILKLFVD | |
| | EKILSSKEAPYYATDNKTPILLSNFEKTRKYGTQSFLSEIQSNYKYSKVEKEN | |
| | IEDYNKKEEIEQKKKSNIEKLQDLKVELHKKWEQNKITEKEIEKYNNTTRKIN | |
| | EYNYLKNKEELQNVYLLHEMLSDLLARNVAFENKWERDFKFIVIAIKQFLREN | |
| | DKEKVNEFLNPPDNSKGKKVYFSVSKYKNTVENIDGIHKNEMNLIFLNNKFMN | |
| | RKIDKMNCAIWVYFRNYIAHFLHLHTKNEKISLISQMNLLIKLESYDKKVQNH | |
| | ILKSTKTLLEKYNIQINFEISNDKNEVEKYKIKNRLYSKKGKMLGKNNKFEIL | |
| | ENEFLENVKAMLEYSE | |
| SEQ | MENKTSLGNNIYYNPFKPQDKSYFAGYFNAAMENTDSVFRELGKRLKGKEYTS | Bergeyella |
| ID | ENFFDAIFKENISLVEYERYVKLLSDYFPMARLLDKKEVPIKERKENFKKNFK | zoohelcum |
| NO: | GIIKAVRDLRNFYTHKEHGEVEITDEIFGVLDEMLKSTVLTVKKKKVKTDKTK | Cas13b |
| 211 | EILKKSIEKQLDILCQKKLEYLRDTARKIEEKRRNORERGEKELVAPFKYSDK | |
| | RDDLIAAIYNDAFDVYIDKKKDSLKESSKAKYNTKSDPQQEEGDLKIPISKNG | |
| | VVFLLSLELTKQEIHAFKSKIAGFKATVIDEATVSEATVSHGKNSICEMATHE | |
| | IFSHLAYKKLKRKVRTAEINYGEAENAEQLSVYAKETLMMQMLDELSKVPDVV | |
| | YQNLSEDVQKTFIEDWNEYLKENNGDVGTMEEEQVIHPVIRKRYEDKENYFAI | |
| | RFLDEFAQFPTLRFQVHLGNYLHDSRPKENLISDRRIKEKITVEGRLSELEHK | |
| | KALFIKNTETNEDREHYWEIFPNPNYDFPKENISVNDKDEPIAGSILDREKQP | |
| | VAGKIGIKVKLLNQQYVSEVDKAVKAHQLKQRKASKPSIQNIIEEIVPINESN | |
| | PKEAIVFGGQPTAYLSMNDIHSILYEFFDKWEKKKEKLEKKGEKELRKEIGKE | |
| | LEKKIVGKIQAQIQQIIDKDTNAKILKPYQDGNSTAIDKEKLIKDLKQEQNIL | |
| | QKLKDEQTVREKEYNDFIAYQDKNREINKVRDRNHKQYLKDNLKRKYPEAPAR | |
| | KEVLYYREKGKVAVWLANDIKREMPTDFKNEWKGEQHSLLOKSLAYYEQCKEE | |
| | LKNLLPEKVFQHLPFKLGGYFQQKYLYQFYTCYLDKRLEYISGLVQQAENFKS | |
| | ENKVFKKVENECFKELKKQNYTHKELDARVQSILGYPIFLERGFMDEKPTIIK | |
| | GKTFKGNEALFADWFRYYKEYQNFQTFYDTENYPLVELEKKQADRKRKTKIYQ | |
| | QKKNDVFTLLMAKHIFKSVFKQDSIDQFSLEDLYQSREERLGNQERARQTGER | |
| | NTNYIWNKTVDLKLCDGKITVENVKLKNVGDFIKYEYDORVQAFLKYEENIEW | |
| | QAFLIKESKEEENYPYVVEREIEQYEKVRREELLKEVHLIEEYILEKVKDKEI | |
| | LKKGDNQNFKYYILNGLLKQLKNEDVESYKVFNLNTEPEDVNINQLKQEATDL | |
| | EQKAFVLTYIRNKFAHNQLPKKEFWDYCQEKYGKIEKEKTYAEYFAEVFKKEK | |
| | EALIK | |
| SEQ | MEDDKKTTDSIRYELKDKHFWAAFLNLARHNVYITVNHINKILEEGEINRDGY | Prevotella |
| ID | ETTLKNTWNEIKDINKKDRLSKLIIKHFPFLEAATYRLNPTDTTKQKEEKQAE | intermedia |
| NO: | AQSLESLRKSFFVFIYKLRDLRNHYSHYKHSKSLERPKFEEGLLEKMYNIFNA | Cas13b |
| 212 | SIRLVKEDYQYNKDINPDEDFKHLDRTEEEFNYYFTKDNEGNITESGLLFFVS | |
| | LFLEKKDAIWMQQKLRGFKDNRENKKKMTNEVFCRSRMLLPKLRLOSTQTQDW | |
| | ILLDMLNELIRCPKSLYERLREEDREKERVPIEIADEDYDAEQEPFKNTLVRH | |
| | QDRFPYFALRYFDYNEIFTNLRFQIDLGTYHFSIYKKQIGDYKESHHLTHKLY | |
| | GFERIQEFTKQNRPDEWRKFVKTENSFETSKEPYIPETTPHYHLENQKIGIRF | |
| | RNDNDKIWPSLKTNSEKNEKSKYKLDKSFQAEAFLSVHELLPMMFYYLLLKTE | |
| | NTDNDNEIETKKKENKNDKQEKHKIEEIIENKITEIYALYDTFANGEIKSIDE | |
| | LEEYCKGKDIEIGHLPKQMIAILKDEHKVMATEAERKQEEMLVDVQKSLESLD | |
| | NQINEEIENVERKNSSLKSGKIASWLVNDMMRFQPVQKDNEGKPLNNSKANST | |
| | EYQLLQRTLAFFGSEHERLAPYFKQTKLIESSNPHPFLKDTEWEKCNNILSFY | |
| | RSYLEAKKNFLESLKPEDWEKNQYFLKLKEPKTKPKTLVQGWKNGENLPRGIF | |
| | TEPIRKWFMKHRENITVAELKRVGLVAKVIPLFFSEEYKDSVQPFYNYHFNVG | |
| | NINKPDEKNFLNCEERRELLRKKKDEFKKMTDKEKEENPSYLEFKSWNKFERE | |
| | LRLVRNQDIVTWLLCMELFNKKKIKELNVEKIYLKNINTNTTKKEKNTEEKNG | |
| | EEKNIKEKNNILNRIMPMRLPIKVYGRENFSKNKKKKIRRNTFFTVYIEEKGT | |
| | KLLKQGNFKALERDRRLGGLFSFVKTPSKAESKSNTISKLRVEYELGEYQKAR | |
| | IEIIKDMLALEKTLIDKYNSLDTDNFNKMLTDWLELKGEPDKASFQNDVDLLI | |
| | AVRNAFSHNQYPMRNRIAFANINPFSLSSANTSEEKGLGIANQLKDKTHKTIE | |
| | KIIEIEKPIETKE | |
| SEQ | MQKQDKLFVDRKKNAIFAFPKYITIMENKEKPEPIYYELTDKHFWAAFLNLAR | Prevotella |
| ID | HNVYTTINHINRRLEIAELKDDGYMMGIKGSWNEQAKKLDKKVRLRDLIMKHF | buccae |
| NO: | PFLEAAAYEMTNSKSPNNKEQREKEQSEALSLNNLKNVLFIFLEKLQVLRNYY | Cas13b |
| 213 | SHYKYSEESPKPIFETSLLKNMYKVFDANVRLVKRDYMHHENIDMQRDFTHLN | |
| | RKKQVGRTKNIIDSPNFHYHFADKEGNMTIAGLLFFVSLFLDKKDAIWMQKKL | |
| | KGFKDGRNLREQMTNEVFCRSRISLPKLKLENVQTKDWMQLDMLNELVRCPKS | |
| | LYERLREKDRESFKVPFDIFSDDYNAEEEPFKNTLVRHQDRFPYFVLRYFDLN | |
| | EIFEQLRFQIDLGTYHESIYNKRIGDEDEVRHLTHHLYGFARIQDFAPQNQPE | |
| | EWRKLVKDLDHFETSQEPYISKTAPHYHLENEKIGIKFCSAHNNLFPSLQTDK | |
| | TCNGRSKFNLGTQFTAEAFLSVHELLPMMFYYLLLTKDYSRKESADKVEGIIR | |
| | KEISNIYAIYDAFANNEINSIADLTRRLQNTNILQGHLPKQMISILKGRQKDM | |
| | GKEAERKIGEMIDDTQRRLDLLCKQTNQKIRIGKRNAGLLKSGKIADWLVNDM | |
| | MRFQPVQKDQNNIPINNSKANSTEYRMLQRALALFGSENERLKAYENQMNLVG | |
| | NDNPHPFLAETQWEHQTNILSFYRNYLEARKKYLKGLKPQNWKQYQHFLILKV | |
| | QKTNRNTLVTGWKNSENLPRGIFTQPIREWEEKHNNSKRIYDQILSFDRVGFV | |
| | AKAIPLYFAEEYKDNVQPFYDYPFNIGNRLKPKKRQFLDKKERVELWQKNKEL | |
| | FKNYPSEKKKTDLAYLDFLSWKKFERELRLIKNQDIVTWLMFKELFNMATVEG | |
| | LKIGEIHLRDIDTNTANEESNNILNRIMPMKLPVKTYETDNKGNILKERPLAT | |
| | FYIEETETKVLKQGNFKALVKDRRLNGLFSFAETTDLNLEEHPISKLSVDLEL | |
| | IKYQTTRISIFEMTLGLEKKLIDKYSTLPTDSERNMLERWLQCKANRPELKNY | |
| | VNSLIAVRNAFSHNQYPMYDATLFAEVKKFTLFPSVDTKKIELNIAPQLLEIV | |
| | GKAIKEIEKSENKN | |
| SEQ | MNTVPASENKGQSRTVEDDPQYFGLYLNLARENLIEVESHVRIKFGKKKLNEE | Porphyromonas |
| ID | SLKQSLLCDHLLSVDRWTKVYGHSRRYLPFLHYFDPDSQIEKDHDSKTGVDPD | gingivalis |
| NO: | SAQRLIRELYSLLDFLRNDFSHNRLDGTTFEHLEVSPDISSFITGTYSLACGR | Cas13b |
| 214 | AQSRFAVFFKPDDEVLAKNRKEQLISVADGKECLTVSGFAFFICLFLDREQAS | |
| | GMLSRIRGFKRTDENWARAVHETFCDLCIRHPHDRLESSNTKEALLLDMLNEL | |
| | NRCPRILYDMLPEEERAQFLPALDENSMNNLSENSLDEESRLLWDGSSDWAEA | |
| | LTKRIRHQDRFPYLMLRFIEEMDLLKGIRFRVDLGEIELDSYSKKVGRNGEYD | |
| | RTITDHALAFGKLSDFQNEEEVSRMISGEASYPVRFSLFAPRYAIYDNKIGYC | |
| | HTSDPVYPKSKTGEKRALSNPQSMGFISVHDLRKLLLMELLCEGSFSRMQSDE | |
| | LRKANRILDETAEGKLQFSALFPEMRHRFIPPQNPKSKDRREKAETTLEKYKQ | |
| | EIKGRKDKLNSQLLSAFDMDQRQLPSRLLDEWMNIRPASHSVKLRTYVKQLNE | |
| | DCRLRLRKFRKDGDGKARAIPLVGEMATFLSQDIVRMIISEETKKLITSAYYN | |
| | EMQRSLAQYAGEENRRQFRAIVAELRLLDPSSGHPFLSATMETAHRYTEGFYK | |
| | CYLEKKREWLAKIFYRPEQDENTKRRISVFFVPDGEARKLLPLLIRRRMKEQN | |
| | DLQDWIRNKQAHPIDLPSHLFDSKVMELLKVKDGKKKWNEAFKDWWSTKYPDG | |
| | MQPFYGLRRELNIHGKSVSYIPSDGKKFADCYTHLMEKTVRDKKRELRTAGKP | |
| | VPPDLAADIKRSFHRAVNEREFMLRLVQEDDRLMLMAINKMMTDREEDILPGL | |
| | KNIDSILDEENQFSLAVHAKVLEKEGEGGDNSLSLVPATIEIKSKRKDWSKYI | |
| | RYRYDRRVPGLMSHFPEHKATLDEVKTLLGEYDRCRIKIFDWAFALEGAIMSD | |
| | RDLKPYLHESSSREGKSGEHSTLVKMLVEKKGCLTPDESQYLILIRNKAAHNQ | |
| | FPCAAEMPLIYRDVSAKVGSIEGSSAKDLPEGSSLVDSLWKKYEMIIRKILPI | |
| | LDPENRFFGKLLNNMSQPINDL | |
| SEQ | MESIKNSQKSTGKTLQKDPPYFGLYLNMALLNVRKVENHIRKWLGDVALLPEK | Bacteroides |
| ID | SGFHSLLTTDNLSSAKWTRFYYKSRKFLPFLEMEDSDKKSYENRRETAECLDT | pyogenes |
| NO: | IDRQKISSLLKEVYGKLQDIRNAFSHYHIDDQSVKHTALIISSEMHRFIENAY | Cas13b |
| 215 | SFALQKTRARFTGVFVETDELQAEEKGDNKKFFAIGGNEGIKLKDNALIFLIC | |
| | LFLDREEAFKFLSRATGFKSTKEKGFLAVRETFCALCCRQPHERLLSVNPREA | |
| | LLMDMLNELNRCPDILFEMLDEKDQKSFLPLLGEEEQAHILENSLNDELCEAI | |
| | DDPFEMIASLSKRVRYKNRFPYLMLRYIEEKNLLPFIRFRIDLGCLELASYPK | |
| | KMGEENNYERSVTDHAMAFGRLTDFHNEDAVLQQITKGITDEVRESLYAPRYA | |
| | IYNNKIGFVRTSGSDKISFPTLKKKGGEGHCVAYTLQNTKSFGFISIYDLRKI | |
| | LLLSFLDKDKAKNIVSGLLEQCEKHWKDLSENLFDAIRTELQKEFPVPLIRYT | |
| | LPRSKGGKLVSSKLADKQEKYESEFERRKEKLTEILSEKDEDLSQIPRRMIDE | |
| | WLNVLPTSREKKLKGYVETLKLDCRERLRVFEKREKGEHPLPPRIGEMATDLA | |
| | KDIIRMVIDQGVKQRITSAYYSEIQRCLAQYAGDDNRRHLDSIIRELRLKDTK | |
| | NGHPFLGKVLRPGLGHTEKLYQRYFEEKKEWLEATFYPAASPKRVPRFVNPPT | |
| | GKQKELPLIIRNLMKERPEWRDWKQRKNSHPIDLPSQLFENEICRLLKDKIGK | |
| | EPSGKLKWNEMFKLYWDKEFPNGMQRFYRCKRRVEVEDKVVEYEYSEEGGNYK | |
| | KYYEALIDEVVRQKISSSKEKSKLQVEDLTLSVRRVFKRAINEKEYQLRLLCE | |
| | DDRLLFMAVRDLYDWKEAQLDLDKIDNMLGEPVSVSQVIQLEGGQPDAVIKAE | |
| | CKLKDVSKLMRYCYDGRVKGLMPYFANHEATQEQVEMELRHYEDHRRRVFNWV | |
| | FALEKSVLKNEKLRRFYEESQGGCEHRRCIDALRKASLVSEEEYEFLVHIRNK | |
| | SAHNQFPDLEIGKLPPNVTSGFCECIWSKYKAIICRIIPFIDPERRFFGKLLE | |
| | QK | |
| SEQ | MTEKKSIIFKNKSSVEIVKKDIFSQTPDNMIRNYKITLKISEKNPRVVEAEIE | Cas13c |
| ID | DLMNSTILKDGRRSARREKSMTERKLIEEKVAENYSLLANCPMEEVDSIKIYK | |
| NO: | IKRFLTYRSNMLLYFASINSELCEGIKGKDNETEEIWHLKDNDVRKEKVKENF | |
| 216 | KNKLIQSTENYNSSLKNQIEEKEKLLRKESKKGAFYRTIIKKLQQERIKELSE | |
| | KSLTEDCEKIIKLYSELRHPLMHYDYQYFENLFENKENSELTKNLNLDIFKSL | |
| | PLVRKMKLNNKVNYLEDNDTLFVLQKTKKAKTLYQIYDALCEQKNGENKFIND | |
| | FFVSDGEENTVFKQIINEKFQSEMEFLEKRISESEKKNEKLKKKFDSMKAHFH | |
| | NINSEDTKEAYFWDIHSSSNYKTKYNERKNLVNEYTELLGSSKEKKLLREEIT | |
| | QINRKLLKLKQEMEEITKKNSLFRLEYKMKIAFGFLFCEFDGNISKFKDEFDA | |
| | SNQEKIIQYHKNGEKYLTYFLKEEEKEKFNLEKMQKIIQKTEEEDWLLPETKN | |
| | NLFKFYLLTYLLLPYELKGDFLGFVKKHYYDIKNVDFMDENQNNIQVSQTVEK | |
| | QEDYFYHKIRLFEKNTKKYEIVKYSIVPNEKLKQYFEDLGIDIKYLTGSVESG | |
| | EKWLGENLGIDIKYLTVEQKSEVSEEKIKKFL | |
| SEQ | MEKDKKGEKIDISQEMIEEDLRKILILFSRLRHSMVHYDYEFYQALYSGKDFV | Cas13c |
| ID | ISDKNNLENRMISQLLDLNIFKELSKVKLIKDKAISNYLDKNTTIHVLGQDIK | |
| NO: | AIRLLDIYRDICGSKNGFNKFINTMITISGEEDREYKEKVIEHFNKKMENLST | |
| 217 | YLEKLEKQDNAKRNNKRVYNLLKQKLIEQQKLKEWEGGPYVYDIHSSKRYKEL | |
| | YIERKKLVDRHSKLFEEGLDEKNKKELTKINDELSKLNSEMKEMTKLNSKYRL | |
| | QYKLQLAFGFILEEFDLNIDTFINNFDKDKDLIISNEMKKRDIYLNRVLDRGD | |
| | NRLKNIIKEYKERDTEDIFCNDRDNNLVKLYILMYILLPVEIRGDFLGFVKKN | |
| | YYDMKHVDFIDKKDKEDKDTFFHDLRLFEKNIRKLEITDYSLSSGFLSKEHKV | |
| | DIEKKINDFINRNGAMKLPEDITIEEFNKSLILPIMKNYQINFKLLNDIEISA | |
| | LFKIAKDRSITFKQAIDEIKNEDIKKNSKKNDKNNHKDKNINFTQLMKRALHE | |
| | KIPYKAGMYQIRNNISHIDMEQLYIDPLNSYMNSNKNNITISEQIEKIIDVCV | |
| | TGGVTGKELNNNIINDYYMKKEKLVENLKLRKQNDIVSIESQEKNKREEFVFK | |
| | KYGLDYKDGEINIIEVIQKVNSLQEELRNIKETSKEKLKNKETLFRDISLING | |
| | TIRKNINFKIKEMVLDIVRMDEIRHINIHIYYKGENYTRSNIIKFKYAIDGEN | |
| | KKYYLKQHEINDINLELKDKFVTLICNMDKHPNKNKQTINLESNYIQNVKFII | |
| | P | |
| SEQ | MENKGNNKKIDFDENYNILVAQIKEYFTKEIENYNNRIDNIIDKKELLKYSEK | Cas13c |
| ID | KEESEKNKKLEELNKLKSQKLKILTDEEIKADVIKIIKIFSDLRHSLMHYEYK | |
| NO: | YFENLFENKKNEELAELLNLNLFKNLTLLRQMKIENKTNYLEGREEFNIIGKN | |
| 218 | IKAKEVLGHYNLLAEQKNGFNNFINSFFVQDGTENLEFKKLIDEHFVNAKKRL | |
| | ERNIKKSKKLEKELEKMEQHYQRLNCAYVWDIHTSTTYKKLYNKRKSLIEEYN | |
| | KQINEIKDKEVITAINVELLRIKKEMEEITKSNSLERLKYKMQIAYAFLEIEF | |
| | GGNIAKFKDEFDCSKMEEVQKYLKKGVKYLKYYKDKEAQKNYEFPFEEIFENK | |
| | DTHNEEWLENTSENNLFKFYILTYLLLPMEFKGDELGVVKKHYYDIKNVDETD | |
| | ESEKELSQVQLDKMIGDSFFHKIRLFEKNTKRYEIIKYSILTSDEIKRYFRLL | |
| | ELDVPYFEYEKGTDEIGIENKNIILTIFKYYQIIFRLYNDLEIHGLFNISSDL | |
| | DKILRDLKSYGNKNINFREFLYVIKONNNSSTEEEYRKIWENLEAKYLRLHLL | |
| | TPEKEEIKTKTKEELEKLNEISNLRNGICHLNYKEIIEEILKTEISEKNKEAT | |
| | LNEKIRKVINFIKENELDKVELGENFINDFFMKKEQFMFGQIKQVKEGNSDSI | |
| | TTERERKEKNNKKLKETYELNCDNLSEFYETSNNLRERANSSSLLEDSAFLKK | |
| | IGLYKVKNNKVNSKVKDEEKRIENIKRKLLKDSSDIMGMYKAEVVKKLKEKLI | |
| | LIFKHDEEKRIYVTVYDTSKAVPENISKEILVKRNNSKEEYFFEDNNKKYVTE | |
| | YYTLEITETNELKVIPAKKLEGKEFKTEKNKENKLMLNNHYCFNVKIIY | |
| SEQ | MEEIKHKKNKSSIIRVIVSNYDMTGIKEIKVLYQKQGGVDTENLKTIINLESG | Cas13c |
| ID | NLEIISCKPKEREKYRYEFNCKTEINTISITKKDKVLKKEIRKYSLELYFKNE | |
| NO: | KKDTVVAKVTDLLKAPDKIEGERNHLRKLSSSTERKLLSKTLCKNYSEISKTP | |
| 219 | IEEIDSIKIYKIKRFLNYRSNELIYFALINDELCAGVKEDDINEVWLIQDKEH | |
| | TAFLENRIEKITDYIFDKLSKDIENKKNQFEKRIKKYKTSLEELKTETLEKNK | |
| | TFYIDSIKTKITNLENKITELSLYNSKESLKEDLIKIISIFTNLRHSLMHYDY | |
| | KSFENLFENIENEELKNLLDLNLFKSIRMSDEFKTKNRTNYLDGTESFTIVKK | |
| | HQNLKKLYTYYNNLCDKKNGFNTFINSFFVTDGIENTDEKNLIILHFEKEMEE | |
| | YKKSIEYYKIKISNEKNKSKKEKLKEKIDLLQSELINMREHKNLLKQIYFFDI | |
| | HNSIKYKELYSERKNLIEQYNLQINGVKDVTAINHINTKLLSLKNKMDKITKQ | |
| | NSLYRLKYKLKIAYSFLMIEFDGDVSKFKNNFDPTNLEKRVEYLDKKEEYLNY | |
| | TAPKNKFNFAKLEEELQKIQSTSEMGADYLNVSPENNLEKFYILTYIMLPVEF | |
| | KGDFLGFVKNHYYNIKNVDEMDESLLDENEVDSNKLNEKIENLKDSSFFNKIR | |
| | LFEKNIKKYEIVKYSVSTQENMKEYFKQLNLDIPYLDYKSTDEIGIENKNMIL | |
| | PIFKYYQNVFKLCNDIEIHALLALANKKOONLEYAIYCCSKKNSLNYNELLKT | |
| | FNRKTYQNLSFIRNKIAHLNYKELFSDLENNELDLNTKVRCLIEFSQNNKFDQ | |
| | IDLGMNFINDYYMKKTRFIFNQRRLRDLNVPSKEKIIDGKRKQQNDSNNELLK | |
| | KYGLSRTNIKDIFNKAWY | |
| SEQ | MKVRYRKQAQLDTFIIKTEIVNNDIFIKSIIEKAREKYRYSFLFDGEEKYHFK | Cas13c |
| ID | NKSSVEIVKNDIFSQTPDNMIRNYKITLKISEKNPRVVEAEIEDLMNSTILKD | |
| NO: | GRRSARREKSMTERKLIEEKVAENYSLLANCPIEEVDSIKIYKIKRFLTYRSN | |
| 220 | MLLYFASINSFLCEGIKGKDNETEEIWHLKDNDVRKEKVKENFKNKLIQSTEN | |
| | YNSSLKNQIEEKEKLSSKEFKKGAFYRTIIKKLQQERIKELSEKSLTEDCEKI | |
| | IKLYSELRHPLMHYDYQYFENLFENKENSELTKNLNLDIFKSLPLVRKMKLNN | |
| | KVNYLEDNDTLFVLQKTKKAKTLYQIYDALCEQKNGFNKFINDFFVSDGEENT | |
| | VFKQIINEKFQSEMEFLEKRISESEKKNEKLKKKLDSMKAHFRNINSEDTKEA | |
| | YFWDIHSSRNYKTKYNERKNLVNEYTKLLGSSKEKKLLREEITKINRQLLKLK | |
| | QEMEEITKKNSLERLEYKMKIAFGFLFCEFDGNISKFKDEFDASNQEKIIQYH | |
| | KNGEKYLTSFLKEEEKEKENLEKMQKIIQKTEEEDWLLPETKNNLFKFYLLTY | |
| | LLLPYELKGDFLGFVKKHYYDIKNVDEMDENQNNIQVSQTVEKQEDYFYHKIR | |
| | LFEKNTKKYEIVKYSIVPNEKLKQYFEDLGIDIKYLTGSVESGEKWLGENLGI | |
| | DIKYLTVEQKSEVSEEKNKKVSLKNNGMENKTILLFVFKYYQIAFKLFNDIEL | |
| | YSLFFLREKSEKPFEVELEELKDKMIGKQLNFGQLLYVVYEVLVKNKDLDKIL | |
| | SKKIDYRKDKSFSPEIAYLRNFLSHLNYSKFLDNEMKINTNKSDENKEVLIPS | |
| | IKIQKMIQFIEKCNLQNQIDFDENFVNDFYMRKEKMFFIQLKQIFPDINSTEK | |
| | QKKSEKEEILRKRYHLINKKNEQIKDEHEAQSQLYEKILSLQKIFSCDKNNFY | |
| | RRLKEEKLLFLEKQGKKKISMKEIKDKIASDISDLLGILKKEITRDIKDKLTE | |
| | KFRYCEEKLLNISFYNHQDKKKEEGIRVFLIRDKNSDNFKFESILDDGSNKIF | |
| | ISKNGKEITIQCCDKVLETLMIEKNTLKISSNGKIISLIPHYSYSIDVKY | |
| SEQ | MVKNPANRHALPKVIISEVDNNNILEFKIKYEKLARLDKVEVKSMHEDNNKQV | 2021Q1_ |
| ID | VFDEVVINGGLIEPTYEDKHKKLVVTAGEKSYSIVGQKVGGKPRLLEDRVSKT | 2.020 |
| NO: | KVQLELTNYVEDKEGKKRVSKTERELIVADNIELYSQIVGREVKTTKEIYLIK | |
| 248 | RFLEYRSDLLFYYGFVDNFFKVAGNGKELWKIDFTNSDSLHLIEYFKFSINDN | |
| | LKNDENYLKNYVSDNTKIENDLVKCQNNFNSLRHALMHFDYDFFEKLFNGEDV | |
| | GFDFDIEFLNIMIDKVDKLNIDTKKEFIDDEEVTLFGEALSLKKLYGLFSHIA | |
| | INRVAFNKLINSFIIEDGIENKELKDFENNKKESQAYEIDIHSNAEYKALYVQ | |
| | HKKLVMATSAMTDGDEIAKKNQEISDLKEKMKVITKENSLARLEHKLRLAFGF | |
| | IYTEYKDYKTFKKHFDQDIKGAKYKGLNVEKLKEYYETTLKNSKPKTDEKLED | |
| | VAKKIDKLSLKELIDDDTLLKFVLLLFIFMPQELKGDELGFIKKYYHDKKHID | |
| | QDTKDKDTEIEELSTGLKLKVLDKNIRSLSILKHSFSFQVKYNRKDKNFYEDG | |
| | NLHGKFYKKLSISHNQEEFNKSVYAPLFRYYSALYKLINDFEIYALAQHVENH | |
| | ETLADQVNKSQFIQKSYFNFRKLLDNTDSISQSSSYNTLIVMRNDISHLSYEP | |
| | LFNYPLDERKSYKKKTQKGVKTFHVELLYISRAKIIELISLQTDMKKLLGYDA | |
| | VNDFNMKVVHLRKRLSVYANKEESIRKMQADAKTPNDFYNIYKVKGVESINQH | |
| | LLKVIGVTEAEKSIEKQINEGNKKHNT | |
| SEQ | MIKKPSNRHALPKVIISKVDNQNILEFKIKYKKLSRLDRVEIKTMHYDDRAIV | 2021Q1_ |
| ID | FDEVIINGGLIDVEYRDNHKTIFVKVGDKSYSISGQKVGGKERLLENRISQTK | 2.018 |
| NO: | VQLELKDEATNRVSKTERELIVDDNIKLYSQIVGRDVKTTKDIYLIKRFLGYR | |
| 249 | SDLLFYYGFVNNFFHVANNRPEFWKIDENDNRNSKLIEYFIFTINDHLKNDEN | |
| | YLKDYISDRGQIVDDLENIKHIFSALRHGLMHFDYDFFEALFNGEDIDIKMDN | |
| | QGNTQPLSSLNIKFLDIMIDKLDKLNIDTKKEFIDAEKITIFGEELSLAKLYR | |
| | FYAHTAINRVAFNKLINSFIIENGVENQSLKEYFNQQAGGIAYEIDIHQNREY | |
| | KNLYNEHKKLVSRVLSISDGQEIATLNQKIVELKEQMKQITKINSIKRLEYKL | |
| | RLAFGFIYTEYKNYEEFKNSFDTDIKNGRFTPKDEDGNKRAFDSRELEHLKGY | |
| | YKATLQTQKPQTDEKMEEVSKRVDRLSLKSLIGDDTLLKFILLMFTFMPQELK | |
| | GEFLGFIKKYYHDTKHIDQDTISDSDDTIEEGLSIGLKLKILDKNIRSLSILK | |
| | HSLSFQTKYNKKDRSYYEDGNIHGKFFKKLGISHNQEEFNKSVYAPLFRYYSA | |
| | LYKLINDFEIYTLSLHIVGNETLSDQVNKPQFLSGRYFNFRKLLTQSYNISNN | |
| | STHSVIFNAVINMRNDISHLSYEPLLDCPLNGKKSYKRKIRNQFRTINIKPLV | |
| | ESRKMIIDFITLQTDMQKVLGCDAVNDFTMKIVQLRTRLKAYANKEQTIEKMI | |
| | TEAKTPNDFYNIYKVKGVEAINKYLLEVIGETQVEKEIREEIERGNIANS | |
| SEQ | MIKNPSNRHSLPKVIISEVDHEKILEFKIKYEKLARLDRFEVKAMHYEGKEIV | 2021Q1_ |
| ID | FDEVLVNGGLIEVEYQDDNKTLFVKVGEKSYSIRGKKVGGKQRLLEDRVSKTK | 2.001 |
| NO: | VQLELSDGVVDNKGNLRKSRTERELIVADNIKLYSQIVGREVTTTKEIYLVKR | |
| 250 | FLAYRSDLLFYYSFVDNFFKVAGNEKELWKINFDDATSAQFMGYIPFMVNDNL | |
| | KNDNAYLKDYVRNDVQIKDDLKKVQTIFSALRHTLLHFNYEFFEKLFNGEDVG | |
| | FDFDIGFLNLLIENIDKLNIDAKKEFIDNEKIRLFGENLSLAKVYRLYSDICV | |
| | NRVGFNKFINSMLIKDGVENQVLKAEFNRKFGGNAYTIDIHSNQEYKRIYNEH | |
| | KKLVIKVSTLKDGQAIRRGNKKISELKEQMKSMTKKNSLARLECKMRLAFGFL | |
| | YGEYNNYKAFKNNEDTNIKNSQFDVNDVEKSKAYFLSTYERRKPRTREKLEKV | |
| | AKDIESLELKTVIANDTLLKFILLMFVFMPQELKGDELGFVKKYYHDVHSIDD | |
| | DTKEQEEDVVEAMSTSLKLKILGRNIRSLTLFKYALSSQVNYNSTDNIFYVEG | |
| | NRYGKIYKKLGISHNQEEFDKTLVVPLLRYYSSLFKLMNDFEIYSLAKANPTA | |
| | VSLQELVDDETSPYKQGNYFNFNKMLRDIYGLTSDEIKSGQVVFMRNKIAHFD | |
| | TEVLLSKPLLGQTKMNLQRKDIVSFIEARGDIKELLGYDAINDFRMKVIHLRT | |
| | KMRVYSDKLQTMMDLLRNAKTPNDFYNVYKVKGVESINKHLLEVLAQTAEERT | |
| | VEKQIRDGNEKYDL | |
| SEQ | MNQYIHANKKENKKRPNKSSIIRIMVSDFDDEYIQEIKVLYIKQGGVDTFKIN | 2021Q1_ |
| ID | KMSYDSASKKIIFEEVATQNMLSVEDSNLNFKRPMVECKNGDIYVVKPSEKVN | 2.003 |
| NO: | EKGQAIEPLRSYKIHGKYLDLTEEIGKDNAEQSGKKQIYLHVEDLLGMHTTAD | |
| 251 | SIDRRRLESETQRTLLSKEVMENYALIMGHEIKLDESDETYKLASSKEIYKAN | |
| | RFLDYRSRLLYYYSFINHFLVGLSKGATYEYAGKSLVAKIPDGEVWQLCELEV | |
| | TAYHGIYINKKRNELVNENSLKIDAIYAQMAKNMKETINEFVDNYNAMIEKQN | |
| | ELKDNKSYKINKKAQNTTKFENFTEEKINQDLHNIVYILSDLRHKLMHFEYHY | |
| | FELLMTGKKMGEKSEVIVCVPDRNSELPASKNEPSKDKERKEKKLSELLDLNI | |
| | LKELDTFVKVKESYQTTYLETNDKIEILGKLKTAKSIYQIYHQICQRKNGENK | |
| | FINSFFTVDGEENTEVKDCINEVFRKEIHYFEMVIAKSNEDTLNDKNKNKSKK | |
| | TRDRMKNQIQECKRYQDDESNIDTWVAYHKDIHYSKRYKKLYCEHITLVDNLN | |
| | SAVSNGLNGQVIKEINDDIAIKKREMNEITKANSKSRLRYKMQMAYGELFVEY | |
| | GLKIPKFLNDEDLSHVRTASKIKGYKSPVKVTQYLTNDEGNKDNENLETLMED | |
| | IDKKSKINFEFLKSNEDNNLIKLYILIYQLLPRELKGDFLGFVKNNYYDLKHV | |
| | DFQTRETEAKDQFFHNMRLFEKNVKAFDLIQYSIGDEMSQLGNETFNFSQALS | |
| | KIVSDDTILNSATEIPNINRLVYGSLLKYYENAFRLSSEIEIRALIKIARGKQ | |
| | IDNHSIDEAYKDALIFQKSGQTVKFSSILKYFNIDDLNKKNDSKIYNKASNLR | |
| | NKIAHCDYTVLFITNIICEAENINVKAKYLIDVSNKIGLNTVDLGNDMVNDYL | |
| | MSYDKQMTYLAKTSEELLKESSLDKSKEKKERRDNLKNETRSHSEIYMEYEWI | |
| | SDYLVKLKDNHEILSKKNKTEDLKLNRAYELIKNYNVALNKKSKIKIRALTTD | |
| | HINNNWVNIWGALTHELQEIKGLYLYKVTPKIKKGIYFVLTDKKNYCLNINLY | |
| | TLKKVPSKDDSEMTEERYLRDISEKYYFTVGEDSIKALKERLQTINDKCIITY | |
| | VNEIDCKNENIKEKPEFKIAEDYNSWDKKYLSISNEELHHWSVETRRDKKYKE | |
| | NLILNLMEKSSFKLNLHQL | |
| SEQ | MSQLKNPSNKNSLPRIIISDFNEIKINEIKIKYHKLDRLDKIIVKEMEIINNK | 2021Q1_ |
| ID | IFFKKILENNQIKDINSENIELENYILAGEVKPSNTKIILNRDGKEKSFIVYD | 2.004 |
| NO: | GFTFKYKPNDKRISETKTNAKYILTIKDKTRHRESSTQRDILKSSIIETYKQI | |
| 252 | SGFENITSKDIYTIKRYIDFKNEMMFYYTFIDDFFFPITGKNKQDKKNNFYNY | |
| | KIKENAKKFISLINYRINDDFKNKNGILYDYLSNKEEIIINDFIHIQTILKDV | |
| | RHAIAHFNFDFIQKLEDNEQAFNSKEDGIEILNILENQKQEKYFEAQTNYIEE | |
| | ETIKILDEKELSFKKLHSFYSQICQKKPAFNKLINSFIIQDGIENKELKDYIS | |
| | QKYNSKFDYYLDIHTCKIYKDIYNQHKKFVADKQFLENQKTDGQKIKKLNDQI | |
| | NQLKTKMNNLTKKNSLKRLEIKFRLAFGFIFTEYQTFKNFNERFIEDIKANKY | |
| | STKIELLDYGKIKEYISITHEEKRFFNYKTENKKTNKNINKTIFQSLEKETFE | |
| | NLVKNDNLIKMMELFQLLLPRELKGEFLGFILKIYHDLKNIDNDTKPDEKSLS | |
| | ELNISTALKLKILVKNIRQINLENYTISNNTKYEEKEKRFYEEGNQWKDIYKK | |
| | LYISHDFDIFDIHLIIPIIKYNINLYKLIGDFEVYLLLKYLERNTNYKTLDKL | |
| | IEAEELKYKGYYNFTTLLSKAINIALNDKEYHNITHLRNNTSHQDIQNIISSE | |
| | KNNKLLEQRENIIELISKESLKKKLHFDPINDFTMKTLQLLKSLEVHSDKSEK | |
| | IENLLKKEPLLPNDVYLLYKLKGIEFIKKELISNIGITKYEEKIQEKIAKGVE | |
| | K | |
| SEQ | MIKNPANRYSLPKVIISEVDSQNILEFKIKYEKLARLDRFEVKAMHYDDGEIV | 2021Q1_ |
| ID | FDEVLVNGGKLDVEYQDEHKTLLVKIEGKEYSIKGQKIGGKQRLLENRISKSK | 2.021 |
| NO: | VQLTIKDNIQTNANGTARQKSTEREFIVPENIKLYSQIVGREITTTKEIYLTK | |
| 253 | RFLGYRSDLLFYYGFVDNFFQVADNKKELWKIDFQNSQFADYFQYMVNDNLKN | |
| | SDNYLKDYLNDSSKITDDLEKVKTVFSKLRHALLHFEYDFFEKLFNGEDVGFD | |
| | LDIGFLNLLIENIDKLNIDAKKEFIADEKIKLFGEELALSKVYALYSSICVNR | |
| | VGFNKFINSLIMVDGVENETLKSFFDDELKEKNPRLFEALGNRAYYVDIHSNR | |
| | AYKRIYNQHKELVSKSSALSDGRKIHQANQEITKLKEKMNEITKRNSLARLEH | |
| | KLRVAFGFLYGEYNDHRAFKDNEDTDIKSKKFETLNSDKSKDYFSSTYQNRKP | |
| | RTREKLEKVESLNLKTLIEDDRLLKFVLLMFLFMPQEVKGEFIGFIKKYYHDT | |
| | KGIGEDTKEKELDVVETMPLSLKLKILGNNIRSLTLFKYALSSEVKYNSSSHL | |
| | FYEEGNRHGRIYKKLGISHNQEEFN | |
| SEQ | MIKNPSNRYALPKVIISKIDNQNILEFKIKYKKLSKLDIVKVKSMHYDDRAII | 2021Q1_ |
| ID | FDEVIVNDGLIDVEYRDNHKTIFVKVGNKSYSISGQKVGGKERLLENRVSKTK | 2.022 |
| NO: | VQLELKDKATNRVSKTERELIVDDNIKIYSQIVGRDVKTTKDIYLIKRFLAYR | |
| 254 | SDLLFYYGFVNNFFHVANNRSEFWKIDFNDSNNSKLIEYFKFTINDHLKNDEN | |
| | YLKDYISDNEKLKNDLIKVKNSFEKIRHALMHFDYDFFVKLFNGEDVGLELDI | |
| | EFLDIMIDKLDKLNIDTKKEFIDDEKITIFGEELSLAKLYRFYAHTAINRVAF | |
| | NKLINSFIIENGVENQSLKEYFNQQAGGIAYEIDIHONREYKNLYNEHKKLVS | |
| | RVLSISDGQEIAILNQKIAKLKDQMKQITKANSIKRLEYKLRLALGFIYTEYE | |
| | NYEEFKNNFDTDIKNGRFTPKDNDGNKRAFDSRELEQLKGYYEATIQTOKPKT | |
| | DEKIEEVSKKIDRLSLKSLIADDILLKFILLMFTFMPQELKGEFLGFIKKYYH | |
| | DTKHIDQDTISDSDDTIETLSIGLKLKILDKNIRSLSILKHSLSFQTKYNKKD | |
| | RNYYEDGNIHGKFFKKLGISHNQEEFNKSVYAPLFRYYSALYKLINDFEIYTL | |
| | SLHIVGSETLTDQVNKSQFLSGRYFNFRKLLTQSYHINNNSTHSTIFNAVINM | |
| | RNDISHLSYEPLFDCPLNGKKSYKRKIRNQFKTINIKPLVESRKIIIDFITLQ | |
| | TDMQKVLGYDAVNDFTMKIVQLRTRLKAYANKEQTIQKMITEAKTPNDFYNIY | |
| | KVQGVEEINKYLLEVIGETQAEKEIREKIERGNIANF | |
| SEQ | MLKKPSNRYALPKVILSTVDHEKILEFKVKYEKLARLDRLVVERMHFDGESVV | 2021Q1_ |
| ID | FDEVIANSGDLEIAYQDDHRKLLIQAAGKSYTITGKKVGGKKRKLEERISRAK | 2.016 |
| NO: | IQLTLTDGQEDQHRRIRATVTEKALLEPKEDRDIYSKISDRKIKTSKEIYLVK | |
| 255 | RFLSYRSDLLFYYFFVDNFFKVGNNKQELWKIKFQNQPELIEYFRFIINDRFK | |
| | NAKNDKFDNYLKNDKAIQEDLEKIQKVFEKLRHALMHYDYGFFEKLFGGEDQG | |
| | FDLDIAFLDNFVKKIDKLNIDTKKEFVDDEKIKIFGEDLNLADLYKLYASISI | |
| | NRVGFNRVVNEMIIKDGIEKSELKRAFEKKLDKTYALDIHSDPSYKKLYNEHK | |
| | RLVTEVSTYTDGNKIKEGNQKIAKLKYEMKEITKKNALVRLECKMRLAFGLIY | |
| | GRYDTHEAFKNGFDTDLKRGEFAQIGSEEAIGYENTTFEKSKPKSKEEIKKIA | |
| | RQIDNLSLSTLIEDDPLMKFIVLMFLFVPRELKGEFLGFWRKYYHDIHSIDSD | |
| | AKSDEMPDEVSLSLKLKILTRNIRRLNLFEYSLSEKIKYSPKNTQFYTDKSPY | |
| | QKVYKRLKISHNKEEFDKTLLVPLFRYYSILFKLINDFEIYSLAKANPDASSL | |
| | SELTKTKHGFRGHYNFTTLMMDAHKVSQGDSKKHFGIRGEIAHINTKDLIYDP | |
| | LFRKSKMAQQRNDVIDFVLKYEKEIKAVLGYDAINDFRMKVVQLRTKLKVYSD | |
| | KTQTIEKLLNEVEAPDDFYVLYKVKGVEAINKYLLEIVSVTQAEEEIERKIIT | |
| | GNKRYNT | |
| SEQ | MTKKPSNRNSLPKVIINKVDESSILEFKIKYEKLARLDRFEVRSMRYDGDGRI | 2021Q1_ |
| ID | IFDEVVANAGLLDVDYEDDNRTIVVKIENKAYNIYGKKVGGEKRLNGKISKAK | 2.029 |
| NO: | VQLILTDSIRKNANDTHRHSLTERELINKNEVDLYSKIAEREISTTKDIYLVK | |
| 256 | RFLAYRSDLLLYYAFINHYVRVNGNKKEFWKTEIDDKIIDYFIYTINDTLKNK | |
| | EGYLEKYIVDRDQIKKDLEKIKQIFSHLRHKLMHYDFRFFTDLFDGKDVDIKV | |
| | DNSIQKISELLDIEFLNIVIDKLEKLNIDAKKEFIDDEKITLFGQEIELKKLY | |
| | SLYAHTSINRVAFNKLINSFLIKDGVENKELKEYFNAHNQGKESYYIDIHQNQ | |
| | EYKKLYIEHKNLVAKLSATTDGKEIAKINRELADKKEQMKQITKANSLKRLEY | |
| | KLRLAFGFIYTEYKDYERFKNSFDTDTKKKKFDAIDNAKIIEYFEATNKAKKI | |
| | EKLEEILKGIDKLSLKTLIQDDILLKFLLLFFTELPQEIKGEFLGFIKKYYHD | |
| | ITSLDEDTKDKDDEITELPRSLKLKIFSKNIRKLSILKHSLSYQIKYNKKESS | |
| | YYEAGNVFNKMFKKQAISHNLEEFGKSIYLPMLKYYSALYKLINDFEIYALYK | |
| | DMDTSETLSQQVDKQEYKRNEYFNFETLLRKKFGNDIEKVLVTYRNKIAHLDF | |
| | NFLYDKPINKFISLYKSREKIVNYIKNHDIQAVLKYDAVNDFVMKVIQLRTKL | |
| | KVYADKEQTIESMIQNTQNPNGFYNIYKVKAVENINRHLLKVIGYTESEKAVE | |
| | EKIRAGNTSKS | |
| SEQ | MEKIKKPSNRNSIPSIIISDYDANKIKEIKVKYLKLARLDKITIQDMEIVDNI | 2020Q3_ |
| ID | VEFKKILLNGVEHTIIDNQKIEFDNYEITGCIKPSNKRRDGRISQAKYVVTIT | 6.008 |
| NO: | DKYLRENEKEKRFKSTERELPNNTLLSRYKQISGFDTLTSKDIYKIKRYIDFK | |
| 257 | NEMLFYFQFIEEFFNPLLPKGKNFYDLNIEQNKDKVAKFIVYRLNDDFKNKSL | |
| | NSYITDTCMIINDFKKIQKILSDFRHALAHFDFDFIQKFFDDQLDKNKFDINT | |
| | ISLIETLLDQKEEKNYQEKNNYIDDNDILTIFDEKGSKFSKLHNFYTKISQKK | |
| | PAFNKLINSFLSQDGVPNEEFKSYLVTKKLDFFEDIHSNKEYKKIYIQHKNLV | |
| | IKKQKEESQEKPDGQKLKNYNDELQKLKDEMNTITKONSLNRLEVKLRLAFGF | |
| | IANEYNYNFKNFNDEFTNDVKNEQKIKAFKNSSNEKLKEYFESTFIEKRFFHF | |
| | SVNFFNKKTKKEETKQKNIFNSIENETLEELVKESPLLQIITLLYLFIPRELQ | |
| | GEFVGFILKIYHHTKNITSDTKEDEISIEDAQNSFSLKFKILAKNLRGLQLFH | |
| | YSLSHNTLYNNKQCFFYEKGNRWQSVYKSFQISHNQDEFDIHLVIPVIKYYIN | |
| | LNKLMGDFEIYALLKYADKNSITVKLSDITSRDDLKYNGHYNFATLLFKTFGI | |
| | DTNYKQNKVSIQNIKKTRNNLAHQNIENMLKAFENSEIFAQREEIVNYLQTEH | |
| | RMQEVLHYNPINDFTMKTVQYLKSLSVHSQKEGKIADIHKKESLVPNDYYLIY | |
| | KLKAIELLKQKVIEVIGESEDEKKIKNAIAKEEQIKKGNN | |
| SEQ | MMTKKPANRHALPKVIISEVDNTNILEFKIKYEKLARLDRVEVKAMHYEDGRI | 2020Q3_ |
| ID | IFDEVVVNGGLIEVEYQDDHKTLFVQVGEKSYSISGQKVGGKQRLLEDRVSKT | 6.001 |
| NO: | KVQLELSDGSSERVSRTERELIVADNIKLYSQIVGHEVKTTKEIYLAKRFLGY | |
| 258 | RSDLLFYYGFVDNFFRESKNLKYGKQPVELWEDKFQVNDKLTAYTKFMFNDDL | |
| | QNSESYLKEYVKDNHKIKNDLESARDIFATFRHNLMHFNYSFFTRLFNGEDVK | |
| | IKNLQTKKFESLSDVLRNVEFLNKVIQSIDKLNIDTRKEFIDKEKITLFNEEL | |
| | DLQQLYGFFAYTAINRVAFNKLINSFIIKDGIENEQLKEYFNQRVDGTAYEID | |
| | IHQNREYKELYKKHKNLVSKVSTLSDGKEIARGNTEISVLKEQMNKITKANSL | |
| | KRLEHKLRLAFGFIYTEYGSYKAFVSRFNEDTKRKKIKNVEFEKIGVEKQKEY | |
| | YESTFTSNNKDKLGELIQEYEKLSLNDLIENDTFLKVILLLFIFMPKEVKGDF | |
| | LGFIKKYYHDTKHIEEDTKEKDEGFTNTLPIGLKLKIVERNIAKLSVLKHSLS | |
| | LKVKYNRGQYEEDNTYRKVFKKLNISHNQEEFHKSMFSPLLRYYASLYKLIND | |
| | FEIYTLSHYITDKYSTLNKVIASEQFHYRYGWNREEKKGELVKTDNYTFSTLL | |
| | SKKYGHKNSQEISEMRNKISHFDEKILFKFPLEEVSSVPKGKGKYKKDEPIKS | |
| | LKEKREEIVSLMEKQTDMQKVLGYDAINDFRMKTVQFQTKLKVYSNKEETIKK | |
| | MIVEAKTPNDYYNIYKVKGVEGINEHLLNVIGETEAEKSIQEQIAEGNKVNV | |
| SEQ | ELCKIDFTDARSSSLIEYFKFAINDNLKNDRGYLKAYVNDVEQIRADLKKVGG | 2021Q1_ |
| ID | KQRNLEDRVSRTKVQLTLTNHIEDREGKQRVSRTERELIVPQNIKLYSQIVGR | 2.026 |
| NO: | EVKTTKEIYLIKRFLEYRSDLLFYYGFVDNFFKVEGNKKELCKIDFTDARSSS | |
| 259 | LIEYFKFAINDNLKNDRGYLKAYVNDVEQIRTDLQKVKTIFSKLRHALMHFDY | |
| | DFFEKLFNGEEVGFDFDIKFLNIMIDKVEKLNIETKKEFIEDEVITLFGERLS | |
| | LKKLYGLFSHIAINRVAFNKFINSFLIKDGIENRALKDFFNDEKGSQAYEIDI | |
| | HSNAEYKALYVQHKKLVMATSAMSDGNEIAKKNQEISELKEKMNAITKANSLA | |
| | RLEYKLRLAFGFIYTEYGDYTAFKNSFDRDVKSAKYKELSVERLKAYYLATFK | |
| | ASKPQSHEKLEEVAKKIDRLSLKQLIENETLLKFVLLLFTFMPQELKGEFLGF | |
| | IKKYYHDKKHIEQDTKEKEEEREGLSTGLKLKVLEKNIRSLSILKHALSFQVK | |
| | YNKKDKNFYEEGNLHGKFYKKLAISHNQEEFNKSVYAPLFRYYVALYKLINDF | |
| | EIYSLAQHIVNNETLADQVGKAQFRQRGYFNFRKLVNCTYATAQNSSYNVLIF | |
| | MRNDISHLSYEPLFNCPLEEKASYKQKIRGREKIISVKPLSESRAEIVRFIAS | |
| | QTDMKKLLGYDAVNDFNMKMVQLRRRLSVYANKQETIEKMINKAKTPNDFYNL | |
| | YKLKGIECINQHLLKVIGVTEAEKRIEKQIEEGNEKY | |
| SEQ | MLKHKRKNKNSLARVVLSNYDSNNIYEIKIKYEKLAKLDKINIIEMDYDADNN | 2021Q1_ |
| ID | VMFKKVLFNNKEIDLSHKDKTKINIELDNKKYNISAKKQIGKTHLVVRNKQTS | 2.017 |
| NO: | KISRIKKIQDTYYRGKDVFILDNNIEILDKKQTKDKFIVTLNDITNNKTTSTE | |
| 260 | AELIDDTKDIFKKISAKKDLKSSDIYKIKRFISIRSNFSFYYTFVDNYFKIFH | |
| | AKKDKNKEELYKIKFKDEINIKPYLENILDNMKNKNGILYNYANDRKKVLNDL | |
| | RNIQYVFKEFRHKLAHFDYNFLDNFFSNSVEEKYKQKVNEIKLLDILLDNIDS | |
| | LNVVPKQNYIEDETISVFDAKDIKLKRLYTYYIKLTINYPGFKKLINSFFIQD | |
| | GIENQELKEYINNKEKDTQVLKELDNKAYYMDISQYRKYKNIYNKHKELVSEK | |
| | ELSSDGKKINSLNQKINKLKIDMKNITKPNALNRLIYRLRVAFGFIYKEYATI | |
| | NNFNKSFLQDTKTKRFENISQQDIKSYLDISYQDKGKFFVKSKKTFKNKTTVK | |
| | YTFEDLDLTLNEIITQDDIFVKVIFLFSIFMPKELNGDFFGFINMYYHKMKNI | |
| | SYDTKDIDMLDTISQNMKLKILEQNIKKTYVFKYYLDLDSSIYSKLVQNIKIT | |
| | EDIDSKKYLYAKIFKYYQHLYKLISDVEIYLLYKYNSKENLSITIDKDELKHR | |
| | GYYNFQSLLIKNNINKDDAYWSIVNMRNNLSHQNIDELVGHFCKGCLRKSTTD | |
| | IAELWLRKDILTITNEIINKIESFKDIKITLGYDCVNDFTQKVKQYKQKLKAS | |
| | NERLAKKIEEKQNQVVDEKNKEELEKNILNMKNIQKINRYILDIL | |
| SEQ | MLKHKRKNKNSLARVVLSNYDSNNIYEIKIKYEKLAKLDKINIIEMDYDADNN | 2021Q1_ |
| ID | VMFKKVLFNNKEIDLSHKDKTKINIELDNKKYNISAKKQIGKTHLVVRDKQTS | 2.002 |
| NO: | KISRIKKIQDTYYRGKDVFILDNNIEILDKKQTKDKFIVTLNDITNDKTTSTE | |
| 261 | AELIDDTKDIFKKISAKKDLKSSDIYKIKRFISIRSNFSFYYTFVDNYFKIFH | |
| | AKKDKNKEELYKIKFKDEINIKPYLENILDNMKNKNGILYDYADDREKVLNDL | |
| | KNIQYVFTEFRHKLAHFDYNFLDNFFSNSVTDQYKQKVNEIKLLDILLDNIDS | |
| | LNVVPKQNYIEDETISVFDAKDIKLKRLYTYYIKLTINYPGFKKLINSFFIQD | |
| | GIENQELKEYINNKEKDTQVLKELDNKAYYMDISQYRKYKNIYNKHKELVSEK | |
| | ELSSDGQKINSLNQKINKLKIEMKNITKPNALNRLIYRLRVAFGFIYKEYATI | |
| | NNFNKSFLQDTKIKRFENISQQDIKNYLDISYQDKGKFFVKSKKTFKNKTTIK | |
| | YTFEDLDLTLNEIITQDDIFVKVIFLFSIFMPKELNGDFFGFINMYYHKMKNI | |
| | SYDTKDIDMLDTISQNMKLKILEQNIKKTYVFKYYLDLDSSIYSKLVQNIKIT | |
| | EDIDSKKYLYAKIFKYYQHLYKLISDVEIYLLYKYNSKENLSITIDKDELKHR | |
| | GYYNFQSLLIKNNINKDDAYWSIVNMRNNLSHQNIDELVGHFCKGCLRKSTTD | |
| | IAELWLRKDILTITNEIINKIESFKDIKITLGYDCVNDFTQKVKQYKQKLKAS | |
| | NERLAKKIEEKQNQVVDEKNKEELEKKILNMKNIQKINRYILDIL | |
| SEQ | IQKFFDNGLDATKYDISTISLLKTLLEKTEEKIYHEKNNYIEDTDTLSIFDEK | 2021Q1_ |
| ID | EIGFSKLHNFYTKISQKKPAFNKLINSFLSKDGVPNEPFKAYLHGKGYDYFED | 2.014 |
| NO: | IHADKSYKAIYVQHKLLVAQKQKEEAEEKPDGYKLKAYNDKLQELKIQMESIT | |
| 262 | KANSLKRLEVKMRLAFGFITNEYKYDFKKFNAEFTLDVKTKEKLDRFKATSDE | |
| | RLQHYFESTFEEKTFFHFTVSSYDKKQKKSVEKVKTIFNLVENETLQTLVQDS | |
| | PLLQIITLLYLFIPKELQGDFIGFILRIYHQIKNITSDTKEDEISIEESQNSF | |
| | ALKLKVLAKSLRGLQLFNYSLSHETLYNKNEHFFYEKGNRWKNIYKALGISHN | |
| | TEEFDIHLVAPIIKYHINLYKLIGDFEIYALLTFAKKSRSHETLSVISKSDAL | |
| | KFKENYNFSTLLSKAFRIDVNNKNNPPYIQTLKQIRNNISHQNIEKMMTAFEQ | |
| | NDISKQRKEIIIYLQTDHQEMQKLLHYNPVNDFTMKTVQYRIMLDKYKTGMAD | |
| | NDERIENRADLIIKQLKKETPNDYYLIYKLKAIELLKQKMIEAIGETEQEKKI | |
| | RKAIAK | |

### 5. Fusion Proteins

In some examples, a CRISPR protein with wild type cleavage activity, or a variant thereof, is fused (conjugated) to a heterologous polypeptide (i.e., one or more heterologous polypeptides) that has an activity of interest to form a fusion protein. The heterologous polypeptide may be referred to as a “fusion partner.” In some embodiments, the fusion protein comprises a Cas protein, such as a Cast protein, fused to a heterologous sequence by a linker. The fusion partner can be fused to the C-terminus, N-terminus, or an internal portion (e.g., a portion other than the N- or C-terminus) of the CRISPR protein.

In some examples, the fusion partner is fused to the programmable nuclease by a linker. A linker can be a peptide linker or a non-peptide linker. In some examples, the linker is an XTEN linker. In some examples, the linker comprises one or more repeats a tri-peptide GGS. In some examples, the linker is from 1 to 100 amino acids in length. In some examples, the linker is more 100 amino acids in length. In some examples, the linker is from 10 to 27 amino acids in length. A non-peptide linker can be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some examples, the CRISPR complex comprises an enzymatically inactive and/or “dead” (abbreviated by “d”) programmable nuclease in combination (e.g., fusion) with a fusion partner. Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g., a programmable nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type CasΦ activity).

In some cases, a fusion protein comprises a heterologous polypeptide that has enzymatic activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, or glycosylase activity). Examples of enzymatic activity that can be provided by the fusion partner may comprise: nuclease activity such as that provided by a restriction enzyme (e.g., FokI nuclease) or DNA damage activity. In some cases, the fusion protein has enzymatic activity that cleaves nucleic acids (e.g., ssRNA, dsRNA, ssDNA, dsDNA) in a host cell comprising the CRISPR protein. In some examples, the fusion partner comprises any domain capable of interacting with ssDNA or ssRNA. In some examples, the fusion partner comprises an endonuclease (e.g., a restriction endonuclease) or a protein or protein domain responsible capable of stimulating DNA or RNA cleavage. In some examples, the fusion partner comprises a restriction enzyme. In some examples, the CRISPR complex comprises an enzymatically inactive CRISPR protein fused to a heterologous polypeptide with nuclease activity. In some examples, the heterologous polypeptide is a restriction enzyme. The restriction enzyme may be HincII. In some examples, the restriction enzyme may be AluI, BamHI, EcoP15I, EcoRI, EcoRII, EcoRV, HaeIII, HgaI, HindII, HindIII, HinFI, KpnI, NotI, PstI, PvuII, SacI, SaII, Sau3, ScaI, SmaI, SpeI, SphI, StuI, TaqI, or XbaI. In some examples, hybridization of the CRISPR protein to the nucleic acid target site induces a conformational change in the CRISPR protein, and the conformational change releases the restriction enzyme. In some examples, the released restriction enzyme cleaves target RNA or DNA in a host cell and induces cell death, cell cycle arrest, apoptosis, or a combination thereof in the host cell.

### B. Guide RNA

In some examples, CRISPR complexes described herein can comprise one or more guide nucleic acid molecules. In some examples, a guide nucleic acid molecule is a nucleic acid molecule that binds to a CRISPR-associated protein described herein, forming a ribonucleoprotein complex (RNP), and targets the complex to a specific location within a target nucleic acid (e.g., DNA, RNA). In some examples, a guide nucleic acid molecule comprises two segments, a targeting segment and a protein-binding segment. The targeting segment of a guide RNA (in some instances, referred to as a “spacer”) may include a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). In some examples, the guide sequence comprises a tracrRNA hybridized to a crRNA which includes a guide sequence that hybridizes to a nucleic acid target site. In some examples, the guide nucleic acid molecule does not comprise a tracrRNA.

In some examples, the guide nucleic acid molecule binds to a nucleic acid target site or portion thereof. For example, the guide nucleic acid can bind to a nucleic acid target site described herein, such as a portion of a viral genome or a gene comprising a mutation unique to a population of cancer cells. The guide nucleic acid may also bind to a DNA molecule associated with an autoimmune disease. In some examples, the guide nucleic acid comprises a segment of nucleic acids that are reverse complementary to the nucleic acid target site. Often the guide nucleic acid binds specifically to the nucleic acid target site. In some examples, the nucleic acid target site comprises a single-stranded DNA or DNA amplicon of a nucleic acid of interest. In some examples, the nucleic acid target site comprises a double-stranded DNA or DNA amplicon of a nucleic acid of interest. In some examples, the nucleic acid target site comprises a single-stranded RNA or RNA amplicon of a nucleic acid of interest. In some examples, the nucleic acid target site comprises a double-stranded RNA or RNA amplicon of a nucleic acid of interest. A guide nucleic acid can comprise RNA, DNA, or a combination thereof. A guide nucleic acid may be a non-naturally occurring guide nucleic acid. A non-naturally occurring guide nucleic acid may comprise an engineered sequence having a repeat and a spacer that hybridizes to the nucleic acid target site. A non-naturally occurring guide nucleic acid may be recombinantly expressed or chemically synthesized. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids.

In some cases, the segment of a guide nucleic acid that comprises a sequence that is reverse complementary to the nuclease acid target site is 20 nucleotides in length. A guide nucleic acid can have at least 1, 5, 10, 11, 12, 13, 14, 15, 16, 17, 18, 19, 20, 21, 22, 23, 24, 25, 26, 27, 28, 29, or 30 nucleotides reverse complementary to a target nucleic acid (used interchangeably with “nucleic acid target site” herein). In some cases, the guide nucleic acid can be 1, 5, 10, 11, 12, 13, 14, 15, 16, 17, 18, 19, 20, 21, 22, 23, 24, 25, 26, 27, 28, 29, or 30 nucleotides in length. For example, a guide nucleic acid may be at least 10 bases. In some embodiments, a guide nucleic acid may be from 10 to 50 bases. In some embodiments, a guide nucleic acid may be at least 25 bases. In some cases, the guide nucleic acid has from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt reverse complementary to a target nucleic acid. In some cases, the guide nucleic acid has from about 10 nt to about 60 nt, from about 20 nt to about 50 nt, or from about 30 nt to about 40 nt reverse complementary to a target nucleic acid. It is understood that the sequence of a guide nucleic acid need not be 100% reverse complementary to that of its target nucleic acid to be specifically hybridizable, hybridizable, or bind specifically. The guide nucleic acid can have a sequence comprising at least one uracil in a region from nucleic acid residue 5 to 20 that is reverse complementary to a modification variable region in the nucleic acid target site. The guide nucleic acid, in some cases, has a sequence comprising at least one uracil in a region from nucleic acid residue 5 to 9, 10 to 14, or 15 to 20 that is reverse complementary to a modification variable region in the nucleic acid target site. The guide nucleic acid can have a sequence comprising at least one uracil in a region from nucleic acid residue 5 to 20 that is reverse complementary to a methylation variable region in the nucleic acid target site. The guide nucleic acid, in some cases, has a sequence comprising at least one uracil in a region from nucleic acid residue 5 to 9, 10 to 14, or 15 to 20 that is reverse complementary to a methylation variable region in the nucleic acid target site. The guide nucleic acid can hybridize with a nucleic acid target site.

In some examples, the guide sequence has 80% or more (e.g., 85% or more, 90% or more, 95% or more, or 100%) complementarity with the nucleic acid target site. In some cases, the guide sequence is 100% complementary to the nucleic acid target site. In some cases, the nucleic acid target site includes at least 15 nucleotides (nt) of complementarity with the guide sequence of the guide RNA.

A programmable nuclease of the present disclosure may be activated to exhibit cleavage activity (e.g., cis-cleavage of a target nucleic acid or trans-cleavage of a collateral nucleic acid) upon binding of a ribonucleoprotein (RNP) complex to a nucleic acid target site, in which the spacer of the crRNA of the gRNA hybridizes to the target nucleic acid.

In some examples, a CRISPR protein disclosed herein (e.g., a Type V or Type VI CRISPR/Cas effector protein) can cleave a precursor guide RNA into a mature guide RNA, e.g., by endoribonucleolytic cleavage of the precursor. In some examples, a CRISPR protein can cleave a precursor guide RNA array (that includes more than one guide RNA arrayed in tandem) into two or more individual guide RNAs. Thus, in some cases a precursor guide RNA array comprises two or more (e.g., 3 or more, 4 or more, 5 or more, 2, 3, 4, or 5) guide RNAs (e.g., arrayed in tandem as precursor molecules). In other words, in some cases, two or more guide RNAs can be present on an array (a precursor guide RNA array). In some examples a CRISPR protein can cleave the precursor guide RNA array into individual guide RNAs

In some cases, a subject guide RNA array includes 2 or more guide RNAs (e.g., 3 or more, 4 or more, 5 or more, 6 or more, or 7 or more guide RNAs). The guide RNAs of a given array can target (i.e., can include guide sequences that hybridize to) different target nucleic acid sites associated with a disease or condition (e.g., single nucleotide polymorphisms (SNPs), different strains of a particular virus, etc.), and as such could be used, for example, to target multiple strains of a virus, multiple viral genes, multiple cancer associated mutations, or multiple target sequences associated with an autoimmune disorder. In some cases, each guide RNA of a precursor guide RNA array has a different guide sequence. In some cases, two or more guide RNAs of a precursor guide RNA array have the same guide sequence.

In some instances, the precursor guide RNA array comprises two or more guide RNAs that target different target sites within the same target DNA molecule. For example, such a scenario can in some cases increase sensitivity of detection by activating a CRISPR protein when either one hybridizes to the target DNA molecule. As such, in some cases, a subject composition (e.g., kit) or method includes two or more guide RNAs (in the context of a precursor guide RNA array, or not in the context of a precursor guide RNA array, e.g., the guide RNAs can be mature guide RNAs).

In some cases, the precursor guide RNA array comprises two or more guide RNAs that target different target DNA molecules. Such an array may be useful for targeting any one of a number of different species, strains, isolates, or variants of a bacterium (e.g., different species, strains, isolates, or variants of Mycobacterium, different species, strains, isolates, or variants of Neisseria, different species, strains, isolates, or variants of Staphylococcus aureus; different species, strains, isolates, or variants of E. coli; etc.). As such, in some cases as subject composition (e.g., kit) or method includes two or more guide RNAs (in the context of a precursor guide RNA array, or not in the context of a precursor guide RNA array, e.g., the guide RNAs can be mature guide RNAs).

### Guide RNA Chemical Modifications

In some embodiments, a guide RNA comprises one or more chemical modifications. Non-limiting examples of chemical modifications include a nucleobase modification and a backbone modification. Chemical modification may provide the nucleic acid with a new or enhanced feature, e.g., improved stability or increased activity. In general, a guide RNA comprising one or more chemical modifications is synthesized to comprise the one or more chemical modifications and thus, it is not naturally occurring.

Exemplary nucleic acid modifications include but are not limited to: 2′ O-methyl modified nucleotides, 2′-fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). The phosphorothioate (PS) bond (i.e., a phosphorothioate linkage) substitutes a sulfur atom for a non-bridging oxygen in the phosphate backbone of a nucleic acid (e.g., an oligo). The normal linkage or backbone of RNA and DNA is a 3′ to 5′ phosphodiester linkage. This modification may render the guide RNA more resistant to nuclease degradation relative to a guide RNA with the same sequence but without the PS linkage. In some instances, PS linkages occur between any of the 5′-most and 3′-most 3-5 nucleotides of the guide RNA.

In some embodiments, a subject nucleic acid has one or more nucleotides that are 2′ O-methyl modified nucleotides. In some instances, the 2′ O-methyl occur on any of the 5′-most and 3′ most 3-5 nucleotides of the guide RNA. In some embodiments, the guide RNA comprises one or more 2′-fluoro modified nucleotides. In some embodiments, the guide RNA comprises one or more LNA bases. In some embodiments, the guide RNA comprises a 5′ cap (e.g., a 7-methylguanylate cap (m7G)). In some embodiments, a guide RNA (e.g., a dsRNA, a siNA, etc.) comprises a combination of modified nucleotides.

Guide RNAs may include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE). A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂. Other suitable sugar substituent groups include methoxy (—O—CH₃), aminopropoxy (—O CH₂ CH₂ CH₂NH₂), allyl (—CH₂—CH═CH₂), —O-allyl CH₂—CH═CH₂) and fluoro (F).

In some instances, the guide RNA comprises a chemical modification of its 5′-most nucleotide. In some instances, the guide RNA comprises a chemical modification of its 3′-most nucleotide. In some instances, the guide RNA comprises a chemical modification of its 5′-most nucleotide and its 3′-most nucleotide. In some instances, the guide RNA comprises chemical modifications of its 1, 2, 3 or 4 5′-most nucleotides. In some instances, the guide RNA comprises chemical modifications of its 1, 2, 3 or 4 3′-most nucleotides. In some instances, the guide RNA comprises chemical modification of its 1, 2, 3, or 4 5′-most nucleotides and its 1, 2, 3 or 4 3′-most nucleotides. In some instances, at least one of the chemical modifications is a 2′ O-methyl modification. In some instances, all of the chemical modifications are 2′ O-methyl modifications.

In some instances, the guide RNA comprises a phosphorothioate linkage between its two 5′-most nucleotides. In some instances, the guide RNA comprises a phosphorothioate linkage between its two 3′-most nucleotides. In some instances, the guide RNA comprises a phosphorothioate linkage between its two 5′-most nucleotides, and a second phosphorothioate linkage between its two 3′-most nucleotides. In some instances, the guide RNA comprises phosphorothioate linkages between its 1, 2, 3 or 4 of its 5′-most nucleotides. In some instances, the guide RNA comprises phosphorothioate linkages between 1, 2, 3 or 4 of its 3′-most nucleotides. In some instances, the guide RNA comprises phosphorothioate linkages between 1, 2, 3, or 4 of its 5′-most nucleotides and between 1, 2, 3, or 4 of its 3′-most nucleotides.

### Base Modifications and Substitutions

In some embodiments, guide RNAs comprise a nucleobase modification. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one) and phenothiazine cytidine (1H-pyrimido(5,4-b)(1,4)benzothiazin-2(3H)-one); and G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), and pyridoindole cytidine (H-pyrido(3′,2′:4,5)pyrrolo(2,3-d)pyrimidin-2-one).

### C. Multiplexing

In some examples, CRISPR complexes described herein can be multiplexed in a number of ways. Multiplexing can comprise targeting multiple different target nucleic acids at the same time. In some examples, the multiple target nucleic acids are targeted using the same programmable nuclease, but different guide nucleic acids. In some examples, at least two different programmable nucleases are used in single reaction multiplexing.

In some examples, CRISPR systems described herein comprise multiple guide RNAS that each specifically target different DNA or RNA molecules. For example, in some instances, CRISPR systems described herein comprise about or more than about 1, 2, 3, 4, 5, 6, 7, 8, 9, 10, 15, 20, 30, 40 or more guide nucleic acid molecules directed to separate nucleic acid target sites. In some cases, the multiple nucleic acid target sites comprise different nucleic acid target sites associated with a disease or condition described herein. In some instances, the multiple nucleic acid target sites comprise multiple nucleic acid target sites associated with a virus, autoimmune disorder, or cancer described herein. For example, in some instances, the multiple nucleic acid target sites comprise different target nucleic acids to a virus, e.g., influenza. In some instances, the multiple nucleic acid target sites comprise different target nucleic acids associated within two different diseases or conditions described herein. For example, in some cases, the multiple nucleic acid target sites comprise nucleic acid target sites associated with influenza and another disease (e.g., sepsis or a respiratory infection, such as an upper respiratory tract virus). In some cases, the multiple nucleic acid target sites comprise target nucleic acids directed to different viruses, bacteria, or pathogens responsible for more than one disease. In some cases, multiplexing allows for discrimination between multiple nucleic acids, such as nucleic acids that comprise different genotypes of the same bacteria or pathogen responsible for a disease, for example, for a wild-type genotype of a bacteria or pathogen. In some cases, multiplexing allows for discrimination between nucleic acids comprising different mutations responsible for a cancer described herein or acting as different biomarkers for an autoimmune disease described herein.

### D. Compositions & Methods for Selective Modification

Disclosed herein, in some aspects, are compositions comprising a CRISPR-associated protein and a guide nucleic acid molecule, wherein the guide nucleic acid molecule comprises a nucleotide sequence that is identical or reverse complementary to an equal length portion of a target nucleic acid that comprises a mutation of at least one nucleotide relative to a corresponding wildtype sequence. The nucleotide sequence that is identical or reverse complementary to the equal length portion of the target nucleic acid may comprise 4, 5, 6, 7, 8, 9, 10, 11, 12, 13, 14, 15, 16, 17, 18, 19 or 20 or more nucleobases. In some instances, the CRISPR-associated protein is a CRISPR associated protein described herein. In some instances, the CRISPR-associated protein is a Cas12, Cas13, Cas14, or CasΦ protein. In some instances, the CRISPR-associated protein is a catalytically active fragment of a Cas12, Cas13, Cas14, or CasΦ protein. In some instances, the CRISPR-associated protein comprises a Cas12a, Cas12b, Cas12c, Cas12d, or Cas12e protein; a Cas13a, Cas13b, Cas13c, Cas13d, or Cas13e protein; or a Cas14a, Cas14b, Cas14c, Cas14d, Cas14e, Cas14f, Cas14g, Cas14h, Cas14i, Cas14j, or Cas14k protein. In some instances, the CRISPR-associated protein comprises an amino acid sequence that is at least 75%, at least 80%, at least 85%, at least 90%, at least 95% or 100% identical to any one of SEQ ID NOs: 1-220, 244, and 248-262. In some instances, the amino acid sequence of the CRISPR-associated protein is at least 75%, at least 80%, at least 85%, at least 90%, at least 95% or 100% identical to any one of SEQ ID NOs: 1-220, 244, and 248-262. Mutations include, but are not limited to one or more nucleotide deletions, one or more nucleotide insertions, one or more nucleotide substitutions, or a combination thereof, relative to a wildtype sequence. In some instances, the mutation is a single nucleotide polymorphism (SNP). In some instances, the mutation is located in a gene associated with cancer. Genes associated with cancer include, but are not limited to, RB1, KRAS, TP53, CDKN2A, EGFR, BRCA1, BRCA2, and HER2. In some instances, the mutation is located in an oncogene. Non-limiting examples of oncogenes are NRAS, TP53, BRAF, MYC, CTNNB1, CREBBP, EGFR, RB1, PTEN, and JAK1. In some instances, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20.

In some instances, these compositions are useful for methods of modifying a target nucleic acid in a cell. In some instances, methods selectively modify a portion of cells within a population of cells, wherein the portion of cells comprises the target nucleic acid that comprises a mutation, and the remaining cells comprise a corresponding wildtype sequence. In some instances, the methods reduce cell viability, reduce cell proliferation, or increase cell death of the cell or the portion of cells. In some instances, the methods induce cell death, cell cycle arrest, or apoptosis of the cell or the portion of cells. In some instances, methods modify the nucleotide sequence of the target nucleic acid. In some instances, methods increase expression of the target nucleic acid relative to the same cells that have not been modified. In other instances, methods reduce expression of the target nucleic acid relative to the same cells that have not been modified.

In some instances, compositions or methods reduce cell viability of a portion of cells in a cell population by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, wherein cell viability of the remaining cells is reduced by no more than 50%, no more than 40%, no more than 30%, no more than 20%, or no more than 10%, as measured with a cell viability assay. A non-limiting example of a cell viability assay is an MTS assay. An MTS assay is described in Example 10.

In some instances, compositions or methods reduce proliferation of a portion of cells in a cell population by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, wherein cell viability of the remaining cells is reduced by no more than 50%, no more than 40%, no more than 30%, no more than 20%, or no more than 10%, as measured with a cell proliferation assay. A non-limiting example of a proliferation assay is a colony forming assay. A colony forming assay is described in Example 10.

### KRAS

The Kirsten rat sarcoma virus (KRAS) gene is mutated in more than 90% of pancreatic cancers and more than 30% of colon and lung cancers. A sequence representing a human wildtype allele of KRAS may be found in the NCBI database with gene accession ID is NC_000012.12. A sequence representing human wildtype KRAS mRNA (also a sense strand of human KRAS cDNA) may be found in the NCBI database with accession number NM_001369786 (SEQ ID NO: 221). KRAS is a GTPase that is involved in checkpoints for cell proliferation. Mutant forms of KRAS may GTP and not GDP, leading to uninhibited proliferation of cells and accumulation of mutations. In some instances, a mutant KRAS allele comprises a mutation in exon 2. In some instances, the KRAS allele comprises a single nucleotide polymorphism. Common mutations in KRAS include, but are not limited, to KRAS p.G12C—c.34G>T; KRAS p.G12D—c.35G>A; and KRAS p.G12V—c.35G>T.

In some instances, compositions and methods disclosed herein are useful for modifying a KRAS allele, as demonstrated in Example 10. In some instances, compositions and methods modify a first allele of a KRAS gene (e.g., a mutant allele), and do not modify a second allele of a KRAS gene (e.g., a wildtype allele). Such compositions and methods are particularly useful for targeting KRAS mutants because many KRAS mutants are not easily targeted with small molecules due to their lack of drug binding pockets. In some instances, the compositions are administered with a therapeutic agent that targets other oncogenes or tumor suppressor genes or the products thereof, e.g., TP53, SMAD4, ZAC1 (also known as PLAGL1), APC, BRCA1, BRCA2, CDKN2A, DCC, DPC4, MADR2, MEN1, CDKN2A, NF1, NF2, PTEN, VHL, WRN, WT1 and RB1. In some instances, the compositions and methods are useful for treating cancer. In some instances, the cancer is pancreatic cancer. In some instances, the cancer is colon cancer or lung cancer. The compositions and methods may be used to selectively reduce the growth, reduce the viability, induce cell death or arrest the cell cycle of a portion of cells in a population of cells, wherein the portion of cells comprises a mutant KRAS allele and the remainder of the population does not comprise a mutant KRAS allele.

In some instances, compositions reduce expression of a first allele of a KRAS gene (e.g., a mutant allele), and do not reduce expression of a second allele of a KRAS gene (e.g., a wildtype allele). In some instances, compositions reduce expression of a mutant allele of a KRAS gene in a cell by at least 10%, at least 20%, at least 30%, at least 40%, at least 50%, at least 60%, at least 70%, at least 80%, at least 90%, at least 95%, or at least 99%, relative to expression of the first allele in a cell that has not been contacted with the composition. In some instances, compositions and methods do not reduce expression of a wildtype allele of a KRAS gene in a cell by more than 10%, more than 20%, more than 30%, more than 40%, or more than 50% relative to expression of the wildtype allele in a cell that has not been contacted with the composition. In some instances, compositions abolish expression of a mutant KRAS allele and do not abolish expression of a wildtype allele.

In some instances, the compositions and methods useful for targeting a mutant KRAS allele comprise a CRISPR-associated protein described herein or a use thereof. In some instances, the CRISPR-associated protein comprises a CasΦ protein or a use thereof. In some instances, the CRISPR-associated protein comprises a Cas13 or a use thereof. In some instances, the guide nucleic acid is identical or reverse complementary to a portion of a KRAS allele that comprises the 34th or 35th nucleotide of the protein coding sequence of human KRAS, and wherein the guide nucleic acid comprises an adenosine or thymine/uracil at a position that base pairs with the 34th or 35th nucleotide. In some instances, the guide nucleic acid comprises 11, 12, 13, 14, 15, 16, 17, 18, 19, or 20 contiguous nucleotides that are identical or reverse complementary to GTTGGAGCTGATGGCGTAGGC (SEQ ID NO: 245) or GTTGGAGCTGTTGGCGTAGGC (SEQ ID NO: 247). In some instances, the length of the guide nucleic acid is 17 to 25 linked nucleotides. In some instances, effector protein is a CasΦ protein and the guide nucleic acid comprises at least 20 contiguous nucleobases of the nucleotide sequence CUUUCAAGACUAAUAGAUUGCUCCUUACGAGGAGAC (SEQ ID NO: 224). In some instances, the guide nucleic acid binds to a portion of the KRAS gene, wherein the 5′ end of the portion of the gene is less than 10, less than 8, less than 7, less than 6, less than 5, less than 4, less than 3, or less than 2 nucleotides away from at least one end of a protospacer adjacent motif (PAM) of TTN (SEQ ID NO: 225), wherein N is any amino acid.

In some instances, the KRAS mutation is KRAS p.G12D—c.35G>A, and the guide nucleic acid comprises a nucleotide sequence selected from UGGUAGUUGGAGCUGAU (SEQ ID NO: 226), GAGCUGAUGGCGUAGGC (SEQ ID NO: 227), and CCUACGCCAUCAGCUCC (SEQ ID NO: 228).

In some instances, the KRAS mutation is KRAS p.G12V—c.35G>T, and the guide nucleic acid comprises a nucleotide sequence selected from TGGTAGTTGGAGCTGTT (SEQ ID NO: 229), GAGCTGTTGGCGTAGGC (SEQ ID NO: 230), and CCTACGCCAACAGCTCC (SEQ ID NO:231).

In some instances, the KRAS mutation is KRAS p.G12C—c.34G>T, and the guide nucleic acid comprises a nucleotide sequence selected from TGGTAGTTGGAGCTTGT (SEQ ID NO: 232), GAGCTTGTGGCGTAGGC (SEQ ID NO: 233), and CCTACGCCACAAGCTCC (SEQ ID NO: 234).

### E. Disease Cell Populations and Target Nucleic Acids

Methods described herein comprising inducing cell death, cell cycle arrest, or apoptosis in a population of cells by administering a CRISPR-associated protein to the population of cells. In some examples, the CRISPR-associated protein induces cell cycle arrest, apoptosis, or cell death in 50% of the cells of the cell population as determined by an in vitro assay. In some examples, the assay is an assay that measures cell viability, proliferation, apoptosis, and/or cell cycle and DNA damage. In some examples, assay is a dye exclusion assay (e.g., a trypan blue assay, eosin assay, Congo red assay, or an erythrosine B assay), a colorimetric assay (e.g., an MTT assay, MTS assay, XTT assay, WST-1 assay, WST-8 assay, LDH assay, SRB assay, NRU assay or crystal violet assay), a fluorometric assay (e.g., an alamarBlue assay or CFDA-AM assay), or a luminometric assay (e.g., an ATP assay or a real-time viability assay). In some examples, the assay is a QPCR DNA damage assay. In some examples, the CRISPR associated protein induces cell death, cell cycle arrest, apoptosis, or a combination thereof in at least 10%, at least 20%, at least 30%, at least 40%, at least 50%, at least 60%, at least 70%, at least 80%, at least 90%, at least 95%, at least 98%, at least 99%, or 100% of the cell population. In some examples, the CRISPR associated protein induces cell death, cell cycle arrest, apoptosis, or a combination thereof in at least about 1×10¹ cells, 1×10² cells, 1×10³ cells, 1×10⁴ cells, 1×10⁵ cells, 1×10⁶ cells, 1×10⁷ cells, 1×10⁸ cells, 1×10⁹ cells, or 1×10¹⁰ cells in the cell population.

In some examples, the population of cells is a disease cell population. In some examples, the disease cell population comprises a cancer cell population, a diseased autoimmune cell population, or an infectious disease cell population. In some examples, the disease cell population comprises a cancer cell population. In some examples, the disease cell population comprises a cell population associated with an autoimmune disorder (e.g., a population of cells causative of the disorder). In some examples, the disease cell population comprises an infectious disease cell population (e.g., a population of cells infected with an infectious agent or an infectious cell). In some examples, the cell population comprises mammalian cells, human cells, fungal cells, parasite cells, or bacterial cells. In some examples, the cell population comprises human cells. In some examples, the cell population comprise immune cells.

In some examples, the target nucleic acid site comprises a DNA or RNA molecule associated with the cancer, infectious disease, or autoimmune disease. In some instances, the nucleic acid target site comprises a double-stranded or single-stranded nucleic acid. In some examples, the nucleic acid target site comprises a single-stranded nucleic acid. In some examples, the nucleic acid target site comprises an RNA molecule. In some examples, the nucleic acid target site comprises a DNA molecule. In some examples, the nucleic acid target site comprises an rmRNA, rRNA, tRNA, non-coding RNA, long non-coding RNA, microRNA (miRNA), or combinations thereof. In some cases, the target nucleic acid comprises mRNA.

In some examples, the systems described herein comprise guide nucleic acid molecules complementary to at least 2 nucleic acid target sites. In some cases, the systems described herein are multiplexed and comprise guides complementary to at least 3, 4, 5, 6, 7, 8, 9, 10, 11, 12, 13, 14, 15, 16, 17, 18, 19, 20, 25, 30, 35, 40, 45, or 40 nucleic acid target sites.

In some examples, the nucleic acid target site is associated with a cancer cell population (e.g., comprises a mutation associated with a cancer or a DNA or RNA molecule unique to a cancer cell population). In some examples, the cancer cell population is associated with any of the following cancers, or combinations thereof: Acute Lymphoblastic Leukemia, Acute Myeloid Leukemia, Adrenocortical Carcinoma, Anal Cancer, Astrocytomas, Bile Duct Cancer, Bladder Cancer, Bone Cancer, Brain Cancer, Breast Cancer, Bronchial Cancer, Burkitt Lymphoma, Carcinoma, Cardiac Tumors, Cervical Cancer, Chordoma, Chronic Lymphocytic Leukemia, Chronic Myelogenous Leukemia, Chronic Myeloproliferative Neoplasms, Colon Cancer, Colorectal Cancer, Craniopharyngioma, Cutaneous T-cell lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumors, Extragonadal Germ Cell Tumors, Fallopian Tube Cancer, Fibrous Histiocytoma, Gallbladder Cancer, Gastric Cancer, Gastrointestinal Cancer, Gastrointestinal Carcinoid Cancer, Gastrointestinal Stromal Tumors, Gestational Trophoblastic Disease, Hairy Cell Leukemia, Head and Neck Cancer, Heart Tumors, Hepatocellular Cancer, Histiocytosis, Hodgkin Lymphoma, Hypopharyngeal Cancer, Intraocular Melanoma, Islet Cell Tumors, Kaposi Sarcoma, Kidney cancer, Langerhans Cell Histiocytosis, Laryngeal Cancer, Leukemia, Lip and Oral Cavity Cancer, Liver Cancer, Lung Cancer, Lymphoma, Malignant Fibrous Histiocytoma, Melanoma, Merkel Cell Carcinoma, Mesothelioma, Metastatic Squamous Neck Cancer, Midline Tract Carcinoma, Mouth Cancer, Multiple Endocrine Neoplasia Syndromes, Multiple Myeloma, Mycosis Fungoides, Myelodysplastic Syndromes, Myelogenous Leukemia, Myeloid Leukemia, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Osteosarcoma, Ovarian Cancer, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm, Pleuropulmonary Blastoma, Primary Central Nervous System (CNS) Lymphoma, Primary Peritoneal Cancer, Prostate Cancer, Rectal Cancer, Recurrent Cancer, Renal Cell Cancer, Retinoblastoma, Rhabdomyosarcoma, Salivary Gland Cancer, Sézary Syndrome, Skin Cancer, Small Cell Lung Cancer, Small Intestine Cancer, Soft Tissue Sarcoma, Squamous Cell Carcinoma, Squamous Neck Cancer with Occult Primary, Stomach Cancer, T-Cell Lymphoma, Testicular Cancer, Throat Cancer, Thymoma and Thymic Carcinoma, Thyroid Cancer, Tracheobronchial Cancer, Transitional Cell Cancer of the Renal Pelvis and Ureter, Ureter Cancer, Renal Pelvis Cancer, Urethral Cancer, Uterine Cancer, Uterine Sarcoma, Vaginal Cancer, Vascular Tumors, Vulvar Cancer, and Wilms Tumor.

In some examples, the CRISPR associated complexes disclosed herein are targeted to a cancer-associated nucleic acid target site, e.g., a nucleic acid molecule expressed by one or more cells in a cancer cell population in an individual. In some examples, the nucleic acid target site is unique or distinct to the cancer cell population as compared to other healthy cell populations in the individual. In some examples, the nucleic acid target site comprises a gene with a mutation associated with cancer. In some examples, the nucleic acid target site encodes for a cancer biomarker, such as a prostate cancer biomarker or non-small cell lung cancer. In some examples, the nucleic acid target site is only expressed in the cancer cell population in the individual and is not expressed in other cell populations in the individual. In some examples, the nucleic acid target site comprises an RNA molecule associated with cancer (e.g., comprising a mutation associated with cancer, whose overexpression is associated with cancer, or encoding a cancer biomarker). In some instances, the nucleic acid target site comprises a gene associated with cancer (e.g., a gene comprising a mutation associated with cancer, a gene only expressed in cancer cells). In some examples, the one or more nucleic acid target sites comprise any of the following genes: ABL, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM BMPR1A, BRCA1, BRCA2, BRIP1, c-MYC, CASR, CDC73, CDH1, CDK4, CDKN1B, CDKN1C, CDKN2A, CEBPA, CHEK2, CTNNA1, DBL, DEK/CAN DICER1, DIS3L2, E2A/PBX1, EGFR, ENL/HRX, EPCAM, ERG/TLS, ERBB, ERBB-2, ETS-1, EWS/FLI-1, FH, FLCN, FMS, FOS, FPS, GATA2, GLI, GPGSP, GREW HER2/neu, HOX11, HOXB13, HST, IL-3, INT-2, JUN KIT, KS3, K-SAM, LBC, LCK, L-MYC, LYL-1, LYT-10, LYT-10/Ca1, MAS, MAX MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PDGFRA, PHOX2B, PIM-1, PMS2, POLD1, POLE, POT1, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHB, SDHC, SDHD, SET/CAN SIS, SKI, SMAD4, SMARCA4, SMARCB1, SMARCE1, SRC, STK11, SUFU, TALL TAL2, TAN-1, TIAM1, TERC, TERT, TMEM127, TP53, TSC1, TSC2, TRK, VHF WRN, and WT1. In some instances, the one or more nucleic acid target sites is located in an oncogene. In some instances, the oncogene is selected from NRAS, TP53, BRAF, MYC, CTNNB1, CREBBP, EGFR, RB1, PTEN, and JAK1. In some instances, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20.

In some examples, the disease cell population comprises an autoimmune disease cell population. In some examples, an autoimmune disease cell population comprises a causative cell population for an autoimmune disease. In some examples, the disease cell population comprise one or more autoantibodies. In some examples, the disease cell population comprises an immune cell population. In some examples, the immune cell population comprises B-lymphocytes or T-lymphocytes. In some examples, the cell population is associated with any of the following autoimmune diseases, or combinations thereof: Addison disease, aplastic anemia, autoimmune anemias, autoimmune pancreatitis, Type 1 diabetes, rheumatoid arthritis, Behcet's Disease, Celiac disease, chronic inflammatory demyelinating polyneuropathy, chronic lymphocytic leukemia, Crohn's disease, psoriasis, psoriatic arthritis, lupus, systemic lupus erythematosus, inflammatory bowel disease, Graves' disease, Guillain-Barre syndrome, Hashimoto thyroiditis, non-Hodgkin's lymphoma, idiopathic thrombocytopenic purpura (ITP), IgA nephropathy, IgA-mediated autoimmune diseases, IgG4-related disease, Inflammatory bowel disease, Juvenile idiopathic arthritis, multiple sclerosis, Sjögren's syndrome, Opsoclonus myoclonus syndrome (OMS), Pemphigoid, Pemphigus, pemphigus vulgaris, Pernicious anemia, polymyositis, Psoriasis, pure red cell aplasia, Reactive arthritis, Rheumatoid arthritis, Sarcoidosis, scleroderma, Sjögren syndrome, Systemic lupus erythematosus, Thrombocytopenic purpura, Thrombotic thrombocytopenic purpura, Ulcerative colitis, Vasculitis (e.g., vasculitis associated with anti-neutrophil cytoplasmic antibody), and Vitiligo.

In some examples, the CRISPR associated complexes disclosed herein are targeted to a nucleic acid target site associated with an autoimmune disease e.g., a nucleic acid molecule expressed by one or more cells in a causative immune cell population for an autoimmune disease. In some examples, the nucleic acid target site encodes, at least in part, a T-cell receptor. In some examples, the nucleic acid target site encodes, at least in part, an antibody (e.g., an autoantibody). In some examples, the T-cell receptor contributes to or causes the autoimmune disease. In some examples, the nucleic acid target site encodes an antibody (e.g., an autoantibody). In some examples, the nucleic acid target site is unique or distinct to the causative immune cell population as compared to other healthy cell populations in the individual. In some examples, the nucleic acid target site is only expressed in the causative immune cell population in the individual and is not expressed in other cell populations in the individual. In some examples, the nucleic acid target site comprises an RNA molecule associated with the autoimmune disease. In some examples, the nucleic acid target site comprises a DNA molecule associated with the autoimmune disease.

In some examples, the disease is a sexually transmitted infection or other contagious disease. In some examples, the disease is any of the following diseases, or a combination thereof: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. In some examples, the disease is a respiratory virus (e.g., COVID-19, SARS, MERS, influenza and the like). In some examples, the disease is an upper respiratory tract infection or a lower respiratory tract infection. In some examples, disease is an influenza virus, such as an influenza A virus (IAV) or influenza B virus (IBV), a rhinovirus, a cold virus, a respiratory virus, an upper respiratory virus, a lower respiratory virus, a respiratory syncytial virus, or any combination thereof.

In some examples, the infectious disease is caused, at least in part, by any of the following pathogens or combinations thereof: viruses, fungi, helminths, protozoa, malarial parasites, Plasmodium parasites, Toxoplasma parasites, and Schistosoma parasites.

In some examples, the Pathogen causing the disease comprises, e.g., Mycobacterium tuberculosis, Streptococcus agalactiae, methicillin-resistant Staphylococcus aureus, Legionella pneumophila, Streptococcus pyogenes, Escherichia coli, Neisseria meningitidis, Pneumococcus, Hemophilus influenzae B, influenza virus, respiratory syncytial virus (RSV), M. pneumoniae, Streptococcus intermdius, Streptococcus pneumoniae, and Streptococcus pyogenes, or combinations thereof. In some examples, the Helminth causing the disease comprises roundworms, heartworms, and phytophagous nematodes, flukes, Acanthocephala, and tapeworms, or combinations thereof. In some examples, protozoan infections causing the disease comprise infections from Giardia spp., Trichomonas spp., African trypanosomiasis, amoebic dysentery, babesiosis, balantidial dysentery, Chaga's disease, coccidiosis, malaria and toxoplasmosis. In some examples, the pathogens comprise any of the following, or any combination thereof: Plasmodium falciparum, P. vivax, Trypanosoma cruzi and Toxoplasma gondii. In some examples, the pathogens comprise any of the following, or any combination thereof: Cryptococcus neoformans, Histoplasma capsulatum, Coccidioides immitis, Blastomyces dermatitidis, Chlamydia trachomatis, and Candida albicans.

In some examples, the disease is caused, at least in part, by any of the pathogenic viruses, or combinations thereof respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g. the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g. warts (including genital, anal), herpes (including oral, genital, anal), molluscum contagiosum); hemorrhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like) disclosed herein. In some examples, the disease is caused, at least in part by any of the following: immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. In some examples, the disease is caused, at least in part, by a pathogen disclosed herein including, e.g., HIV virus, Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritides, M. hyorhinis, M. orale, M. arginini, Acholeplasma laidlawii, M. salivarium, M pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. In some examples, the CRISPR associated complexes disclosed herein are targeted to a nucleic acid target site associated with a pathogen, for example a viral pathogen e.g., a nucleic acid molecule of a pathogen or expressed by a host cell infected with a pathogen. In some examples, the nucleic acid target site is unique or distinct to the infected cell population as compared to other healthy cell populations in the individual. In some examples, the nucleic acid target site is only expressed in the infected immune cell population in the individual and is not expressed in other cell populations in the individual. In some examples, the nucleic acid target site comprises an RNA molecule associated with an infectious disease. In some examples, the nucleic acid target site comprises a DNA molecule associated with an infectious disease. In some examples, the target nucleic acid site comprises, at least in part, a viral gene. In some examples, the viral gene is contained in any of the viruses disclosed herein. In some examples, the viral gene is an HIV gene (e.g., gag, pol, env, tat, rev, nef, vpr, vif, or vpu), an HBV gene, or an HCV gene. In some examples, the target nucleic acid site comprises a viral gene comprised in an individual host cell. In some examples, the target nucleic acid site comprises a viral gene incorporated into the genome of an individual host cell. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus from any of diseases disclosed herein, or combinations thereof.

F. Introducing Components into Target Cell

A guide RNA (or a nucleic acid comprising a nucleotide sequence encoding same) and/or a CRISPR-protein described herein can be introduced into a host cell by any of a variety of well-known methods. As a non-limiting example, a guide RNA and/or CRISPR protein can be combined with a lipid. As another non-limiting example, a guide RNA and/or CRISPR protein can be combined with a particle, or formulated into a particle.

Methods of introducing a nucleic acid and/or protein into a host cell are known in the art, and any convenient method can be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: 50169-409X(12)00283-9. doi: 10.1016/j.addr.2012.09.023), and the like. In some examples, the nucleic acid and/or protein are introduced into a disease cell comprised in a pharmaceutical composition comprising the guide RNA and/or CRISPR protein and a pharmaceutically acceptable excipient.

A guide RNA can be introduced, e.g., as a DNA molecule encoding the guide RNA, or can be provided directly as an RNA molecule (or a hybrid molecule when applicable). In some cases, a CRISPR protein (e.g., a type V CRISPR/Cas effector protein) is provided as a nucleic acid (e.g., an mRNA, a DNA, a plasmid, an expression vector, a viral vector, etc.) that encodes the protein. In some cases, the CRISPR protein is provided directly as a protein (e.g., without an associated guide RNA or with an associated guide RNA, i.e., as a ribonucleoprotein complex (RNP)). Like a guide RNA, a CRIPSR protein can be introduced into a cell (provided to the cell) by any convenient method; such methods are known to those of ordinary skill in the art. As an illustrative example, a CRISPR protein can be injected directly into a cell (e.g., with or without a guide RNA or nucleic acid encoding a guide RNA). As another example, a preformed complex of a CRISPR protein and a guide RNA (an RNP) can be introduced into a cell (e.g., eukaryotic cell) (e.g., via injection, via nucleofection; via a protein transduction domain (PTD) conjugated to one or more components, e.g., conjugated to the CRISPR protein, conjugated to a guide RNA, etc.).

In some examples, a nucleic acid (e.g., a guide RNA; a nucleic acid comprising a nucleotide sequence encoding a type V CRISPR/Cas effector protein, etc.) and/or a polypeptide (e.g., a type V CRISPR/Cas effector protein) is delivered to a cell (e.g., a target host cell) in a particle, or associated with a particle. The terms “particle” and “nanoparticle” can be used interchangeably, as appropriate.

This can be achieved, e.g., using particles or lipid envelopes. For example, a ribonucleoprotein (RNP) complex can be delivered via a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC); and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5).

A CRISPR protein (e.g., a type V CRISPR/Cas effector protein) (or an mRNA comprising a nucleotide sequence encoding the protein) and/or guide RNA (or a nucleic acid such as one or more expression vectors encoding the guide RNA) may be delivered simultaneously using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self-assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used. Doses of about 5 mg/kg can be used, with single or multiple doses, depending on various factors, e.g., the target tissue.

Lipidoid compounds (e.g., as described in US patent publication 20110293703) are also useful in the administration of polynucleotides and can be used. In one aspect, aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used, sugar-based particles may be used, for example GalNAc, as described with reference to WO2014118272 (incorporated herein by reference) and Nair, J K et al., 2014, Journal of the American Chemical Society 136 (49), 16958-16961). In some cases, lipid nanoparticles (LNPs) are used. Spherical Nucleic Acid (SNA™) constructs and other nanoparticles (particularly gold nanoparticles) can be used to a target cell. See, e.g., Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192. Semi-solid and soft nanoparticles are also suitable for delivery. An exosome can be used for delivery. Exosomes are endogenous nano-vesicles that transport RNAs and proteins, and which can deliver RNA to the brain and other target organs. Supercharged proteins can be used for delivery to a cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo. Cell Penetrating Peptides (CPPs) can be used for delivery. CPPs typically have an amino acid composition that either contains a high relative abundance of positively charged amino acids such as lysine or arginine or has sequences that contain an alternating pattern of polar/charged amino acids and non-polar, hydrophobic amino acids. In some instances, a CRISPR-associated protein or a nucleic acid encoding a CRISPR-associated protein; a guide RNA; or a combination thereof are introduced to a cell via an LNP. In some instances, the nucleic acid encoding a CRISPR-associated protein is an mRNA. In some instances, cell is in vitro. In some instances, the cell is in vivo. In some instances, the cell is ex vivo.

A guide RNA (or a nucleic acid comprising a nucleotide sequence encoding same) and/or a CRISPR-protein described herein can be administered in one dose, continuously or intermittently throughout the course of treatment. Methods of determining the most effective means and dosage of administration can be known to those of skill in the art and can vary with the composition used for therapy, the purpose of the therapy, the target cell population being treated, and the subject being treated. Single or multiple administrations can be carried out with the dose level and pattern being selected by the treating physician. Suitable dosage formulations and methods of administering the agents can be known in the art. Routes of administration can also be determined and method of determining the most effective routes of administration can be known to those of skill in the art and can vary with the composition used for treatment, the purpose of the treatment, the health condition or disease stage of the subject being treated, and target cell or tissue. Non-limiting examples of routes of administration include oral administration, nasal administration, injection, and topical application. Administration or application of a composition disclosed herein can be performed for a duration of at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, or 1000 consecutive days or nonconsecutive days. In some cases, the composition can be administered for life.

### G. Additional Therapeutic Agents

In some examples, methods described herein comprise administering a CRISPR system described herein with an additional therapeutic agent. Also described herein, in certain examples, are methods of using a CRISPR system for treating a disease or condition described herein in combination with an additional therapeutic agent.

In some examples, the additional therapeutic agent is an anti-cancer agent. In some examples, the additional therapeutic agent is a vascular endothelial growth factor (VEGF) pathway inhibitor or a VEGF receptor inhibitor (e.g., bevacizumab, CP 547632, or AZD2171). In some examples, the additional therapeutic agent is a poly(ADP-ribose) polymerase (PARP) inhibitor (e.g., olaparib, rucaparib, niraparib, talazoparib, veliparib, pamiparib, CEP 9722, E7016, Iniparib, or 3-aminobenzamide). In some examples, the anti-cancer agent is an mTOR inhibitor (e.g., rapamycin, everolimus, AP23573, CCI-779 and SDZ-RAD). In some examples, the anti-cancer agent is a taxane (e.g., paclitaxel, docetaxel, larotaxel, cabazitaxel). In some examples, the anti-cancer agent is an anthracycline (e.g., daunorubicin, doxorubicin epirubicin, valrubicin, mitoxatrone and idarubicin). In some examples, the anti-cancer agent is a platinum-based agent (e.g., cisplatin, carboplatin, oxaliplatin). In some examples, the anti-cancer agent is an antifolate (e.g., floxuridine, pemetrexed, raltitrexed). In some examples, the anti-cancer agent is a pyrimidine analogue (e.g., 5FU, capecitabine, cytrarabine, gemcitabine). In some examples, the anti-cancer agent comprises any of the following agents or combinations thereof: a FLT-3 inhibitor, a VEGFR inhibitor, an EGFR TK inhibitor, an aurora kinase inhibitor, a PIK-1 modulator, a Bc1-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, and a VEGF trap antibody. In some examples, the anti-cancer agent comprises an anti-PD1 agent, an anti-PD-L1 agent, Pembrolizumab, Nivolumab, Cemiplimab, Atezolizumab, Avelumab, Durvalumab, an anti-CTLA-4 agent, Ipilimumab, or combinations thereof. In some examples, the anti-cancer agent is interleukin-12, interleukin-11, interleukin-2, or combinations thereof.

In some examples, the additional therapeutic treats an autoimmune disorder. In some examples, the additional therapeutic agent comprises any of the following, or combinations thereof: TNF inhibitors, fliximab, adalimumab, etanercept, golimumab, ertolizumab pepol, Interleukin inhibitors, T-Cell inhibitors, B-Cell inhibitors, mTOR inhibitors, sirolimus, everolimus, IMDH inhibitors, azathioprine, leflunomide, mycophenolate, Calcineurin inhibitors, cyclopsroine, tacrolimus, corticosteroids, prednisone, budesonide, prednisolone, COX-2 inhibitors, COX-1 inhibitors, methotrexate, leflunomide, sulfasalazine, azathioprine, cyclophosphamide, antimalarials, d-penicillamine, cyclosporine, infliximab, etanercept, adalimumab, golimumab, certolizumab pegol, abatacept, adalimumab, anakinra, certolizumab, etanercept, golimumab, infliximab, ixekizumab, natalizumab, rituximab, secukinumab, tocilizumab, ustekinumab, basiliximab, daclizumab, vedolizumab, hydroxychloroquine, and methylprednisolone.

In some examples, the additional therapeutic agent is an antiviral agent. In some examples, the additional therapeutic agent comprises any of the following, or combinations thereof: Abacavir, Acyclovir, Adefovir, Amantadine, Ampligen, Amprenavir, Brivudin, Cidofovir, Famciclovir, Fomivirsen, Foscarnet, Ganciclovir, Penciclovir, Valacyclovir, Valganciclovir, Tipranavir, Vidarabine, Norvir, M2 Inhibitors, Amantadine, Rimantadine, Tromantadine, Moroxydine, Pleconaril, Letermovir, Remdesivir, Neuraminidase Inhibitors, Oseltamivir, Truvada, Peramivir, Zanamivir, Umifenovir, Interferons, Ribavirin, Telaprevir, protease inhibitors, tubercidin, Vicriviroc, Vidarabine, Trizivir, Zalcitabine, Zidovudine, and Boceprevir.

### EXAMPLES

The following examples are included for illustrative purposes only and are not intended to limit the scope of the invention.

### Example 1: Inducing Cell Death, Apoptosis, or Cell Cycle Arrest of a Cancer Cell Population Using Non-Specific Cleavage of a CRISPR Protein

A CRISPR protein of the present disclosure (e.g., any of the CRISPR proteins listed in the Tables herein), and a guide nucleic acid molecule capable of hybridizing to a target nucleic acid site are administered to an individual having cancer. The target nucleic acid site comprises a mutation associated with the cancer and considered to be a biomarker for the cancer. The nucleic acid target site is expressed by a population of cancer cells. The CRISPR protein and the guide nucleic acid molecule are administered as a ribonucleoprotein complex or as separate nucleic acids encoding for each component. Upon binding of the guide nucleic acid to the nucleic acid target site, a non-specific cleavage activity of the CRISPR protein is activated, inducing non-specific cleavage (e.g. trans cleavage) of single-stranded DNA molecules in the cells comprising the nucleic acid target site. The non-specific cleavage of single-stranded DNA molecules leads to cell death, apoptosis, or cell cycle arrest within the population of cancer cells, i.e., cells comprising the cancer associated mutations within the nucleic acid target sites.

### Example 2: Inducing Cell Death, Apoptosis, or Cell Cycle Arrest of a Cancer Cell Population Using Non-Specific Cleavage of a Multiplexed CRISPR Protein

A CRISPR protein of the present disclosure (e.g., any of the CRISPR proteins listed in the Tables herein), and two guide nucleic acid molecules capable of hybridizing to two separate target nucleic acid sites are administered to an individual having cancer. Each nucleic acid target site is a DNA molecule comprising a different mutation associated with the cancer. Both target nucleic acids are expressed by a population of cancer cells. The CRISPR protein and the guide nucleic acid molecules are administered as a ribonucleoprotein complex or as separate nucleic acids encoding for each component. Upon binding of the guide nucleic acids to the nucleic acid target sites, a non-specific cleavage activity of the CRISPR protein is activated, inducing non-specific cleavage (e.g., trans cleavage) of single-stranded DNA molecules in the cells comprising the nucleic acid target sites. The non-specific cleavage of single-stranded DNA molecules leads to cell death, apoptosis, or cell cycle arrest within the population of cancer cells, i.e., cells comprising the cancer associated mutations within the nucleic acid target sites.

### Example 3: Inducing Cell Death, Apoptosis, or Cell Cycle Arrest of a Cancer Cell Population Using Non-Specific Cleavage of a CRISPR Protein in Combination with an Additional Therapeutic Agent

A CRISPR protein of the present disclosure (e.g., any of the CRISPR proteins listed in the Tables herein), and a guide nucleic acid molecule capable of hybridizing to a target nucleic acid site are administered to an individual having cancer. The nucleic acid target site comprises a mutation associated with the cancer and considered to be a biomarker for the cancer. The target nucleic acid is expressed by a population the cancer cells. The CRISPR protein and the guide nucleic acid molecule are administered as a ribonucleoprotein complex or as separate nucleic acids encoding for each component. Also administered to the individual is a PARP inhibitor. The PARP inhibitor is administered to the individual orally, pursuant to an administration schedule determined based on weight on other factors known to skilled artisans. Upon binding of the guide nucleic acid to the nucleic acid target site, a non-specific cleavage activity of the CRISPR protein is activated, inducing non-specific cleavage (e.g., trans cleavage) of single-stranded DNA molecules in the cells comprising the nucleic acid target site. The non-specific cleavage of single-stranded DNA molecules, in combination with the activity of the PARP inhibitor, leads to cell death, apoptosis, or cell cycle arrest of within the population of cancer cells, i.e., cells comprising the cancer associated mutations within the nucleic acid target sites.

### Example 4: Inducing Cell Death, Apoptosis, or Cell Cycle Arrest of an Infected Cell Population Using Non-Specific Cleavage of a CRISPR Protein

A CRISPR protein of the present disclosure (e.g., any of the CRISPR proteins listed in the Tables herein), and a guide nucleic acid molecule capable of hybridizing to a target nucleic acid site are administered to an individual having a viral infection. The target nucleic acid site comprises a portion of the viral genome. The nucleic acid target site is in a population of cells infected with the virus. The CRISPR protein and the guide nucleic acid molecule are administered as a ribonucleoprotein complex or as separate nucleic acids encoding for each component. Upon binding of the guide nucleic acid to the nucleic acid target site, a non-specific cleavage activity of the CRISPR protein is activated, inducing non-specific cleavage (e.g., trans cleavage) of single-stranded DNA molecules in the cells infected with the virus. The non-specific cleavage of single-stranded DNA molecules leads to cell death, apoptosis, or cell cycle arrest within the population of infected cells.

### Example 5: Inducing Cell Death, Apoptosis, or Cell Cycle Arrest of an Infected Cell Population Using Non-Specific Cleavage of a Multiplexed CRISPR Protein

A CRISPR protein of the present disclosure (e.g., any of the CRISPR proteins listed in the Tables herein), and two guide nucleic acid molecules capable of hybridizing to two separate target nucleic acid sites are administered to an individual having a viral infection. Each target nucleic acid site is a DNA molecule comprising a different portion of the viral genome. Both target nucleic acids are comprised within the population of virally infected cells. The CRISPR protein and the guide nucleic acid molecules are administered as a ribonucleoprotein complex or as separate nucleic acids encoding for each component. Upon binding of the guide nucleic acids to the nucleic acid target sites, a non-specific cleavage activity of the CRISPR protein is activated, inducing non-specific cleavage (e.g., trans cleavage) of single-stranded DNA molecules in the cells comprising the nucleic acid target sites. The non-specific cleavage of single-stranded DNA molecules leads to cell death, apoptosis, or cell cycle arrest of within the population of virally infected cells, i.e., cells comprising the portions of the viral genome within the nucleic acid target sites.

### Example 6: Inducing Cell Death, Apoptosis, or Cell Cycle Arrest of an Infected Cell Population Using Non-Specific Cleavage of a CRISPR Protein in Combination with an Additional Therapeutic Agent

A CRISPR protein of the present disclosure (e.g., any of the CRISPR proteins listed in the Tables herein), and a guide nucleic acid molecule capable of hybridizing to a target nucleic acid site are administered to an individual having a viral infection. The target nucleic acid site comprises a portion of the viral genome. The nucleic acid target site is in a population of cells infected with the virus. The CRISPR protein and the guide nucleic acid molecule are administered as a ribonucleoprotein complex or as separate nucleic acids encoding for each component. Also administered to the individual is an antiviral agent pursuant to an administration schedule determined based on factors known to skilled artisans. Upon binding of the guide nucleic acid to the nucleic acid target site, a non-specific cleavage activity of the CRISPR protein is activated, inducing non-specific cleavage (e.g., trans cleavage) of single-stranded DNA molecules in the cells comprising the nucleic acid target site. The non-specific cleavage of single-stranded DNA molecules, in combination with the activity of the antiviral agent, leads to cell death, apoptosis, or cell cycle arrest of within the population of infected cells, i.e., cells comprising the portions of the viral genome within the nucleic acid target sites.

### Example 7: Inducing Cell Death, Apoptosis, or Cell Cycle Arrest of an Autoimmune Cell Population Using Non-Specific Cleavage of a CRISPR Protein

A CRISPR protein of the present disclosure (e.g., any of the CRISPR proteins listed in the Tables herein), and a guide nucleic acid molecule capable of hybridizing to a target nucleic acid site are administered to an individual having an autoimmune disease. The target nucleic acid site encodes, at least in part an auto-antibody contributing to the autoimmune disease. The nucleic acid target site is in a population of a causative immune cell population. The CRISPR protein and the guide nucleic acid molecule are administered as a ribonucleoprotein complex or as separate nucleic acids encoding for each component. Upon binding of the guide nucleic acid to the nucleic acid target site, a non-specific cleavage activity of the CRISPR protein is activated, inducing non-specific cleavage (e.g., trans cleavage) of single-stranded DNA molecules in the causative cell population. The non-specific cleavage of single-stranded DNA molecules leads to cell death, apoptosis, or cell cycle arrest within the cell population.

### Example 8: Inducing Cell Death, Apoptosis, or Cell Cycle Arrest of an Infected Cell Population Using Non-Specific Cleavage of a Multiplexed CRISPR Protein

A CRISPR protein of the present disclosure (e.g., any of the CRISPR proteins listed in the Tables herein), and two guide nucleic acid molecules capable of hybridizing to two separate target nucleic acid sites are administered to an individual having an autoimmune disease. Each target nucleic acid site encodes, at least in part an auto-antibody contributing to the autoimmune disease. Both target nucleic acids are comprised within the causative cell population. The CRISPR protein and the guide nucleic acid molecules are administered as a ribonucleoprotein complex or as separate nucleic acids encoding for each component. Upon binding of the guide nucleic acids to the nucleic acid target sites, a non-specific cleavage activity of the CRISPR protein is activated, inducing non-specific cleavage (e.g., trans cleavage) of single-stranded DNA molecules in the cells comprising the nucleic acid target sites. The non-specific cleavage of single-stranded DNA molecules leads to cell death, apoptosis, or cell cycle arrest within the population of causative cells.

### Example 9: Inducing Cell Death, Apoptosis, or Cell Cycle Arrest of an Infected Cell Population Using Non-Specific Cleavage of a CRISPR Protein in Combination with an Additional Therapeutic Agent

A CRISPR protein of the present disclosure (e.g., any of the CRISPR proteins listed in the Tables herein), and a guide nucleic acid molecule capable of hybridizing to a target nucleic acid site are administered to an individual having an autoimmune disease. The target nucleic acid site encodes, at least in part, an auto-antibody contributing to the autoimmune disease. The nucleic acid target site is comprised in a population of a causative immune cell population. The CRISPR protein and the guide nucleic acid molecule are administered as a ribonucleoprotein complex or as separate nucleic acids encoding for each component. Also administered to the individual is an additional therapeutic agent, e.g., Rituximab, pursuant to an administration schedule determined based on factors known to skilled artisans. Upon binding of the guide nucleic acid to the nucleic acid target site, a non-specific cleavage activity of the CRISPR protein is activated, inducing non-specific cleavage (e.g., trans cleavage) of single-stranded DNA molecules in the cells comprising the nucleic acid target site. The non-specific cleavage of single-stranded DNA molecules, in combination with the activity of the additional therapeutic agent, leads to cell death, apoptosis, or cell cycle arrest within the population of causative cells.

### Example 10: CasPhi.12 Selectively Reduces Growth/Viability of Pancreatic Cancer Cells Expressing Mutant KRAS

The following experiments were carried out to assess the specificity of Cas0.12 knockout of the oncogene KRAS in a human pancreatic adenocarcinoma cell line Panc08.13 (KRAS-G12D) by measuring cell viability and colony formation.

KRAS (Kirsten rat sarcoma 2 viral oncogene homolog) is a proto-oncogene and one of the most common driver oncogenes, which is mutated in over 90% of pancreatic cancers and over 30% of lung and colon cancers. It functions as a GTPase and is attached to the inner surface of the cell membrane. The most common mutations include KRAS p.G12C—c.34G->T; KRAS p.G12D—c.35G->A (12th a.a. is mutated from Gly to Asp; 35th nucleotide is mutated from G to A; and KRAS p.G12V—c.35G->T. Selective knockdown of the KRAS-G12D mutant leads to tumor cell death. The Panc08.13 cell line (ATCC—Cat #CRL-2551) is a homozygous KRAS-G12D mutant.

Panc08.13 cells were seeded in T-75 flasks and grown to −70-80% confluence. Approximately 48 hours later, cells were trypsinized and resuspended in a buffer at a concentration of 1×10⁷ cells/ml. Cells were electroporated, using a ThermoFisher Scientific Neon™ Transfection System according to manufacturer's protocol, with RNPs containing Casφ.12—Aldevron—Lot #M22612-01 and one of the following guide nucleic acids:

KRAS-WT guide for CasΦ.12 (R5677 KRAS_3):
(SEQ ID NO: 235)
AUUGCUCCUUACGAGGAGACCCUACGCCACCAGCUCC
KRAS-G12D guide for CasΦ.12 (R5680 KRAS_G12D_3):
(SEQ ID NO: 236)
AUUGCUCCUUACGAGGAGACCCUACGCCAUCAGCUCC
KRAS-WT guide for Cas9 (R5681 KRAS_WT_Cas9):
(SEQ ID NO: 237)
GUAGUUGGAGCUGGUGGCGU
KRAS-G12D guide for Cas9 (KRAS_G12D_Cas9)
(SEQ ID NO: 238)
GUAGUUGGAGCUGAUGGCGU

RNPs were formed by mixing Casφ.12 with KRAS-WT or KRAS-G12D Casφ.12 guides at a ratio of 2:1, in separate tubes, and incubating at RT for 30 mins.

Electroporated cells were transferred to plates for MTS assays and colony formation. Electroporated cells were incubated for 1-4 days at 37° C. and 5% CO₂ before performing MTS assays. Electroporated cells were incubated for 15 days at 37° C. and 5% CO₂ before assessing colony formation.

### MTS Assays

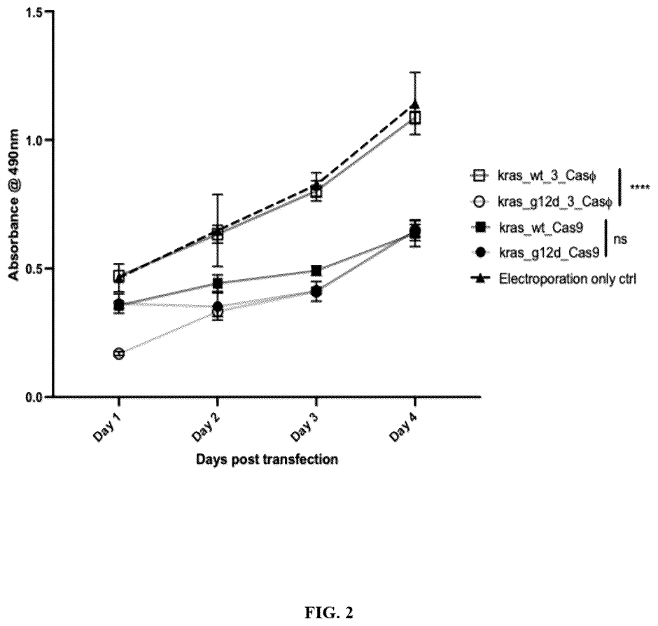

An MTS assay may be used to assess cell proliferation, cell viability and cytotoxicity. MTS assays were performed with CellTiter 96 ® AQᵤₑₒᵤₛ One Solution Cell Proliferation Assay, a colorimetric method for determining the number of viable cells, according to manufacturer's instructions. Absorbance was read at 24 h, 48h, 72h and 96h after adding assay reagent post transfection. Results are shown in FIG. 2. FIG. 2 shows CasΦ.12 KRAS-G12D guides are more specific to the KRAS-G12D mutant cell line. FIG. 2 shows that KRAS_G12D guide transfected cells grow slower. FIG. 2 shows that both the KRAS WT and KRAS G12D Cas9 guides knockout the KRAS-G12D mutation, leading to cell death.

### Colony Formation Assays

Colony formation assays were performed to assess proliferation of electroporated Panc08.13 cells. The presence of colonies means that the proliferative capabilities of the cells have not been affected because the KRAS-G12D gene has not been knocked out in those cells. 15 days after electroporation, cells were washed and placed on ice. To fix cells, ice-cold 100% methanol was added to each well and incubated on ice for 10 minutes. After removing methanol and removing cells from ice, 2 ml of 0.5% crystal violet solution was added to each well and incubated at room temperature for 10 minutes. Fixed cells were then washed thoroughly with water. Images of plates were captured and stained cell colonies quantified. The experiment was performed in duplicate. Results are shown in TABLE 5.

TABLE 5
Colony Formation from Pancreatic Cancer Cells Electroporated with RNPs containing
wildtype or mutant specific KRAS guide RNAs: Cas9 versus CasΦ.12

| RNP | Experiment 1 Colony # | Experiment 2 Colony # |
|---|---|---|
| CasΦ.12 + KRAS-WT guide | 63 | 72 |
| CasΦ.12 + KRAS-G12D guide | 27 | 36 |
| Cas9 + KRAS-WT guide | 5 | 3 |
| Cas9 + KRAS-G12D guide | 2 | 2 |

Results show that knocking out KRAS-G12D leads to cell death and inability to form cell colonies on a plate. Consistent with observations in the MTS assay above, while both Cas9 guides non-specifically knockout the KRAS-G12D gene in Panc08.13 cells, the Casφ.12 KRAS_G12D_3 guide knocked out the KRAS-G12D gene, while the Casφ.12 KRAS_WT_3 guide did not. Hence, Casφ.12 was more specific for point mutations than Cas9.

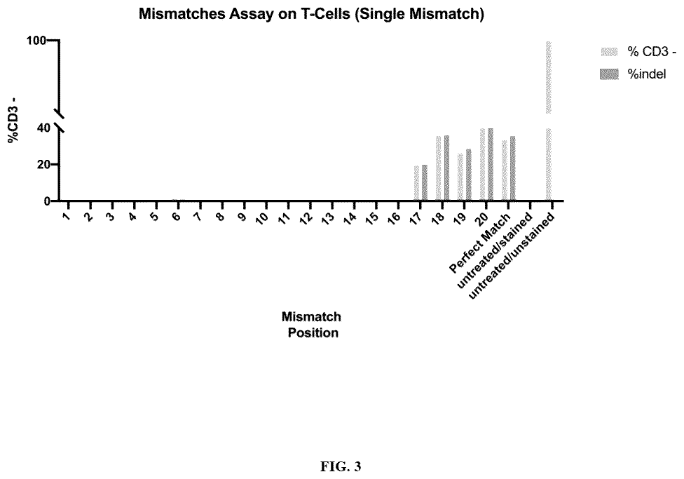

The Casφ.12 “seed” region, that is the region of the guide RNA that hybridizes to a target nucleic acid, was determined to be the first 16 nucleotides of the guide RNA. Casφ.12 was intolerant of one or two nucleotide mismatches in the first 16 nucleotides of the guide RNA. See FIG. 3. In contrast, the seed region of Cas9 is only 5 nucleotides in length, and hybridizes to the 5 nucleotides upstream of a PAM. Without being bound by theory, the presence of a longer seed region in Casφ.12 guide RNAs confers an advantage of higher specificity for target DNA sequences.

### Activity in KRAS Wildtype Cells

To determine the extent to which Casφ.12 and Cas9 mediated editing is specific for mutant KRAS, cells expressing wildtype KRAS, (BxPC3 and HEK293T), were electroporated with RNPs containing the following guide RNAs, and % indels in KRAS quantified by next generation sequencing (NGS). Electroporation was performed under three different conditions with ThermoFisher Scientific Neon™ Transfection System according to manufacturer's protocol.

KRAS-WT guide for CasΦ.12:
(SEQ ID NO: 239)
UUGGAGCUGGUGGCGUAGGC
KRAS-G12D guide for CasΦ.12:
(SEQ ID NO: 240)
UUGGAGCUGAUGGCGUAGGC
KRAS-WT guide for Cas9 (R5681 KRAS_WT_Cas9):
(SEQ ID NO: 241)
GUAGUUGGAGCUGGUGGCGU
KRAS-G12D guide for Cas9 (KRAS_G12D_Cas9):
(SEQ ID NO: 242)
GUAGUUGGAGCUGAUGGCGUAGG

NGS was run 72 hours after electroporation. Results are shown in TABLE 6. Cas9 shows editing using the KRAS-G12D guide RNA in wildtype cells, whereas CasΦ.12 shows negligible editing with a KRAS-G12D guide RNA.

TABLE 6
% Indels in wildtype KRAS gene with RNPs containing wildtype
or mutant specific KRAS guide RNAs: Cas9 versus CasΦ.12

| | | | |
|---|---|---|---|
| CasΦ.12 + KRAS-WT | CasΦ.12 + KRAS-G12D | Cas9 + KRAS-WT | Cas9 + KRAS- |
| guide RNA | guide RNA | guide RNA | G12D guide RNA |

BxPC3 cells expressing wildtype KRAS

| | | | |
|---|---|---|---|
| 68.1% | 0.4% | 74.7% | 4% |
| 66.2% | 0.4% | 75.6% | 3.5% |
| 64.9% | 0.3% | 89.3% | 12.5% |

HEK293T cells expressing wildtype KRAS

| | | | |
|---|---|---|---|
| 62.5% | 0.4% | 73.6% | 12.4% |
| 58.6% | 0.3% | 72.6% | 7.4% |
| 53.3% | 0.3% | 91.7% | 13.3% |

### Example 11. CasΦ.12 Selectively Modifies Mutant KRAS in a Pancreatic Cancer Cell Line

CasΦ.12 knockout of the oncogene KRAS in the human pancreatic adenocarcinoma cell lines BxPC3 (KRAS-WT) and AsPC1 (KRAS-G12D) were assessed by performing next generation sequencing (NGS) post transfection of CasΦ.12 RNPs.

48h before electroporation with a Neon™ Transfection System, BxPC-3 and AsPC1 cells were seeded in T-75 flasks and cells were grown to approximately 70-80% confluence. Cells were then trypsinized and resuspended in R Buffer (provided in the Neon™ 10 μl kit) at a concentration of 1×10\hat{ }⁷ cells/ml.

To form Casφ.12 RNPs, 300 pmol of Casφ.12 was mixed with 600 pmol of wildtype KRAS-targeting guide RNA or G12D mutant KRAS targeting guide RNA (RNA:Nuclease ratio 2:1), in separate tubes. 1× Casφ.12 protein buffer was used as a diluent. Casφ.12 RNPs were incubated at RT for 30 mins.

Cas9 RNPs were formed as well, to serve as controls. 25 pmol of Cas9 was mixed with 75 pmol of wildtype KRAS-targeting guide RNA or G12D mutant KRAS targeting guide RNA for an RNA:Nuclease ratio of 3:1. The total volume of the mixture for each reaction was 3 μl. R Buffer (Neon™) was used as a diluent. Cas9 RNPs were incubated at room temperature (RT) for 20 mins.

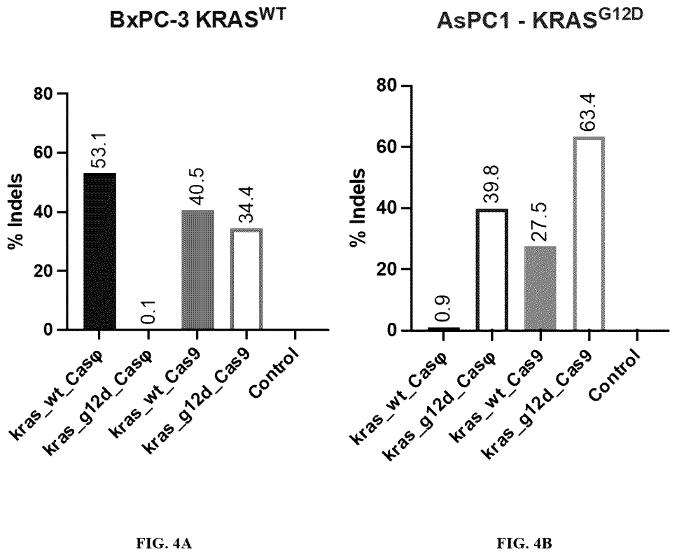

Electroporation was performed according to manufacturer's instructions. Following electroporation, cells were incubated at 37° C. and 5% CO2 for a week before NGS analysis. DNA was extracted from cells and barcoded for sequencing, and indel formation as indicated by sequencing results was quantified and analyzed. Guide sequences and results are provided in TABLE 7, and shown in FIG. 4A (BxPC-3) and FIG. 4B (AsPC1). While a Cas9 RNP with a KRAS-G12D targeting guide RNA produced 34.4% indels in BxPC-3 cells expressing wildtype KRAS, a Casφ.12 RNP with a G12D mutant KRAS targeting guide RNA only produced 0.1% indels in BxPC-3 cells. In contrast, a Casφ.12 RNP with a G12D mutant KRAS targeting guide RNA produced 39.8% indels in AsPC1 cells harboring the G12D mutant KRAS. This experiment demonstrated that Cas9 has reduced specificity relative to that of Casφ.12, and that a Casφ.12 RNP can distinguish between a wildtype and mutant allele of KRAS.

TABLE 7
Indel Formation

| | | BxPC3 | AsPC1 |
|---|---|---|---|
| | Guide RNA | KRAS | KRAS |
| RNP Components | Sequence | WT | G12D |
| Casφ.12; WT | AUUGCUCCUUACGAGG | 53.1% | 0.9% |
| KRAS targeting | AGACGAGCUGGUGGCG | | |
| guide RNA | UAGGC | | |
| | (SEQ ID NO: 263) | | |
| Casφ.12; KRAS- | AUUGCUCCUUACGAGG | 0.1% | 39.8% |
| G12D targeting | AGACGAGCUGAUGGCG | | |
| guide RNA | UAGGC | | |
| | (SEQ ID NO: 264) | | |
| Cas9; WT KRAS | GUAGUUGGAGCUGGUG | 40.5% | 27.5% |
| targeting | GCGU | | |
| guide RNA | (SEQ ID NO: 237) | | |
| Cas9; KRAS-G12D | GUAGUUGGAGCUGAUG | 34.4% | 63.4% |
| targeting | GCGU | | |
| guide RNA | (SEQ ID NO: 238) | | |

### Example 12. Casφ.12 Editing of KRAS with Chemically Modified Guide RNA

CasΦ.12 RNP generated knockout of KRAS in the human pancreatic adenocarcinoma cell lines BxPC3 (KRAS-WT) and AsPC1 (KRAS-G12D) was assessed by performing next generation sequencing (NGS) post transfection of Cas0.12 mRNA and various guide nucleic acids. Cells transfected with Cas9 mRNA and corresponding Cas9 guide nucleic acids served as controls and comparators.

48h before electroporation with a Neon™ Transfection System, BxPC-3 and AsPC1 cells were seeded in T-75 flasks and cells were grown to approximately 70-80% confluence. Cells were then trypsinized and resuspended in R Buffer (provided in the Neon™ 10 ul kit) at a concentration of 1×10\hat{ }⁷ cells/ml.

CasΦ.12 mRNA (5 μg) and KRAS CasΦ.12 guides (500 pmol) were resuspended at 500 μM. Four different guide RNAs were tested with CasΦ.12 as shown in TABLE 8 below: (1) a wildtype KRAS targeting guide RNA with 2′ O methyl modifications of the last 3 nucleotides (Casφ.12_M1_WT-KRAS Guide RNA); (2) a G12D mutant KRAS targeting guide RNA with 2′ O methyl modifications of the last 3 nucleotides (Casφ.12_M1_KRAS-G12D Guide RNA); (3) a wildtype KRAS targeting guide RNA with 2′ O methyl modifications of the first 3 nucleotides and last 3 nucleotides, as well as phosphorothioate linkages between the first 4 nucleotides, and phosphorothioate linkages between the last 3 nucleotides (Casφ.12_M6_WT-KRAS Guide RNA); and (4) a G12D mutant KRAS targeting guide RNA with 2′ O methyl modifications of the first 3 nucleotides and last 3 nucleotides, as well as phosphorothioate linkages between the first 4 nucleotides, and phosphorothioate linkages between the last 3 nucleotides (Casφ.12_M6_G12D-KRAS Guide RNA; Casφ.12 mRNA). As a control, Cas9 mRNA (1 μg) was mixed with 200 pmol KRAS Cas9 guides. These RNA mixtures were added to 100,000 cells for each reaction.

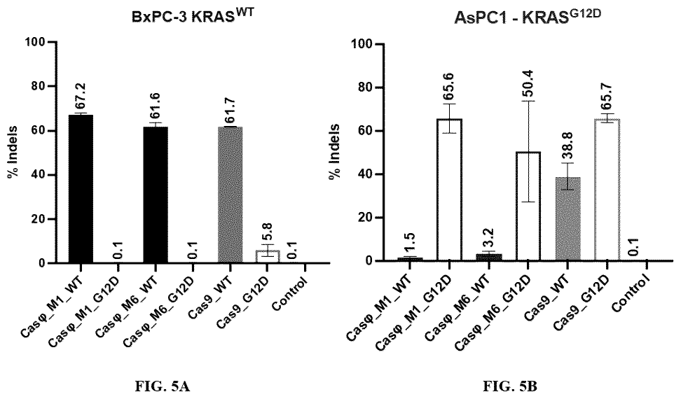

Electroporation was performed according to the manufacturer's instructions. Following electroporation, cells were incubated at 37° C. and 5% CO2 for 72 hours before NGS analysis. DNA was extracted from cells and barcoded for sequencing, and indel formation (as indicated by sequencing results) was quantified and analyzed. Results are provided in TABLE 8 and shown in FIG. 5A (BxPC-3) and FIG. 5B (AsPC1). While a Cas9 and a KRAS-G12D targeting guide RNA produced 5.8% indels in BxPC-3 cells expressing wildtype KRAS, Casφ.12 and both G12D mutant KRAS targeting guide RNAs only produced 0.1% indels in BxPC-3 cells. In contrast, Casφ.12 produced 65.6% indels and 50.4% indels with Casφ.12_M1_KRAS-G12D Guide RNA and Casφ.12_M6_G12D-KRAS Guide RNA, respectively in AsPC1 cells harboring the G12D mutant KRAS. This experiment demonstrated that Cas9 has reduced specificity relative to that of Casφ.12, and that a Casφ.12 can distinguish between a wildtype and mutant allele of KRAS.

TABLE 8
Indel Formation

| Transfected | | BxPC3 | AsPC1 |
|---|---|---|---|
| Guide RNA | Guide RNA | KRAS | KRAS |
| and mRNA | Sequence | WT | G12D |
| Casφ.12_M1_WT-KRAS | CUUUCAAGACUAAUAG | 67.2% | 1.5% |
| Guide RNA; Casφ.12 | AUUGCUCCUUACGAGG | | |
| mRNA | AGACGAGCUGGUGGCG | | |
| | UAmGmGmC | | |
| | (SEQ ID NO: 265) | | |
| Casφ.12_M1_KRAS- | CUUUCAAGACUAAUAG | 0.1% | 65.6% |
| G12D Guide RNA; | AUUGCUCCUUACGAGG | | |
| Casφ.12 mRNA | AGACGAGCUGAUGGCG | | |
| | UAmGmGmC | | |
| | (SEQ ID NO: 266) | | |
| Casφ.12_M6_WT-KRAS | mC*mU*mU*UCAAGAC | 61.6% | 3.2% |
| Guide RNA; Casφ.12 | UAAUAGAUUGCUCCUU | | |
| mRNA | ACGAGGAGACGAGCUG | | |
| | GUGGCGUAmG*mG*mC | | |
| | (SEQ ID NO: 285) | | |
| Casφ.12_M6_G12D- | mC*mU*mU*UCAAGAC | 0.1% | 50.4% |
| KRAS Guide RNA; | UAAUAGAUUGCUCCUU | | |
| Casφ.12 mRNA | ACGAGGAGACCCUACG | | |
| | CCAUCAGCmU*mC*mC | | |
| | (SEQ ID NO: 267) | | |
| Cas9_WT-KRAS Guide | mG*mT*mA*GTTGGAG | 61.7% | 38.8% |
| RNA; Cas9 mRNA | CTGGTGGmC*mG*mT | | |
| | (SEQ ID NO: 268) | | |
| Cas9_G12D-KRAS Guide | mG*mT*mA*GTTGGAG | 5.8% | 65.7% |
| RNA; Cas9 mRNA | CTGATGGmC*mG*mT | | |
| | (SEQ ID NO: 269) | | |
| Control- | N/A | 0.1% | 0.1% |
| electroporated cells, | | | |
| no mRNA or guide | | | |

m = a 2′ O-methyl modification of the subsequent indicated nucleotide
* = a phosphorothioate linkage

### Example 13. LNP Delivery of Casφ.12 mRNA and KRAS Guide RNAs

In this Example, the efficiency and specificity of Cas0.12 knockout of the oncogene KRAS in the human pancreatic adenocarcinoma cell lines BxPC3 (KRAS-WT) and AsPC1 (KRAS-G12D) using lipid nanoparticle (LNP) formulations are tested by performing next generation sequencing post transfection of CasΦ.12 mRNA and KRAS targeting guides.

24h before LNP transfection, BxPC3 and AsPC1 cells are seeded in a 96-well plate at 15,000 cells/well. 72h after electroporation, cells are collected from 96-well plates by trypsinization and transferred to U-bottom 96-well plates (plate maps below). DNA is extracted from cells and barcoded for sequencing. Indel formation is quantified by analyzing sequencing results.

### Example 14. Cas13 Knockdown of KRAS RNA

In this Example, the ability of multiple Cas13 orthologs and KRAS targeting guide nucleic acids (provided in TABLE 9) are assessed in mammalian cells (e.g., HEK293T cells). LwaCas13a is used as a positive control. Cells are plated at 25,000 cells per well in a 96 well plate 24 hours prior to transfection. All constructs are transfected into HEK293T cells in triplicate. Cells are harvested 48 hours post transfection and KRAS RNA is quantified via qPCR.

TABLE 9
Cas13 orthologs and KRAS Guide Nucleic Acid Sequences

| | Cas13 | | |
|---|---|---|---|
| | ortholog | | |
| | Amino | | |
| Cas13 | Acid | | KRAS Guide |
| ortholog | Sequence | KRAS Guide Repeat Sequence | Spacer Sequence |
| 2021Q1_ | SEQ ID | GTTAGAATATAACCCTGTTTGTAG | CCAGCTCCAACTACCACAA |
| 2.020 | NO: 248 | GGGTAATAAAAC (SEQ ID NO: 270) | GTTTAT (SEQ ID NO: 273) |
| 2021Q1_ | SEQ ID | GTTAGAATATAACCCTGTTTGTAG | ACCAGCTCCAACTACCACA |
| 2.018 | NO: 249 | GGGTAATAAAAC (SEQ ID NO: 270) | AGTTTA (SEQ ID NO: 274) |
| 2021Q1_ | SEQ ID | GTTAGAATATAACCCTGTTTGTAG | CACCAGCTCCAACTACCAC |
| 2.001 | NO: 250 | GGGTAATAAAAC (SEQ ID NO: 270) | AAGTTT (SEQ ID NO: 275) |
| 2021Q1_ | SEQ ID | GTTAGAATATAACCCTGTTTGTAG | CCACCAGCTCCAACTACCA |
| 2.003 | NO: 251 | GGGTAATAAAAC (SEQ ID NO: 270) | CAAGTT (SEQ ID NO: 276) |
| 2021Q1_ | SEQ ID | GTTAGAATATAACCCTGTTTGTAG | GCCACCAGCTCCAACTACC |
| 2.004 | NO: 252 | GGGTAATAAAAC (SEQ ID NO: 270) | ACAAGT (SEQ ID NO: 277) |
| 2021Q1_ | SEQ ID | GTTAGAATATAACCCTGTTTGTAG | CGCCACCAGCTCCAACTAC |
| 2.021 | NO: 253 | GGGTAATAAAAC (SEQ ID NO: 270) | CACAAG (SEQ ID NO: 278) |
| 2021Q1_ | SEQ ID | GTTAGAATATAACCCTGTTTGTAG | ACGCCACCAGCTCCAACTA |
| 2.022 | NO: 254 | GGGTAATAAAAC (SEQ ID NO: 270) | CCACAA (SEQ ID NO: 279) |
| 2021Q1_ | SEQ ID | GTTAGAATATAACCCTGTTTGTAG | TACGCCACCAGCTCCAACT |
| 2.016 | NO: 255 | GGGTAATAAAAC (SEQ ID NO: 270) | ACCACA (SEQ ID NO: 280) |
| 2021Q1_ | SEQ ID | GTTAGAATATAACCCTGTTTGTAG | CTACGCCACCAGCTCCAAC |
| 2.029 | NO: 256 | GGGTAATAAAAC (SEQ ID NO: 270) | TACCAC (SEQ ID NO: 281) |
| 2020Q3_ | SEQ ID | GTTAGAATATAACCCTGTTTGTAG | CCTACGCCACCAGCTCCAA |
| 6.008 | NO: 257 | GGGTAATAAAAC (SEQ ID NO: 270) | CTACCA (SEQ ID NO: 282) |
| 2020Q3_ | SEQ ID | GTTAGAATATAACCCTGTTTGTAG | GCCTACGCCACCAGCTCCA |
| 6.001 | NO: 258 | GGGTAATAAAAC (SEQ ID NO: 270) | ACTACC (SEQ ID NO: 283) |
| 2021Q1_ | SEQ ID | GTTAGAATATAACCCTGTTTGTAG | TGCCTACGCCACCAGCTCC |
| 2.026 | NO: 259 | GGGTAATAAAAC (SEQ ID NO: 270) | AACTAC (SEQ ID NO: 284) |
| 2021Q1_ | SEQ ID | TTGACTACACTCTCTATCTCTTAG | CCAGCTCCAACTACCACAA |
| 2.017 | NO: 260 | GGAGACTGAAAC (SEQ ID NO: 271) | GTTTAT (SEQ ID NO: 273) |
| 2021Q1_ | SEQ ID | TTGACTACACTCTCTATCTCTTAG | ACCAGCTCCAACTACCACA |
| 2.002 | NO: 261 | GGAGACTGAAAC (SEQ ID NO: 271) | AGTTTA (SEQ ID NO: 274) |
| 2021Q1_ | SEQ ID | ACTAGACTATACCCCCATTTGAGA | CCAGCTCCAACTACCACAA |
| 2.014 | NO: 262 | GGGGACTAAAAC (SEQ ID NO: 272) | GTTTAT (SEQ ID NO: 273) |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of inducing cell cycle arrest, apoptosis, cell death, or a combination thereof, in a cell, the method comprising: contacting a CRISPR-associated protein or an mRNA encoding the CRISPR-associated protein, and a guide nucleic acid molecule to a nucleic acid target site within the cell, wherein the CRISPR-associated protein comprises an amino acid sequence that is at least 95% identical to any one of SEQ ID NOS: 1-220, 244, and 248-262, wherein the guide nucleic acid molecule is complementary to at least a portion of the nucleic acid target site, and wherein hybridization of the guide nucleic acid molecule to the nucleic acid target site activates non-specific cleavage of DNA, RNA, or a combination thereof in the cell and induces cell cycle arrest, apoptosis, cell death, or a combination thereof, of the cell.

2-3. (canceled)

4. The method of claim 1, wherein the CRISPR-associated protein induces cell cycle arrest, apoptosis, or cell death of at least 50% of the cells in the cell population as determined by an in vitro viability assay, proliferation assay, apoptosis assay, or cell cycle or DNA damage assay.

5-39. (canceled)

40. The method of claim 1 further comprising contacting an additional therapeutic agent, wherein the additional therapeutic agent is an anti-PD1 agent or a PARP inhibitor.

41-65. (canceled)

66. A composition comprising a CRISPR-associated protein, or a nucleic acid encoding the CRISPR-associated protein, and a guide nucleic acid molecule, wherein
a) the CRISPR-associated protein, and
b) the guide nucleic acid molecule comprises a nucleotide sequence that is identical or reverse complementary to a target sequence of a target nucleic acid,
wherein the target sequence comprises a mutation of at least one nucleotide relative to a corresponding wildtype sequence, and
wherein the CRISPR-associated protein comprises an amino acid sequence that is at least 95% identical to any one of SEQ ID NOs: 1-220, 244, and 248-262.

67-73. (canceled)

74. The composition of claim 66, wherein the target nucleic acid is a gene selected from RB1, KRAS, p53, CDKN2A, EGFR, BRCA1, BRCA2, and HER2.

75. (canceled)

76. The composition of claim 74, wherein the target nucleic acid is KRAS, and wherein the mutation is selected from KRAS p.G12C—c.34G>T; KRAS p.G12D—c.35G>A; and KRAS p.G12V—c.35G>T.

77. (canceled)

78. The composition of claim 76, wherein the mutation is KRAS p.G12D.

79. The composition of claim 76, wherein the mutation is KRAS p.G12D—c.35G>A, and the guide nucleic acid molecule comprises a nucleotide sequence selected from SEQ ID NOS: 226, 227, 228, 236, 238, 240, 242, 264, 266, 267, and 269.

80. The composition of claim 76, wherein the mutation is KRAS p.G12V—c.35G>T, and the guide nucleic acid molecule comprises a nucleotide sequence selected from TGGTAGTTGGAGCTGTT (SEQ ID NO: 229); GAGCTGTTGGCGTAGGC (SEQ ID NO: 230); and CCTACGCCAACAGCTCC (SEQ ID NO: 231).

81. The composition of claim 76, wherein the mutation is KRAS p.G12C—c.34G>T, and the guide nucleic acid molecule comprises a nucleotide sequence selected from TGGTAGTTGGAGCTTGT (SEQ ID NO: 232); GAGCTTGTGGCGTAGGC (SEQ ID NO: 233); and CCTACGCCACAAGCTCC (SEQ ID NO: 234).

82. The composition of claim 76, wherein:
a) the CRISPR-associated protein comprises an amino acid sequence that is at last 95% identical to SEQ ID NO: 166, and wherein the guide nucleic acid comprises a nucleotide sequence that is at least 90% identical to a sequence selected from SEQ ID NOS: 236, 240, 264, 266, and 267;
b) the CRISPR-associated protein comprises an amino acid sequence that is at least 95% identical to SEQ ID NO: 248; and the guide nucleic acid molecule comprises a repeat sequence that is at least 90% identical to SEQ ID NO: 270 and a spacer sequence that is at least 90% identical to SEQ ID NO: 273;
c) the CRISPR-associated protein comprises an amino acid sequence that is at least 95% identical to SEQ ID NO: 249; and the guide nucleic acid molecule comprises a repeat sequence that is at least 90% identical to SEQ ID NO: 270 and a spacer sequence that is at least 90% identical to SEQ ID NO: 274;
d) the CRISPR-associated protein comprises an amino acid sequence that is at least 95% identical to SEQ ID NO: 250; and the guide nucleic acid molecule comprises a repeat sequence that is at least 90% identical to SEQ ID NO: 270 and a spacer sequence that is at least 90% identical to SEQ ID NO: 275;
e) the CRISPR-associated protein comprises an amino acid sequence that is at least 95% identical to SEQ ID NO: 251; and the guide nucleic acid molecule comprises a repeat sequence that is at least 90% identical to SEQ ID NO: 270 and a spacer sequence that is at least 90% identical to SEQ ID NO: 276;
f) the CRISPR-associated protein comprises an amino acid sequence that is at least 95% identical to SEQ ID NO: 252; and the guide nucleic acid molecule comprises a repeat sequence that is at least 90% identical to SEQ ID NO: 270 and a spacer sequence that is at least 90% identical to SEQ ID NO: 277;
g) the CRISPR-associated protein comprises an amino acid sequence that is at least 95% identical to SEQ ID NO: 253; and the guide nucleic acid molecule comprises a repeat sequence that is at least 90% identical to SEQ ID NO: 270 and a spacer sequence that is at least 90% identical to SEQ ID NO: 278;
h) the CRISPR-associated protein comprises an amino acid sequence that is at least 95% identical to SEQ ID NO: 254; and the guide nucleic acid molecule comprises a repeat sequence that is at least 90% identical to SEQ ID NO: 270 and a spacer sequence that is at least 90% identical to SEQ ID NO: 279;
i) the CRISPR-associated protein comprises an amino acid sequence that is at least 95% identical to SEQ ID NO: 255; and the guide nucleic acid molecule comprises a repeat sequence that is at least 90% identical to SEQ ID NO: 270 and a spacer sequence that is at least 90% identical to SEQ ID NO: 280;
j) the CRISPR-associated protein comprises an amino acid sequence that is at least 95% identical to SEQ ID NO: 256; and the guide nucleic acid molecule comprises a repeat sequence that is at least 90% identical to SEQ ID NO: 270 and a spacer sequence that is at least 90% identical to SEQ ID NO: 281;
k) the CRISPR-associated protein comprises an amino acid sequence that is at least 95% identical to SEQ ID NO: 257; and the guide nucleic acid molecule comprises a repeat sequence that is at least 90% identical to SEQ ID NO: 270 and a spacer sequence that is at least 90% identical to SEQ ID NO: 282;
l) the CRISPR-associated protein comprises an amino acid sequence that is at least 95% identical to SEQ ID NO: 258; and the guide nucleic acid molecule comprises a repeat sequence that is at least 90% identical to SEQ ID NO: 270 and a spacer sequence that is at least 90% identical to SEQ ID NO: 283;
m) the CRISPR-associated protein comprises an amino acid sequence that is at least 95% identical to SEQ ID NO: 259; and the guide nucleic acid molecule comprises a repeat sequence that is at least 90% identical to SEQ ID NO: 270 and a spacer sequence that is at least 90% identical to SEQ ID NO: 284;
n) the CRISPR-associated protein comprises an amino acid sequence that is at least 95% identical to SEQ ID NO: 260; and the guide nucleic acid molecule comprises a repeat sequence that is at least 90% identical to SEQ ID NO: 271 and a spacer sequence that is at least 90% identical to SEQ ID NO: 273;
o) the CRISPR-associated protein comprises an amino acid sequence that is at least 95% identical to SEQ ID NO: 261; and the guide nucleic acid molecule comprises a repeat sequence that is at least 90% identical to SEQ ID NO: 271 and a spacer sequence that is at least 90% identical to SEQ ID NO: 274; or
p) the CRISPR-associated protein comprises an amino acid sequence that is at least 95% identical to SEQ ID NO: 262; and the guide nucleic acid molecule comprises a repeat sequence that is at least 90% identical to SEQ ID NO: 272 and a spacer sequence that is at least 90% identical to SEQ ID NO: 273.

83-87. (canceled)

88. A method of selectively modifying a portion of cells within a population of cells, the method comprising contacting the population of cells with the composition of claim 66, wherein the portion of cells comprises the target nucleic acid that comprises the mutation, and the remaining cells comprise the corresponding wildtype sequence.

89-120. (canceled)

121. A method of inducing death of a human cell comprising at least one allele with a genetic mutation, the method comprising: contacting the human cell with a Cas13 protein and a guide nucleic acid molecule that hybridizes to a target sequence of a target mRNA, wherein the target sequence is identical, complementary, or reverse complementary to a portion of the allele comprising the mutation, wherein the at least one allele is an allele of KRAS, and wherein the genetic mutation is selected from: p.G12D—c.35G>A; p.G12V—c.35G>T; and p.G12C—c.34G>T.

122-124. (canceled)

125. The method of claim 121, wherein:
a) the Cas13 protein is at least 95% identical to SEQ ID NO: 248; and the guide nucleic acid molecule comprises a repeat sequence that is at least 90% identical to SEQ ID NO: 270 and a spacer sequence that is at least 90% identical to SEQ ID NO: 273;
b) the Cas13 protein is at least 95% identical to SEQ ID NO: 249; and the guide nucleic acid molecule comprises a repeat sequence that is at least 90% identical to SEQ ID NO: 270 and a spacer sequence that is at least 90% identical to SEQ ID NO: 274;
c) the Cas13 protein is at least 95% identical to SEQ ID NO: 250; and the guide nucleic acid molecule comprises a repeat sequence that is at least 90% identical to SEQ ID NO: 270 and a spacer sequence that is at least 90% identical to SEQ ID NO: 275;
d) the Cas13 protein is at least 95% identical to SEQ ID NO: 251; and the guide nucleic acid molecule comprises a repeat sequence that is at least 90% identical to SEQ ID NO: 270 and a spacer sequence that is at least 90% identical to SEQ ID NO: 276;
e) the Cas13 protein is at least 95% identical to SEQ ID NO: 252; and the guide nucleic acid molecule comprises a repeat sequence that is at least 90% identical to SEQ ID NO: 270 and a spacer sequence that is at least 90% identical to SEQ ID NO: 277;
f) the Cas13 protein is at least 95% identical to SEQ ID NO: 253; and the guide nucleic acid molecule comprises a repeat sequence that is at least 90% identical to SEQ ID NO: 270 and a spacer sequence that is at least 90% identical to SEQ ID NO: 278;
g) the Cas13 protein is at least 95% identical to SEQ ID NO: 254; and the guide nucleic acid molecule comprises a repeat sequence that is at least 90% identical to SEQ ID NO: 270 and a spacer sequence that is at least 90% identical to SEQ ID NO: 279;
h) the Cas13 protein is at least 95% identical to SEQ ID NO: 255; and the guide nucleic acid molecule comprises a repeat sequence that is at least 90% identical to SEQ ID NO: 270 and a spacer sequence that is at least 90% identical to SEQ ID NO: 280;
i) the Cas13 protein is at least 95% identical to SEQ ID NO: 256; and the guide nucleic acid molecule comprises a repeat sequence that is at least 90% identical to SEQ ID NO: 270 and a spacer sequence that is at least 90% identical to SEQ ID NO: 281;
j) the Cas13 protein is at least 95% identical to SEQ ID NO: 257; and the guide nucleic acid molecule comprises a repeat sequence that is at least 90% identical to SEQ ID NO: 270 and a spacer sequence that is at least 90% identical to SEQ ID NO: 282;
k) the Cas13 protein is at least 95% identical to SEQ ID NO: 258; and the guide nucleic acid molecule comprises a repeat sequence that is at least 90% identical to SEQ ID NO: 270 and a spacer sequence that is at least 90% identical to SEQ ID NO: 283;
l) the Cas13 protein is at least 95% identical to SEQ ID NO: 259; and the guide nucleic acid molecule comprises a repeat sequence that is at least 90% identical to SEQ ID NO: 270 and a spacer sequence that is at least 90% identical to SEQ ID NO: 284;
m) the Cas13 protein is at least 95% identical to SEQ ID NO: 260; and the guide nucleic acid molecule comprises a repeat sequence that is at least 90% identical to SEQ ID NO: 271 and a spacer sequence that is at least 90% identical to SEQ ID NO: 273;
n) the Cas13 protein is at least 95% identical to SEQ ID NO: 261; and the guide nucleic acid molecule comprises a repeat sequence that is at least 90% identical to SEQ ID NO: 271 and a spacer sequence that is at least 90% identical to SEQ ID NO: 274; or
o) the Cas13 protein is at least 95% identical to SEQ ID NO: 262; and the guide nucleic acid molecule comprises a repeat sequence that is at least 90% identical to SEQ ID NO: 272 and a spacer sequence that is at least 90% identical to SEQ ID NO: 273.

126. The composition of claim 66, wherein the CRISPR-associated protein is a fusion protein, wherein the fusion protein comprises an enzymatically inactive CRISPR protein and a polypeptide that exhibits nuclease activity.

127. The composition of claim 126, wherein the polypeptide that exhibits nuclease activity comprises a restriction enzyme.

128. The method of claim 88 further comprising contacting a second guide nucleic acid molecule complementary to a second target sequence.

129. The method of claim 128 further comprising contacting a third guide nucleic acid molecule complementary to a third target sequence.

130. The method of claim 88, wherein the cell is a cancer cell or wherein the cell population is a cancer cell population.

131. The method of claim 130, wherein the cancer cell population is associated with retinoblastoma, glioblastoma, lung cancer, or liver cancer.
